(19) United States
(12) Patent Application Publication
(10) Pub. No.: US 2005/0177901 A1
(43) Pub. Date: Aug. 11, 2005

(54) Identification and characterization of plant genes

(75) Inventors: Tong Zhu, Research Triangle Park, NC (US); Wengiong Chen, San Diego, CA (US); Steven P. Briggs, Del Mar, CA (US); Bret Cooper, La Jolla, CA (US); Stephen A. Goff, Research Triangle Park, NC (US); Todd Moughamer, Research Triangle Park, NC (US); Jane GlazeBrook, San Diego, CA (US); Fumiaki Katagiri, St. Paul, CA (US); Joel Kreps, San Diego, CA (US); Nicolas J. Provart, Toronto (CA); Darrell Ricke, Research Triangle Park, NC (US)

Correspondence Address: JENKINS, WILSON & TAYLOR, P. A., 3100 TOWER BLVD, SUITE 1400, DURHAM, NC, 27707, US

(73) Assignee: SYNGENTA PARTICIPATIONS AG

(21) Appl. No.: 10/481,032

(22) PCT Filed: Jun. 21, 2002
(86) PCT No.: PCT/IB02/02450

(51) Int. Cl. C12Q 1/68; C07H 21/04; C12N 9/20; C12N 9/24; A01H 1/00; C12N 15/82

(52) U.S. Cl. 800/281; 435/6; 435/198; 435/200; 536/23.2; 435/419; 435/468

(57) ABSTRACT

The invention discloses a set of genes the expression products of which are up-regulated during the grain filling process in rice and active in different metabolic pathways involved in nutrient partitioning. The invention also discloses the use of said genes to modify the compositional and nutritional characteristics of the plant grain.

The present invention is in the area of plant biotechnology. In particular, the invention relates to a set of genes the expression products of which are up-regulated during the grain filling process in rice and active in different metabolic pathways involved in nutrient partitioning. The invention also relates to the use of said genes to modify the compositional and nutritional characteristics of the plant grain.

It has been long recognized that the value of agricultural products such as cereal grains and the like are affected by the quality of their inherent constituent components: In particular, cereal grains with improved protein, oil, starch, fiber, and moisture content and desirable levels of carbohydrates and other constituents are of economic interest.

In rice, for example, yield, nutritional characteristics and eating quality are the most important economic traits. The first two traits are mostly determined by the composition and accumulation of carbohydrates, proteins, and minerals during grain filling, and the latter by the interaction of various Xs enzymes to produce the final structure of the starch at the molecular and granule levels. Manipulation of these pathways results in significant improvement in the nutritional value. For example, reduction of the amounts of even one enzyme, granule-bound starch synthase, in the starch biosynthetic pathway can dramatically affect the eating quality, resulting in softer, less sticky cooked rice. Some genes participating in nutrient partitioning during rice grain filling and affecting starch quality have been previously identified. However, genes participated in these processes and their transcriptional controls are poorly understood.

Within the scope of the present invention a set of genes is now provided which were shown to be involved in the grain filling process based on their mRNA expression characteristics. The genes within this subset are preferentially up-regulated and share a similar expression pattern during the process of grain filling. The expression levels of those genes increase synchronously during grain development while the encoded gene products are active in different pathways. The genes within this subgroup, representative examples of which are provided in the Sequence Listing, are thus useful tools for generating plants which produce grain with modified compositional characteristics leading to improved nutritional properties.

One of the main objectives of the present invention is thus to provide a polynucleotide comprising a nucleotide sequence encoding a polypeptide the expression of which is up-regulated during grain filling and the use of said molecule for modifying the nutritional composition and quality of plant grain.

The majority of the genes within this group encode protein products that are directly involved in or associated with three major pathways of nutrition partitioning: the synthesis and transport of (1) carbohydrates, (2) proteins, and (3) fatty acids.

The most dramatic increase in relative mRNA expression levels is shown by those genes whose products control the synthesis of carbohydrates and proteins and can be found in the endosperm of the developing seed, which is the main sink for plant nutrients.

The other group of genes which shows a significant increase in relative mRNA expression levels comprises genes that are involved in and in control of fatty acid biosynthesis. These genes have a more balanced expression between the embryo and endosperm.

In one embodiment the invention thus relates to a subset of isolated nucleic acid molecules comprising a nucleotide sequence encoding a polypeptide that is involved in at least one of the major pathways of nutrition partitioning selected from the group consisting of synthesis, transport, metabolism or degradation of carbohydrates, proteins, and fatty acids.

Another subset of nucleic acid molecules provided herein comprises a number of nucleic acids that encode different transporters, such as sugar transporters, ABC transporters, amino acid/peptide transporters, phosphate transporters, and nitrate transporters.

Still another subset of nucleic acid molecules that is provided as part of the invention comprises nucleic acid molecules that are involved in the transcriptional control of the highly coordinated grain filling process.

Further subsets of nucleic acid molecules provided herein comprise nucleic acid molecules the expression products of which are associated with amino acid metabolism; signal transduction; and stress regulation, respectively.

In a collective embodiment applicable to all of the nucleic acid molecules disclosed herein, the invention relates to the use of the nucleic acid molecules according to the invention as hybridization probes, for chromosome and gene mapping, in PCR technologies, in the production of sense or antisense nucleic acids, in screening for new therapeutic molecules, in production of plants and seeds having desirable, inheritable, commercially useful phenotypes, or in discovery of inhibitory compounds.

The invention further relates to any polypeptides encoded by the nucleic acid molecules according to the invention, or any antigene sequences thereof, which have numerous applications using techniques that are known to those skilled in the art of molecular biology, biotechnology, biochemistry, genetics, physiology or pathology.

In a further collective embodiment, the present invention provides the ability to modulate the grain filling process, by over-expressing, under-expressing or knocking out one or more of the genes disclosed herein or their gene products, in a plant cell, in vitro or in planta. Expression vectors comprising at least one nucleic acid molecule according to the invention, or any antigenes thereof, operably linked to at least one suitable promoter and/or regulatory sequence can be used to study the role of polypeptides encoded by said sequences, for example by transforming a host cell with said expression vector and measuring the effects of overexpression and underexpression of said nucleic acid molecules. Suitable promoter and/or regulatory sequences include especially those that are preferentially or specifically active in plant grain tissue such as, for example, the grain endosperm or the grain embryo. A host cell transformed with at least one expression vector comprising at least one nucleic acid molecule of the invention, operably linked to suitable promoters and/or regulatory sequences, can be useful to produce a plant grain with improved nutritional or dietary properties.

In a further collective embodiment, the present invention provides a transformed plant host cell, or one obtained through breeding, capable of over-expressing, under-expressing, or having a knock out of at least one of the genes according to the invention and/or their gene products.

Such a plant cell, transformed with at least one expression vector comprising a nucleic acid molecule of the invention, operably linked to suitable promoters and/or regulatory sequences, can be used to regenerate plant tissue or an entire plant, or seed there from, in which the effects of expression, including overexpression or underexpression, of the introduced sequence or sequences can be measured in vitro or in planta.

In a further embodiment the present invention provides nucleotide sequences including regions of nucleotide sequence encoding polypeptides having homology to at least one functional protein domain (FPD). Embodiments of the invention further provide polypeptides including regions of amino acid sequence having homology to an FPD. In cases where the polypeptide has homology to an FPD in the same or closely related species, the polypeptide may represent a paralogous sequence or paralog, or may represent a variant allele of a gene encoding the FPD. In cases where the polypeptide has homology to an FPD in another species, including other plant species and especially non-plant species, polypeptides may represent orthologous sequences, or orthologs, of the FPD.

In a further collective embodiment of the invention the nucleic acid molecules disclosed herein or respresentative parts thereof can be used in hybridization-based assays for detecting and identifying nucleic acid molecules that encode protein products that are involved in the grain filling process, more particularly in at least one of the major pathways of nutrition partitioning selected from the group consisting of synthesis, transport, metabolism or degradation of carbohydrates, proteins, and fatty acids, in plants other than rice, but especially in plants belonging to the cereal group.

Embodiments of the present invention provide a unique oligonucleotide having a sequence identical to or complementary to a region of a polynucleotide sequence encoding at least a portion of a homologue of a protein according to the invention representatives of which are identified by SEQ ID NOs 2-462, 502-512, and 514-642 provided in the Sequence Listing and/or an FPD thereof, the oligonucleotide being identified by the methods disclosed herein. In one embodiment, the unique oligonucleotide has a length of between 12 and 250 nucleotide bases.

Embodiments of the present invention also provide a nucleotide microarray comprising the unique oligonucleotide having a sequence identical to or complementary to a region of polynucleotide sequence encoding at least a portion of a homologue of a protein according to the invention representatives of which are identified by SEQ ID NOs: 2-462, 502-512, and 514-642 provided in the Sequence Listing and/or an FPD thereof. Preferably, the microarray includes a plurality of different, unique oligonucleotides, the sequences corresponding to a plurality of homologues of a protein according to the invention representatives of which are identified by the SEQ ID NOs provided in the Sequence Listing and/or an FPD thereof. Equally preferably, the microarray contains at least about 96 different unique oligonucleotides, wherein each of the 96 different unique oligonucleotides has a sequence that is identical, complementary, or substantial similarity to a segment of a nucleotide sequence as given in SEQ ID NOs: 1-461, 501-511, and 513-641 provided in the Sequence Listing.

Embodiments of the present invention also provide a kit for detecting the presence of a polynucleotide, the kit containing a first nucleotide probe which can hybridize with a region of a nucleotide sequence including the nucleotide sequences of SEQ ID NOs: 1-461 provided in the Sequence Listing, a fragment or a variant thereof, and a complementary sequence thereto, the kit further containing at least one additional component such as, for example: a second nucleotide probe, a buffer, an enzyme, a label, a molecular weight standard, a reaction chamber, and a micropipette tip.

Embodiments of the present invention further provide a kit for detecting the presence of a polypeptide, the kit containing a first probe which can hybridize with a region of a polypeptide including the amino acid sequences of SEQ ID NOs: 2-462, 502-512, and 514-642 provided in the Sequence Listing, a fragment or a variant thereof, and optionally, the kit further containing at least one additional component such as, for example: a probe, a buffer, an enzyme, a label, a molecular weight standard, a reaction chamber, and a micropipette tip. Probes useful in kit embodiments include antibodies, affinity tags, protein A, protein G, or protein-binding substances including chromatographic media.

An additional aspect provides a method for selecting plants, for example cereals, having an altered carbohydrate, protein or fatty acid content and/or composition of the grain comprising obtaining nucleic acid molecules from the plants to be selected, contacting the nucleic acid molecules with one or more probes that selectively hybridize under stringent or highly stringent conditions to a nucleic acid sequence selected from the group consisting of SEQ ID NOs. 1-461, 501-511, and 513-641; detecting the hybridization of the one or more probes to the nucleic acid sequences wherein the presence of the hybridization indicates the presence of a gene associated with altered carbohydrate, protein or fatty acid content and/or composition of the grain; and selecting plants on the basis of the presence or absence of such hybridization. In one embodiment, marker-assisted selection is accomplished in rice. In another embodiment, marker assisted selection is accomplished in wheat using one or more probes which selectively hybridize under stringent or highly stringent conditions to sequences selected from the group consisting of SEQ ID NOs. 951-1105. In yet another embodiment, marker assisted selection is accomplished in maize or corn using one or more probes which selectively hybridize under stringent or highly stringent conditions to sequences selected from the group consisting of SEQ ID NOs. 1106-1201. In still another embodiment, marker assisted selection is accomplished in banana using one or more probes which selectively hybridize under stringent or highly stringent conditions to sequences selected from the group consisting of SEQ ID NOs. 884-950. In each case marker-assisted selection can be accomplished using a probe or probes to a single sequence or multiple sequences. If multiple sequences are used they can be used simultaneously or sequentially.

In a further embodiment of the invention a computer readable medium containing one or more of the nucleotide sequences of the invention is provided as well as methods of use for the computer readable medium. This medium allows a nucleotide sequence corresponding to at least one of the sequences selected from the group consisting of SEQ ID NOs: 1-461, 501-511, and 513-641 and 884-1201 provided in the Sequence Listing (open reading frames or fragments thereof), to be used as a reference sequence to search against a database. This medium also allows for computer-based manipulation of a nucleotide sequence corresponding to at least one of the sequences selected from the group consisting of SEQ ID NOs: 1-461, 501-511, and 513-641, 884-1201 provided in the Sequence Listing.

Further aspects, features and advantages of this invention will become apparent from the detailed description of the preferred embodiments that follow.

A further aspect provides a computer readable medium having stored thereon computer executable instructions for performing a method comprising receiving data on nucleotide sequence expression in a test plant of at least one nucleic acid molecule having at least 70%, at least 80%, at least 90% or at least 95%, sequence identity to a nucleotide sequence selected from the group consisting of SEQ ID NOs: 1-461, 501-511, and 513-641; and 884-1201 and comparing expression data from said test plant to expression data for the same nucleotide sequence or sequences in a plant during grain filling.

### BRIEF DESCRIPTION OF THE SEQUENCE LISTING

In the following, a brief description of the sequences in the Sequence Listing is provided:

Odd numbered SEQ ID NOs:1-461 are representing a first sub-group (sub-group I) of polynucleotides comprising nucleotide sequences which encode polypeptides that are up-regulated during grain filling and are described in Tables 1-11 below.

Even numbered SEQ ID NOs:2-462 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

Odd numbered SEQ ID NOs: 501-511 are representing a second sub-group (sub-group II) of polynucleotides comprising rice cDNA sequences. The correlation between the sequences in sub-groups I and II is illustrated in Table 13.

Even numbered SEQ ID NOs:502-512 are protein sequences encoded by the immediately preceding nucleotide sequence.

Odd numbered SEQ ID NOs: 513-641 are representing a third sub-group (sub-group III) of polynucleotides comprising nucleotide sequences that have homologies between 80% and 99.90% to the nucleotide sequences of sub-group I and possible variants or familiy members of rice sequences provided in SEQ ID NOs: 1-461. The correlation between the sequences in sub-groups I and III is illustrated in Table 12.

Even numbered SEQ ID NOs:514-642 are protein sequences encoded by the immediately preceding nucleotide sequence.

SEQ ID NOs: 643-883 are promoter sequences.

SEQ ID NOs: 884-950 are banana sequences which show homology to rice “grain filling” genes.

SEQ ID NOs: 951-1105 are wheat sequences which show homology to rice “grain filling” genes.

SEQ ID NOs: 1106-1201 are maize sequences which show homology to rice “grain filling” genes.

Definitions

For clarity, certain terms used in the specification are defined and presented as follows:

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “native” or “wild type” gene refers to a gene that is present in the genome of an untransformed cell, i.e., a cell not having a known mutation.

A “marker gene” encodes a selectable or screenable trait.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a normative organism, or chimeric genes. The term “endogenous gene” refers to a native gene in its natural location in the genome of an organism. A “foreign” gene refers to a gene not normally found in the host organism but that is introduced by gene transfer.

An “oligonucleotide” corresponding to a nucleotide sequence of the invention, e.g., for use in probing or amplification reactions, may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16 to 24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

The terms “protein,” “peptide” and “polypeptide” are used interchangeably herein.

The nucleotide sequences of the invention can be introduced into any plant. The genes to be introduced can be conveniently used in expression cassettes for introduction and expression in any plant of interest. Such expression cassettes will comprise the transcriptional initiation region of the invention linked to a nucleotide sequence of interest. Preferred promoters include constitutive, tissue-specific, development-specific, inducible and/or viral promoters. Such an expression cassette is provided with a plurality of restriction sites for insertion of the gene of interest to be under the transcriptional regulation of the regulatory regions. The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al., 1991; Proudfoot, 1991; Sanfacon et al., 1991; Mogen et al., 1990; Munroe et al., 1990; Ballas et al., 1989; Joshi et al., 1987.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA.

“Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ noncoding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters.

“5′ noncoding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., 1989.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position+1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Specific expression” is the expression of gene products which is limited to one or a few plant tissues (spatial limitation) and/or to one or a few plant developmental stages (temporal limitation). It is acknowledged that hardly a true specificity exists: promoters seem to be preferably switch on in some tissues, while in other tissues there can be no or only little activity. This phenomenon is known as leaky expression. However, with specific expression in this invention is meant preferable expression in one or a few plant tissues.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates.

The rate of degradation can however be considered to proceed at a fixed rate when the transcribed sequences are identical, and thus this value can serve as a measure of synthesis rates. When promoters are compared in this way techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA.

The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This includes also part of the transcribed sequences. The direct fusion of promoter to reporter sequences would therefore lead to suboptimal levels of transcription.

A commonly used procedure to analyze expression patterns and levels is through determination of the ‘steady state’ level of protein accumulation in a cell. Commonly used candidates for the reporter gene, known to those skilled in the art are β-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

“Overexpression” refers to the level of expression in transgenic cells or organisms that exceeds levels of expression in normal or untransformed (nontransgenic) cells or organisms.

“Antisense inhibition” refers to the production of antisense RNA transcripts capable of suppressing the expression of protein from an endogenous gene or a transgene.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., 1996). Gene silencing includes virus-induced gene silencing (Ruiz et al. 1998).

The terms “heterologous DNA sequence,” “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include no-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequence of two nucleic acid molecules or between the amino acid sequences of two protein molecules. As used herein, “homology” and “homologous” refer to an evaluation of the similarity between two sequences based on measurements of sequence identity adjusted for variables including gaps, insertions, frame shifts, conservative substitutions, and sequencing errors, as described below. Two nucleotide sequences or polypeptides are the to be “identical” if the sequence of nucleotides or amino acid residues, respectively, in the two sequences is the same when aligned for maximum correspondence as described below. The term “complementary to” is used herein to mean that the sequence can form a Watson-Crick base pair with a reference polynucleotide sequence. Complementary sequences can include nucleotides, such as inosine, that neither disrupt Watson-Crick base pairing nor contribute to the pairing. A “reverse complement” of a sequence corresponds to the complementary sequence, but in the opposite orientation of bases from 5′ to 3′, or to the complement of the primary sequence, if the primary sequence is in a reverse orientation of bases from 5′ to 3′.

Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, Proc Natl Acad Sci (USA) 85:2444 (1988); Altschul et al., J. Mol Biol 215:403 (1990)). In a particularly preferred embodiment, protein and nucleic acid sequence homologies are evaluated using the Basic Local Aligment Search Tool (“BLAST”) which is well known in the art (Karlin and Altschul, Proc Natl Acad Sci USA 87:2264 (1990); Altschul et al. (1990) supra, Altschul et al., Nucleic Acids Res 25:3389 (1997)). In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.
   The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (aligned) by means of a scoring matrix selected from the many scoring matrices known in the art. Preferably, the scoring matrix used is the BLOSUM62 matrix (Gonnet et al., Science 256:1443 (1992); Henikoff and Henikoff, Proteins 17:49 (1993)). Likewise, the PAM or PAM250 matrices may also be used (Schwartz and Dayhoff, In Atlas of protein Sequence and Structure, Dayhoff, ed., Natl Biomed. Res. Found., pp. 353-358 (1978)). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (Karlin and Altschul (1990) supra).

“Percentage of sequence identity” can be determined from alignments performed using algorithms known in the art. Alignment of nucleotide or polypeptide sequences for comparison may be conducted by the local homology algorithm of Smith and Waterman (Add APL Math 2:482 (1981)), by the homology alignment algorithm of Needleman and Wunsch (J. Mol Biol 48:443 (1970)), by the search for similarity method of Pearson and Lipman (Proc Natl Acad Sci USA 85:2444 (1988)), by computerized implementations of these algorithms (GAP, BESTFIT, BLAST, PASTA, and TFASTA in the Wisconsin Genetics Software Package, Genetics Computer Group), or by inspection. When two sequences have been identified for comparison, GAP and BESTFIT are preferably employed to determine their optimal alignment. Typically, the default values of 5.00 for gap weight and 0.30 for gap weight length are used. In a preferred embodiment, percenty identity is determined using the GAP program for global alignment using default parameters, using the version of GAP found in the GCG package (Wisconsin Package Version 10.1, Genetics Computer Group, 575 Science Dr., Madison, Wis.).

“Percentage of sequence identity” is determined by comparing two optimally aligned sequences over a comparison window, wherein the portion of the sequence in the comparison window may include additions or deletions, including for example gaps or overhangs, as compared to the reference sequence (which does not include additions or deletions) for optimal alignment of the two sequences. The percentage is calculated by determining the number of positions at which the identical nucleotide base or amino acid residue occurs in both sequences to yield the number of matched positions, dividing the number of matched positions by the total number of positions in the window of comparison and multiplying the result by 100 to yield the percentage of sequence identity.

In a broad sense, the term “substantially similar”, when used herein with respect to a nucleotide sequence, means a nucleotide sequence corresponding to a reference nucleotide sequence, wherein the corresponding sequence encodes a polypeptide having substantially the same structure as the polypeptide encoded by the reference nucleotide sequence. Desirably, the substantially similar nucleotide sequence encodes the polypeptide encoded by the reference nucleotide sequence. Preferably, “substantially similar” refers to nucleotide sequences having at least 50% sequence identity, preferably at least 60%, 70%, 80% or 85%, more preferably at least 90% or 95%, and even more preferably, at least 96%, 97% or 99% sequence identity compared to a reference sequence containing nucleotide sequences of Table 1, that encode a protein having at least 50% identity, more preferably at least 85% identity, yet still more preferably at least 90% identity to a region of sequence of a BIOPATH protein and/or an FPD, wherein the protein sequence comparisons are conducted using GAP analysis as described below. Also, “substantially similar” preferably also refers to nucleotide sequences having at least 50% identity, more preferably at least 80% identity, still more preferably 95% identity, yet still more preferably at least 99% identity, to a region of nucleotide sequence encoding a BIOPATH protein and/or an FPD, wherein the nucleotide sequence comparisons are conducted using GAP analysis as described below. The term “substantially similar” is specifically intended to include nucleotide sequences wherein the sequence has been modified to optimize expression in particular cells.

A polynucleotide including a nucleotide sequence “substantially similar” to the reference nucleotide sequence preferably hybridizes to a polynucleotide including the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

The term “substantially similar”, when used herein with respect to a protein or polypeptide, means a protein or polypeptide corresponding to a reference protein, wherein the protein has substantially the same structure and function as the reference protein, where only changes in amino acids sequence that do not materially affect the polypeptide function occur. When used for a protein or an amino acid sequence the percentage of identity between the substantially similar and the reference protein or amino acid sequence desirably is preferably at least 30%, more preferably at least 40%, 50%, 60%, 70%, 80%, 85%, or 90%, still more preferably at least 95%, still more preferably at least 99% with every individual number falling within this range of at least 30% to at least 99% also being part of the invention, using default GAP analysis parameters with the University of Wisconsin GCG (version 10), SEQWEB application of GAP, based on the algorithm of Needleman and Wunsch (1970), supra. As used herein the term “polypeptide of the present invention,” or any similar term refers to an amino acid sequence encoded by a DNA molecule including a nucleotide sequence substantially similar to an AC sequence. Homologs of BIOPATH protein and/or FPDs include amino acid sequences that are at least 30% identical to BIOPATH protein and/or FPD sequences found in searchable databases, as measured using the parameters described above.

“Target gene” refers to a gene on the replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

The term “altered plant trait” means any phenotypic or genotypic change in a transgenic plant relative to the wild-type or nor-transgenic plant host.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The term “transformation” refers to the transfer of a nucleic acid fragment into the genome of a host cell, resulting in genetically stable inheritance. Host cells containing the transformed nucleic acid fragments are referred to as “trausgenic” cells, and organisms comprising transgenic cells are referred to as “transgenic organisms”. Examples of methods of transformation of plants and plant cells include Agrobacterium-mediated transformation (De Blaere et al., 1987) and particle bombardment technology (Klein et al. 1987; U.S. Pat. No. 4,945,050). Whole plants may be regenerated from transgenic cells by methods well known to the skilled artisan (see, for example, Fromm et al., 1990).

“Transformed,” “transgenic,” and “recombinant” refer to a host organism such as a bacterium or a plant into which a heterologous nucleic acid molecule has been introduced. The nucleic acid molecule can be stably integrated into the genome generally known in the art and are disclosed in Sambrook et al., 1989. See also Innis et al., 1995 and Gelfand, 1995; and Innis and Gelfand, 1999. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Transient expression” refers to expression in cells in which a virus or a transgene is introduced by viral infection or by such methods as Agrobacterium-mediated transformation, electroporation, or biolistic bombardment, but not selected for its stable maintenance.

“Genetically stable” and “heritable” refer to chromosomally-integrated genetic elements that are stably maintained in the plant and stably inherited by progeny through successive generations.

“Primary transformant” and “T0 generation” refer to transgenic plants that are of the same genetic generation as the tissue which was initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

“Wild-type” refers to a virus or organism found in nature without any known mutation.

“Genome” refers to the complete genetic material of an organism.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al. 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

Thus, by “variants” is intended substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The nucleic acid molecules of the invention can be “optimized” for enhanced expression in plants of interest. See, for example, EPA 035472; WO 91/16432; Perlak et al., 1991; and Murray et al., 1989. In this manner, the open reading frames in genes or gene fragments can be synthesized utilizing plant-preferred codons. See, for example, Campbell and Gowri, 1990 for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, 1994; Stemmer, 1994; Crameri et al., 1997; Moore et al., 1997; Zhang et al., 1997; Crameri et al., 1998; and U.S. Pat. Nos. 5,605,793 and 5,837,458.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, 1985; Kunkel et al., 1987; U.S. Pat. No. 4,873,192; Walker and Gaastra, 1983 and the references cited therein. Guidance as to appropriate amino acid substitutions that do not affect biological activity of the protein of interest may be found in the model of Dayhoffet al. (1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bifunctional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

A “transgenic plant” is a plant having one or more plant cells that contain an expression vector.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, nonlimiting examples of such mathematical algorithms are the algorithm of Myers and Miller, 1988; the local homology algorithm of Smith et al. 1981; the homology alignment algorithm of Needleman and Wunsch 1970; the search for-similarity-method of Pearson and Lipman 1988; the algorithm of Karlin and Altschul, 1990, modified as in Karlin and Altschul, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. 1988; Higgins et al. 1989; Corpet et al. 1988; Huang et al. 1992; and Pearson et al. 1994. The ALIGN program is based on the algorithm of Myers and Miller, supra. The BLAST programs of Altschul et al., 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, Nc=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point 1. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% A sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

“Recombinant DNA molecule” is a combination of DNA sequences that are joined together using recombinant DNA technology and procedures used to join together DNA sequences as described, for example, in Sambrook et al., 1989.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold or greater.

Within the scope of the present invention a set of nucleic acid molecules is provided which comprises polynucleotides relating to genes which are shown to be preferentially up-regulated and to share a similar expression pattern during the process of grain filling. The polynucleotides within this subgroup are useful tools for generating plants which produce grain with modified compositional characteristics leading to improved nutritional properties.

In one embodiment, the present invention thus relates to an isolated nucleic acid molecule comprising a nucleotide sequence encoding a polypeptide the expression of which is up-regulated during grain filling and the use of said molecule for modifying the nutritional composition and quality of the plant grain.

The majority of the polynucleotides within this group encode protein products that are directly involved in or associated with three major pathways of nutrition partitioning: the synthesis and transport of (1) carbohydrates, (2) proteins, and (3) fatty acids.

Carbohydrates are the most abundant organic molecules in nature and modulation of their synthesis, accumulation, and storage presents a vast template of possibilities for improving the quality and quantity of agricultural plants, food crops, consumer health products such as dietary supplements, and many industrial applications. In plants, carbohydrates occur as mono-, di, or polysaccharides and have the essential functions of providing the plant with chemical energy and structural stability. Although sugar uptake from external sources generally is not a relevant process, the redistribution of sugar (usually glucose) from photosynthesizing tissues to non-green cells is of major importance. Once translocated to terminal sink storage tissues, sugars are converted to starch and stored in the leucoplasts of seeds, fruits, tubers and roots, as well as actively growing photosynthetic tissues. These plant tissues provide the bulk of human dietary intake, and as such, the anabolic pathways of synthesis and assimilation (starch, fatty acids, and nitrogen) are of particular importance to agriculture and commercial industry.

As major contributors to the global carbon cycle, plants and algae bind 100 billion metric tons of carbon into carbohydrates each year. Nucleotide sequences encoding at least one polypeptide involved in sugar and carbohydrate metabolism and their end products, as well as the polypeptides encoded thereby, or an antigene sequences thereof, are commercially useful materials that can be used to study these processes and to modify these processes to elicit desired modifications in the compositional and nutritional characteristics of the plant grain.

In particular, the subset of nucleic acid molecules provided herein, which comprises polynucleotides relating to genes that are up-regulated during grain filling and involved in carbohydrate transport, synthesis, metabolism, or degradation is a valuable tool box from which an appropriate nucleic acid molecule can be chosen for modifying the quantity and quality of the carbohydrate and sugar content of the grain, respectively. This can be achieved by introducing and overexpressing at least one polynucleotide from the various subsets of nucleic acid molecules provided herein in the plant, but preferentially in the approproate tissues of the plant grain such as, for example, the plant endosperm or by reducing the expression level of the corresponding endogenous gene by methods known in the art including antisense and dsRNAi techniques.

It is thus one of the major objectives of the present invention to identify and provide a subset of nucleic acid molecules comprising at least one polynucleotide which encodes a protein that is involved in the metabolism of carbohydrates during grain filling. By modifying the expression level of at least one of the polynucleotides from this subgroup in a plant, but preferably in the approproate tissues of the plant grain such as, for example, the plant endosperm, and even more preferably at an early stage in seed development, it is possible to modify the carbohydrate composition of the plant grain accordingly.

In one embodiment, the invention thus relates to a polynucleotide comprising a nucleotide sequence encoding a polypeptide the activity of which is involved in or associated with the synthesis, metabolism or degradation of carbohydrates in the plant grain and the expression of which is up-regulated during grain filling, which nucleotide sequence is substantially similar to a sequence encoding a polypeptide as given in the SEQ ID NOs of table 7 such as SEQ ID NOs: 70-210.

In particular, the invention relates to polynucleotide comprising a nucleotide sequence encoding a polypeptide the activity of which is involved in or associated with the synthesis, metabolism or degradation of carbohydrates in the plant grain and the expression of which is up-regulated during grain filling, and which is substantially similar, and preferably has at least between 70%, and 99% amino acid sequence identity to at least one polypeptide of SEQ ID NOs given in table 7 such as SEQ ID NOs: 70-210, with any individual number within this range of between 70% and 99% A also being part of the invention.

The invention further relates to polynucleotide comprising a nucleotide sequence encoding a polypeptide the activity of which is involved in or associated with the synthesis, metabolism or degradation of carbohydrates in the plant grain and the expression of which is up-regulated during grain filling, and which is immunologically reactive with antibodies raised against a polypeptide as given in the SEQ ID NOs of table 7 such as SEQ ID NOs: 70-210.

More particularly, the invention relates to polynucleotide comprising a nucleotide sequence
a) as given in any one of SEQ ID NOs of table 7 such as SEQ ID NOs: 69-209 or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NOs of table 7 such as SEQ ID NOs: 69-209 or the complement thereof;
e) complementary to (a), (b) or (c); and
f) which is the reverse complement of (a), (b) or (c).

One of the defining questions in assimilate partitioning is understanding how plants regulate the allocation of photosynthate between competing sink organs. In addition to the number of competing organs, and the sink strength of each, exogenous factors such as abiotic stress or pathogen infection may also influence partitioning (Bush, Current Opinions in Plant Biology 2:187. (1999)).

Within the present invention a subset of genes could be identified that are known to be involved in the plant's response to abiotic and/or biotic stresses and demonstrated to be up-regulated during grain filling. By providing these genes it is now possible to regulate the expression levels of the encoded protein products in the plant grain during the grain filling process by applying methods known in the art including overexpressing or down-regulating the nucleic acid molecule in a plant, or preferably a plant seed, thereby modifying the partitioning in the developing grain.

In one aspect, the present invention relates to polynucleotide comprising a nucleotide sequence encoding a polypeptide the expression of which is up-regulated during grain filling and the activity of which is involved in or associated with the plant's response to abiotic and/or biotic stresses, which nucleotide sequence is substantially similar to a sequencen encoding a polypeptide as given in any one of the SEQ ID NOs of table 4 such as SEQ ID NOs: 2-18.

In particular, the invention relates to a polynucleotide comprising a nucleotide sequence encoding a polypeptide the expression of which is up-regulated during grain filling and the activity of which is involved in or associated with the plant's response to abiotic and/or biotic stresses, and which is substantially similar, and preferably has at least between 70%, and 99% amino acid sequence identity to at least one polypeptide as given in any one of the SEQ ID NOs of table 4 such as SEQ ID NOs: 2-18, with any individual number within this range of between 70% and 99% also being part of the invention.

The invention further relates to a polynucleotide comprising a nucleotide sequence encoding a polypeptide the expression of which is up-regulated during grain filling and the activity of which is involved in or associated with the plant's response to abiotic and/or biotic stresses, and which is immunologically reactive with antibodies raised against a polypeptide as given in any one of the SEQ ID NOs of table 4 such as SEQ ID NOs: 2-18.

More particularly, the invention relates to a polynucleotide comprising a nucleotide sequence
a) as given in in any one of the SEQ ID NOs of table 4 such as SEQ ID NOs: 1-17 or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in any one of the SEQ ID NOs of table 4 such as SEQ ID NOs 1-17 or the complement thereof;
e) complementary to (a), (b) or (c); and
f) which is the reverse complement of (a), (b) or (c).

The regulation of source-sink pathways encompasses complex mechanisms that integrate the expression of enzymes involved in carbohydrate production in source tissue with those involved with utilization in sink tissue. The elucidation of the underlying signal transduction pathways of sink-source regulation is of critical importance to the genetic manipulation of source-sink relations in transgenic plants.

Within the scope of the present invention a subset of genes was identified comprising genes that are up-regulated during grain filling and encode polypeptides with a kinase or phosphatase activity which are known to be involved in signal transduction pathways.

In a specific embodiment, the present invention provides nucleic acid molecules such as those represented in SEQ ID NOs: 19-29 that encode enzymes which exhibit a kinase or phosphatase activity and/or are involved in a signalig pathway and are thus key to the ability of regulating utilization of carbon/sugar sources, and partitioning of assimilates between source and sink tissues.

The invention thus relates to a polynucleotide comprising a nucleotide sequence encoding a polypeptide which exhibits a kinase or phosphatase activity and/or are involved in a signal transduction pathway, the expression of which is up-regulated during grain filling, which nucleotide sequence is substantially similar to a sequence encoding a polypeptide as given in any one of the SEQ ID NOs of table 5 such as SEQ ID Nos: 20-30.

More specifically, the invention relates to a polynucleotide comprising a nucleotide sequence encoding a polypeptide which exhibit a kinase or phosphatase activity and is up-regulated during grain filling and has at least between 70%, and 99% amino acid sequence identity to at least one polypeptide as given in any one of the SEQ ID NOs of table 5 such as SEQ ID NOs: 20-30, with any individual number within this range of between 70% and 99% also being part of the invention.

The invention further relates to a polynucleotide comprising a nucleotide sequence encoding a polypeptide which exhibit a kinase or phosphatase activity and is up-regulated during gain filling and immunologically reactive with antibodies raised against a polypeptide as given in any one of the SEQ ID NOs of table 5 such as SEQ ID NOs: 20-30.

More particularly, the invention relates to a polynucleotide comprising a nucleotide sequence
a) as given in any one of the SEQ ID NOs of table 5 such as SEQ ID NOs: 19-29 or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof,
d) capable of hybridizing to a nucleic acid comprising 50 to 200 or more consecutive nucleotides of a nucleotide sequence as given in any one of the SEQ ID NOs of table 5 such as SEQ ID NOs: 19-29 or the complement thereof,
e) complementary to (a), (b) or (c); and
f) which is the reverse complement of (a), (b) or (c).

Regulating the environment-induced carbon status in crop plants, particularly the partitioning in storage organs, provides industry with the ability to limit or expand growing seasons to better suit commercial markets, to enhance the quality and content of food products derived from storage organs or other tissue specific components of crop plants, and modulate many other metabolic pathways in plants (such as nitrogen assimilation, phosphorylation and the activation of regulatory proteins) that effect consumer end use.

Another possibility for modifying the carbohydrate content of the grain is through regulation of the transport of sugars and carbohydrates during grain filling.

Supplying carbohydrates to sink tissues via apoplastic mechanisms involves the release of sucrose into the apoplast by an exporter, cleavage by an extracellular invertase, and uptake of hexose monomers by monosaccharide transporters.

In one specific embodiment the present invention thus relates to a polynucleotide comprising a nucleotide sequence encoding a polypeptide with an activity which is involved in or associated with sugar transport and up-regulated during grain filling, which nucleotide sequence is substantially similar to a sequence encoding a polypeptide as given in any one of the SEQ ID NOs of table 6 such as SEQ ID NOs: 36; 50, and 58.

In particular, the invention relates to a polynucleotide comprising a nucleotide sequence encoding a polypeptide with an activity which is involved in or associated with sugar transport and up-regulated during grain filling and is substantially similar, and preferably has at least between 70%, and 99% amino acid sequence identity to at least one polypeptide as given in any one of the SEQ ID NOs of table 6 such as SEQ ID NOs: 36; 50, and 58, with any individual number within this range of between 70% and 99% also being part of the invention.

The invention further relates to a polynucleotide comprising a nucleotide sequence encoding a polypeptide with an activity which is involved in or associated with sugar transport and up-regulated during grain filling and is immunologically reactive with antibodies raised against a polypeptide as given in any one of the SEQ ID NOs of table 6 such as SEQ ID NOs: 36; 50, and 58.

More particularly, the invention relates to a polynucleotide comprising a nucleotide sequence
a) as given in any one of the SEQ ID NOs of table 6 such as SEQ ID NOs: 35; 49, and 57 or a pail thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in any one of the SEQ ID NOs of table 6 such as SEQ ID NOs: 35; 49, and 57 or the complement thereof;
e) complementary to (a), (b) or (c); and
f) which is the reverse complement of (a), (b) or (c).

Transmembrane transport of sugars has been demonstrated by the presence of transporter genes for a few crop species (spinach, potato). For the uses and application of modifying sugar transport mechanisms with regard to controlling the timing and extent of grain fill durations, we incorporate all relevant sections of PCT Publication WO9953068 to Allen et al., and for uses and application of modifying cells or plastids involved in hexose carrier proteins we incorporate all relevant sections of PCT Publication WO9953082 to Allen et al.

Glucosyl equivalents for starch biosynthesis are found within the scope of the present invention to be transported into the plastid (amyloplast) either as glucose-1-phosphate via a hexose-phosphate-Pi transporter (a representative example of which is given in SEQ ID NO: 35), as triose phosphates via a triose-phosphate-Pi translocator (a representative example of which are given in SEQ ID NO: 163), as phosphoenolpyruvate via a PEP-Pi translocator (SEQ ID NOs: 175), or as ADP-glucose via a Brittle-like adenylate translocator or via an oxoglutarate/malate transporter. One isoform of a triose-phosphate/phosphate translocator (SEQ ID NO: 163) is expressed to a slightly higher level during earlier stages of grain development.

Pyruvate appears to play a more important role during early stages of grain development in that a gene encoding an isoform of a PEP-Pi translocator (SEQ ID NO: 175) is relatively more highly expressed at this stage. In maize endosperm, the majority of glucosyl moieties are transported to the amyloplast during the linear phase of starch accumulation as ADP-glucose (J. C. Shannon et al., Plant Physiol. 117, 1235 (1998)).

For uses and application of modifying amyloplasts in the regulation of starch production via an ADP glucose transporter, we incorporate all relevant sections of PCT Publication WO9947681 to Emes et al.

Further examples of genes encoding a sugar transporter are provided in SEQ ID NOs: 35; 49, and 57. By providing the nucleic acid molecules according to the invention encoding sugar transporters the expression of which is upregulated during grain filling such as those given in SEQ ID NOs: 36; 50, and 58; 36385; 53483; it is now possible to manipulate the translocation and storage of sugars and their carbohydrate end products in the plant grain.

In still another embodiment the present invention provides further subset of nucleic acid molecules which are up-regulated during grain filling comprising a nucleotide sequence encoding a polypeptide that has a transmembrane domain and assists in the transport of amino acids and inorganic compounds including nitrate and various cations, which nucleotide sequence is substantially similar to a sequence encoding a polypeptide as given in SEQ ID NOs: 32; 38; 40; 42; 44; 46; 48; 52; 54; 56; 60; 62; 64, 66; and 68.

In particular, the invention relates to a polynucleotide comprising a nucleotide sequence encoding a polypeptide, that has a transmembrane domain and assists in the transport of amino acids and inorganic compounds including nitrate and various cations and is up-regulated during grain filling and is substantially similar, and preferably has at least between 70%, and 99% amino acid sequence identity to at least one polypeptide of SEQ ID NOs: 32; 38; 40; 42; 44; 46; 48; 52; 54; 56; 60; 62; 64, 66; and 68, with any individual number within this range of between 70% and 99% also being part of the invention.

The invention further relates to a polynucleotide comprising a nucleotide sequence encoding a polypeptide, that has a transmembrane domain and assists in the transport of amino acids and inorganic compounds including nitrate and various cations and is up-regulated during grain filling and is immunologically reactive with antibodies raised against a polypeptide of SEQ ID NOs: 32; 38; 40; 42; 44; 46; 48; 52; 54; 56; 60; 62; 64, 66; and 68.

More particularly, the invention relates to a polynucleotide comprising a nucleotide sequence
a) as given in any one of SEQ ID NOs: 31; 37; 39; 41; 43; 45; 47; 51; 53; 55; 59; 612; 63, 65; and 67 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NO: 31; 37; 39; 41; 43; 45; 47; 51; 53; 55; 59; 612; 63, 65; and 67, or the complement thereof;
e) complementary to (a), (b) or (c); and

f) which is the reverse complement of (a), (b) or (c).

In particular, the invention provides a nucleic acid molecule which is up-regulated during grain filling and comprises a nucleotide sequence encoding a polypeptide that belongs to the POT or PTR family.

Proteins of the POT family (also called the PTR (peptide transport) family) consists of proteins from animals, plants, yeast, archaea, and both Gram-negative and Gram-positive bacteria. Several of these organisms possess multiple POT family paralogues. The proteins are of about 450-600 amino acyl residues in length with the eukaryotic proteins in general being longer than the bacterial proteins. They exhibit 12 putative or established transmembrane ?-helical spanners. Some members of the POT family exhibit limited sequence similarity to protein members of the major facilitator superfamily (MFS; TC #2.A.1). (Comparison scores of up to 8 standard deviations for segments in excess of 60 residues in length.) Thus the POT family is probably a family within the MFS.

While most members of the POT family catalyze peptide transport, one is a nitrate permease and one can transport histidine as well as peptides. Some of the peptide transporters can also transport antibiotics. They function by proton symport, but the substrate:H⁺ stoichiometry is variable: the high affinity rat PepT2 carrier catalyzes uptake of 2 and 3H⁺ with neutral and anionic dipeptides, respectively, while the low affinity PepT1 carrier catalyzes uptake of one H+ per neutral peptide. In eukaryotes, some of these transporters may be in organellar membranes such as the lysosomes.

The generalized transport reaction catalyzed by the proteins of the POT family is:
substrate (out)+nH⁺(out)--->substrate (in)+nH⁺(in).

In a specific embodiment, the present invention relates to an isolated nucleic acid molecule which is up-regulated during grain filling and comprises a nucleotide sequence encoding a polypeptide that belongs to the POT or PTR family, which nucleotide sequence is substantially similar to a sequence encoding a polypeptide as given in SEQ ID NOs: 38; 52, and 68.

In particular, the invention relates to an isolated nucleic acid molecule comprising a nucleotide sequence encoding a polypeptide, which belongs to the POT or PTR family and up-regulated during grain filling and is substantially similar, and preferably has at least between 70%, and 99% amino acid sequence identity to at least one polypeptide of SEQ ID NOs: 38; 52, and 68, with any individual number within this range of between 70% and 99% also being part of the invention.

The invention further relates to an isolated nucleic acid molecule comprising a nucleotide sequence encoding a polypeptide, which belongs to the POT or PTR family and up-regulated during grain filling and is immunologically reactive with antibodies raised against a polypeptide of SEQ ID NOs: 38; 52, and 68.

More particularly, the invention relates to an isolated nucleic acid molecule comprising a nucleotide sequence
a) as given in any one of SEQ ID NOs: 37; 51, and 67 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NO: 37; 51, and 67 or the complement thereof;
e) complementary to (a), (b) or (c); and
f) which is the reverse complement of (a), (b) or (c).

One of the economically most important and valuable carbohydrate end products is starch, which is an essential component of many food, feed, and industrial products. It consists of two types of glucan polymers: relatively long chained polymers with few branches known as amylose, and shorter chained but highly branched molecules called amylopectin.

Its biosynthesis depends on the complex interaction of multiple enzymes (Smith, A. et al., (1995) Plant Physio. 107:673-677; Preiss, J., (1988) Biochemistry of Plants 14:181-253). One of the key enzymes in starch biosynthesis is ADP-glucose pyrophosphorylase, which catalyzes the formation of ADP-glucose; a series of starch synthases which use ADP glucose as a substrate for polymer formation using alpha.-1-4 linkages; and several starch branching enzymes, which modify the polymer by transferring segments of polymer to other parts of the polymer using alpha.-1-6 linkages, creating branched structures. However, based on data from starch forming plants such as potato, and corn, it is becoming clear that other enzymes also play a role in the determination of the final structure of starch. In particular, debranching and disproportionating enzymes not only participate in starch degradation, but also in modification of starch structure during its biosynthesis. Different models for this action have been proposed, but all share the concept that such activities, or lack thereof, change the structure of the starch produced.

In plants used typically for the production of starch, such as maize or potato, the synthesized starch consists of approximately 25% amylose-starch and of about 75% amylopectin-starch.

With respect to the homogeneity of the basic component starch for its use in the industrial area, starch-producing plants are needed which contain, for example, only the component amylopectin or only the component amylose. For a number of other uses plants are needed that synthesize amylopectin types with different degrees of branchings.

Such plants may for example be obtained by breeding or by means of mutagenesis techniques. It is known for various plant species, such as for maize, that by means of mutagenesis varieties may be produced in which only amylopectin is formed. Also in the case of potato a genotype was produced from a haploid line by means of chemical mutagenesis. Said genotype does not form amylose (Hovenkamp-Hermelink, Theor. Appl. Genet. 75 (1987), 217-221).

Apart from conventional breeding and mutagenesis techniques, recombinant DNA techniques are now increasingly used in order to specifically interfere with the starch metabolism of starch storing plants. A prerequisite for this is that DNA sequences be provided which encode enzymes involved in the starch metabolism.

The present invention now provides a subset of nucleic acid molecules that are involved in the starch biosynthesis pathway and were shown to be up-regulated during grain filling. Representative examples of those subset genes are provided in SEQ ID NOs: 69-187 of the Sequence Listing.

In a particular embodiment, the present invention relates to a polynucleotide comprising a nucleotide sequence encoding a polypeptide which is involved in associated with starch biosynthsis and up-regulated during grain filling, which nucleic acid molecule is substantially similar to a nucleic acid encoding a polypeptide as given in any one of the SEQ ID NOs of table 7 such as SEQ ID NOs: 70-188.

More specifically, the invention relates to a polynucleotide comprising a nucleotide sequence encoding a polypeptide, which is involved in or associated with starch biosynthesis and up-regulated during grain filling and is substantially similar, and preferably has at least between 70%, and 99% amino acid sequence identity to at least one polypeptide as given in any one of the SEQ ID NOs of table 7 such as SEQ ID NOs: 70-188, with any individual number within this range of between 70% and 99% also being part of the invention.

The invention further relates to a polynucleotide comprising a nucleotide sequence encoding a polypeptide, which is involved in or associated with starch biosynthesis and up-regulated during grain filling and is immunologically reactive with antibodies raised against a polypeptide as given in any one of the SEQ ID NOs of table 7 such as SEQ ID NOs: 70-188.

More particularly, the invention relates to a polynucleotide comprising a nucleotide sequence
a) as given in any one of the SEQ ID NOs of table 7 such as SEQ ID NOs: 69-187 or a part thereof which still encodes a partial-length polypeptide having substantially the same activity as the full-length polypeptide, e.g., at least 50%, more preferably at least 80%, even more preferably at least 90% to 95% the activity of the fill-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in any one of the SEQ ID NOs of table 7 such as SEQ ID NOs: 69-187, or the complement thereof,
e) complementary to (a), (b) or (c); and
f) which is the reverse complement of (a), (b) or (c).

By providing a subset of genes encoding polypeptides that are involved in starch metabolism it is now possible to interfere with starch metabolism to produce starch with modified physico/chemical characteristics.

A gene encoding the small subunit of ADPG pyrophosphorylase (SEQ ID NO: 138); is expressed at early stages of grain development in conjunction with a single gene encoding a large subunit (SEQ ID NO: 140). Three other large subunits (SEQ ID NOs: 136; 142); are up-regulated at a later stage in development from 4 days after anthesis, in conjunction with the up regulation of the starch synthase genes (SEQ ID NOs: 129; 131; and 133) and two genes for branching enzymes (SEQ ID NOs: 70; and 72) (involved in amylose and amylopectin biosynthesis, respectively). Only one (distinct from the two mentioned above) of the small subunit genes increases in this time period. The expression of different isoforms may be related to the shift to storage starch production and a postulated concomitant shift to cytoplasmic ADP-glucose production (Stark, D. M., et al., “Regulation of the Amount of Starch in Plant Tissues by ADP Glucose Pyrophosphorylase”, Science, 258,287-291 (Oct. 9, 1992)).

In one embodiment the present invention provides a nucleic acid molecule comprising a nucleotide sequence which encodes a small subunit of ADPG pyrophosphorylase. In another embodiment the invention provides a nucleic acid molecule comprising a nucleotide sequence which encodes a large subunit of ADPG pyrophosphorylase.

In particular, the invention relates to a polynucleotide comprising a nucleotide sequence encoding a polypeptide with an activity of a small and large subunit ADPG pyrophosphorylase, respectively, which nucleotide sequence is substantially similar to a nucleic acid sequence encoding a polypeptide as given in SEQ ID NOs: 136-142.

More specifically, the invention relates to a polynucleotide comprising a nucleotide sequence encoding a polypeptide with an activity of a small and large subunit ADPG pyrophosphorylase, respectively, which is up-regulated during grain filling and has at least between 70%, and 99% amino acid sequence identity to at least one polypeptide of SEQ ID NOs: 136-142, with any individual number within this range of between 70% and 99% also being part of the invention.

The invention further relates to a polynucleotide comprising a nucleotide sequence encoding a polypeptide with an activity of a small and large subunit ADPG pyrophosphorylase, respectively, which is up-regulated during grain and immunologically reactive with antibodies raised against a polypeptide of SEQ ID NOs: 136-142.

More particularly, the invention relates to a polynucleotide comprising a nucleotide sequence
a) as given in any one of SEQ ID NOs: SEQ ID NOs: 135-141 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of nucleotides given in SEQ ID NO: SEQ ID NOs: 135-141, or the complement thereof;
e) complementary to (a), (b) or (c); and
f) which is the reverse complement of (a), (b) or (c).

The nucleic acid molecules of the instant invention may be used to create transgenic plants in which the small and/or large subunits of ADPG pyrophosphorylase are present at higher or lower levels than normal or in cell types or developmental stages in which it is not normally found. This may have the effect of altering starch structure in those cells or tissues but especially in the developing grain.

For a further targeted modification of the starch in plants, in particular of the degree of branching of starch synthesized in plants by means of recombinant DNA techniques, it is still necessary to identify DNA sequences that encode enzymes participating in the starch metabolism, particularly in the branching of starch molecules.

In the case of potato, for example, DNA sequences have by now been described which encode a granule-bound starch synthase or a branching enzyme (Q enzyme), and they have been used in order to genetically modify plants.

Apart from the Q enzymes that introduce branchings into starch molecules, enzymes occur in plants which are capable of dissolving branchings. These enzymes are called debranching enzymes.

In the case of sugar beet, Li et al. (Plant Physiol. 98 (1992), 1277-1284) could only prove the occurrence of one debranching enzyme, apart from five endo- and two exoamylases. This enzyme having a size of approximately 100 kD and an optimum pH value of 5.5 is located within the chloroplasts. A debranching enzyme was also described for spinach. The debranching enzyme from spinach as well as that from sugar beet exhibit a fivefold lower activity in a reaction with amylopectin as substrate when compared to a reaction with pullulan as a substrate (Ludwig et al., Plant Physiol. 74 (1984), 856-861; Li et al., Plant Physiol. 98 (1992), 1277-1284). The isolation of a cDNA encoding a debranching enzyme was described for spinach (Renz et al., Plant Physiol. 108 (1995), 1342).

The existence of a debranching enzyme for maize has been described in the prior art. The corresponding mutant was designated su (sugary). The gene of the sugary locus was cloned recently (see James et al., Plant Cell 7 (1995), 417-429). In the case of the agriculturally significant starch storing cultured plant potato, the activity of a debranching enzyme was examined by Hobson et al. (J. Chem. Soc., (1951), 1451). It was proven that the respective enzyme, contrary to the Q enzyme, does not exhibit any activities leading to an elongation of the polysaccharide chain, but merely hydrolyses .alpha.-1,6-glycosidic bonds.

Within the scope of the present invention a subset of genes is provided that encode polypeptides the activity of which is associated with the structural shaping of the starch granule. In particular, the invention provides a subset of genes that encode polypeptides the activity of which is associated the branching/debranching (representative examples of wich are given in SEQ ID NOs: 69-73/75; 77 (isoamylase debranching enzyme)) and/or degradation of starch (a-amylase (SEQ ID NO: 79-91), pullulanase (SEQ ID NO: 109) [the last gene in the a-amylase series], a-amylase inhibitor (SEQ ID NOs: 93-99); β-amylase (SEQ ID NO101-107), a-glucosidase (SEQ ID NO: 111-117). By modulating the expression of the polypeptides according to the invention, the amylose amylopectin ratio can be changed in order to accommodate the varying quality standards for food and/or feed applications or specific processing requirements. For example, by over-expressing and inhibiting the expression of endogeneous branching and/or debranching enzyme genes in rice or any other cereal crop plant, respectively, a plant can be produced that exhibits increased or reduced amounts of branching/debranching enzyme activity for the purpose of modifying the degree of branching of the amylopectin starch.

By inhibiting the expression of endogeneous branching and/or debranching enzyme genes, plants are produced that exhibit a reduced activity of these enzymes, which leads to the synthesis of a modified starch. Inhibition of branching/debranching gene expression can be achieved by applying method known in the art such as, for example, antisense or dsRNAi techniques. By applying these techniques it is possible to produce plants in which the expression of an endogeneous branching/debranching enzyme gene in rice or any other cereal crop plant is inhibited to different degrees within the range of 0.1% to 100%, which all individual numbers within this range also being part of the invention. This enables in particular the production of cereal plants synthesizing amylopectin starch with most various variations of the degree of branching. This constitutes an advantage with regard to conventional breeding and mutagenesis techniques in which a lot of time and costs are required in order to provide such a variety. Highly branched amylopectin has a particularly large surface and is therefore particularly suitable as a copolymer. A high degree of branching furthermore leads to an improvement of the amylopectin's solubility in water. This property is very advantageous for certain technical applications.

Another way of modifying the branching characteristics of starch is by overexpressing the nucleic acid molecule according to the invention encoding a branching/debranching enzyme activity in rice in a transgenic plant, but especially a plant seed.

The expression of a novel or additional branching/debranching enzyme activity from rice in the transgenic plant cells and plants of the invention influences the degree of branching of the amylopectin synthesized in the cells and plants. Therefore, a starch synthesized in these plants exhibits modified physical and/or chemical properties when compared to starch from wildtype plants.

Genes encoding products involved in starch structure rearrangement (debranching enzyme is (SEQ ID NO: 75-77 (isoamylase debranching enzyme)); branching enzyme (SEQ ID NOs: 69-73)) and starch degradation (a-amylase (SEQ ID NOs 79-91), a-amylase inhibitor (SEQ ID NOs: 93-99); pullulanase (SEQ ID NOs 109) [the last gene in the a-amylase series], β-amylase (SEQ ID NOs 101-107), a-glucosidase (SEQ ID NOs 111-117)) are all strongly expressed towards the end of grain development, reflecting their involvement in the final stages of shaping the starch granule. Genes encoding isoforms of an a-amylase inhibitor (SEQ ID NOs: 93 and 95) are expressed most strongly in the aleurone and seed coat layers, and endosperm and not (or to a reduced extent) in the embryo. The embryo also shows a different expression of genes encoding starch synthase and branching enzymes, perhaps reflecting its status as an energy-requiring sink organ rather than as a storage tissue. Myers et al. discuss the interaction of starch synthases, branching enzymes, debranching enzymes and disproportionating enzymes in producing and trimming glucan molecules so that a final transition may take place to a crystalline form (A. M. Myers, M. K. Morell, M. G. James, S. G. Ball. Plant Physiol. 122, 989 (2000)).

In a further embodiment, the present invention provides the ability to modulate the shape and the physico/chemical properties of the starch granule by modifying expression level and pattern of those genes that encode products involved in starch structure rearrangement such as, for example, SEQ ID NO: 75-77 (isoanylase debranching enzyme); branching enzyme (SEQ ID NOs: 69-73) and starch degradation (a-amylase (SEQ ID NOs 79-91)), a-amylase inhibitor (SEQ ID NOs: 93-99); pullulanase (SEQ ID NO: 109), β-amylase (SEQ ID NO: 101-107), and/or a-glucosidase (SEQ ID NO: 111-117).

The invention thus also relates to a polynucleotide comprising a nucleotide sequence encoding a polypeptide involved in starch structure rearrangement, which nucleic acid molecule is substantially similar to a nucleic acid encoding a polypeptide as given in the SEQ ID NOs of table 7 such as SEQ ID NOs: 75-77 exhibiting isoamylase debranching enzyme activity, 69-73 exhibiting a branching enzyme activity, 80-92 exhibiting an a-amylase activity; 94-100 exhibiting an a-amylase inhibitor activity; 110 exhibiting a pullulanase activity; 102-108, exhibiting a β-amylase activity; 112-118, exhibiting a a-glucosidase activity.

More specifically, the invention relates to a polynucleotide comprising a nucleotide sequence encoding a polypeptide which is involved in starch structure rearrangement and up-regulated during grain filling and has at least between 70%, and 99% amino acid sequence identity to at least one polypeptide as given in the SEQ ID NOs of table 7 such as SEQ ID NOs: 75-77 exhibiting isoamylase debranching enzyme activity, 69-73 exhibiting a branching enzyme activity, 80-92, 80-92 exhibiting an a-amylase activity; 94-100 exhibiting an a-amylase inhibitor activity; 110 exhibiting a pullulanase activity; 102-108, exhibiting a β-amylase activity; 112-118, exhibiting a a-glucosidase activity with any individual number within this range of between 70% and 99% also being part of the invention.

The invention further relates to a polynucleotide comprising a nucleotide sequence encoding a polypeptide which is involved in starch structure rearrangement and up-regulated during grain filling and immunologically reactive with antibodies raised against a polypeptide as given in the SEQ ID NOs of table 7 such as SEQ ID NOs: 75-77 exhibiting isoamylase debranching enzyme activity, 69-73 exhibiting a branching enzyme activity, 80-92, 80-92 exhibiting an a-amylase activity; 94-100 exhibiting an a-amylase inhibitor activity; 110 exhibiting a pullulanase activity; 102-108, exhibiting a β-amylase activity; 112-118, exhibiting a a-glucosidase activity.

More particularly, the invention relates to a polynucleotide comprising a nucleotide sequence
a) as given in the SEQ ID NOs of table 7 such as SEQ ID NOs: 75-77 exhibiting isoamylase debranching enzyme activity, 69-73 exhibiting a branching enzyme activity, 79-91 exhibiting an a-amylase activity; 93-99 exhibiting an a-amylase inhibitor activity; 109 exhibiting a pullulanase activity; 101-107, exhibiting a 6-amylase activity; 111-117, exhibiting a a-glucosidase activity or a part thereof which still encodes a partial-length polypeptide having substantially the same activity as the fill-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given as given in the SEQ ID NOs of table 7 such as SEQ ID NOs: 75-77 exhibiting isoamylase debranching enzyme activity; 69-73 exhibiting a branching enzyme activity, 79-91 exhibiting an a-amylase activity; 93-99 exhibiting an a-amylase inhibitor activity; 109 exhibiting a pullulanase activity; 101-107, exhibiting a β-amylase activity; 111-117, exhibiting a a-glucosidase activity, or the complement thereof;
e) complementary to (a), (b) or (c); and
f) which is the reverse complement of (a), (b) or (c).

The identification of a defined subset of genes that are involved in carbohydrate metabolism but especially in starch metabolism and the expression of which is coordinately up- or down-regulated during the grain filling process makes it now possible to improve grain quality by overexpressing and/or underexpressing or completely knocking out genes that are known to positively contribute to the nutritional or processing properties of grains such as, for example, genes encoding products involved in starch structure rearrangement and starch degradation as mentioned hereinbefore.

The expression of a-amylase, which is central in the starch biosynthesis pathway, may further be modified to obtain plants producing a desirable content of reducing sugars. For, example, a high content of reducing sugar resulting from a high α-amylase activity is desirable when rice or other cereal plants are to be used for the production of alcohol. This can be achieved by modifying the expression of the plant endogenous genes encoding an α-amylase or α-amylase inhibitor activity, for example, by introducing and overexpressing in a target plant a nucleic acid molecule comprising a nucleotide sequence that encodes a polypeptide the amino acid sequence of which is substantially similar to any one of those given in SEQ ID NOs: 80-92 exhibiting an a-amylase activity; and 94-100 exhibiting an a-amylase inhibitor activity.

In the specific embodiment, the invention thus also relates to a polynucleotide comprising a nucleotide sequence encoding a polypeptide exhibiting an amylase or an amylase inhibitor activity, which nucleic acid molecule is substantially similar to a nucleic acid encoding a polypeptide as given in the SEQ ID NOs of table 7 such as SEQ ID NOs: 80-92 exhibiting an a-amylase activity; and 94-100 exhibiting an a-amylase inhibitor activity.

More specifically, the invention relates to a polynucleotide comprising a nucleotide sequence encoding a polypeptide which has an activity of an amylase and is up-regulated during grain filling and has at least between 70%, and 99% amino acid sequence identity to at least one polypeptide as given in the SEQ ID NOs of table 7 such as SEQ ID NOs: 80-92 exhibiting an a-amylase activity; and 94-100 exhibiting an a-amylase inhibitor activity, with any individual number within this range of between 70% and 99% also being part of the invention.

The invention further relates to a polynucleotide comprising a nucleotide sequence encoding a polypeptide which which has an activity of an amylase and is up-regulated during grain filling and immunologically reactive with antibodies raised against a polypeptide as given in the SEQ ID NOs of table 7 such as SEQ ID NOs: 80-92 exhibiting an a-amylase activity; and 94-100 exhibiting an a-amylase inhibitor activity.

More particularly, the invention relates to a polynucleotide comprising a nucleotide sequence
a) as given in the SEQ ID NOs of table 7 such as SEQ ID NOs: 79-91 exhibiting an a-amylase activity; and 93-99 exhibiting an a-amylase inhibitor activity or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in the SEQ ID NOs of table 7 such as SEQ ID NOs: 79-91 exhibiting an a-amylase activity; and 93-99 exhibiting an a-amylase inhibitor activity or the complement thereof;
e) complementary to (a), (b) or (c); and
f) which is the reverse complement of (a), (b) or (c).

Different isoforms often show distinct spatial expression patterns. For example, three different sucrose synthase isoforms (SEQ ID NOs: 119-123) are expressed in developing grain tissue, two of which (SEQ ID NOs: 121 and 123) are expressed more highly at the start of grain development (0 days post anthesis) and one (SEQ ID NO: 119) which is up-regulated towards the end of grain development. The spatial distribution of each differs. Other isoforms (SEQ ID NOs: 125 and 127), showing low expression in the grain, are expressed strongly in stems or roots.

The invention thus also relates to a polynucleotide comprising a nucleotide sequence encoding a polypeptide exhibiting a sucrose synthase activity, which nucleic acid molecule is substantially similar to a nucleic acid encoding a polypeptide as given in SEQ ID NOs: 120-128.

More specifically, the invention relates to a polynucleotide comprising a nucleotide sequence encoding a polypeptide which has an activity of an sucrose synthase and is up-regulated during grain filling and has at least between 70%, and 99% amino acid sequence identity to at least one polypeptide of SEQ ID NOs: 120-128, with any individual number within this range of between 70% and 99% also being part of the invention.

The invention further relates to a polynucleotide comprising a nucleotide sequence encoding a polypeptide which which has an activity of a sucrose synthase and is up-regulated during grain filling and immunologically reactive with antibodies raised against a polypeptide of SEQ ID NOs: 120-128.

More particularly, the invention relates to a polynucleotide comprising a nucleotide sequence
a) as given in any one of SEQ ID NOs: 119-127 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NOs: 119-127 or the complement thereof,
e) complementary to (a), (b) or (c); and
f) which is the reverse complement of (a), (b) or (c).

In a further embodiment, the present invention provides the ability to regulate glucanases (as represented by SEQ ID NO: 191). Glucanases can be used to minimize wet droppings in high wheat, or barley, poultry and swine diets by breaking down and reducing the viscosity of β-glucans and other non-starch polysaccharides and thus can provide benefit as a processing aid in animal feed. For uses and application of modifying crop plants by creating transgenic monocots and monocot seeds expressing rice β-glucanase enzymes and genes we incorporate all relevant section of PCT Publication WO9859046 to Rodriguez.

The invention thus also relates to a polynucleotide comprising a nucleotide sequence encoding a polypeptide exhibiting a glucanase activity, which nucleic acid molecule is substantially similar to a nucleic acid encoding a polypeptide as given in SEQ ID NOs: 192.

More specifically, the invention relates to a polynucleotide comprising a nucleotide sequence encoding a polypeptide which has an activity of an glucanase and is up-regulated during grain filling and has at least between 70%, and 99% amino acid sequence identity to at least one polypeptide of SEQ ID NOs: 192, with any individual number within this range of between 70% and 99% also being part of the invention.

The invention further relates to a polynucleotide comprising a nucleotide sequence encoding a polypeptide which which has an activity of a glucanase and is up-regulated during grain filling and immunologically reactive with antibodies raised against a polypeptide of SEQ ID NOs: 192.

More particularly, the invention relates to a polynucleotide comprising a nucleotide sequence
a) as given in SEQ ID NO: 191 or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof,
d) capable of hybridizing to a nucleic acid comprising 50 to 200 or more consecutive nucleotides of nucleotides given in SEQ ID NO: 191 or the complement thereof;
e) complementary to (a), (b) or (c); and
f) which is the reverse complement of (a), (b) or (c).

Thus, in an embodiment applicable to all of the above stated provisions, the present invention provides nucleotide sequences encoding at least one polypeptide involved in the synthesis, metabolism, transport or storage of carbohydrates, as well as any polypeptides encoded thereby, or any antigene sequences thereof, which have numerous applications using techniques that are known to those skilled in the art of molecular biology, biotechnology, biochemistry, genetics, physiology or pathology. These techniques include the use of nucleotide molecules as hybridization probes, for chromosome and gene mapping, in PCR technologies, in the production of sense or antisense nucleic acids, in screening for new therapeutic molecules, in production of plants and seeds having desirable, inheritable, commercially useful phenotypes, or in discovery of inhibitory compounds.

In a further collective embodiment, the present invention provides the ability to modulate carbohydrates, sugars and their transporters in plant tissues, by over-expressing, under-expressing or knocking out one or more cell cycle genes or their gene products, in a plant cell, in vitro or in planta. Expression vectors comprising at least one nucleotide sequence involved in carbohydrate or sugar synthesis, metabolism, transport or storage, or any antigenes thereof, operably linked to at least one suitable promoter and/or regulatory sequence can be used to study the role of polypeptides encoded by said sequences, for example by transforming a host cell with said expression vector and measuring the effects of overexpression and underexpression of sequences. A host cell transformed with at least one expression vector comprising nucleotide sequences involved in carbohydrate modulation, operably linked to suitable promoters and/or regulatory sequences, can be useful to produce a dietary supplement comprising a polypeptide having a defined amino acid profile.

In a further collective embodiment, the present invention provides a transformed plant host cell, or one obtained through breeding, capable of over-expressing, under-expressing, or having a knock out of said metabolic genes and/or their gene products.

Such a plant cell, transformed with at least one expression vector comprising nucleotide sequences involved in carbohydrate synthesis, metabolism, transport or storage, operably linked to suitable promoters and/or regulatory sequences, can be used to regenerate plant tissue or an entire plant, or seed there from, in which the effects of expression, including overexpression or underexpression, of the introduced sequence or sequences can be measured in vitro or in planta.

A further subset of genes provided herein comprises genes that encode polypeptides with an activity that is involved in or associated with the production of seed storage proteins.

In seeds of higher plants, proteins are contained in an amount of 20-30% by weight in case of beans, and in an amount of about 10% by weight in case of cereals, based on dry weight. Among the proteins in seeds, 70-80% by weight are storage proteins. Particularly, in rice seeds, about 80% by weight of the seed storage proteins is glutelin which is only soluble in dilute acids and dilute alkalis. The remainders are prolamin (10-15% by weight) soluble in organic solvents and globulin (5-10% by weight) solubilized by salts.

Seed storage proteins are important as a protein source in foods and feeds, so that they have been well studied from the view points of nutrition and protein chemistry. As a result, in cereals, storage protein genes of maize, wheat, barley and the like have been cloned, amino acid sequences of the proteins have been deduced from the nucleotide sequence, and regulatory regions of the genes have been analyzed.

The present invention provides a subset of nucleic acid molecules that is up-regulated during grain filling and comprises a nucleotide sequence encoding a seed storage protein. Representative examples of these genes are given in SEQ ID NOs: 211-249.

The invention thus also relates to a polynucleotide comprising a nucleotide sequence encoding a seed storage protein, which nucleic acid molecule is substantially similar to a nucleic acid encoding a polypeptide as given in any one of the SEQ ID NOs of table 8 such as SEQ ID NOs: 212-250.

More specifically, the invention relates to a polynucleotide comprising a nucleotide sequence encoding a seed storage protein which is up-regulated during grain filling and has at least between to 70%, and 99% amino acid sequence identity to at least one polypeptide as given in any one of the SEQ ID NOs of table 8 such as SEQ ID NOs: 212-250, with any individual number within this range of between 70% and 99% also being part of the invention.

The invention further relates to a polynucleotide comprising a nucleotide sequence encoding a seed storage protein, which is up-regulated during grain filling and immunologically reactive with antibodies raised against a polypeptide as given in any one of the SEQ ID NOs of table 8 such as SEQ ID NOs: 212-250.

More particularly, the invention relates to a polynucleotide comprising a nucleotide sequence
a) as given in any one of the SEQ ID NOs of table 8 such as SEQ ID NOs: 211-249 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof,
d) capable of hybridizing to a nucleic acid comprising 50 to 200 or more consecutive nucleotides of a nucleotide sequence as given in any one of the SEQ ID NOs of table 8 such as SEQ ID NOs: 211-249 or the complement thereof;
e) complementary to (a), (b) or (c); and
f) which is the reverse complement of (a), (b) or (c).

By providing the above subset of genes, the protein content and composition in the plant grain can be modified by up- or down-regulating the expression of at least one nucleic acid molecule within this subgroup giving rise to altered levels or an altered composition of seed storage protein in the plant grain.

For rice grains to be processed, it is advantageous that the protein content is small. In case of rice to be used for preparing fermented alcoholic beverage, this can be attained through well defined refinement measures, thereby removing the proteins in the peripheral portion of endosperm which contains large amounts of storage proteins. In producing rice starch, in order to promote the purity, proteins are removed by treatments with alkalis, surfactants and ultrasonication.

The protein content in the rice grain also influences the taste of rice. Good tasting rice grains have usually low contents of proteins. Rice varieties with a low protein content have been developed by the conventional cross-breeding or by mutation-breeding. (U.S. Pat. No. 5,516,668; Maruta).

U.S. Pat. No. 5,516,668 describes a method for decreasing the amount of glutelin in plant seeds, comprising introducing into a rice plant a gene which is a template for the transcription of an antisense RNA against rice glutelin; and transcribing said gene in seeds from said rice plant to inhibit translation of mRNA of glutelin, thereby decreasing the amount of glutelin in said seeds in comparison to the amount of glutelin contained in seeds from unmodified wild-type rice plants.

The cDNA of glutelin which is a seed storage protein in rice has been cloned and complete primary structure of the protein has been determined by sequencing the cDNA. The gene of this protein has been isolated by using the cDNA as a probe (Japanese Laid-open Patent Application (Kokai) No. 63-91085).

Rice plants with a low glutelin content in the rice grain can now be produced more efficiently by down-regulating two or more of the the endogenous glutelin genes in rice seeds such as those provided in SEQ ID NOs: 223, 235, and 239 using methods known in the art including antisense and dsRNAi techniques.

The invention thus also relates to a polynucleotide comprising a nucleotide sequence encoding a glutelin protein the expression of which is up-regulated during grain filling, which nucleic acid molecule is substantially similar to a nucleic acid encoding a polypeptide as given in SEQ ID NOs: 224, 236, and 240.

More specifically, the invention relates to a polynucleotide comprising a nucleotide sequence encoding a glutelin protein the expression of which is up-regulated during grain filling and which has at least between 70%, and 99% amino acid sequence identity to at least one polypeptide of SEQ ID NOs: 224, 236, and 240, with any individual number within this range of between 70% and 99% also being part of the invention.

The invention further relates to a polynucleotide comprising a nucleotide sequence encoding a seed glutelin protein, the expression of which is up-regulated during grain filling and which is immunologically reactive with antibodies raised against a polypeptide of SEQ ID NOs: 224, 236, and 240.

More particularly, the invention relates to a polynucleotide comprising a nucleotide sequence
a) as given in any one of SEQ ID NOs: 223,235, and 239 or a part thereof which still encodes a partial length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 223, 235, and 239, or the complement thereof;
e) complementary to (a), (b) or (c); and
f) which is the reverse complement of (a), (b) or (c).

Another class of seed storage proteins are the prolamins, which are naturally rich in the essential amino acids lysine and methionine. Overexpressing said genes can thus increase the nutritional value of feeds and foods by producing said proteins at higher levels than those found in the unmodified wild-type plants. Another aspect of the present invention thus relates to providing genes that encode rice prolamin protein such as those given in SEQ ID NOs: 217, 219, 225 and 241.

The invention thus also relates to a polynucleotide comprising a nucleotide sequence encoding a prolamin protein the expression of which is up-regulated during grain filling, which nucleotide sequence is substantially similar to a nucleic acid sequence encoding a polypeptide as given in SEQ ID NOs: 218, 220, 226 and 242.

More specifically, the invention relates to a polynucleotide comprising a nucleotide sequence encoding a prolamin protein, the expression of which is up-regulated during grain filling and which has at least between 70%, and 99% amino acid sequence identity to at least one polypeptide of SEQ ID NOs: 218, 220, 226 and 242, with any individual number within this range of between 70% and 99% also being part of the invention.

The invention further relates to a polynucleotide comprising a nucleotide sequence encoding a prolamin protein, the expression of which is up-regulated during grain filling and which is immunologically reactive with antibodies raised against a polypeptide of SEQ ID NOs: 218, 220, 226 and 242.

More particularly, the invention relates to a polynucleotide comprising a nucleotide sequence
a) as given in any one of SEQ ID NOs: 217, 219, 225 and 241 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 217, 219, 225 and 241, or the complement thereof;
e) complementary to (a), (b) or (c); and
f) which is the reverse complement of (a), (b) or (c).

Gliadins are a further group of seed storage proteins that are of economic importance. Gliadin is a single-chained protein having an average molecular weight of about 30,000-40,000, with an isoelectric of pH 4.0-5.0. Gliadin proteins are extremely sticky when hydrated and have little or no resistance to extension. Gliadin is responsible for giving gluten dough its characteristic cohesiveness. Gliadin is a premium products, when available.

Gliadin is known to improve the freeze-thaw stability of frozen dough and also improves microwave stability. This product is also used as an all-natural chewing gum base replacer, a pharmaceutical binder, and improves the texture and mouth feel of pasta products and has been found to improve cosmetic products.

The invention provides a further subset of genes comprising a nucleotide sequence that encodes gliadin storage proteins. By overexpressing said genes in the plant, but preferably in the plant seed, the plant produces grain with an increased concentration of gliadin as compared to the unmodified wild-type plant.

In a particular embodiment, the invention thus relates to a polynucleotide comprising a nucleotide sequence encoding a gliadin protein, the expression of which is up-regulated during grain filling, which nucleotide sequence is substantially similar to a nucleic acid sequence encoding a polypeptide as given in SEQ ID NOs: 212, 219; 234, 248; and 250.

More specifically, the invention relates to a polynucleotide comprising a nucleotide sequence encoding a gliadin protein, the expression of which is up-regulated during grain filling and which has at least between 70%, and 99% amino acid sequence identity to at least one polypeptide of SEQ ID NOs: 212, 219; 234, 248; and 250, with any individual number within this range of between 700/o and 99% also being part of the invention.

The invention further relates to a polynucleotide comprising a nucleotide sequence encoding a seed gliadin protein, the expression of which is up-regulated during grain filling and which is immunologically reactive with antibodies raised against a polypeptide of SEQ ID NOs: 212, 219; 234, 248; and 250.

More particularly, the invention relates to a polynucleotide comprising a nucleotide sequence
g) as given in any one of SEQ ID NOs: 211, 220; 233, 247; and 249 or a part thereof which still encodes a partial length polypeptide having substantially the same activity as the full-length polypeptide, e.g., at least 50%, more preferably at least 80%, even more preferably at least 90% to 95% the activity of the full-length polypeptide;
h) having substantial similarity to (a);
i) capable of hybridizing to (a) or the complement thereof;
j) capable of hybridizing to a nucleic acid comprising 50 to 200 or more consecutive nucleotides of a nucleotide sequence given in any one of SEQ ID NOs: 211, 220; 233, 247; and 249, or the complement thereof;
k) complementary to (a), (b) or (c); and
l) which is the reverse complement of (a), (b) or (c).

In a further embodiment the invention provides a subset of genes which encode polypeptides that are involved in or associated with the metabolism of fatty acids in the rice grain.

Seed oil content has traditionally been modified by plant breeding. The use of recombinant DNA technology to alter seed oil composition can accelerate this process and in some cases alter seed oils in a way that cannot be accomplished by breeding alone. The oil composition of Brassica has been significantly altered by modifying the expression of a number of lipid metabolism genes. Such manipulations of seed oil composition have focused on altering the proportion of endogenous component fatty acids. For example, antisense repression of the .DELTA.12-desaturase gene in transgenic rapeseed has resulted in an increase in oleic acid of up to 83%. (Topfer et al. 1995 Science 268:681-686).

There have been some successful attempts at modifying the composition of seed oil in transgenic plants by introducing new genes that allow the production of a fatty acid that the host plants were not previously capable of synthesizing. Van de Loo, et al. (1995 Proc. Natl. Acad. Sci USA 92:6743-6747) have been able to introduce a .DELTA.12-hydroxylase gene into transgenic tobacco, resulting in the introduction of a novel fatty acid, ricinoleic acid, into its seed oil. The reported accumulation was modest from plants carrying constructs in which transcription of the hydroxylase gene was under the control of the cauliflower mosaic virus (CaMV) 35S promoter. Similarly, tobacco plants have been engineered to produce low levels of petroselinic acid by expression of an acyl-ACP desaturase from coriander (Cahoon et al. 1992 Proc. Natl. Acad. Sci USA 89:11184-11188).

The long chain fatty acids (C18 and larger), have significant economic value both as nutritionally and medically important foods and as industrial commodities (Ohlrogge, J. B. 1994 Plant Physiol. 104:821-826). Linoleic (18:2.DELTA.9,12) and alpha.-linolenic acid (18:3 .DELTA.9,12,15) are essential fatty acids found in many seed oils. The levels of these fatty-acids have been manipulated in oil seed crops through breeding and biotechnology (Ohlrogge, et al. 1991 Biochim. Biophys. Acta 1082:1-26; Topfer et al. 1995 Science 268:681-686). Additionally, the production of novel fatty acids in seed oils can be of considerable use in both human health and industrial applications.

Consumption of plant oils rich in .gamma.-linolenic acid (GLA) (18:3.DELTA.6,9,12) is thought to alleviate hypercholesterolemia and other related clinical disorders which correlate with susceptibility to coronary heart disease (Brenner R. R. 1976 Adv. Exp. Med. Biol. 83:85-101). The therapeutic benefits of dietary GLA may result from its role as a precursor to prostaglandin synthesis (Weete, J. D. 1980 in Lipid Biochemistry of Fungi and Other Organisms, eds. Plenum Press, New York, pp. 59-62). Linoleic acid(18:2) (LA) is transformed into gamma linolenic acid (18:3) (GLA) by the enzyme .DELTA.6-desaturase.

Few seed oils contain GLA despite high contents of the precursor linoleic acid. This is due to the absence of .DELTA.6-desaturase activity in most plants. For example, only borage (Borago officinalis), evening primrose (Oenothera biennis), and currants (Ribes nigrum) produce appreciable amounts of linolenic acid. Of these three species, only Oenothera and Borage are cultivated as a commercial source for GLA. It would be beneficial if agronomic seed oils could be engineered to produce GLA in significant quantities by introducing a heterologous .DELTA.6-desaturase gene. It would also be beneficial if other expression products associated with fatty acid synthesis and lipid metabolism could be produced in plants at high enough levels so that commercial production of a particular expression product becomes feasible.

As disclosed in U.S. Pat. No. 5,552,306, a cyanobacterial .DELTA.sup.6-desaturase gene has been recently isolated. Expression of this cyanobacterial gene in transgenic tobacco resulted in significant but low level GLA accumulation. (Reddy et al. 1996 Nature Biotech. 14:639-642).

The present invention now provides a subset of genes encoding polypeptides that are involved in or associated with fatty acid metabolism, the expression of which is up-regulated during grain filling.

In particular, the invention relates to a polynucleotide the expression of which is up-regulated during grain filling comprising a nucleotide sequence encoding a polypeptide that is involved in or associated with fatty acid synthesis or lipid metabolism, which nucleotide sequence is substantially similar to a nucleic acid sequence encoding a polypeptide as given in any one of the SEQ ID NOs of table 9 such as SEQ ID NOs: 252-280.

More specifically, the invention relates to a polynucleotide the expression of which is up-regulated during grain filling comprising a nucleotide sequence encoding a polypeptide that is involved in or associated with fatty acid synthesis or lipid metabolism and has at least between 70%, and 99% amino acid sequence identity to at least one polypeptide as given in any one of the SEQ ID NOs of table 9 such as SEQ ID NOs: 252-280, with any individual number within this range of between 70% and 99% also being part of the invention.

The invention further relates to a polynucleotide the expression of which is up-regulated during grain filling comprising a nucleotide sequence encoding a polypeptide that is involved in or associated with fatty acid synthesis or lipid metabolism and immunologically reactive with antibodies raised against a polypeptide as given in any one of the SEQ ID NOs of table 9 such as SEQ ID NOs: 252-280.

More particularly, the invention relates to a polynucleotide comprising a nucleotide sequence
a) as given in any one of the SEQ ID NOs of table 9 such as SEQ ID NOs: 251-279 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of nucleotides as given in any one of the SEQ ID NOs of table 9 such as SEQ ID NOs: 251-279 or the complement thereof,
e) complementary to (a), (b) or (c); and
f) which is the reverse complement of (a), (b) or (c).

By providing this subset of genes it is now possible to modify the level and composition of grain lipids by modulating the expression of those genes in the plant seed. Expression can be modulated either by introducing at least one of the nucleic acid molecules from this subset into the plant, preferably under control of a seed specific promoter, and overexpressing said at least one to nucleic acid molecule in the plant seed, or, by down-regulating expression of the corresponding endogenous gene applying techniques know in the art including anti sense and dsRNAi techniques.

In a specific embodiment, the invention relates to a subset of genes encoding oleosins as represented by SEQ ID NOs: 257 and 259.

Oleosins are abundant seed proteins associated with the phospholipid monolayer membrane of oil bodies, which are a means for storing lipids in the plant cell. Analysis of the contents of lipid bodies has demonstrated that in addition to triglyceride and membrane lipids, there are also several polypeptides/proteins associated with the surface or lumen of the oil body (Bowman-Vance and Huang, 1987, J. Biol. Chem., 262:11275-11279, Murphy et al., 1989, Biochem. J., 258:285-293, Taylor et al., 1990, Planta, 181:18-26). Oil-body proteins have been identified in a wide range of taxonomically diverse species (Moreau et al., 1980, Plant Physiol., 65:1176-1180; Qu et al., 1986, Biochem. J., 235:57-65) and have been shown to be uniquely localized in oil-bodies and not found in organelles of vegetative tissues. In Brassica napus (rapeseed, canola) there are at least three polypeptides associated with the oil-bodies of developing seeds (Taylor et al., 1990, Planta, 181:18-26).

One of the most abundant proteins associated with the phospholipid monolayer membrane of oil bodies are the oleosins. The first oleosin gene, L3, was cloned from maize by selecting clones whose in vitro translated products were recognized by an anti-L3 antibody (Vance et al. 1987 J. Biol. Chem. 262:11275-11279). Subsequently, different isoforms of oleosin genes from such different species as Brassica, soybean, carrot, pine, and Arabidopsis have been cloned (Huang, A. H. C., 1992, Ann. Reviews Plant Phys. and Plant Mol. Biol. 43:177-200; Kirik et al., 1996 Plant Mol. Biol. 31:413-417; Van Rooijen et al., 1992 Plant Mol. Biol. 18:1177-1179; Zou et al., Plant Mol. Biol. 31:429-433. Oleosin protein sequences predicted from these genes are highly conserved, especially for the central hydrophobic domain. All of these oleosins have the characteristic feature of three distinctive domains. An amphipathic domain of 40-60 amino acids is present at the N-terminus; a totally hydrophobic domain of 68-74 amino acids is located at the center; and an amphipathic .alpha.-helical domain of 33-40 amino acids is situated at the C-terminus (Huang, A. H. C. 1992).

A maize oleosin has been expressed in seed oil bodies in Brassica napus transformed with a Zea mays oleosin gene. The gene was expressed under the control of regulatory elements from a Brassica gene encoding napin, a major seed storage protein. The temporal regulation and tissue specificity of expression was reported to be correct for a napin gene promoter/terminator (Lee et al., 1991, Proc. Natl. Acad. Sci. U.S.A., 88:6181-6185).

By providing a subset of genes encoding oleosins, it is now possible to modify the oleosin content in the phospholipid monolayer membrane of oil bodies by either introducing the genes provided herein into a plant and overexpressing said gene in said plant or, in the alternative, by down-regulating expression of the endogenous oleosin encoding genes in the plant using method known in the art including anti-sense or dsRNAi techniques.

In one specific embodiment, the present invention thus relates to a polynucleotide comprising a nucleotide sequence encoding an oleosin protein, which nucleotide sequence is substantially similar to a nucleic acid sequence encoding a polypeptide as given in SEQ ID NOs: 258 and 260.

More specifically, the invention relates to a polynucleotide comprising a nucleotide sequence encoding an oleosin protein, which is up-regulated during grain filling and has at least between 70%, and 99% amino acid sequence identity to at least one polypeptide of SEQ ID NOs: 258 and 260, with any individual number within this range of between 70% and 99% also being part of the invention.

The invention further relates to a polynucleotide comprising a nucleotide sequence encoding an oleosin protein, which is up-regulated during grain filling and immunologically reactive with antibodies raised against a polypeptide of SEQ ID NOs: 258 and 260.

More particularly, the invention relates to a polynucleotide comprising a nucleotide sequence
a) as given in any one of SEQ ID NOs: 257 and 259 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 257 and 259, or the complement thereof;
e) complementary to (a), (b) or (c); and
f) which is the reverse complement of (a), (b) or (c).

At least one of the genes provided herein, which is up-regulated during grain filling, encodes a phytoene dehydrogenase polypeptide that is involved in carotenoid biosynthesis and can thus be used to modify caroteinoid production in grain.

Carotenoids are natural pigments that are essential to microbial, plant, and animal life. In photosynthetic organisms, they act as potent antioxidants that negate the lethal effects of singlet oxygen and superoxide formed during oxygen production. As human dietary constituents, these lipophilic antioxidants provide our cells with chemical protectants against the damaging effects of oxidation. Acting as chemical scavengers, carotenoids play roles in the prevention of cancer and chronic maladies, including heart disease.

Phytoene (7,8,11,12,7′,8′,11′,12′-.omega. octahydro-.omega., omega.-carotene) is the first carotenoid in the carotenoid biosynthesis pathway and is produced by the dimerization of a 20-carbon atom precursor, geranylgeranyl pyrophosphate (GGPP). Phytoene has useful applications in treating skin disorders (U.S. Pat. No. 4,642,318) and is itself a precursor for colored carotenoids. Aside from certain mutant organisms, such as Phycomyces blakesleeanus carB, no current methods are available for producing phytoene via any biological process.

In some organisms, the red carotenoid lycopene (.omega.,.omega.-carotene) is the next carotenoid produced in the phytoene in the pathway. Lycopene imparts the characteristic red color to ripe tomatoes.

Lycopene has utility as a food colorant. It is also an intermediate in the biosynthesis of other carotenoids in some bacteria, fungi and green plants.

Lycopene is prepared biosynthetically from phytoene through four sequential dehydrogenation reactions by the removal of eight atoms of hydrogen. The enzymes that remove hydrogen from phytoene are phytoene dehydrogenases. One or more phytoene dehydrogenases can be used to convert phytoene to lycopene and dehydrogenated derivatives of phytoene intermediate to lycopene are also known. For example, some strains of Rhodobacter sphaeroides contain a phytoene dehydrogenase that removes six atoms of hydrogen from phytoene to produce neurosporene.

Lycopene is an intermediate in the biosynthesis of caaotenoids in some bacteria, fungi, and all green plants. Carotenoid-specific genes that can be used for synthesis of lycopene from the ubiquitous precursor farnesyl pyrophosphate include those for the enzymes GGPP synthase, phytoene synthase, and phytoene dehydrogenase-4H.

In one specific embodiment the present invention relates to a polynucleotide comprising a nucleotide sequence encoding a polypeptide the activity of which is involved in or associated with the dehydrogenation of phytoene and the expression of which is up-regulated during grain filling, which nucleotide sequence is substantially similar to a nucleic acid sequence encoding a polypeptide as given in SEQ ID NO: 278.

More specifically, the invention relates to a polynucleotide comprising a nucleotide sequence encoding a polypeptide the activity of which is involved in or associated with the dehydrogenation of phytoene and the expression of which is up-regulated during grain filling and which has at least between 70%, and 99/o amino acid sequence identity to at least one polypeptide of SEQ ID NOs: 278, with any individual number within this range of between 70% and 99% also being part of the invention.

The invention further relates to a polynucleotide comprising a nucleotide sequence encoding a polypeptide the activity of which is involved in or associated with the dehydrogenation of phytoene and the expression of which is up-regulated during grain filling and which is immunologically reactive with antibodies raised against a polypeptide of SEQ ID NOs: 278.

More particularly, the invention relates to a polynucleotide comprising a nucleotide sequence
a) as given in any one of SEQ ID NOs: 277 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 277, or the complement thereof;
e) complementary to (a), (b) or (c); and
f) which is the reverse complement of (a), (b) or (c).

Another subset of genes that is provided as part of the invention comprises nucleic acid molecules that are involved in the transcriptional control of the highly coordinated grain filling process.

Transcription factors are proteins that bind to the enhancer or promoter regions and interact such that transcription occurs from only a small group of promoters in any cell. Most transcription factors can bind to specific DNA sequences, and these trans-regulatory proteins can be grouped together in families based on similarities in structure. Within such a family, proteins share a common framework structure in their respective DNA-binding sites, and slight differences in the amino acids at the binding site can alter the sequence of the DNA to which it binds. In addition to having this sequence-specific DNA-binding domain, transcription factors contain a domain involved in activating the transcription of the gene whose promoter or enhancer it has bound. Usually, this trans-activating domain enables that transcription factor to interact with proteins involved in binding RNA polymerase. This interaction often enhances the efficiency with which the basal transcriptional complex can be built and bind RNA polymerase E. There are several families of transcription factors, and those discussed here are just some of the main types.

The gene subset provided herein includes a gene which encodes a polypeptide that is similar to the CREB-binding protein from Mus sp (as represented by SEQ ID NO: 301), and is highly expressed in aleurone and endosperm tissues during grain filling. CREB-binding protein (CBP) is a necessary component of the CREB/PKA paradigm of gene regulation. The acetylation of histones and other proteins has been linked to gene regulation, and CBP has a potent intrinsic acetyltransferase (AT) enzymatic domain. CREB belongs to a class of proteins whose phosphorylation appears specifically to enhance their trans-activation potential (Arias J, et al Nature 1994 Jul. 21;370(6486):226-9).

CBP possesses intrinsic histone acetyltransferase activity, and can acetylate not only histones but also certain transcriptional factors such as GATA1; p53 and also myb-type transcription factors such as c-Myb (Yuji Sano and Shunsuke Ishii J. Biol. Chem., Vol. 276, Issue 5, 3674-3682, Feb. 2, 2001). Acetylation of c-Myb by CBP increases the trans-activating capacity of c-Myb by enhancing its association with CBP. These results demonstrate a novel molecular mechanism of regulation of c-Myb activity.

In rice, 70 known and putative MYB genes could be identified, some of which show interesting expression patterns such as those given in SEQ ID NOs: 311-321. The expression pattern of these transcription factors suggests that they play a key role during rice grain filling.

Another transcription factor gene (as represented by SEQ ID NOs: 305) included in this subset encodes a protein that has structural similarity to the yeast HAP5 transcriptional activator protein. In yeast, the HAP5 protein is a component of the HAP (Hap2p-Hap3p-Hap4pHap5p) CCAAT-box-binding transcriptional activation complex and is essential for the binding activity of the complex.

A further transcription factor gene within this subset is represented by SEQ ID NO: 307 which encodes a bZIP-type transcription factor similar to the plant G-box binding factor GBF4, that was found in Arabidopsis. GBF4, in a manner reminiscent of the Fos-related oncoproteins of mammalian systems, cannot bind to DNA as a homodimer, although it contains a basic region capable of specifically recognizing the G-box and G-box-like elements. However, GBF4 can interact with GBF2 and GBF3 to bind DNA as heterodimers. Mutagenesis of the leucine zipper of GBF4 indicates that the mutation of a single amino acid confers upon the protein the ability to recognize the G-box as a homodimer, apparently by altering the charge distribution within the leucine zipper (A E Menkens and A R Cashmore (1994) PNAS 91: 2522-2526).

Another of the transcription factor genes within this subset encodes a protein that has a zinc finger domain and is similar to a zinc-finger type transcription factor found in Arabidopsis (gi|6899934).

Zinc finger proteins include WT-1 (a important transcription factor critical in the formation of the kidney and gonads); the ubiquitous transcription factor Sp1; Xenopus 5S rRNA transcription factor TFIIIA; Krox 20 (a protein that regulates gene expression in the developing hindbrain); Egr-1 (which commits white blood cell development to the macrophage lineage); Krippel (a protein that specifes abdominal cells in Drosophila); and numerous steroid-binding transcription factors. Each of these proteins has two or more “DNA-binding fingers,” a-helical domains whose central amino acids tend to be basic. These domains are linked together in tandem and are each stabilized by a centrally located zinc ion coordinated by two cysteines (at the base of the helix) and two internal histidines. The crystal structure shows that the zinc fingers bind in the major groove of the DNA.

The expression pattern of these transcription factors during grain filling suggests that they play a key role during rice grain development. This is further supported by the fact that the AACA promoter element, which is known to be conserved in many seed storage protein genes, is over-represented in the promoters of the grain filling sub-set genes according to the invention. This subset comprises genes the protein products of which are involved in diverse cellular functions, including carbohydrate, protein and fatty acid metabolism, nutrient transportation, and transcription and translation. The ACCA promoter element was thus demonstrated to be likely one of the key elements in the coordination of different major pathways during grain development.

In one embodiment the invention thus relates to a polynucleotide comprising a nucleotide sequence that encodes a polypeptide that acts as a transcription factor and the expression of which is up-regulates during grain filling, which nucleotide sequence is substantially similar to a nucleic acid sequence encoding a polypeptide as given in any one of the SEQ ID NOs of table 11 such as SEQ ID NOs: 302-328.

More specifically, the invention relates to a polynucleotide comprising a nucleotide sequence encodes a polypeptide that acts as a transcription factor and the expression of which is up-regulated during grain filling and which has at least between 700%, and 99% amino acid sequence identity to at least one polypeptide as given in any one of the SEQ ID NOs of table 11 such as SEQ ID NOs: 302-328, with any individual number within this range of between 70% and 99% also being part of the invention.

The invention further relates to a polynucleotide comprising a nucleotide sequence encodes a polypeptide that acts as a transcription factor and the expression of which is up-regulated during grain filling and which is immunologically reactive with antibodies raised against a polypeptide as given in any one of the SEQ ID NOs of table 11 such as SEQ ID NOs: 302-328.

More particularly, the invention relates to a polynucleotide comprising a nucleotide sequence
a) as given in any one of the SEQ ID NOs of table 11 such as SEQ ID NOs: 301-327 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in any one of the SEQ ID NOs of table 11 such as SEQ ID NOs: 301-327, or the complement thereof;
e) complementary to (a), (b) or (c); and
f) which is the reverse complement of (a), (b) or (c).

By changing the expression level and/or pattern of at least one transcription factor as provided herein, which is involved in the regulation and coordination of grain filling in plants, it is possible to modify the grain filling process to obtain grain with a modified nutritional composition and/or quality characteristics.

A further subset of genes which is provided herein comprises genes encoding polypeptides the activity of which is involved in or associated with amino acid metabolism.

In particular, the invention relates to a polynucleotide comprising a nucleotide sequence encoding a polypeptide the activity of which is involved or associated with the metabolism of amino acids and the expression of which is up-regulated during grain filling, which nucleotide sequence is substantially similar to a nucleic acid sequence encoding a polypeptide as given in any one of the SEQ ID NOs of table 10 such as SEQ ID NOs: 282-300.

More specifically, the invention relates to a polynucleotide comprising a nucleotide sequence encoding a polypeptide the activity of which is involved or associated with the metabolism of amino acids and the expression of which is up-regulated during grain filling, which polypeptide has at least between 70%, and 99% amino acid sequence identity to at least one polypeptide as given in any one of the SEQ ID NOs of table 10 such as SEQ ID NOs: 282-300, with any individual number within this range of between 70% and 99% also being part of the invention.

The invention further relates to a polynucleotide comprising a nucleotide sequence encoding a polypeptide the activity of which is involved or associated with the metabolism of amino acids and the expression of which is up-regulated during grain filling, which polypeptide is immunologically reactive with antibodies raised against a polypeptide as given in any one of the SEQ ID NOs of table 10 such as SEQ ID NOs: 282-300.

More particularly, the invention relates to a polynucleotide comprising a nucleotide sequence
a) as given in any one of the SEQ ID NOs of table 10 such as SEQ ID NOs: 281-299 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence as given in any one of the SEQ ID NOs of table 10 such as SEQ ID NOs: 281-299, or the complement thereof;
e) complementary to (a), (b) or (c); and

f) which is the reverse complement of (a), (b) or (c).

In a final embodiment, the present invention provides a subset of genes encoding polypeptides for which no biological function is known so far. It is within the scope of this invention, that the expression products of these genes, respresentative examples of which are provided in column B of table 3, can for the first time be associated with a biological function. Based on their mRNA expression characteristics and their specific expression pattern during grain filling it is suggested that they are involved in or associated with nutrient partitioning during the grain filling process.

By modifying the expression of at least one of the genes within this subgroup it is, therefore, possible to modify the compositional characteristics and thus the nutritional properties of the plant grain.

The present invention provides a set of genes, which were shown to be preferentially up-regulated and to share a similar expression pattern during the process of grain filling as specified hereinbefore. The genes within this subgroup are useful tools for generating plants which produce grain with modified compositional characteristics leading to improved nutritional properties.

According to one embodiment, the present invention is directed to a nucleic acid molecule comprising a nucleotide sequence isolated or obtained from any plant which encodes a polypeptide that has at least 70% amino acid sequence identity to a polypeptide encoded by a gene comprising any one of SEQ ID NOs provided in the Sequence Listing.

Based on the Oryza nucleic acid sequences of the present invention as given in the SEQ ID NOs of the Sequence Listing, orthologs may be identified or isolated from the genome of any desired organism, preferably from another plant, according to well known techniques based on their sequence similarity to the Orya nucleic acid sequences, e.g., hybridization, PCR or computer generated sequence comparisons. For example, all or a portion of a particular Oryza nucleic acid sequence is used as a probe that selectively hybridizes to other gene sequences present in a population of cloned genomic DNA fragments or cDNA fragments (i.e., genomic or cDNA libraries) from a chosen source organism. Further, suitable genomic and cDNA libraries may be prepared from any cell or tissue of an organism. Such techniques include hybridization screening of plated DNA libraries (either plaques or colonies; see, e.g., Sambrook et al., 1989) and amplification by PCR using oligonucleotide primers preferably corresponding to sequence domains conserved among related polypeptide or subsequences of the nucleotide sequences provided herein (see, e.g., Innis et al., 1990). These methods are particularly well suited to the isolation of gene sequences from organisms closely related to the organism from which the probe sequence is derived. The application of these methods using the Oryza sequences as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by is labeling synthetic oligonucleotides based on the sequence of the invention. Methods for preparation of probes for hybridization and for construction of cDNA and genomic libraries are generally known in the art and are disclosed in Sambrook et al. (1989). In general, sequences that hybridize to the sequences disclosed herein will have at least 40% to 50%, about 60% to 70% and even about 80% 85%, 90%, 95% to 98% or more identity with the disclosed sequences. That is, the sequence similarity of sequences may range, sharing at least about 40% to 50%, about 60% to 70%, and even about 80%, 85%, 900/0, 95% to 98% sequence similarity, with each individual number within the ranges given above also being part of the invention.

The nucleic acid molecules of the invention can also be identified by, for example, a search of known databases for genes encoding polypeptides having a specified amino acid sequence identity or DNA having a specified nucleotide sequence identity. Methods of alignment of sequences for comparison are well known in the art and are described hereinabove.

In a further embodiment, the invention provides isolated nucleic acid molecules comprising a plant nucleotide sequence that induces transcription of a linked nucleic acid segment in a plant or plant cell, e.g., a linked nucleic acid molecule comprising an open reading frame for or encoding a structural or regulatory gene, in a tissue specific or tissue preferential manner.

In a specific embodiment, the invention provides isolated nucleic acid molecules comprising a plant nucleotide sequence that induces transcription of a linked nucleic acid segment in a plant or plant cell, e.g., a linked nucleic acid molecule comprising an open reading frame for or encoding a structural or regulatory gene, in a seed-specific or seed-preferential manner. In particular, the plant nucleotide sequence according to the invention is substantially less active in vegetative tissue as compared to seed and is most active in the endosperm. The transcription inducing activity icreases during seed development and reaches its peak at or around the time of grain filling.

In particular, the nucleotide sequence of the invention directs seeds- (e.g. endosperm) specific or seeds- (e.g. endosperm) preferential transcription of a linked nucleic acid segment in a plant or plant cell and is preferably obtained or obtainable from plant genomic DNA having a gene comprising an open reading frame (ORF) encoding a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, and 99%, amino acid sequence identity, to a polypeptide encoded by an Oryza, e.g., Oryza sativa, gene comprising any one of SEQ ID NOs: 2-462 (e.g., including a promoter obtained or obtainable from any one of SEQ ID NOs: 643-883) which directs seed-specific (or seed-preferential) transcription of a linked nucleic acid segment.

The promoters of the invention include a consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, of any one of SEQ ID NOs: 643-883, or the promoter orthologs thereof, which include the minimal promoter region.

In a particular embodiment of the invention said consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, has at least 75%, preferably 80%, more preferably 90% and most preferably 95%, nucleic acid sequence identity with a corresponding consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, of any one of SEQ ID NOs: 643-883 or the promoter orthologs thereof, which include the minimal promoter region. The above defined stretch of contiguous nucleotides preferably comprises one or more promoter motifs, e.g., for seed-specific promoters, motifs selected from the group consisting of the P box and GCNA elements, including but not limited to TGTAAAG and TGA(G/C)TCA and a transcription start site.

In case of promoters directing tissue-specific transcription of a linked nucleic acid segment in a plant or plant cell such as, for example, a promoter directing seed-specific or seed-preferential, but especially endosperm-specific or endosperm-preferential transcription, it is further preferred that previously defined stretch of contiguous nucleotides comprises further motifs that participate in the tissue specificity of said stretch(es) of nucleotides.

Generally, the promoters of the invention may be employed to express a nucleic acid segment that is operably linked to said promoter such as, for example, an open reading frame, or a portion is thereof, an anti-sense sequence, or a transgene in plants. The open reading frame may be obtained from an insect resistance gene, a disease resistance gene such as, for example, a bacterial disease resistance gene, a fungal disease resistance gene, a viral disease resistance gene, a nematode disease resistance gene, a herbicide resistance gene, a gene affecting grain composition or quality, a nutrient utilization gene, a mycotoxin reduction gene, a male sterility gene, a selectable marker gene, a screenable marker gene, a negative selectable marker, a positive selectable marker, a gene affecting plant agronomic characteristics, i.e., yield, standability, and the like, or an environment or resistance gene, i.e., one or more genes that confer herbicide resistance or tolerance, insect resistance or tolerance, disease resistance or tolerance (viral, bacterial, fungal, oomycete, or nematode), stress tolerance or resistance (as exemplified by resistance or tolerance to drought, heat, chilling, fleezing, excessive moisture, salt stress, or oxidative stress), increased yields, food content and makeup, physical appearance, male sterility, drydown, standability, prolificacy, starch properties or quantity, oil quantity and quality, amino acid or protein composition, and the like. By “resistant” is meant a plant which exhibits substantially no phenotypic changes as a consequence of agent administration, infection with a pathogen, or exposure to stress. By “tolerant” is meant a plant which, although it may exhibit some phenotypic changes as a consequence of infection, does not have a substantially decreased reproductive capacity or substantially altered metabolism.

For instance, seed-specific promoters may be useful for expressing genes as well as for producing large quantities of protein, for expressing oils or proteins of interest, e.g., antibodies, genes for increasing the nutritional value of the seed and the like. In particular, the seed-specific or seed-preferential promoters accroding to the invention such as those provided in SEQ ID NOs: 643-883 may be useful for expressing the Open Reading Frames which are represented by the nucleotide sequences of SEQ ID NOs: 1-461 and 501-511, respectively.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

It is specifically contemplated by the present invention that one could use any one of the promoters according to the present invention in unaltered or altered form. Mutagenization of a promoter of the present invention such as those provided in SEQ ID NOs: 643-883 may potentially improve the utility of the elements for the expression of transgenes in plants. The mutagenesis of these elements can be carried out at random and the mutagenized promoter sequences screened for activity in a trial-by-error procedure.

Alternatively, particular sequences which provide the promoter with desirable expression characteristics, or the promoter with expression enhancement activity, could be identified and these or similar sequences introduced into the sequences via mutation. It is further contemplated that one could mutagenize these sequences in order to enhance their expression of transgenes in a particular species.

The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding un-modified sequences.

Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art.

Double stranded plasmids also are routinely employed in site directed mutagenesis which eliminates the step of transferring the gene of interest from a plasmid to a phage.

In general, site-directed mutagenesis in accordance herewith is performed by first obtaining a single-stranded vector or melting apart of two strands of a double stranded vector which includes within its sequence a DNA sequence which encodes the promoter. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation.

This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme is devised by Kunkel et al. (1987) to enrich for clones incorporating mutagenic oligonucleotides. Alternatively, the use of PCR with commercially available thermostable enzymes such as Taq polymerase may be used to incorporate a mutagenic oligonucleotide primer into an amplified DNA fragment that can then be cloned into an appropriate cloning or expression vector. The PCR-mediated mutagenesis procedures of Tomic et al. (1990) and Upender et al. (1995) provide two examples of such protocols. A PCR employing a thermostable ligase in addition to a thermostable polymerase also may be used to incorporate a phosphorylated mutagenic oligonucleotide into an amplified DNA fragment that may then be cloned into an appropriate cloning or expression vector. The mutagenesis procedure described by Michael (1994) provides an example of one such protocol.

The preparation of sequence variants of the selected promoter-encoding DNA segments using site-directed mutagenesis is provided as a means of producing potentially useful species and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Ramstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224. A number of template dependent processes are available to amplify the target sequences of interest present in a sample, such methods being well known in the art and specifically disclosed herein below.

Where a clone comprising a promoter has been isolated in accordance with the instant invention, one may wish to delimit the essential promoter regions within the clone. One efficient, targeted means for preparing mutagenizing promoters relies upon the identification of putative regulatory elements within the promoter sequence. This can be initiated by comparison with promoter sequences known to be expressed in similar tissue-specific or developmentally unique manner. Sequences which are shared among promoters with similar expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter sequence is provided, any of a number of different deletion mutants of the starting promoter could be readily prepared.

As indicated above, deletion mutants, deletion mutants of the promoter of the invention also could be randomly prepared and then assayed. With this strategy, a series of constructs are prepared, each containing a different portion of the clone (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters.

The present invention further provides a composition, an expression cassette or a recombinant vector containing the nucleic acid molecule of the invention as discosed herinbefore, and host cells comprising the expression cassette or vector, e.g., comprising a plasmid.

In particular, the present invention provides an expression cassette or a recombinant vector comprising a suitable promoter linked to a nucleic acid segment of the invention, representative examples of which are provided in the SEQ ID NOs of the Sequence Listing, which, when present in a plant, plant cell or plant tissue, results in transcription of the linked nucleic acid segment.

Promoters which are useful for plant transgene expression include those that are inducible, viral, synthetic, constitutive (Odell et al., 1985), temporally regulated, spatially regulated, tissue-specific, and spatio-temporally regulated.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

Suitable promoter and/or regulatory sequences further include those that are preferentially or specifically active in plant grain tissue such as, for example, the grain endosperm or the grain embryo.

Further, the invention provides isolated polypeptides encoded by any one of the open reading frames of the invention, representative examples of which are provided in the SEQ ID NOs of the Sequence Listing, or a fragment thereof, which encodes a polypeptide which has substantially the same activity as the corresponding polypeptide encoded by an ORF given in the SEQ ID NOs of the Sequence Listing, or the orthologs thereof.

Virtually any DNA composition may be used for delivery to recipient plant cells, e.g., monocotyledonous cells, to ultimately produce fertile transgenic plants in accordance with the present invention. For example, DNA segments or fragments in the form of vectors and plasmids, or linear DNA segments or fragments, in some instances containing only the DNA element to be expressed in the plant, and the like, may be employed. The construction of vectors which may be employed in conjunction with the present invention will be known to those of skill of the art in light of the present disclosure (see, e.g., Sambrook et al., 1989; Gelvin et al., 1990).

It is one of the objects of the present invention to provide recombinant DNA molecules comprising a nucleotide sequence which directs transcription according to the invention operably linked to a nucleic acid segment or sequence of interest.

The nucleic acid segment of interest can, for example, code for a ribosomal RNA, an antisense RNA or any other type of RNA that is not translated into protein. In another preferred embodiment of the invention, the nucleic acid segment of interest is translated into a protein product. The nucleotide sequence which directs transcription and/or the nucleic acid segment may be of homologous or heterologous origin with respect to the plant to be transformed. A recombinant DNA molecule useful for introduction into plant cells includes that which has been derived or isolated from any source, that may be subsequently characterized as to structure, size and/or function, chemically altered, and later introduced into plants. An example of a nucleotide sequence or segment of interest “derived” from a source, would be a nucleotide sequence or segment that is identified as a useful fragment within a given organism, and which is then chemically synthesized in essentially pure form. An example of such a nucleotide sequence or segment of interest “isolated” from a source, would be nucleotide sequence or segment that is excised or removed from said source by chemical means, e.g., by the use of restriction endonucleases, so that it can be further manipulated, e.g., amplified, for use in the invention, by the methodology of genetic engineering. Such a nucleotide sequence or segment is commonly referred to as “recombinant.”

Therefore a useful nucleotide sequence, segment or fragment of interest includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that is involved in carbohydrate metabolism or any other gene of interest as provided in the SEQ ID NOs of the sequence listing.

The introduced recombinant DNA molecule includes but is not limited to, DNA from plant genes, and non-plant genes such as those from bacteria, yeasts, animals or viruses. The introduced DNA can include modified genes, portions of genes, or chimeric genes, including genes from the same or different genotype. The term “chimeric gene” or “chimeric DNA” is defined as a gene or DNA sequence or segment comprising at least two DNA sequences or segments from species which do not combine DNA under natural conditions, or which DNA sequences or segments are positioned or linked in a manner which does not normally occur in the native genome of untransformed plant.

The introduced recombinant DNA molecule used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant.

Generally, the introduced recombinant DNA molecule will be relatively small, i.e., less than about 30 kb to minimize any susceptibility to physical, chemical, or enzymatic degradation which is known to increase as the size of the nucleotide molecule increases. As noted above, the number of proteins, RNA transcripts or mixtures thereof which is introduced into the plant genome is preferably preselected and defined, e.g., from one to about 5-10 such products of the introduced DNA may be formed.

This expression cassette or vector may be contained in a host cell. The expression cassette or vector may augment the genome of a transformed plant or may be maintained extrachromosomally. The expression cassette may be operatively linked to a structural gene, the open reading frame thereof, or a portion thereof. The expression cassette may further comprise a Ti plasmid and be contained in an Agrobacterium tumefaciens cell; it may be carried on a microparticle, wherein the microparticle is suitable for ballistic transformation of a plant cell; or it may be contained in a plant cell or protoplast. Further, the expression cassette or vector can be contained in a transformed plant or cells thereof, and the plant may be a dicot or a monocot. In particular, the plant may be a cereal plant.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

For example, for overexpression, a plant promoter fragment may be employed which will direct expression of the gene in all tissue; of a regenerated plant. Such promoters are referred to herein as “constitutive” promoters and are active under most environmental conditions and states of development or cell differentiation. Examples of constitutive promoters include the cauliflower mosaic virus (CaMV) ³⁵S transcription initiation region, the 1′- or 2′-promoter derived from T-DNA of Agrobacterium tumafaciens, and other transcription initiation regions from various plant genes known to those of skill. Such genes include for example, the AP2 gene, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol. 208:551-565 (1989)), and Gpc2 from maize (GenBank No. U45855, Manjunath et al., Plant Mol. Biol. 33:97-112 (1997)).

Alternatively, the plant promoter may direct expression of the nucleic acid molecules of the invention in a specific tissue or may be otherwise under more precise environmental or developmental control. Examples of environmental conditions that may effect transcription by inducible promoters include anaerobic conditions, elevated temperature, or the presence of light. Such promoters are referred to here as “inducible” or “tissue-specific” promoters. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of promoters under developmental control include promoters that initiate transcription only (or primarily only) in certain tissues, such as fruit, seeds, or flowers. Promoters that direct expression of nucleic acids in ovules, flowers or seeds are particularly useful in the present invention. As used herein a seed-specific or preferential promoter is one which directs expression specifically or preferentially in seed tissues, such promoters may be, for example, ovule-specific, embryo-specific, endosperm-specific, integument-specific, seed coat-specific, or some combination thereof. Examples include a promoter from the ovule-specific BEL1 gene described in Reiser et al. Cell 83:735-742 (1995) (GenBank No. U39944). Other suitable seed specific promoters are derived from the following genes: MAC1 from maize (Sheridan et al. Genetics 142:1009-1020 (1996), Cat3 from maize (GenBank No. L05934, Abler et al., Plant Mol. Biol. 22:10131-1038 (1993), the gene encoding oleosin 18 kD from maize (GenBank No, J05212, Lee et al., Plant Mol. Biol. 26:1981-1987 (1994)), vivparous-1 from Arabidopsis (Genbank No. U93215), the gene encoding oleosin from Arabidopsis (Genbank No. Z17657), Atmycl from Arabidopsis (Urao et al., Plant Mol. Biol. 32:571-576 (1996), the 2 s seed storage protein gene family from Arabidopsis (Conceicao et al. Plant 5:493-505 (1994)) the gene encoding oleosin 20 kD from Brassica napus (GenBank No. M63985), napA from Brassica napus (GenBank No. J02798, Josefsson et al. JBL 26:12196-1301 (1987), the napin gene family from Brassica napus (Sjodahl et al. Planta 197:264-271 (1995), the gene encoding the 2 S storage protein from Brassica napus (Dasgupta et al. Gene 133:301-302 (1993)), the genes encoding oleosin A (Genbank No. U09118) and oleosin B (Genbank No. U09119) from soybean and the gene encoding low molecular weight sulphur rich protein from soybean (Choi et al. Mol Gen, Genet. 246:266-268 (1995)).

It is specifically contemplated that one could use one of the promoters that are disclosed in co-pending provisional U.S. application Ser. No. 60/325,448, filed Sep. 26, 2001 in unaltered or altered form. Especially preferred are promoters that direct transcription of an associated nucleic acid molecule specifically or preferentially in tissues of the plant grain such as those provided in SEQ ID NOs: 2275-2672.

Mutagenization of a promoter such as those mentioned hereinbefore or those provided in provisional U.S. application Ser. No. 60/325,448 may potentially improve the utility of the elements for the expression of transgenes in plants. The mutagenesis of these elements can be carried out at random and the mutagenized promoter sequences screened for activity in a trial-by-error procedure.

Alternatively, particular sequences which provide the promoter with desirable expression characteristics, or the promoter with expression enhancement activity, could be identified and these or similar sequences introduced into the sequences via mutation. It is further contemplated that one could mutagenize these sequences in order to enhance their expression of transgenes in a particular species.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters.

A variety of 5N and 3N transcriptional regulatory sequences are available for use in the present invention. Transcriptional terminators are responsible for the termination of transcription and correct mRNA polyadenylation. The 3N nontranslated regulatory DNA sequence preferably includes from about 50 to about 1,000, more preferably about 100 to about 1,000, nucleotide base pairs and contains plant transcriptional and translational termination sequences. Appropriate transcriptional terminators and those which are known to function in plants include the CaMV 35S terminator, the tml terminator, the nopaline synthase terminator, the pea rbcS E9 terminator, the terminator for the T7 transcript from the octopine synthase gene of Agrobacterium tumefaciens, and the 3N end of the protease inhibitor 1 or 11 genes from potato or tomato, although other 3N elements known to those of skill in the art can also be employed. Alternatively, one also could use a gamma coixin, oleosin 3 or other terminator from the genus Coix.

Preferred 3N elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor 1 or 11 genes from potato or tomato.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will be most preferred.

Other sequences that have been found to enhance gene expression in transgenic plants include intron sequences (e.g., from Adh1, bronze1, actin1, actin 2 (WO 00/760067), or the sucrose synthase intron) and viral leader sequences (e.g., from TMV, MCMV and AMV). For example, a number of non-translated leader sequences derived from viruses are known to enhance expression. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include but are not limited to: Picornavirus leaders, for example, EMCV leader (Encephalomyocarditis 5 noncoding region) (Elroy-Stein et al., 1989); Potyvirus leaders, for example, TEV leader (Tobacco Etch Virus); MDMV leader (Maize Dwarf Mosaic Virus); Human immunoglobulin heavy-chain binding protein (BiP) leader, (Macejak et al., 1991); Untranslated leader from the coat protein mRNA of alfalfa mosaic virus (AMV RNA 4), (Jobling et al., 1987; Tobacco mosaic virus leader (TMV), (Gallie et al., 1989; and Maize Chlorotic Mottle Virus leader (MCMV) (Lommel et al., 1991. See also, Della-Cioppa et al., 1987.

Regulatory elements such as Adh intron 1 (Callis et al., 1987), sucrose synthase intron (Vasil et al., 1989) or TMV omega element (Gallie, et al., 1989), may further be included where desired.

Examples of enhancers include elements from the CaMV 35S promoter, octopine synthase genes (Ellis et al., 1987), the rice actin I gene, the maize alcohol dehydrogenase gene (Callis et al., 1987), the maize shrunken I gene (Vasil et al., 1989), TMV omega element (Gallie et al., 1989) and promoters from non-plant eukaryotes (e.g. yeast; Ma et al., 1988).

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”. Both of these methods lead to an inhibition of expression of the target gene.

Within the scope of the present invention, the alteration in expression of the nucleic acid molecule of the present invention may be achieved in one of the following ways:

(1) “Sense” Suppression

Alteration of the expression of a nucleotide sequence of the present invention, preferably reduction of its expression, is obtained by “sense” suppression (referenced in e.g. Jorgensen et al. (1996) Plant Mol. Biol. 31, 957-973). In this case, the entirety or a portion of a nucleotide sequence of the present invention is comprised in a DNA molecule. The DNA molecule is preferably operatively linked to a promoter functional in a cell comprising the target gene, preferably a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a preferred embodiment, the nucleotide sequence is fully translatable and all the genetic information comprised in the nucleotide sequence, or portion thereof, is translated into a polypeptide. In another preferred embodiment, the nucleotide sequence is partially translatable and a short peptide is translated. In a preferred embodiment, this is achieved by inserting at least one premature stop codon in the nucleotide sequence, which bring translation to a halt. In another more preferred embodiment, the nucleotide sequence is transcribed but no translation product is being made. This is usually achieved by removing the start codon, e.g. the “ATG”, of the polypeptide encoded by the nucleotide sequence. In a further preferred embodiment, the DNA molecule comprising the nucleotide sequence, or a portion thereof, is stably integrated in the genome of the plant cell. In another preferred embodiment, the DNA molecule comprising the nucleotide sequence, or a portion thereof, is comprised in an extrachromosomally replicating molecule. In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(2) “Antisense” Suppression

In another preferred embodiment, the alteration of the expression of a nucleotide sequence of the present invention, preferably the reduction of its expression is obtained by “anti-sense” suppression. The entirety or a portion of a nucleotide sequence of the present invention is comprised in a DNA molecule. The DNA molecule is preferably operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes noncoding strand) of the nucleotide sequence can be transcribed. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem. 55:569-597 (1986); van der Krol, A. R. et al, Antisense Nuc. Acids & Proteins, pp. 125-141 (1991); Abel, P. P. et al., Proc. Natl. Acad. Sci. USA 86:6949-6952 (1989); Ecker, J. R. et al., Proc. Natl. Acad. Sci. USA 83:5372-5376 (August 1986)).

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(3) Homologous Recombination

In another preferred embodiment, at least one genomic copy corresponding to a nucleotide sequence of the present invention is modified in the genome of the plant by homologous recombination as further illustrated in Paszkowski et al., EMBO Journal 7:4021-26 (1988). This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature 389:802-803 (1997) and Miao and Lam, Plant J., 7:359-365 (1995).

In another preferred embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al. (1999) Proc. Natl. Acad. Sci. USA 96: 8768-8773.

(4) Ribozymes

In a further embodiment, the RNA coding for a polypeptide of the present invention is cleaved by a catalytic RNA, or ribozyme, specific for such RNA. The ribozyme is expressed in transgenic plants and results in reduced amounts of RNA coding for the polypeptide of the present invention in plant cells, thus leading to reduced amounts of polypeptide accumulated in the cells. This method is further illustrated in U.S. Pat. No. 4,987,071.

(5) Dominant-Negative Mutants

In another preferred embodiment, the activity of the polypeptide encoded by the nucleotide sequences of this invention is changed. This is achieved by expression of dominant negative mutants of the proteins in transgenic plants, leading to the loss of activity of the endogenous protein.

(6) Aptamers

In a further embodiment, the activity of polypeptide of the present invention is inhibited by expressing in transgenic plants nucleic acid ligands, so-called aptamers, which specifically bind to the protein. Aptamers are preferentially obtained by the SELEX (Systematic Evolution of Ligands by EXponential Enrichment) method. In the SELEX method, a candidate mixture of single stranded nucleic acids having regions of randomized sequence is contacted with the protein and those nucleic acids having an increased affinity to the target are partitioned from the remainder of the candidate mixture. The partitioned nucleic acids are amplified to yield a ligand enriched mixture. After several iterations a nucleic acid with optimal affinity to the polypeptide is obtained and is used for expression in transgenic plants. This method is further illustrated in U.S. Pat. No. 5,270,163.

(7) Zinc Finger Proteins

A zinc finger protein that binds a nucleotide sequence of the present invention or to its regulatory region is also used to alter expression of the nucleotide sequence. Preferably, transcription of the nucleotide sequence is reduced or increased. Zinc finger proteins are for example described in Beerli et al. (1998) PNAS 95:14628-14633, or in WO 95/19431, WO 98/54311, or WO 96/06166, all incorporated herein by reference in their entirety.

(8) dsRNA

Alteration of the expression of a nucleotide sequence of the present invention is also obtained by dsRNA interference as described for example in WO 99/32619, WO 99/53050 or WO 99/61631, all incorporated herein by reference in their entirety.

(9) Insertion of a DNA Molecule (Insertional Mutagenesis)

In another preferred embodiment, a DNA molecule is inserted into a chromosomal copy of a nucleotide sequence of the present invention, or into a regulatory region thereof. Preferably, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. An example of this method is set forth in Example 2. Methods of insertional mutagenesis using T-DNA, transposons, oligonucleotides or other methods known to those skilled in the art are also encompassed. Methods of using T-DNA and transposon for insertional mutagenesis are described in Winkler et al. (1989) Methods Mol. Biol. 82:129-136 and Martienssen (1998) PNAS 95:2021-2026, incorporated herein by reference in their entireties.

(10) Deletion Mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al, (1995) Plant J. 7:359.

In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al, (1998) Plant Cell, 10:155-169; Bruggemann et al., (1996) Plant J., 10:755-760; Redei and Koncz in Methods in Arabidopsis Research, World Scientific Press (1992), pp. 16-82). Deletion mutations in a gene of the present invention can be recovered in a reverse genetics strategy using PCR with pooled sets of genomic DNAs as has been shown in C. elegans (Liu et al., (1999), Genome Research, 9:859-867.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

(11) Overexpression in a Plant Cell

In yet another preferred embodiment, a nucleotide sequence of the present invention encoding a polypeptide comprising a 3′-5′ exonuclease domain and/or activity in a plant cell is overexpressed. Examples of nucleic acid molecules and expression cassettes for overexpression of a nucleic acid molecule of the present invention are described above. Methods known to those skilled in the art of over-expression of nucleic acid molecules are also encompassed by the present invention.

In still another embodiment, the expression of the nucleotide sequence of the present invention is altered in every cell of a plant. This is for example obtained though homologous recombination or by insertion in the chromosome. This is also for example obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for overexpression of a nucleotide sequence of the present invention, are prepared and transformed into a plant cell according to the teachings of the present invention, e.g. as described infra.

The invention hence also provides sense and anti-sense nucleic acid molecules corresponding to the open reading flames identified in the SEQ ID NOs of the Sequence Listing as well as their orthologs.

The genes and open reading frames according to the present invention which are substantially similar to a nucleotide sequence encoding a polypeptide as given in any one of the SEQ ID NOs of the Sequence Lisiting including any corresponding antisense constructs can be operably linked to any promoter that is functional within the plant host including the promoter sequences according to the invention or mutants thereof.

The present invention further provides a method of augmenting a plant genome by contacting plant cells with a nucleic acid molecule of the invention, e.g., one having a nucleotide sequence that directs tissue-specific, tissue-preferential transcription of a linked nucleic acid segment isolatable or obtained from a plant gene encoding a polypeptide that is substantially similar to a polypeptide encoded by the an Oryza gene having a sequence according to any one of SEQ ID NOs provided in the Sequence Listing so as to yield transformed plant cells; and regenerating the transformed plant cells to provide a differentiated transformed plant, wherein the differentiated transformed plant expresses the nucleic acid molecule in the cells of the plant, preferably in the appropriate tissues of the plant grain. The nucleic acid molecule may be present in the nucleus, chloroplast, mitochondria and/or plastid of the cells of the plant.

Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and nor-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell in planta or ex planta, including a transformed plastid or other organelle, e.g., nucleus, mitochondria or chloroplast. The present invention may be used for transformation of any plant species, including, but not limited to, cells from corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L.turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (Wl. ultila, Wl. ultilanen, Wl. gladiala, Wl. ultila, Wl. lingulata, Wl. repunda, Wl. rotunda, and Wl. neotropica). Any other genera or species of Lemnaceae, if they exist, are also aspects of the present invention. Lemna gibba, Lemnaminor, and Lemna miniscula are preferred, with Lemnaminor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiflung Rubel, Zurich (1986)).

Vegetables within the scope of the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Other plants within the scope of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Ornamental plants within the scope of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Other plants within the scope of the invention are shown in Table 1 (above).

Preferably, transgenic plants of the present invention are crop plants and in particular cereals (for example, corn, alfalfa, sunflower, rice, Brassica, canola, soybean, barley, soybean, sugarbeet, to cotton, safflower, peanut, sorghum, wheat, millet, tobacco, etc.), and even more preferably corn, rice and soybean.

The present invention also provides a transgenic plant prepared by this method, a seed from such a plant and progeny plants from such a plant including hybrids and inbreds. Preferred transgenic plants are transgenic maize, soybean, barley, alfalfa, sunflower, canola, soybean, cotton, peanut, sorghum, tobacco, sugarbeet, rice, wheat, rye, turfgrass, millet, sugarcane, tomato, or potato.

A transformed (transgenic) plant of the invention includes plants, the genome of which is augmented by a nucleic acid molecule of the invention, or in which the corresponding gene has been disrupted, e.g., to result in a loss, a decrease or an alteration, in the function of the product encoded by the gene, which plant may also have increased yields and/or produce a better-quality product than the corresponding wild-type plant. The nucleic acid molecules of the invention are thus useful for targeted gene disruption, as well as markers and probes.

The invention also provides a method of plant breeding, e.g., to prepare a crossed fertile transgenic plant. The method comprises crossing a fertile transgenic plant comprising a particular nucleic acid molecule of the invention with itself or with a second plant, e.g., one lacking the particular nucleic acid molecule, to prepare the seed of a crossed fertile transgenic plant comprising the particular nucleic acid molecule. The seed is then planted to obtain a crossed fertile transgenic plant. The plant may be a monocot or a dicot. In a particular embodiment, the plant is a cereal plant.

The crossed fertile transgenic plant may have the particular nucleic acid molecule inherited through a female parent or through a male parent. The second plant may be an inbred plant. The crossed fertile transgenic may be a hybrid. Also included within the present invention are seeds of any of these crossed fertile transgenic plants.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Preferably expression vectors are introduced into intact tissue. General methods of culturing plant tissues are provided for example by Maki et al., (1993); and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues, (Lindsey et al., 1993; Auch & Reth et al.).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., 1985: Byrne et al., 1987; Sukhapinda et al., 1987; Lorz et al., 1985; Potrykus, 1985; Park et al., 1985: Hiei et al., 1994). The use of T-DNA to transform plant cells has received extensive study and is amply described (EP 120516; Hoekema, 1985; Knauf, et al., 1983; and An et al., 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm et al., 1986) or high velocity ballistic bombardment with metal particles coated with the nucleic acid constructs (Kine et al., 1987, and U.S. Pat. No. 4,945,050). Once transformed, the cells can be regenerated by those skilled in the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1989; Christou et al., 1989; EP 301749), rice (Hiei et al., 1994), and corn (Gordon Kamm et al., 1990; Fromm et al., 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc., Madison, Wis. And BioRad, Hercules, Calif. (see, for example, Sanford et al., U.S. Pat. No. 4,945,050; and McCabe et al., 1988). Also see, Weissinger et al., 1988; Sanford et al., 1987 (onion); Christou et al., 1988 (soybean); McCabe et al., 1988 (soybean); Datta et al., 1990 (rice); Klein et al., 1988 (maize); Klein et al., 1988 (maize); Klein et al., 1988 (maize); Fromm et al., 1990 (maize); and Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (European Patent Application EP 0 292 435, U.S. Pat. No. 5,350,689).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application no. WO 95/16783, and in McBride et al., 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., 1990; Staub et al., 1992). This resulted in stable homoplasmic as transformants at a frequency of approximately one per 100 bombardments of target leaves. The presence of cloning sites between these markers allowed creation of a plastid targeting vector for introduction of foreign genes (Staub et al., 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3N-adenyltransferase (Svab et al., 1993). Other selectable markers useful for plastid transformation are known in the art and encompassed within the scope of the invention. Typically, approximately 15-20 cell division cycles following transformation are required to reach a homoplastidic state. Plastid expression, in which genes are inserted by orthologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

For example, vectors are available for transformation using Agrobacterium tumefaciens. These typically carry at least one T-DNA border sequence and include vectors such as pBIN19 (Bevan, 1984). In one preferred embodiment, the expression cassettes of the present invention may be inserted into either of the binary vectors pCIB200 and pCIB2001 for use with Agrobacterium. These vector cassettes for Agrobacterium-mediated transformation wear constructed in the following manner. PTJS75kan was created by Narl digestion of pTJS75 (Schmidhauser & Helinski, 1985) allowing excision of the tetracycline-resistance gene, followed by insertion of an Accl fragment from pUC4K carrying an NPTh (Messing & Vierra, 1982; Bevan et al., 1983; McBride et al., 1990). XhoI linkers were ligated to the EcoRV fragment of pCIB7 which contains the left and right T-DNA borders, a plant selectable nos/nptII chimeric gene and the pUC polylinker (Rothstein et al., 1987), and the XhoI-digested fragment was cloned into SalI-digested pTJS75kan to create pCIB200 (see also EP 0 332 104, example 19). PCIB200 contains the following unique polylinker restriction sites: EcoRI, SstI, KpnI, BgfII, XbaI, and SalI. The plasmid pCIB2001 is a derivative of pCIB200 which was created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. PCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OnT and OriV functions also from RK2. The pCIB2001 polylinker is suitable for the cloning of plant expression cassettes containing their own regulatory signals.

An additional vector useful for Agrobacterium-mediated transformation is the binary vector pCIB 10, which contains a gene encoding kanamycin resistance for selection in plants, T-DNA right and left border sequences and incorporates sequences from the wide host-range plasmid pRK252 allowing it to replicate in both E. coli and Agrobacterium. Its construction is described by Rothstein et al., 1987. Various derivatives of pCIB10 have been constructed which incorporate the gene for hygromycin B phosphotransferase described by Gritz et al., 1983. These derivatives enable selection of transgenic plant cells on hygromycin only (pCIB743), or hygromycin and kananycin (pCIB715, pCIB717).

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptlI gene which confers resistance to kanamycin and related antibiotics (Messing & Vierra, 1982; Bevan et al., 1983), the bar gene which confers resistance to the herbicide phosphinothricin (White et al., 1990, Spencer et al., 1990), the hph gene which confers resistance to the antibiotic hygromycin (Blochinger & Diggelmann), and the dhfr gene, which confers resistance to methotrexate (Bourouis et al., 1983).

Selection markers resulting in positive selection, such as a phosphomannose isomerase gene, as described in patent application WO 93/05163, are also used. Other genes to be used for positive selection are described in WO 94/20627 and encode xyloisomerases and phosphomanno-isomerases such as mannose-6-phosphate isomerase and mannose-1-phosphate isomerase; phosphomanno mutase; mannose epimerases such as those which convert carbohydrates to mannose or mannose to carbohydrates such as glucose or galactose; phosphatases such as mannose or xylose phosphatase, mannose-6-phosphatase and mannose-1-phosphatase, and permeases which are involved in the transport of mannose, or a derivative, or a precursor thereof into the cell. The agent which reduces the toxicity of the compound to the cells is typically a glucose derivative such as methyl-3-O-glucose or phloridzin. Transformed cells are identified without damaging or killing the non-transformed cells in the population and without co-introduction of antibiotic or herbicide resistance genes. As described in WO 93/05163, in addition to the fact that the need for antibiotic or herbicide resistance genes is eliminated, it has been shown that the positive selection method is often far more efficient than traditional negative selection.

One vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278, herein incorporated by reference. One gene useful for conferring resistance to phosphinothricin is the bar gene from Streptomyces viridochromogenes (Thompson et al., 1987). This vector is suitable for the cloning of plant expression cassettes containing their own regulatory signals.

An additional transformation vector is pSOG35 which utilizes the E. coli gene dihydrofolate reductase (DHFR) as a selectable marker conferring resistance to methotrexate. PCR was used to amplify the 35S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene was also amplified by PCR and these two PCR fragments were assembled with a SacI-PstI fragment from pB1221 (Clontech) which comprised the pUC19 vector backbone and the nopaline synthase terminator. Assembly of these fragments generated pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

Binary backbone vector pNOV2117 contains the T-DNA portion flanked by the right and left border sequences, and including the Positcch™ (Syngenta) plant selectable marker and the “grain filling candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function; plant part assays, such as seed assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The presence of nucleic acid elements introduced through the methods of this invention may be determined by polymerase chain reaction (PCR). Using this technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced preselected DNA segment.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer et al., 1992); Laursen et al., 1994) indicating stable inheritance of the gene. The nonchimeric nature of the callus and the parental transformants (R₀) was suggested by germline transmission and the identical Southern blot hybridization patterns and intensities of the transforming DNA in callus, R₀ plants and R₁ progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical chemical, structural, functional, or other properties of the proteins. Unique physicachemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

The compositions of the invention include plant nucleic acid molecules, and the amino acid sequences for the polypeptides or partial-length polypeptides encoded by the nucleic acid molecule which comprises an open reading frame. These sequences can be employed to alter expression of a particular gene corresponding to the open reading frame by decreasing or eliminating expression of that plant gene or by overexpressing a particular gene product. Methods of this embodiment of the invention include stably transforming a plant with the nucleic acid molecule of the invention which includes an open reading frame operably linked to a promoter capable of driving expression of that open reading frame (sense or antisense) in a plant cell. By “portion” or “fragment”, as it relates to a nucleic acid molecule which comprises an open reading frame or a fragment thereof encoding a partial-length polypeptide having the activity of the full length polypeptide, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention. Thus, to express a particular gene product, the method comprises introducing to a plant, plant cell, or plant tissue an expression cassette comprising a promoter linked to an open reading frame so as to yield a transformed differentiated plant, transformed cell or transformed tissue. Transformed cells or tissue can be regenerated to provide a transformed differentiated plant. The transformed differentiated plant or cells thereof preferably expresses the open reading frame in an amount that alters the amount of the gene product in the plant or cells thereof, which product is encoded by the open reading frame. The present invention also provides a transformed plant prepared by the method, progeny and seed thereof.

The invention further includes a nucleotide sequence which is complementary to one (hereinafter “test” sequence) which hybridizes under stringent conditions with a nucleic acid molecule of the invention as well as RNA which is transcribed from the nucleic acid molecule. When the hybridization is performed under stringent conditions, either the test or nucleic acid molecule of invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree to of stringency required such reduced concentration buffers are typically single strength SC containing 0.1% SDS, half strength SC containing 0.1% SDS and one-tenth strength SC containing 0.1% SDS.

In a further embodiment, the present invention provides a transformed plant host cell, or one obtained through breeding, capable of over-expressing, under-expressing, or having a knock out of amino acid genes and/or their gene products. The plant cell is transformed with at least one such expression vector wherein the plant host cell can be used to regenerate plant tissue or an entire plant, or seed there from, in which the effects of expression, including overexpression or underexpression, of the introduced sequence or sequences can be measured in vitro or in planta.

Polynucleotides derived from the nucleic acid molecules of the present invention having any of the nucleotide sequences of SEQ ID NO: 1 to 461 and 501 to 511, respectively, encoding a polypeptide the expression of which is up-regulated during grain filling, are useful to detect the presence in a test sample of at least one copy of a nucleotide sequence containing the same or substantially the same sequence, or a fragment, complement, or variant thereof. The sequence of the probes and/or primers of the instant invention need not be identical to those provided in the Sequence Listing or the complements thereof. Some variation in probe or primer sequence and/or length can allow additional family members to be detected, as well as orthologous genes and more taxonomically distant related sequences. Similarly probes and/or primers of the invention can include additional nucleotides that serve as a label for detecting duplexes, for isolation of duplexed polynucleotides, or for cloning purposes.

Preferred probes and primers of the invention include isolated, purified, or recombinant polynucleotides containing a contiguous span of between at least 12 to at least 1000 nucleotides of any nucleotid sequence which is substantially similar, and preferably has at least between 70% and 99% sequence identity to any one of SEQ ID NO: 1 to 461, 501-511, and 513-641, respectively, encoding a polypeptide the expression of which is up-regulated during grain filing, or the complements thereof, with each individual number of nucleotides within this range also being part of the invention. Preferred are isolated, purified, or recombinant polynucleotides containing a contiguous span of at least 12, 15, 18, 20, 25, 30, 35, 40, 50, 60, 70, 80, 90, 100, 150, 200, 300, 400, 500, 750, or 1000 nucleotides of any nucleotide sequence which is substantially similar, and preferably has at least between 70% and 99%, sequence identity to any one of SEQ ID NO: 1 to 461, 501-511, and 513-641, respectively, encoding a polypeptide the expression of which is up-regulated during grain filling, or the complements thereof. The appropriate length for primers and probes will vary depending on the application. For use as PCR primers, probes are 12-40 nucleotides, preferably 18-30 nucleotides long. For use in mapping, probes are 50 to 500 nucleotides, preferably 100-250 nucleotides long. For use in Southern hybridizations, probes as long as several kilobases can be used. The appropriate length for primers and probes under a particular set of assay conditions may be empirically determined by one of skill in the art.

The primers and probes can be prepared by any suitable method, including, for example, cloning and restriction of appropriate sequences and direct chemical synthesis by a method such as the phosphodiester method of Narang et al. (Meth Enzymol 68: 90 (1979)), the diethylphosphoramidite method, the triester method of Matteucci et al. (J Am Chem Soc 103: 3185 (1981)), or according to Urdea et al. (Proc Natl Acad 80: 7461 (1981)), the solid support method described in EP 0 707 592, or using commercially available automated oligonucleotide synthesizers.

Detection probes are generally nucleotide sequences or uncharged nucleotide analogs such as, for example peptide nucleotides which are disclosed in International Patent Application WO 92/20702, morpholino analogs which are described in U.S. Pat. Nos. 5,185,444, 5,034,506 and 5,142,047. The probe may have to be rendered “non-extendable” such that additional dNTPs cannot be added to the probe. Analogs are usually nonextendable, and nucleotide probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified so as to render the probe non-extendable.

Any of the polynucleotides of the present invention can be labeled, if desired, by incorporating a label detectable by spectroscopic, photochemical, biochemical, immunochemical, or chemical means. For example, useful labels include radioactive substances (³²P, ³⁵S, ³H, ²⁵I), fluorescent dyes (5-bromodesoxyuridine, fluorescein, acetylaminofluorene, digoxigenin) or biotin. Preferably, polynucleotides are labeled at their 3′ and 5′ ends. Examples of non-radioactive labeling of nucleotide fragments are described in the French patent No. FR-7810975 and by Urdea et al. (Nuc Acids Res 16:4937 (1988)). In addition, the probes according to the present invention may have structural characteristics such that they allow the signal amplification, such structural characteristics being, for example, branched DNA probes as described in EP 0 225 807.

A label can also be used to capture the primer so as to facilitate the immobilization of either the primer or a primer extension product, such as amplified DNA, on a solid support. A capture label is attached to the primers or probes and can be a specific binding member that forms a binding pair with the solid's phase reagent's specific binding member, for example biotin and streptavidin. Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleotide sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “ail” that is not complementary to the target. In the case where a polynucleotide primer itself serves as the capture label, at least a portion of the primer will be free to hybridize with a nucleotide on a solid phase. DNA labeling techniques are well known in the art.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleotides on solid phases include ionic, hydrophobic, covalent interactions and the like. A solid support, as used herein, refers to any material that is insoluble, or can be made insoluble by a subsequent reaction. The solid support can be chosen for its intrinsic ability to attract and immobilize the capture reagent. Alternatively, the solid phase can retain an additional receptor that has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

The polynucleotides of the invention that are expressed or repressed in response to environmental stimuli such as, for example, biotic or abiotic stress or treatment with chemicals or pathogens or at different developmental stages can be identified by employing an array of nucleic acid samples, e.g., each sample having a plurality of oligonucleotides, and each plurality corresponding to a different plant gene, on a solid substrate, e.g., a DNA chip, and probes corresponding to nucleic acid expressed in, for example, one or more plant tissues and/or at one or more developmental stages, e.g., probes corresponding to nucleic acid expressed in seed of a plant relative to control nucleic acid from sources other than seed. Thus, genes that are upregulated or downregulated in the majority of tissues at a majority of developmental stages, or upregulated or downregulated in one tissue such as in seed, can be systematically identified. The probes may also correspond to nucleic acid expressed in respone to a defined treatment such as, for example, a treatment with a variety of plant hormones or the exposure to specific environmental conditions involving, for example, an abiotic stress or exposure to light.

Specifically, labeled rice cRNA probes were hybridized to the rice DNA array, expression levels were determined by laser scanning and then rice genes were identified that had a particular expression pattern. The rice oligonucleotide probe array consists of probes from over 18,000 unique rice genes, which covers approximately 40-50% of the genome. This genome array permits a broader, more complete and less biased analysis of gene expression.

As described herein, GeneChip® technology was utilized to discover rice genes that are preferentially (or exclusively) expressed during the grain filling process in specific tissues of the plant grain such as, for example, the aleurone, embryo, endosperm, seed coat, etc.

Using this approach, 461 genes were identified, the expression of which was specifically elevated during the grain filling process.

Consequently, the invention also deals with a method for detecting the presence of a polynucleotide including a nucleotide sequence which is substantially similar, and preferably has at least between 70% and 99% sequence identity to any one of SEQ ID NO: 1 to 461, 501-511, and 513-641, respectively, encoding a polypeptide the expression of which is up-regulated during grain filing, or a fragment or a variant thereof, or a complementary sequence thereto in a sample, the method including the following steps of:
(a) bringing into contact a nucleotide probe or a plurality of nucleotide probes which can hybridize with polynucleotide having a nucleotide sequence which is substantially similar, and preferably has at least between 70% and 99% sequence identity to any one of SEQ ID NO: 1 to 461, 501-511, and 513-641, respectively, encoding a polypeptide the expression of which is up-regulated during grain filling, or a fragment or a variant thereof, or a complementary sequence thereto and the sample to be assayed.
(b) detecting the hybrid complex formed between the probe and a nucleotide in the sample.

The invention further concerns a kit for detecting the presence of a polynucleotide including a nucleotide sequence which is substantially similar, and preferably has at least between 70% and 99% sequence identity to any one of SEQ ID NO: 1 to 461, 501-511, and 513-641, respectively, encoding a polypeptide the expression of which is up-regulated during grain filling, or a fragment or a variant thereof, or a complementary sequence thereto in a sample, the kit including a nucleotide probe or a plurality of nucleotide probes which can hybridize with a nucleotide sequence included in a polynucleotide including a nucleotide sequence which is substantially similar, and preferably has at least between 70% and 99% sequence identity to any one of SEQ ID NO: 1 to 461, 501-511, and 513-641, respectively, encoding a polypeptide the expression of which is up-regulated during grain filing, or a fragment or a variant thereof, or a complementary sequence thereto and, optionally, the reagents necessary for performing the hybridization reaction.

In a first preferred embodiment of this detection method and kit, the nucleotide probe or the plurality of nucleotide probes are labeled with a detectable molecule. In a second preferred embodiment of the method and kit, the nucleotide probe or the plurality of nucleotide probes has been immobilized on a substrate.

The isolated polynucleotides of the invention can be used to create various types of genetic and physical maps of the genome of rice or other plants. Such maps are used to devise positional cloning strategies for isolating novel genes from the mapped crop species. The sequences of the present invention are also useful for chromosome mapping, chromosome identification, tagging of genes that are involved in the grain filling process.

The isolated polynucleotides of the invention can further be used as probes for identifying polymorphisms associated with phenotypes of interest such as, for example, enhanced phosphate utilization, and higher yield. Briefly, total DNA is isolated from an individual or isogenic line, cleaved with one or more restriction enzymes, separated according to mass, transferred to a solid support, and hybridized with a probe molecule according to the invention. The pattern of fragments hybridizing to a probe molecule is compared for DNA from different individuals or lines, where differences in fragment size signals a polymorphism associated with a particular nucleotide sequence according to the present invention. After identification of polymorphic sequences, linkage studies can be conducted. After identification of many polymorphisms using a nucleotide sequence according to the invention, linkage studies can be conducted by using the individuals showing polymorphisms as parents in crossing programs. Recombinants, F₂ progeny recombinants or recombinant inbreds, can then be analyzed using the same restriction enzyme/hybridization procedure. The order of DNA polymorphisms along the chromosomes can be inferred based on the frequency with which they are inherited together versus inherited independently. The closer together two polymorphisms occur in a chromosome, the higher the probability that they are inherited together. Integration of the relative positions of polymorphisms and associated marker sequences produces a genetic map of the species, where the distances between markers reflect the recombination frequencies in that chromosome segment. Preferably, the polymorphisms and marker sequences are sufficiently numerous to produce a genetic map of sufficiently high resolution to locate one or more loci of interest.

The use of recombinant inbred lines for such genetic mapping is described for rice (Oh et al, Mol Cells 8:175 (1998); Nandi et al, Mol Gen Genet 255:1 (1997); Wang et al, Genetics 136:1421 (1994)), sorghum (Subudhi et al, Genome 43:240 (2000)), maize (Burr et al., Genetics 118:519 (1998); Gardineret al, Genetics 134:917 (1993)), and Arabidopsis (Methods in Molecular Biology, Martinez-Zapater and Salinas, eds., 82:137-146, (1998)). However, this procedure is not limited to plants and can be used for other organisms such as yeast or other fungi, or for oomycetes or other protistans.

The nucleotide sequences of the present invention can also be used for simple sequence tppeat identification, also known as single sequence repeat, (SSR) mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within an sequence of the invention are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs were used to map a molecular marker closely linked to a nuclear restorer gene for fertility in rice as described by Akagi et al. (Genome 39:205 (1996)).

The nucleotide sequences of the present invention can be used to identify and develop a variety of microsatellite markers, including the SSRs described above, as genetic markers for comparative analysis and mapping of genomes. The nucleotide sequences of the present invention can be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers derived from the nucleotide sequences disclosed in the Sequence Listing, or substantially similar sequences or allelic variants thereof, may be used to detect the appearance or disappearance of markers indicating genomic instability as described by Leroy et al. (Electron. J. Biotechnol, 3(2), at http://www.ejb.org (2000)), where alteration of a fingerprinting pattern indicated loss of a marker corresponding to a part of a gene involved in the regulation of cell proliferation. Microsatellite markers derived from nucleotide sequences as provided in the Sequence Listing will be useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, the nucleotide sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

The various types of maps discussed above can be used with the nucleotide sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved regulating the content and composition of the plant grain, enables marker-assisted breeding to enhance the nutritional value of the grain or suppress undesirable traits that interfere with an efficient grain filling process. The nucleotide sequences as provided in the Sequence Listing can be used to generate markers, including single-sequence repeats (SSRs) and microsatellite markers for QTLs and utilization to assist marker-assisted breeding. The nucleotide sequences of the invention can be used to identify QTLs regulating the grain filling process and isolate alleles as described by Li et al. in a study of QTLs involved in resistance to a pathogen of rice. (Li et al., Mol Gen Genet 261:58 (1999)). In addition to isolating QTL alleles in rice, other cereals, and other monocot and dicot crop species, the nucleotide sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

In another embodiment the nucleotide sequences of the invention can be used to help create physical maps of the genome of maize, Arabidopsis and related species. Where the nucleotide sequences of the invention have been ordered on a genetic map, as described above, then the nucleotide sequences of the invention can be used as probes to discover which clones in large libraries of plant DNA fragments in YACs, PACs, etc. contain the same nucleotide sequences of the invention or similar sequences, thereby facilitating the assignment of the large DNA fragments to chromosomal positions. Subsequently, the large BACs, YACs, etc. can be ordered unambiguously by more detailed studies of their sequence composition and by using their end or other sequence to find the identical sequences in other cloned DNA fragments (Mozo et al., Nat Genet 22:271 (1999)). Overlapping DNA sequences in this way allows assembly of large sequence contigs that, when sufficiently extended, provide a complete physical map of a chromosome. The nucleotide sequences of the invention themselves may provide the means of joining cloned sequences into a contig, and are useful for constructing physical maps.

In another embodiment, the nucleotide sequences of the present invention may be useful in mapping and characterizing the genomes of other cereals. Rice has been proposed as a model for cereal genome analysis Havukkala, Curr Opin Genet Devel 6:711 (1996)), based largely on its smaller genome size and higher gene density, combined with the considerable conserved gene order among cereal genomes (Ahn et al., Mol Gen Genet 241:483 (1993)). The cereals demonstrate both general conservation of gene order (synteny) and considerable sequence homology among various cereal gene families. This suggests that studies on the functions of genes or proteins from rice according to the present invention could lead to elucidation of the functions of orthologous genes or proteins in other cereals, including maize, wheat, secale, sorghum, barley, millet, teff, milo, triticale, flax, gramma grass, Tripsacum sp., and teosinte. The nucleotide sequences according to the invention can also be used to physically characterize homologous chromosomes in other cereals, as described by Sarma et al. (Genome 43:191 (2000)), and their use can be extended to non-cereal monocots such as sugarcane, grasses, and lilies.

Given the synteny between rice and other cereal genomes, the nucleotide sequences of the present invention can be used to obtain molecular markers for mapping and, potentially, for positional cloning. Kilian et al. described the use of probes from the rice genomic region of interest to isolate a saturating number of polymorphic markers in barley, which were shown to map to syntenic regions in rice and barley, suggesting that the nucleotide sequences of the invention derived from the rice genome would be useful in positional cloning of syntenic grain-filling genes of interest from other cereal species. (Kilian, et al., Nucl Acids Res 23:2729 (1995); Kilian, et al, Plant Mol Biol 35:187 (1997)). Synteny between rice and barley has recently been reported in the area of the carrying malting quality QTLs (Han, et al., Genome 41:373 (1998)), and use of synteny between cereals for positional cloning efforts is likely to add considerable value to rice genome analysis. Likewise, mapping of the ligules region of sorghum was facilitated using molecular markers from a syntenic region of the rice genome. (Zwick, et al., Genetics 148:1983 (1998)).

Rice marker technology utilizing the nucleotide sequences of the present invention can also be used to identify QTL alleles from a wild relative of cultivated rice, for example as described by Xiao, et al. (Genetics 150:899 (1998)). Wild relatives of domesticated plants represent untapped pools of genetic resources for abiotic and biotic stress resistance, apomixis and other breeding strategies, plant architecture, determinants of yield, secondary metabolites, and other valuable traits. In rice, Xiao et al. (supra) used molecular markers to introduce an average of approximately 5% of the genome of a wild relative, and the resulting plants were scored for phenotypes such as plant height, panicle length and 1000-grain weight. Trait-improving alleles were found for all phenotypes except plant height, where any change is considered negative. Of the 35 trait-improving alleles, Xiao et al. found that 19 had no effect on other phenotypes whereas 16 had deleterious effects on other traits. The nucleotide sequences of the invention such as those provided in the Sequence Listing can be employed as molecular markers to identify QTL alleles involved in the regulation of the grain filling process from a wild relative, by which these valuable traits can be introgressed from wild relatives using methods including, but not limited to, that described by Xiao et al. ((1998) supra). Accordingly, the nucleotide sequences of the invention can be employed in a variety of molecular marker technologies for yield improvement.

Following the procedures described above to identify polymorphisms, and using a plurality of the nucleotide sequences of the invention, any individual (or line) can be genotyped. Genotyping a large number of DNA polymorphisms such as single nucleotide polymorphisms (SNPs), in breeding lines makes it possible to find associations between certain polymorphisms or groups of polymorphisms, and certain phenotypes. In addition to sequence polymorphisms, length polymorphisms such as triplet repeats are studied to find associations between polymorphism and phenotype. Genotypes can be used for the identification of particular cultivars, varieties, lines, ecotypes, and genetically modified plants or can serve as tools for subsequent genetic studies of complex traits involving multiple phenotypes.

The patent publication WO95/35505 and U.S. Pat. Nos. 5,445,943 and 5,410,270 describe scanning multiple alleles of a plurality of loci using hybridization to arrays of oligonucleotides. The nucleotide sequences of the invention are suitable for use in genotyping techniques useful for each of the types of mapping discussed above.

In a preferred embodiment, the nucleotide sequences of the invention are useful for identifying and isolating a least one unique stretch of protein-encoding nucleotide sequence. The nucleotide sequences of the invention are compared with other coding sequences having sequence similarity with the sequences provided in the Sequence Listing, using a program such as BLAST. Comparison of the nucleotide sequences of the invention with other similar coding sequences permits the identification of one or more unique stretches of coding sequences encoding polypeptides that are up-regulated during grain filling that are not identical to the corresponding coding sequence being screened. Preferably, a unique stretch of coding sequence of about 25 base pairs (bp) long is identified, more preferably 25 bp, or even more preferably 22 bp, or 20 bp, or yet even more preferably 18 bp or 16 bp or 14 bp. In one embodiment, a plurality of nucleotide sequences is is screened to identify unique coding sequences accroding to the invention. In one embodiment, one or more unique coding sequences accroding to the invention can be applied to a chip as part of an array, or used in a non-chip array system. In a further embodiment, a plurality of unique coding sequences accroding to the invention is used in a screening array. In another embodiment, one or more unique coding sequences accroding to the invention can be used as immobilized or as probes in solution. In yet another embodiment, one or more unique coding sequences accroding to the invention can be used as primers for PCR. In a further embodiment, one or more unique coding sequences accroding to the invention can be used as organism specific primers for PCR in a solution containing DNA from a plurality of sources.

In another embodiment unique stretches of nucleotide sequences according to the invention are identified that are preferably about 30 bp, more preferably 50 bp or 75 bp, yet more preferably 100 bp, 150 bp, 200 bp, 250, 500 bp, 750 bp, or 1000 bp. The length of an unique coding sequence may be chosen by one of skill in the art depending on its intended use and on the characteristics of the nucleotide sequence being used. In one embodiment, unique coding sequences accroding to the invention may be used as probes to screen libraries to find homologs, orthologs, or paralogs. In another embodiment, unique coding sequences accroding to the invention may be used as probes to screen genomic DNA or cDNA to find homologs, orthologs, or paralogs. In yet another embodiment, unique coding sequences accroding to the invention may be used to study gene evolution and genome evolution.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described in detail in Sambrook et al. (Molecular Cloning: A Laboratory Manual, Cold Spring Harbor Laboratory Press (1989)) and by Ausubel et al. (Current Protocols in Molecular Biology, Greene Publishing (1992)).

### Example 1

### Isolation and Sequencing of DNA Fragments

1.1 Isolation and Sequencing of Genomic DNA Fragments

Genomic DNA was isolated from nuclei of Oryza sativa L. ssp japonica cv Nipponbare and then sheared to produce fragments of approximately 500 bp. Using a method derived from the method of Mao et al. (Genome Res 10:982 (2000)), seeds were germinated on cheese cloth immersed in water and grown for 4-6 weeks under greenhouse conditions. After plants reached a height of approximately 5-8 inches, the upper parts of the green leaves were harvested and wrapped in aluminum foil at 4° C. overnight. Leaf material was then stored at −80° C. or directly used for extraction of nuclei. Intact nuclei were isolated by homogenization (in a blender for flesh material or by grinding with mortar and pestle for frozen material) in a buffer containing 10 mM Trizra base, 80 mM KCl, 10 mM EDTA, 1 mM spermidine, 1 mM spermine, 0.5 M sucrose, 0.5% Triton-X-100, 0.15% β-mercaptoethanol pH 9.5. The homogenate was filtered and nuclei recovered by gentle centrifugation using a fixed-angle rotor at 1,800 g at 4 C for 20 minutes. The pellet recovered after centrifugation was gently resuspended with the assistance of a small paint brush soaked in ice cold wash buffer and wash buffer added. Particulate matter remaining in the suspension was removed by filtering the resuspended nuclei into a 50 ml centrifuge tube through two layers of miracloth by gravity and centrifuging the filtrate at 57 g (500 rpm), 4 C for 2 minutes to remove intact cells and tissue residues. The supernatant fluid was transferred into a fresh centrifuge tube and nuclei were pelleted by centrifugation at 1,800 g, 4 C for 15 minutes in a swinging bucket centrifuge.

DNA was isolated from the nuclear preparation by phenolchloroform extraction, as in Sambrook et al (supra). Isolated total genomic DNA was physically sheared (Hydroshear) to generate for generating random DNA fragments, and fragments of approximately 500 bp were recovered. DNA was eluted and the ends filled in using T₄ DNA polymerase, Klenow fragments, and dNTPs. Double-stranded DNA was Tinkered and cloned into a Novartis proprietary medium-copy vector derived from pSC101.

Vector inserts were amplified by PCR and sequenced using the MegaBACE sequencing system (Molecular Dynamics, Amersham). The amplification reaction was diluted before use and was not purified using an exonuclease/alkaline phosphatase procedure. Sequencing reactions were performed using DYEnamic ET Terminator Kit. The reactions contained approximately 50 ng of amplicon, DYEnamic ET Terminator premix, and 5 pmol of −40 M13 forward primer. The sequencing reaction is amplified for 30 cycles, and reaction products are concentrated and purified using ethanol precipitation. The sample was electrokinetically injected into the capillary at 3 kV for 45 sec and separated via electrophoresis at 9 kV for 120 min.

1.2 Isolation and Sequencing of cDNA Fragments

Construction of rice cDNA library. Total RNA was purified from rice plant tissue using standard total RNA purification methods. PolyA+ RNA was isolated from the total RNA using the Qiagen Oligotex mRNA purification system (Qiagen, Valencia, Calif.), and cDNA was generated using cDNA synthesis reagents from Life Technologies (Rockville, Md.). First strand cDNA synthesis was catalyzed by reverse transcriptase using oligo dT primers with a NotI restriction site. Second strand synthesis was catalyzed by DNA polymerase. An oligonucleotide linker with a SalI restriction endonuclease site was attached to the 5′ end of the cDNAs using DNA ligase. The cDNAs were digested with NotI and SalI restriction endonucleases and inserted into an E. coli-replicating plasmid harboring a selectable marker. E. coli was transfected with the recombinant plasmids and grown on selectable media. E. coli colonies were individually picked off the selectable media and placed into storage plates.

Sequencing the rice cDNA library, The DNA sequence of the cDNA cloned into the plasmid purified from an E. coli colony was determined using standard dideoxy sequencing methods. Oligonucleotide primers respectively corresponding to plasmid DNA regions upstream of the 5′ end of the cDNA insert (Forward reaction) and downstream of the 3′ end of the cDNA insert (Reverse reaction) were used in the dideoxy sequencing reactions. If the DNA sequence determined as a result of the Forward and Reverse reactions from the cDNA overlapped, the two sequences could be merged into a contig using computerized analysis software (Consed, University of Washington, Seattle), to assemble a full-length sequence of the cDNA. In cases case where DNA sequence from the Forward and Reverse reactions from a single clone did not overlap sufficiently to be assembled into a contig, such that there was a region of unsequenced DNA to bridge the DNA from the Forward and Reverse reaction in order to form a contig, the DNA sequence of the separating region was determined using one of two dideoxy sequencing methods. In a “primer walking” approach, a primer specifically corresponding to the 3′ end of the DNA sequence determined from the Forward reaction was used in a second dedeoxy sequencing reaction. The primer walking procedure was repeated until the DNA sequence that separated the original Forward and Reverse was resolved and a contig could be assembled. Alternatively, the clone harboring the cDNA was subjected to transposon in vitro insertion dideoxysequencing (Epicentre, Madison, Wis.). In this procedure, the insertion process was random and the result was multiple DNA sequence coverage over the targeted cDNA, where the sequences thus obtained were assembled into a contig.

### Example 2

### GeneChip® Standard Protocol

The standard protocol for using the GeneChip® to quantitatively measure plant gene expression was carried out as outlined below:

Quantitation of total RNA

30 Total RNA from plant tissue was extracted and quantified. Quantified total RNA using
GeneQuant
IOD₂₆₀=40 mg RNA/ml; A₂₆₀/A₂₈₀=1.9 to about 2.1

2: Ran gel to check the integrity and purity of the extracted RNA

Synthesis of Double-Stranded cDNA

Gibco/BRL SuperScript Choice System for cDNA Synthesis (Cat#IB090-019) was employed to prepare cDNAs. T7-(dT)₂₄ oligonucleotides were prepared and purified by HPLC.

SEQ ID NO: 4709)

(5′- GGCCAGTGAATTGTAATACGACTCACTATAGGGAGGCGG-
(dT)₂₄-3′;.

Step 1. Primer Hybridization:
Incubated at 70° C. for 10 minutes
Spun quickly and put on ice briefly

Step 2. Temperature Adjustment:
Incubated at 42° C. for 2 minutes

Step 3. First Strand Synthesis Carried Out Using:
DEPC-water—1:1
RNA (10:g final)—10:1
T7=(dT)₂₄ Primer (100 pmol final)—1:1 pmol
5× 1^{st} strand cDNA buffer—4:1
0.1 M DTT(10 mM final)—2:1
10 mM dNTP mix (500:M final)—1:1
Superscript II RT 200 U/:l—1:1
Total of 20:1
Mixed well
Incubated at 42° C. for 1 hour

Step 4. Second Strand Synthesis:
Placed reactions on ice, quick spin
   DEPC-water—91:1
   5× 2^{nd} strand cDNA buffer—30:1
   10 mM dNTP mix (250 mM final)—3:1
   E. coli DNA ligase (10U/:1)—1:1
   E. coli DNA polymerase 1-10 U/:l—4:1
   RnaseH 2U/:l—1:1
   T4 DNA polymerase 5 U/:l—2:1
   0.5 M EDTA (0.5 M final)—10:1
   Total 162:1
Mixed/spun down/incubated 16° C. for 2 hours

Step 5. Completing the Reaction:
Incubated at 16° C. for 5 minutes
   Purification of Double Stranded cDNA
1. Centrifuged PLG (Phase Lock Gel, Eppendorf 5 Prime Inc., pl-188233) at 14,000×, transfered 162:1 of cDNA to PLG
2. Added 162:1 of Phenol:Chloroform:Isoamyl alcohol (pH 8.0), centrifuge 2 minutes

3. Transfered the supernatant to a fresh 1.5 ml tube, add

| | |
|---|---|
| Glycogen (5 mg/ml) | 2 |
| 0.5 M NR₄OAC (0.75 × Vol) | 120 |
| ETOH (2.5 × Vol, −20° C.) | 400 |

4. Mixed well and centrifuge at 14,000× for 20 minutes
5. Removed supernatant, added 0.5 ml 80% EtOH (−20° C.)
6. Centrifuged for 5 minutes, air dry or by speed vac for 5-10 minutes
7. Added 44:1 DEPC H₂O
   Analyzed quantity and size distribution of cDNA

Ran a gel using 1:1 ratio of the double-stranded synthesis product to loading Buffer

Synthesis of biotinylated cRNA
(used Enzo BioArray High Yield
RNA Transcript Labeling Kit Cat#900182)

| | |
|---|---|
| Purified cDNA | 22:1 |
| 10× Hy buffer | 4:1 |
| 10× biotin ribonucleotides | 4:1 |
| 10× DTT | 4:1 |
| 10× Rnase inhibitor mix | 4:1 |
| 20× T7 RNA polymerase | 2:1 |
| Total | 40:1 |

Centrifuged 5 seconds, and incubated for 4 hours at 37° C.

Gently mixed every 30-45 minutes

Purification and quantification of cRNA
(used Qiagen Rneasy Mini kit Cat# 74103)

| | | |
|---|---|---|
| cRNA | 40:1 | |
| DEPC H₂O | 60:1 | |
| RLT buffer | 350:1 | mix by vortexing |
| EtOH | 250:1 | mix by pipetting |
| Total | 700:1 | |

Waited 1 minute or more for the RNA to stick

Centrifuged at 2000 rpm for 5 minutes

| | |
|---|---|
| RPE buffer | 500:1 |

Centrifuged at 10,000 rpm for 1 minute

| | |
|---|---|
| RPE buffer | 500:1 |

Centrifuged at 10,000 rpm for 1 minute

Centrifuged at 10,000 rpm for 1 minute to dry the column

| | |
|---|---|
| DEPC H₂O | 30:1 |

Waited for 1 minute, then elute cRNA from by centrifugation, 10 K 1 minute

| | |
|---|---|
| DEPC H₂O | 30:1 |

Repeated previous step

Determined concentration and dilute to 1:g/:l concentration

Fragmentation of cRNA

| | |
|---|---|
| cRNA (1:g/:1) | 15:1 |
| 5× Fragmentation Buffer* | 6:1 |
| DEPC H₂O | 9:1 |
| | 30:1 |

*5× Fragmentation Buffer

| | | |
|---|---|---|
| 1 M Tris (pH8.1) | 4.0 | ml |
| MgOAc | 0.64 | g |
| KOAC | 0.98 | g |
| DEPC H₂O | | |
| Total | 20 | ml |
| Filter Sterilize | | |

Array washed and stained in:
Stringent Wash Buffer**
Non-Stringent Wash Buffer**
SAPE Stain****
Antibody Stain*****
   Washed on Fluidics Station Using the Appropriate Antibody Amplification Protocol
**Stringent Buffer. 12×MES 83.3 ml, 5 M NaCl 5.2 ml, 10% Tween 1.0 ml, H₂O 910 ml,
   Filter Sterilize
***Non-Stringent Buffer. 20×SSPE 300 ml, 10% Tween 1.0 ml, H₂O 698 ml, Filter Sterilize, Antifoam 1.0.
   ****SAPE stain: 2× Stain Buffer 600:1, BSA 48:1, SAPE 12:1, H₂O 540:1.
*****Antibody Stain: 2× Stain Buffer 300:1, H₂O 266.4:1, BSA 24:1, Goat IgG 6:1, Biotinylated Ab 3.6:1

### Example 3

### Profiling of Genes Involved in Nutrition partitioning During Grain Development

A GeneChip® Rice Genome Array (Affymetrix, Santa Clara, Calif.) was used to examine how accumulation of carbohydrates, storage protein and fatty acids is coordinated at RNA level during grain development.

RNA expression of three major pathways and associated genes involving nutrition partitioning was examined, including synthesis and transport of carbohydrates, proteins, and fatty acids. A total of 491 genes involved in these pathways were first selected based on their sequence annotation and functional classification. RNA expression was determined in 39 samples representing different developmental stages including samples collected before and during grain filling.

3.1 Plant Growth Conditions and Sampling

Nipponbare rice was grown in the greenhouse with 12 hr light cycle and temperature of 29° C. during the day and 21° C. during the night. Humidity was maintained at 30%. Plants were grown in pots containing 50% sunshine mix and 50% nitrohumus. The descriptions of the samples collected for this analysis are listed in table 1. Individual tissues were collected from a minimum of five plants and pooled. Total RNA was extracted from one gram of tissue using the Qiagen RNA Easy Maxikit (Qiagen, Valencia, Calif.).

The experiments were carried out as described in T. Zhu e al. Plant Physiol. Biochem. 39, 221 (2001).

TABLE 1
Rice samples included in the study of genes involved
in nutrition partitioning during grain development

| | Days after | developmental | | |
|---|---|---|---|---|
| Description | germination | stage | Rank | Category |
| germinating | 5 | 11 | 1 | root |
| seedling (root) | | | | |
| germinating | 5 | 12 | 1 | leaf |
| seedling [LEAF] | | | | |
| 3-4 leaf arial | 18 | 13 | 2 | arial |
| tillering | 49 | 14 | 3 | root |
| stage (root) | | | | |
| tillering | 49 | 15 | 3 | leaf |
| stage (leaf) | | | | |
| tillering | 49 | 16 | 3 | arial |
| stage (arial) | | | | |
| Booting Stage | 60 | 17 | 4 | repr |
| panicle 1-3 cm | | | | |
| Booting stage | 62 | 18 | 5 | repr |
| panicle 4-7 cm | | | | |
| Booting Stage | 64 | 19 | 6 | repr |
| panicle 8-14 cm | | | | |
| Booting Stage | 66 | 20 | 7 | repr |
| panicle 15-20 cm | | | | |
| Booting Stage root | 60 | 22 | 6 | root |
| Booting Stage leaf | 60 | 23 | 6 | leaf |
| Booting stage arial | 60 | 24 | 6 | arial |
| panicle emergence- | 78 | 25 | 8 | root |
| root | | | | |
| panicle emergence - | 78 | 26 | 8 | stem |
| stem | | | | |
| panicle emergence- | 78 | 21 | 8 | repr |
| panicle | | | | |
| Seed milk | 88 | 39 | | repr |
| stage [˜9DAF] | | | | |
| Seed -soft | 94 | 40 | 14 | repr |
| dough [˜14DAF] | | | | |
| Seed hard | 100 | 41 | 15 | repr |
| dough [˜21DAF] | | | | |
| inflorescence- no seeds | 88 | 30 | 9 | repr |
| maturation stem | 90 | 27 | 15 | stem |
| maturation root | 90 | 28 | 15 | root |
| maturation leaf | 90 | 29 | 15 | leaf |
| embryo | 88 | 42 | 14 | embryo |
| endosperm | 88 | 43 | 14 | endospm |
| seed coat | 88 | 44 | 14 | coat |
| Senescence -stem | 100 | 31 | 16 | stem |
| Senescence [LEAF] | 100 | 32 | 16 | leaf |
| aleurone | 88 | 45 | 14 | aleurone |
| pollen mixed | 55 | 33 | | pollen |
| seed day 0 | 79 | 34 | 9 | repr |
| post anthesis | | | | |
| seed day 2 | 81 | 35 | 10 | repr |
| post anthesis | | | | |
| seed day 4 | 83 | 36 | 11 | repr |
| post anthesis | | | | |
| seed day 7 | 86 | 37 | 12 | repr |
| post anthesis | | | | |
| seed day 8 | 87 | 38 | 13 | repr |
| post anthesis | | | | |

### Example 4

### Characterization of Gene Expression Profiles

4.1 Data Analysis 1

A rice gene array and probes derived from rice RNA extracted from different tissues and developmental stages were used to identify the expression profile of genes on the chip. The rice array contains over 23,000 genes (approximately 18,000 unique genes) or roughly 50% of the rice genome and is similar to the Arabidopsis GeneChip®) (Affymetrix) with the exception that the 16 oligonucleotide probe sets do not contain mismatch probe sets. The level of expression is therefore determined by internal software that analyzes the intensity level of the 16 probe sets for each gene. The highest and lowest probes are removed if they do not fit into a set of predefined statistical criteria and the remaining sets are averaged to give an expression value. The final expression values are normalized by software, as described below. The advantages of a gene chip in such an analysis include a global gene expression analysis, quantitative results, a highly reproducible system, and a higher sensitivity than Northern blot analyses.

4.2 Data Analysis II

Data analysis was done using GeneSpring (Silicon Genetics, Redwood, Calif.) and AlignAce. The genechip sequence was blasted to the AC rice contig sequences. The contig with the best alignment was extracted and five gene prediction programs were run on each contig. The programs used were Genscan trained on arabidopsis and maize, Gmhmm trained on rice and Arabidopsis, and Fgenesh and Glimmer trained on rice. All of the predicted CDSs were blasted against the genechip sequence again to extract the top hit predicted CDS. A Perl script was utilized to extract up to 2 kb of the putative promoter sequence. In some of the genechip sequences there was more than one perfect alignment to a predicted CDS; in such cases, both of the perfect alignments were accepted as the putative genes.

TABLE 2
Table 2 provides provides a subset of rice genes the expression
of which is up-regulated during grain filling. Further identified
are SSR sequences in the coding region of the rice genes.

A = Genes involved in rice grain filling, which belong to the functional category of
Carborhydrate Metabolism
B = Genes involved in rice grain filling, which belong to the functional category of
transmembrane proteins
C = Genes involved in rice grain filling, which belong to the functional category of storage
proteins
D = Genes involved in rice grain filling, which belong to the functional category of stress
response proteins
E = 345 Grain Filling Genes
F = Genes involved in rice grain filling, which belong to the functional category of signaling
molecules
G = Genes involved in rice grain filling, which belong to the functional category of
transcription factors
H = Genes involved in rice grain filling, which belong to the functional category of amino
acid Metabolism
I = Genes involved in rice grain filling, which belong to the functional category of Fatty Acid
Metabolism
J = Cereal_Grain_Filling_QTLs (a description of the respective QTLs is provided
in Table . . . below)
K = Beginning of the SSR
L = End of the SSR
M = Nucleotide Sequence of the tri- and tetra-nucleotide repeat units

| SEQ | | | | | | | | | | | | | |
|---|---|---|---|---|---|---|---|---|---|---|---|---|---|
| ID | A | B | C | D | E | F | G | H | I | J | K | L | M |
| 101 | X | — | — | — | X | — | — | — | — | | | | |
| 113 | X | — | — | — | X | — | — | — | — | | 42 | 59 | CCT |
| 1 | — | — | — | X | X | — | — | — | — | | | | |
| 317 | X | — | — | — | X | — | X | — | — | | | | |
| 329 | — | — | — | — | X | — | — | — | — | OS-FLLEN-9-1, | | | |
| | | | | | | | | | | OS-GPL-4-1, | | | |
| | | | | | | | | | | OS-GPP-4-1, | | | |
| | | | | | | | | | | OS-GW100-4-1, | | | |
| | | | | | | | | | | OS-GYLD-4-1 | | | |
| 173 | X | — | — | — | X | — | — | — | — | | | | |
| 331 | — | — | — | — | X | — | — | — | — | OS-GW-5-1 | 5 | 19 | CGG |
| | | | | | | | | | | OS-YLD-5-1, | | | |
| | | | | | | | | | | ZM-MOIST-4-3, | | | |
| | | | | | | | | | | ZM-DMY-4-3, | | | |
| | | | | | | | | | | ZM-YLD-4-1 | | | |
| 333 | — | — | — | — | X | — | — | — | — | | | | |
| 233 | — | — | X | — | X | — | — | — | — | | | | |
| 335 | — | — | — | — | X | — | — | — | — | | | | |
| 119 | X | — | — | — | X | — | — | — | — | | | | |
| 311 | X | — | — | — | X | — | X | — | — | | 358 | 372 | CGC |
| | | | | | | | | | | | 661 | 675 | CGG |
| 149 | X | — | — | — | X | — | — | — | — | | | | |
| 337 | — | — | — | — | X | — | — | — | — | | | | |
| 59 | — | X | — | — | X | — | — | — | — | | | | |
| 339 | — | — | — | — | X | — | — | — | — | | | | |
| 155 | X | — | — | — | X | — | — | — | — | | 1207 | 1221 | CTG |
| 143 | X | — | — | — | X | — | — | — | — | | | | |
| 307 | — | — | — | — | X | — | X | — | — | | 155 | 175 | CTG |
| 341 | — | — | — | — | X | — | — | — | — | | | | |
| 193 | X | — | — | — | X | — | — | — | — | SMS015-9, | 1401 | 1415 | CGT |
| | | | | | | | | | | ZM-MOIST-4-2, | | | |
| | | | | | | | | | | ZM-DMY-4-1 | | | |
| 131 | X | — | — | — | X | — | — | — | — | | | | |
| 199 | X | — | — | — | X | — | — | — | — | OS-AE-1-1, | 207 | 221 | CGC |
| | | | | | | | | | | OS-AE-5-1, | | | |
| | | | | | | | | | | OS-APDF-9-1, | | | |
| | | | | | | | | | | OS-REGEN-3-1, | | | |
| | | | | | | | | | | OS-RGT-5-1, | | | |
| | | | | | | | | | | OS-VGT-2-2, | | | |
| | | | | | | | | | | OS-VGT-5-1, | | | |
| | | | | | | | | | | OS-GC-2-1, | | | |
| | | | | | | | | | | OS-GYLD-1-1, | | | |
| | | | | | | | | | | SMS021-80, | | | |
| | | | | | | | | | | ZM-CPC-5-1, | | | |
| | | | | | | | | | | ZM-ID-5-1, | | | |
| | | | | | | | | | | ZM-IVDOM-5-1, | | | |
| | | | | | | | | | | ZM-IVDOM-5-2, | | | |
| | | | | | | | | | | ZM-IVDOM-5-3, | | | |
| | | | | | | | | | | ZM-MOIST-5-2, | | | |
| | | | | | | | | | | ZM-MOIST-5-2, | | | |
| | | | | | | | | | | ZM-MOIST-5-3, | | | |
| | | | | | | | | | | ZM-BIOM-5-1, | | | |
| | | | | | | | | | | ZM-DMC-6-2, | | | |
| | | | | | | | | | | ZM-DMY-5-1, | | | |
| | | | | | | | | | | ZM-GYLD-5-1, | | | |
| | | | | | | | | | | ZM-GYLD-5-3, | | | |
| | | | | | | | | | | ZM-GYLD-5-3, | | | |
| | | | | | | | | | | ZM-GYLD-6-4, | | | |
| | | | | | | | | | | ZM-GYLD-6-4, | | | |
| | | | | | | | | | | ZM-KW300-5-1, | | | |
| | | | | | | | | | | ZM-TW-5-1, | | | |
| | | | | | | | | | | ZM-YLD-6-1 | | | |
| 301 | — | — | — | — | X | — | X | — | — | OS-VGT-2-2, | | | |
| | | | | | | | | | | OS-GC-2-1 | | | |
| 343 | — | — | — | — | X | — | — | — | — | OS-FLLEN-3-1, | | | |
| | | | | | | | | | | OS-GPL-2-1, | | | |
| | | | | | | | | | | OS-GYLD-2-1, | | | |
| | | | | | | | | | | ZM-ID-5-2, | | | |
| | | | | | | | | | | ZM-MOIST-4-3, | | | |
| | | | | | | | | | | ZM-MOIST-5-4, | | | |
| | | | | | | | | | | ZM-PC-5-1, | | | |
| | | | | | | | | | | ZM-STC-5-1, | | | |
| | | | | | | | | | | ZM-DMC-5-1, | | | |
| | | | | | | | | | | ZM-DMY-4-3, | | | |
| | | | | | | | | | | ZM-GYLD-5-2 | | | |
| 287 | — | — | — | — | X | — | — | X | — | | | | |
| 191 | X | — | — | — | X | — | — | — | — | | | | |
| 215 | — | — | X | — | X | — | — | — | — | | 373 | 387 | TCG |
| | | | | | | | | | | | 972 | 986 | CCG |
| 23 | — | — | — | — | X | X | — | — | — | ZM-MOIST-2-3, | | | |
| | | | | | | | | | | ZM-STC-2-2, | | | |
| | | | | | | | | | | ZM-DMY-2-3, | | | |
| | | | | | | | | | | ZM-DMY-2-4, | | | |
| | | | | | | | | | | ZM-GYLD-2-3 | | | |
| 147 | X | — | — | — | X | — | — | — | — | | | | |
| 345 | — | — | — | — | X | — | — | — | — | | | | |
| 347 | X | — | — | — | X | — | — | — | — | OS-GPDF-1-1, | | | |
| | | | | | | | | | | SMS015-16, | | | |
| | | | | | | | | | | ZM-CL-9-1, | | | |
| | | | | | | | | | | ZM-CPC-3-1, | | | |
| | | | | | | | | | | ZM-CPC-3-3, | | | |
| | | | | | | | | | | ZM-CPC-8-1, | | | |
| | | | | | | | | | | ZM-ID-8-1, | | | |
| | | | | | | | | | | ZM-ID-8-1, | | | |
| | | | | | | | | | | ZM-ID-8-1, | | | |
| | | | | | | | | | | ZM-IVDOM-3-1, | | | |
| | | | | | | | | | | ZM-IVDOM-3-3, | | | |
| | | | | | | | | | | ZM-MOIST-8-1, | | | |
| | | | | | | | | | | ZM-MOIST-8-2, | | | |
| | | | | | | | | | | ZM-MOIST-9-2, | | | |
| | | | | | | | | | | ZM-PC-8-1, | | | |
| | | | | | | | | | | ZM-PC-9-1, | | | |
| | | | | | | | | | | ZM-PR-9-1, | | | |
| | | | | | | | | | | ZM-STC-8-1, | | | |
| | | | | | | | | | | ZM-BIOM-8-1, | | | |
| | | | | | | | | | | ZM-DMC-8-1, | | | |
| | | | | | | | | | | ZM-DMC-8-2, | | | |
| | | | | | | | | | | ZM-DMY-3-2, | | | |
| | | | | | | | | | | ZM-DMY-3-3, | | | |
| | | | | | | | | | | ZM-DMY-8-1, | | | |
| | | | | | | | | | | ZM-DMY-8-2, | | | |
| | | | | | | | | | | ZM-GWE-9-1, | | | |
| | | | | | | | | | | ZM-GWM2-3-1, | | | |
| | | | | | | | | | | ZM-GYHA-8-1, | | | |
| | | | | | | | | | | ZM-GYLD-8-2, | | | |
| | | | | | | | | | | ZM-GYLD-9-1, | | | |
| | | | | | | | | | | ZM-HI-3-1, | | | |
| | | | | | | | | | | ZM-HI-8-1, | | | |
| | | | | | | | | | | ZM-KW100-9-1, | | | |
| | | | | | | | | | | ZM-KW300-3-2, | | | |
| | | | | | | | | | | ZM-KW300-8-2, | | | |
| | | | | | | | | | | ZM-KW300-9-2, | | | |
| | | | | | | | | | | ZM-TGW-9-1, | | | |
| | | | | | | | | | | ZM-TW-8-1, | | | |
| | | | | | | | | | | ZM-YLD-9-1, | | | |
| | | | | | | | | | | ZM-YLD-9-1 | | | |
| 157 | X | — | — | — | X | — | — | — | — | MAS24-2, | 126 | 140 | CCT |
| | | | | | | | | | | ZM-CPC-1-4, | | | |
| | | | | | | | | | | ZM-CPC-1-6, | | | |
| | | | | | | | | | | ZM-MOIST-4-3, | | | |
| | | | | | | | | | | ZM-MOIST-7-3, | | | |
| | | | | | | | | | | ZM-MOIST-7-4, | | | |
| | | | | | | | | | | ZM-MOIST-9-2, | | | |
| | | | | | | | | | | ZM-MOIST-9-2, | | | |
| | | | | | | | | | | ZM-PC-9-1, | | | |
| | | | | | | | | | | ZM-BIOM-3-1, | | | |
| | | | | | | | | | | ZM-DMC-1-2, | | | |
| | | | | | | | | | | ZM-DMY-1-3, | | | |
| | | | | | | | | | | ZM-DMY-1-5, | | | |
| | | | | | | | | | | ZM-DMY-4-3, | | | |
| | | | | | | | | | | ZM-GWM2-3-2, | | | |
| | | | | | | | | | | ZM-GYLD-3-3, | | | |
| | | | | | | | | | | ZM-GYLD-9-1, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-KW100-9-1, | | | |
| | | | | | | | | | | ZM-KW300-9-1, | | | |
| | | | | | | | | | | ZM-KW300-9-2, | | | |
| | | | | | | | | | | ZM-YLD-9-1 | | | |
| 349 | — | — | — | — | X | — | — | — | — | | | | |
| 139 | X | — | — | — | X | — | — | — | — | | | | |
| 175 | X | — | — | — | X | — | — | — | — | | | | |
| 5 | — | — | — | X | X | — | — | — | — | | | | |
| 351 | — | — | — | — | X | — | — | — | — | | | | |
| 353 | X | — | — | — | X | — | — | — | — | | | | |
| 309 | — | — | — | — | X | — | X | — | — | OS-RGT-2-1, | 378 | 392 | CAA |
| | | | | | | | | | | OS-VGT-2-1 | | | |
| 355 | — | — | — | — | X | — | — | — | — | | | | |
| 255 | — | — | — | — | X | — | — | — | X | OS-GW-9-1, | | | |
| | | | | | | | | | | MAS13-24, | | | |
| | | | | | | | | | | MAS13-31, | | | |
| | | | | | | | | | | ZM-CPC-1-3, | | | |
| | | | | | | | | | | ZM-CPC-1-5, | | | |
| | | | | | | | | | | ZM-CPC-7-2, | | | |
| | | | | | | | | | | ZM-CPC-7-3, | | | |
| | | | | | | | | | | ZM-IVDOM-1-2, | | | |
| | | | | | | | | | | ZM-IVDOM-1-4, | | | |
| | | | | | | | | | | ZM-MOIST-1-4, | | | |
| | | | | | | | | | | ZM-MOIST-1-5, | | | |
| | | | | | | | | | | ZM-MOIST-7-1, | | | |
| | | | | | | | | | | ZM-MOIST-7-2, | | | |
| | | | | | | | | | | ZM-PC-1-1, | | | |
| | | | | | | | | | | ZM-STC-7-2, | | | |
| | | | | | | | | | | ZM-BIOM-7-1, | | | |
| | | | | | | | | | | ZM-DMC-1-1, | | | |
| | | | — | | | | | | | ZM-DMY-1-4, | | | |
| | | | | | | | | | | ZM-GWM2-7-1, | | | |
| | | | | | | | | | | ZM-GYLD-7-3, | | | |
| | | | | | | | | | | ZM-GYUP-1-2, | | | |
| | | | | | | | | | | ZM-HI-7-1, | | | |
| | | | | | | | | | | ZM-KW300-1-2, | | | |
| | | | | | | | | | | ZM-TW-1-1 | | | |
| 75 | X | — | — | — | X | — | — | — | — | | | | |
| 357 | — | — | — | — | X | — | — | — | — | | | | |
| 359 | — | — | — | — | X | — | — | — | — | OS-GW-5-1, | | | |
| | | | | | | | | | | OS-YLD-5-1, | | | |
| | | | | | | | | | | ZM-MOIST-4-3, | | | |
| | | | | | | | | | | ZM-DMY-4-3, | | | |
| | | | | | | | | | | ZM-YLD-4-1 | | | |
| 361 | — | — | — | — | X | — | — | — | — | | | | |
| 363 | — | — | — | — | X | — | — | — | — | OS-GW-3-1, | | | |
| | | | | | | | | | | ZM-CPC-1-2, | | | |
| | | | | | | | | | | ZM-IVDOM-1-1, | | | |
| | | | | | | | | | | ZM-IVDOM-9-1, | | | |
| | | | | | | | | | | ZM-IVDOM-9-2, | | | |
| | | | | | | | | | | ZM-MOIST-1-2, | | | |
| | | | | | | | | | | ZM-MOIST-1-2, | | | |
| | | | | | | | | | | ZM-MOIST-9-3, | | | |
| | | | | | | | | | | ZM-DMY-9-1, | | | |
| | | | | | | | | | | ZM-GYHA-1-3, | | | |
| | | | | | | | | | | ZM-GYHA-1-4, | | | |
| | | | | | | | | | | ZM-GYLD-1-1, | | | |
| | | | | | | | | | | ZM-GYLD-9-2, | | | |
| | | | | | | | | | | ZM-GYLD-9-2, | | | |
| | | | | | | | | | | ZM-GYUP-1-1, | | | |
| | | | | | | | | | | ZM-GYUP-1-1, | | | |
| | | | | | | | | | | ZM-HI-1-1, | | | |
| | | | | | | | | | | ZM-KW100-1-2, | | | |
| | | | | | | | | | | ZM-KW100-9-1, | | | |
| | | | | | | | | | | ZM-TGW-9-2, | | | |
| | | | | | | | | | | ZM-TW-9-1, | | | |
| | | | | | | | | | | ZM-YLD-1-1 | | | |
| 365 | — | — | — | — | X | — | — | — | — | | | | |
| 181 | X | — | — | — | X | — | — | — | — | | | | |
| 367 | — | — | — | — | X | — | — | — | — | | | | |
| 261 | — | — | — | — | X | — | — | — | X | | | | |
| 221 | — | — | X | — | X | — | — | — | — | | | | |
| 57 | — | X | — | — | X | — | — | — | — | | | | |
| 25 | — | — | — | — | X | X | — | — | — | | 1047 | 1061 | CGC |
| 369 | — | — | — | — | X | — | — | — | — | OS-CHALK-10-1, | | | |
| | | | | | | | | | | ZM-MOIST-2-3, | | | |
| | | | | | | | | | | ZM-DMY-2-3, | | | |
| | | | | | | | | | | ZM-GYLD-2-3 | | | |
| 39 | — | X | — | — | X | — | — | — | — | | | | |
| 87 | X | — | — | — | X | — | — | — | — | OS-APDF-9-1, | 30 | 44 | CCT |
| | | | | | | | | | | MAS13-24, | 1391 | 1411 | CCG |
| | | | | | | | | | | ZM-CPC-1-3, | | | |
| | | | | | | | | | | ZM-CPC-1-5, | | | |
| | | | | | | | | | | ZM-IVDOM-1-2, | | | |
| | | | | | | | | | | ZM-IVDOM-1-4, | | | |
| | | | | | | | | | | ZM-MOIST-1-4, | | | |
| | | | | | | | | | | ZM-MOIST-1-5, | | | |
| | | | | | | | | | | ZM-MOIST-2-3, | | | |
| | | | | | | | | | | ZM-PC-1-1, | | | |
| | | | | | | | | | | ZM-STC-2-2, | | | |
| | | | | | | | | | | ZM-DMC-1-1, | | | |
| | | | | | | | | | | ZM-DMY-1-1, | | | |
| | | | | | | | | | | ZM-DMY-2-3, | | | |
| | | | | | | | | | | ZM-DMY-2-4, | | | |
| | | | | | | | | | | ZM-GYLD-2-1, | | | |
| | | | | | | | | | | ZM-GYLD-2-3, | | | |
| | | | | | | | | | | ZM-GYUP-1-2, | | | |
| | | | | | | | | | | ZM-KW300-1-2, | | | |
| | | | | | | | | | | ZM-TW-1-1, | | | |
| | | | | | | | | | | ZM-YLD-2-1, | | | |
| | | | | | | | | | | ZM-YLD-2-2 | | | |
| 371 | — | — | — | — | X | — | — | — | — | | | | |
| 163 | X | — | — | — | X | — | — | — | — | | | | |
| 373 | — | — | — | — | X | — | — | — | — | | | | |
| 313 | — | — | — | — | X | — | X | — | — | OS-GW-5-1, | | | |
| | | | | | | | | | | OS-YLD-5-1 | | | |
| 375 | — | — | — | — | X | — | — | — | — | | | | |
| 315 | X | — | — | — | X | — | X | — | — | OS-GPL-4-1, | 683 | 703 | CCG |
| | | | | | | | | | | OS-GPP-4-1, | | | |
| | | | | | | | | | | OS-GYLD-4-1, | | | |
| | | | | | | | | | | MAS24-2, | | | |
| | | | | | | | | | | ZM-CPC-3-2, | | | |
| | | | | | | | | | | ZM-ID-10-1, | | | |
| | | | | | | | | | | ZM-ID-2-1, | | | |
| | | | | | | | | | | ZM-MOIST-10-1, | | | |
| | | | | | | | | | | ZM-MOIST-2-2, | | | |
| | | | | | | | | | | ZM-MOIST-3-2, | | | |
| | | | | | | | | | | ZM-MOIST-9-2, | | | |
| | | | | | | | | | | ZM-PC-9-1, | | | |
| | | | | | | | | | | ZM-STC-10-1, | | | |
| | | | | | | | | | | ZM-BIOM-3-1, | | | |
| | | | | | | | | | | ZM-DMC-10-1, | | | |
| | | | | | | | | | | ZM-DMC-10-2, | | | |
| | | | | | | | | | | ZM-DMC-2-3, | | | |
| | | | | | | | | | | ZM-DMY-10-1, | | | |
| | | | | | | | | | | ZM-DMY-3-1, | | | |
| | | | | | | | | | | ZM-EWT-2-1, | | | |
| | | | | | | | | | | ZM-GWM2-10-1, | | | |
| | | | | | | | | | | ZM-GWM2-3-2, | | | |
| | | | | | | | | | | ZM-GYHA-3-1, | | | |
| | | | | | | | | | | ZM-GYLD-2-2, | | | |
| | | | | | | | | | | ZM-GYLD-3-3, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-HI-10-1, | | | |
| | | | | | | | | | | ZM-KW300-3-3, | | | |
| | | | | | | | | | | ZM-KW300-9-1, | | | |
| | | | | | | | | | | ZM-KW300-9-2, | | | |
| | | | | | | | | | | ZM-TW-10-2, | | | |
| | | | | | | | | | | ZM-TW-2-3 | | | |
| 89 | X | — | — | — | X | — | — | — | — | | | | |
| 377 | — | — | — | — | X | — | — | — | — | | | | |
| 289 | — | — | — | — | X | — | — | X | — | | | | |
| 49 | — | X | — | — | X | — | — | — | — | | | | |
| 153 | X | X | — | — | X | — | — | — | — | | | | |
| 81 | X | — | — | — | X | — | — | — | — | | | | |
| 379 | — | — | — | — | X | — | — | — | — | | 707 | 721 | CGC |
| | | | | | | | | | | | 882 | 902 | GGA |
| 305 | — | — | — | — | X | — | X | — | — | OS-BDV-1-1, | | | |
| | | | | | | | | | | OS-CHALK-1-1, | | | |
| | | | | | | | | | | OS-CPV-1-1, | | | |
| | | | | | | | | | | OS-CSV-1-1, | | | |
| | | | | | | | | | | OS-SBV-1-1, | | | |
| | | | | | | | | | | OS-GP-1-1, | | | |
| | | | | | | | | | | OS-GW-1-2, | | | |
| | | | | | | | | | | OS-YLD-1-1, | | | |
| | | | | | | | | | | ZM-MOIST-1-1, | | | |
| | | | | | | | | | | ZM-MOIST-1-2, | | | |
| | | | | | | | | | | ZM-GYHA-1-2, | | | |
| | | | | | | | | | | ZM-GYHA-1-3, | | | |
| | | | | | | | | | | ZM-GYUP-1-1, | | | |
| | | | | | | | | | | ZM-HI-1-1, | | | |
| | | | | | | | | | | ZM-KW100-1-2 | | | |
| 381 | — | — | — | — | X | — | — | — | — | OS-GPL-4-1, | | | |
| | | | | | | | | | | OS-GPP-4-1, | | | |
| | | | | | | | | | | OS-GYLD-4-1, | | | |
| | | | | | | | | | | MAS24-2, | | | |
| | | | | | | | | | | ZM-CPC-3-2, | | | |
| | | | | | | | | | | ZM-ID-10-1, | | | |
| | | | | | | | | | | ZM-ID-2-1, | | | |
| | | | | | | | | | | ZM-MOIST-10-1, | | | |
| | | | | | | | | | | ZM-MOIST-2-2, | | | |
| | | | | | | | | | | ZM-MOIST-3-2, | | | |
| | | | | | | | | | | ZM-MOIST-9-2, | | | |
| | | | | | | | | | | ZM-PC-9-1, | | | |
| | | | | | | | | | | ZM-STC-10-1, | | | |
| | | | | | | | | | | ZM-BIOM-3-1, | | | |
| | | | | | | | | | | ZM-DMC-10-1, | | | |
| | | | | | | | | | | ZM-DMC-10-2, | | | |
| | | | | | | | | | | ZM-DMC-2-3, | | | |
| | | | | | | | | | | ZM-DMY-10-1, | | | |
| | | | | | | | | | | ZM-DMY-3-1, | | | |
| | | | | | | | | | | ZM-EWT-2-1, | | | |
| | | | | | | | | | | ZM-GWM2-10-1, | | | |
| | | | | | | | | | | ZM-GWM2-3-2, | | | |
| | | | | | | | | | | ZM-GYHA-3-1, | | | |
| | | | | | | | | | | ZM-GYLD-2-2, | | | |
| | | | | | | | | | | ZM-GYLD-3-3, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-HI-10-1, | | | |
| | | | | | | | | | | ZM-KW300-3-3, | | | |
| | | | | | | | | | | ZM-KW300-9-1, | | | |
| | | | | | | | | | | ZM-KW300-9-2, | | | |
| | | | | | | | | | | ZM-TW-10-2, | | | |
| | | | | | | | | | | ZM-TW-2-3 | | | |
| 197 | X | — | — | — | X | — | — | — | — | | | | |
| 45 | — | X | — | — | X | — | — | — | — | | | | |
| 97 | X | — | — | — | X | — | — | — | — | | | | |
| 383 | — | — | — | — | X | — | — | — | — | | | | |
| 135 | X | — | — | — | X | — | — | — | — | | | | |
| 267 | X | — | — | — | X | — | — | — | X | | 217 | 234 | CCG |
| 385 | — | — | — | — | X | — | — | — | — | | 90 | 107 | CGG |
| | | | | | | | | | | | 575 | 592 | CCG |
| 33 | — | X | — | — | X | — | — | — | — | | | | |
| 283 | — | — | — | — | X | — | — | X | — | | 391 | 408 | CGG |
| 53 | — | X | — | — | X | — | — | — | — | | | | |
| 253 | — | — | — | — | X | — | — | — | X | | | | |
| 387 | — | — | — | — | X | — | — | — | — | | | | |
| 295 | — | — | — | — | X | — | — | X | — | OS-GPL-4-1, | | | |
| | | | | | | | | | | OS-GPP-4-1, | | | |
| | | | | | | | | | | OS-GYLD-4-1, | | | |
| | | | | | | | | | | MAS24-2, | | | |
| | | | | | | | | | | ZM-CPC-3-2, | | | |
| | | | | | | | | | | ZM-ID-10-1, | | | |
| | | | | | | | | | | ZM-ID-2-1, | | | |
| | | | | | | | | | | ZM-MOIST-10-1, | | | |
| | | | | | | | | | | ZM-MOIST-2-2, | | | |
| | | | | | | | | | | ZM-MOIST-3-2, | | | |
| | | | | | | | | | | ZM-MOIST-9-2, | | | |
| | | | | | | | | | | ZM-PC-9-1, | | | |
| | | | | | | | | | | ZM-STC-10-1, | | | |
| | | | | | | | | | | ZM-BIOM-3-1, | | | |
| | | | | | | | | | | ZM-DMC-10-1, | | | |
| | | | | | | | | | | ZM-DMC-10-2, | | | |
| | | | | | | | | | | ZM-DMC-2-3, | | | |
| | | | | | | | | | | ZM-DMY-10-1, | | | |
| | | | | | | | | | | ZM-DMY-3-1, | | | |
| | | | | | | | | | | ZM-EWT-2-1, | | | |
| | | | | | | | | | | ZM-GWM2-10-1, | | | |
| | | | | | | | | | | ZM-GWM2-3-2, | | | |
| | | | | | | | | | | ZM-GYHA-3-1, | | | |
| | | | | | | | | | | ZM-GYLD-2-2, | | | |
| | | | | | | | | | | ZM-GYLD-3-3, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-HI-10-1, | | | |
| | | | | | | | | | | ZM-KW300-3-3, | | | |
| | | | | | | | | | | ZM-KW300-9-1, | | | |
| | | | | | | | | | | ZM-KW300-9-2, | | | |
| | | | | | | | | | | ZM-TW-10-2, | | | |
| | | | | | | | | | | ZM-TW-2-3 | | | |
| 389 | — | — | — | — | X | — | — | — | — | | | | |
| 225 | — | — | X | — | X | — | — | — | — | | | | |
| 391 | — | — | — | — | X | — | — | — | — | | | | |
| 167 | X | — | — | — | X | — | — | — | — | OS-GW-3-1, | | | |
| | | | | | | | | | | MAS19-14, | | | |
| | | | | | | | | | | SMS021-79, | | | |
| | | | | | | | | | | ZM-CL-9-1, | | | |
| | | | | | | | | | | ZM-CPC-1-2, | | | |
| | | | | | | | | | | ZM-CPC-6-2, | | | |
| | | | | | | | | | | ZM-ID-8-1, | | | |
| | | | | | | | | | | ZM-ID-8-1, | | | |
| | | | | | | | | | | ZM-IVDOM-1-1, | | | |
| | | | | | | | | | | ZM-IVDOM-1-3, | | | |
| | | | | | | | | | | ZM-IVDOM-9-1, | | | |
| | | | | | | | | | | ZM-IVDOM-9-2, | | | |
| | | | | | | | | | | ZM-MOIST-1-2, | | | |
| | | | | | | | | | | ZM-MOIST-1-3, | | | |
| | | | | | | | | | | ZM-MOIST-4-3, | | | |
| | | | | | | | | | | ZM-MOIST-9-3, | | | |
| | | | | | | | | | | ZM-PC-8-1, | | | |
| | | | | | | | | | | ZM-PC-9-1, | | | |
| | | | | | | | | | | ZM-PR-9-1, | | | |
| | | | | | | | | | | ZM-BIOM-8-1, | | | |
| | | | | | | | | | | ZM-DMC-6-1, | | | |
| | | | | | | | | | | ZM-DMC-8-1, | | | |
| | | | | | | | | | | ZM-DMY-1-2, | | | |
| | | | | | | | | | | ZM-DMY-4-3, | | | |
| | | | | | | | | | | ZM-DMY-8-2, | | | |
| | | | | | | | | | | ZM-DMY-9-1, | | | |
| | | | | | | | | | | ZM-GWE-9-1, | | | |
| | | | | | | | | | | ZM-GYHA-1-1, | | | |
| | | | | | | | | | | ZM-GYHA-1-4, | | | |
| | | | | | | | | | | ZM-GYHA-8-1, | | | |
| | | | | | | | | | | ZM-GYLD-1-1, | | | |
| | | | | | | | | | | ZM-GYLD-1-2, | | | |
| | | | | | | | | | | ZM-GYLD-6-1, | | | |
| | | | | | | | | | | ZM-GYLD-6-4, | | | |
| | | | | | | | | | | ZM-GYLD-9-2, | | | |
| | | | | | | | | | | ZM-GYLD-9-2, | | | |
| | | | | | | | | | | ZM-GYUP-1-1, | | | |
| | | | | | | | | | | ZM-HI-1-1, | | | |
| | | | | | | | | | | ZM-HI-8-1, | | | |
| | | | | | | | | | | ZM-KW100-9-1, | | | |
| | | | | | | | | | | ZM-KW300-8-2, | | | |
| | | | | | | | | | | ZM-TGW-9-1, | | | |
| | | | | | | | | | | ZM-TGW-9-2, | | | |
| | | | | | | | | | | ZM-TW-9-1, | | | |
| | | | | | | | | | | ZM-YLD-1-1, | | | |
| | | | | | | | | | | ZM-YLD-9-1 | | | |
| 137 | X | — | — | — | X | — | — | — | — | | | | |
| 393 | — | — | — | — | X | — | — | — | — | | | | |
| 195 | X | — | — | — | X | — | — | — | — | | | | |
| 263 | — | — | — | — | X | — | — | — | X | | | | |
| 41 | — | X | — | — | X | — | — | — | — | | | | |
| 303 | — | — | — | — | X | — | X | — | — | | | | |
| 223 | — | — | X | — | X | — | — | — | — | | | | |
| 85 | X | — | — | — | X | — | — | — | — | | | | |
| 395 | — | — | — | — | X | — | — | — | — | | | | |
| 129 | X | — | — | — | X | — | — | — | — | OS-ASS-6-2, | | | |
| | | | | | | | | | | MAS24-2, | | | |
| | | | | | | | | | | ZM-ID-5-2, | | | |
| | | | | | | | | | | ZM-MOIST-5-4, | | | |
| | | | | | | | | | | ZM-MOIST-9-2, | | | |
| | | | | | | | | | | ZM-PC-5-1, | | | |
| | | | | | | | | | | ZM-PC-9-1, | | | |
| | | | | | | | | | | ZM-STC-5-1, | | | |
| | | | | | | | | | | ZM-DMC-5-1, | | | |
| | | | | | | | | | | ZM-GYLD-5-2, | | | |
| | | | | | | | | | | ZM-GYUI-9-1, | | | |
| | | | | | | | | | | ZM-GYUI-9-1, | | | |
| | | | | | | | | | | ZM-GYUI-9-2, | | | |
| | | | | | | | | | | ZM-GYUP-9-1, | | | |
| | | | | | | | | | | ZM-GYUP-9-2, | | | |
| | | | | | | | | | | ZM-KW300-9-1, | | | |
| | | | | | | | | | | ZM-KW300-9-2 | | | |
| 103 | X | — | — | — | X | — | — | — | — | | | | |
| 51 | — | X | — | — | X | — | — | — | — | | | | |
| 99 | — | — | — | — | X | — | — | — | — | | | | |
| 69 | X | — | — | — | X | — | — | — | — | | | | |
| 397 | — | — | — | — | X | — | — | — | — | | | | |
| 229 | — | — | X | — | X | — | — | — | — | | | | |
| 399 | — | — | — | — | X | — | — | — | — | | | | |
| 241 | — | — | X | — | X | — | — | — | — | | | | |
| 91 | X | — | — | — | X | — | — | — | — | | | | |
| 401 | — | — | — | — | X | — | — | — | — | | | | |
| 121 | X | — | — | — | X | — | — | — | — | | | | |
| 403 | — | — | — | — | X | — | — | — | — | | | | |
| 187 | X | — | — | — | X | — | — | — | — | | | | |
| 405 | — | — | — | — | X | — | — | — | — | | | | |
| 13 | — | — | — | X | X | — | — | — | — | | | | |
| 243 | — | — | X | — | X | — | — | — | — | | | | |
| 203 | X | — | — | — | X | — | — | — | — | | 441 | 455 | CGG |
| 407 | — | — | — | — | X | — | — | — | — | | | | |
| 409 | — | — | — | — | X | — | — | — | — | | | | |
| 411 | — | — | — | — | X | — | — | — | — | | 243 | 260 | CAG |
| 105 | X | — | — | — | X | — | — | — | — | | | | |
| 107 | X | — | — | — | X | — | — | — | — | | 235 | 255 | GAG |
| 115 | X | — | — | — | X | — | — | — | — | | 1449 | 1463 | CGG |
| 15 | — | — | — | X | X | — | — | — | — | | | | |
| 165 | X | — | — | — | X | — | — | — | — | | | | |
| 123 | X | — | — | — | X | — | — | — | — | | | | |
| 205 | X | — | — | — | X | — | — | — | — | | | | |
| 63 | — | X | — | — | X | — | — | — | — | | | | |
| 413 | — | — | — | — | X | — | — | — | — | | 146 | 160 | CGG |
| 209 | X | — | — | — | X | — | — | — | — | | | | |
| 323 | — | — | — | — | X | — | X | — | — | | 129 | 143 | CGG |
| | | | | | | | | | | | 368 | 385 | CCG |
| 77 | X | — | — | — | X | — | — | — | — | | | | |
| 415 | — | — | — | — | X | — | — | — | — | | | | |
| 141 | X | — | — | — | X | — | — | — | — | | 128 | 148 | CCT |
| 27 | — | — | — | — | X | X | — | — | — | | | | |
| 65 | — | X | — | — | X | — | — | — | — | | | | |
| 185 | X | — | — | — | X | — | — | — | — | | | | |
| 299 | — | — | — | — | X | — | — | X | — | | 5 | 22 | CGG |
| 67 | — | X | — | — | X | — | — | — | — | | | | |
| 17 | — | — | — | X | X | — | — | — | — | | | | |
| 279 | — | — | — | — | X | — | — | — | X | | | | |
| 71 | X | — | — | — | X | — | — | — | — | | | | |
| 207 | X | — | — | — | X | — | — | — | — | | 8 | 25 | CCG |
| 417 | — | — | — | — | X | — | — | — | — | | | | |
| 127 | X | — | — | — | X | — | — | — | — | | | | |
| 125 | X | — | — | — | X | — | — | — | — | | | | |
| 117 | X | — | — | — | X | — | — | — | — | | | | |
| 183 | X | — | — | — | X | — | — | — | — | | | | |
| 419 | — | — | — | — | X | — | — | — | — | | | | |
| 421 | — | — | — | — | X | — | — | — | — | | | | |
| 29 | — | — | — | — | X | X | — | — | — | | | | |
| 297 | — | — | — | — | X | — | — | X | — | | | | |
| 423 | — | — | — | — | X | — | — | — | — | | 921 | 936 | AG |
| 425 | — | — | — | — | X | — | — | — | — | | | | |
| 245 | — | — | X | — | X | — | — | — | — | | | | |
| 427 | — | — | — | — | X | — | — | — | — | | | | |
| 429 | — | — | — | — | X | — | — | — | — | | | | |
| 247 | — | — | X | — | X | — | — | — | — | | | | |
| 249 | — | — | X | — | X | — | — | — | — | | | | |
| 159/171 | — | — | X | — | — | — | — | | | | | | |
| X | | | | | | | | | | | | | |
| 31 | — | X | — | — | X | — | — | — | — | | | | |
| 275 | — | — | — | — | X | — | — | — | X | | 217 | 234 | GGC |
| | | | | | | | | | | | 753 | 767 | CGG |
| 19 | — | — | — | — | X | X | — | — | — | | | | |
| 151 | X | — | — | — | X | — | — | — | — | | | | |
| 213/227- | X | — | X | — | — | — | — | | | OS-FLLEN-9-1, | 339 | 353 | GTC |
| | | | | | | | | | | OS-GW100-4-1, | 434 | 448 | AGC |
| | | | | | | | | | | MAS24-2, | | | |
| | | | | | | | | | | MAS24-3, | | | |
| | | | | | | | | | | ZM-CPC-1-4, | | | |
| | | | | | | | | | | ZM-CPC-1-6, | | | |
| | | | | | | | | | | ZM-CPC-10-1, | | | |
| | | | | | | | | | | ZM-IVDOM-10-1, | | | |
| | | | | | | | | | | ZM-IVDOM-10-2 | | | |
| | | | | | | | | | | ZM-MOIST-1-1, | | | |
| | | | | | | | | | | ZM-MOIST-9-2, | | | |
| | | | | | | | | | | ZM-PC-9-1, | | | |
| | | | | | | | | | | ZM-STC-10-2, | | | |
| | | | | | | | | | | ZM-STC-2-2, | | | |
| | | | | | | | | | | ZM-DMC-1-2, | | | |
| | | | | | | | | | | ZM-DMY-1-3, | | | |
| | | | | | | | | | | ZM-DMY-1-5, | | | |
| | | | | | | | | | | ZM-DMY-2-4, | | | |
| | | | | | | | | | | ZM-GYHA-1-2, | | | |
| | | | | | | | | | | ZM-GYUI-9-1, | | | |
| | | | | | | | | | | ZM-GYUI-9-1, | | | |
| | | | | | | | | | | ZM-GYUI-9-2, | | | |
| | | | | | | | | | | ZM-GYUP-9-1, | | | |
| | | | | | | | | | | ZM-GYUP-9-2, | | | |
| | | | | | | | | | | ZM-HI-1-1, | | | |
| | | | | | | | | | | ZM-KW300-9-1, | | | |
| | | | | | | | | | | ZM-KW300-9-2 | | | |
| 237 | — | — | X | — | X | — | — | — | — | | | | |
| 133 | X | — | — | — | X | — | X | — | — | | | | |
| 239 | — | — | X | — | X | — | — | — | — | | | | |
| 161 | X | — | — | — | X | — | — | — | — | | | | |
| 61 | X | — | — | — | X | — | — | — | — | | | | |
| 47 | — | X | — | — | X | — | — | — | — | | | | |
| 219 | — | — | X | — | X | — | — | — | — | | | | |
| 259/271- | — | — | X | — | — | — | X | | | | | | |
| 93 | X | — | — | — | X | — | — | — | — | OS-AE-12-1 | | | |
| 111 | X | — | — | — | X | — | — | — | — | | 275 | 289 | GCG |
| 73 | X | — | — | — | X | — | — | — | — | | 54 | 74 | CGG |
| 235 | — | — | X | — | X | — | — | — | — | | | | |
| 217 | — | — | X | — | X | — | — | — | — | | | | |
| 257 | — | — | — | — | X | — | — | — | X | | | | |
| 201 | X | — | — | — | X | — | — | — | — | OS-AMY-6-1, | | | |
| | | | | | | | | | | OS-AMY-6-2, | | | |
| | | | | | | | | | | OS-ASS-6-1, | | | |
| | | | | | | | | | | OS-GC-6-1, | | | |
| | | | | | | | | | | OS-BDV-6-1, | | | |
| | | | | | | | | | | OS-CHALK-6-1, | | | |
| | | | | | | | | | | OS-CPV-6-1, | | | |
| | | | | | | | | | | OS-CPV-6-2, | | | |
| | | | | | | | | | | OS-CSV-6-1, | | | |
| | | | | | | | | | | OS-CSV-6-2, | | | |
| | | | | | | | | | | OS-HPV-6-1, | | | |
| | | | | | | | | | | OS-HPV-6-2, | | | |
| | | | | | | | | | | OS-SBV-6-1, | | | |
| | | | | | | | | | | OS-WC-6-1, | | | |
| | | | | | | | | | | OS-DM-6-1, | | | |
| | | | | | | | | | | OS-GP-6-1, | | | |
| | | | | | | | | | | OS-Y-6-1, | | | |
| | | | | | | | | | | MAS24-2, | | | |
| | | | | | | | | | | ZM-CPC-6-2, | | | |
| | | | | | | | | | | ZM-ID-10-1, | | | |
| | | | | | | | | | | ZM-MOIST-10-1, | | | |
| | | | | | | | | | | ZM-MOIST-9-2, | | | |
| | | | | | | | | | | ZM-MOIST-9-2, | | | |
| | | | | | | | | | | ZM-PC-9-1, | | | |
| | | | | | | | | | | ZM-STC-10-1, | | | |
| | | | | | | | | | | ZM-DMC-10-1, | | | |
| | | | | | | | | | | ZM-DMC-10-2, | | | |
| | | | | | | | | | | ZM-DMC-6-1, | | | |
| | | | | | | | | | | ZM-DMC-6-2, | | | |
| | | | | | | | | | | ZM-DMY-10-1, | | | |
| | | | | | | | | | | ZM-GWM2-10-1, | | | |
| | | | | | | | | | | ZM-GYLD-6-1, | | | |
| | | | | | | | | | | ZM-GYLD-6-4, | | | |
| | | | | | | | | | | ZM-GYLD-6-4, | | | |
| | | | | | | | | | | ZM-GYLD-9-1, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-HI-10-1, | | | |
| | | | | | | | | | | ZM-KW100-9-1, | | | |
| | | | | | | | | | | ZM-KW300-9-1, | | | |
| | | | | | | | | | | ZM-KW300-9-2, | | | |
| | | | | | | | | | | ZM-TW-10-2, | | | |
| | | | | | | | | | | ZM-YLD-6-1, | | | |
| | | | | | | | | | | ZM-YLD-9-1 | | | |
| 281 | — | — | — | — | X | — | — | X | — | | | | |
| 251 | — | — | — | — | X | — | — | — | X | | | | |
| 3 | — | — | — | X | X | — | — | — | — | OS-AE-11-1, | 24 | 38 | CGC |
| | | | | | | | | | | ZM-MOIST-1-6, | | | |
| | | | | | | | | | | ZM-MOIST-5-1, | | | |
| | | | | | | | | | | ZM-PC-1-2, | | | |
| | | | | | | | | | | ZM-GWM2-1-1, | | | |
| | | | | | | | | | | ZM-GYHA-5-1, | | | |
| | | | | | | | | | | ZM-GYLD-5-3, | | | |
| | | | | | | | | | | ZM-HI-1-2, | | | |
| | | | | | | | | | | ZM-KW100-1-2 | | | |
| 21 | — | — | — | — | X | X | — | — | — | OS-AE-12-1 | | | |
| 179 | X | — | — | — | X | — | — | — | — | | | | |
| 319 | X | — | — | — | X | — | X | — | — | | 41 | 55 | CCG |
| 7 | — | — | — | X | X | — | — | — | — | | | | |
| 291 | — | — | — | — | X | — | — | X | — | | 10 | 24 | GAG |
| 169 | X | — | — | — | X | — | — | — | — | | | | |
| 83 | X | — | — | — | X | — | — | — | — | | | | |
| 269 | — | — | — | — | X | — | — | — | X | | | | |
| 9 | — | — | — | X | X | — | — | — | — | OS-GPL-4-1, | | | |
| | | | | | | | | | | OS-GPP-4-1, | | | |
| | | | | | | | | | | OS-GYLD-4-1, | | | |
| | | | | | | | | | | MAS24-2, | | | |
| | | | | | | | | | | MAS24-28, | | | |
| | | | | | | | | | | ZM-CPC-3-2, | | | |
| | | | | | | | | | | ZM-ID-10-1, | | | |
| | | | | | | | | | | ZM-ID-2-1, | | | |
| | | | | | | | | | | ZM-MOIST-10-1, | | | |
| | | | | | | | | | | ZM-MOIST-2-2, | | | |
| | | | | | | | | | | ZM-MOIST-3-2, | | | |
| | | | | | | | | | | ZM-MOIST-4-3, | | | |
| | | | | | | | | | | ZM-MOIST-5-3, | | | |
| | | | | | | | | | | ZM-MOIST-9-2, | | | |
| | | | | | | | | | | ZM-PC-9-1, | | | |
| | | | | | | | | | | ZM-STC-10-1, | | | |
| | | | | | | | | | | ZM-BIOM-3-1, | | | |
| | | | | | | | | | | ZM-DMC-10-1, | | | |
| | | | | | | | | | | ZM-DMC-10-2, | | | |
| | | | | | | | | | | ZM-DMC-2-3, | | | |
| | | | | | | | | | | ZM-DMY-10-1, | | | |
| | | | | | | | | | | ZM-DMY-3-1, | | | |
| | | | | | | | | | | ZM-DMY-4-3, | | | |
| | | | | | | | | | | ZM-EWT-2-1, | | | |
| | | | | | | | | | | ZM-GWM2-10-1, | | | |
| | | | | | | | | | | ZM-GWM2-3-2, | | | |
| | | | | | | | | | | ZM-GYHA-3-1, | | | |
| | | | | | | | | | | ZM-GYLD-2-2, | | | |
| | | | | | | | | | | ZM-GYLD-3-3, | | | |
| | | | | | | | | | | ZM-GYLD-5-2, | | | |
| | | | | | | | | | | ZM-GYUI-9-1, | | | |
| | | | | | | | | | | ZM-GYUI-9-2, | | | |
| | | | | | | | | | | ZM-GYUP-9-2, | | | |
| | | | | | | | | | | ZM-HI-10-1, | | | |
| | | | | | | | | | | ZM-KW300-3-3, | | | |
| | | | | | | | | | | ZM-KW300-9-1, | | | |
| | | | | | | | | | | ZM-KW300-9-2, | | | |
| | | | | | | | | | | ZM-TW-10-2, | | | |
| | | | | | | | | | | ZM-TW-2-3 | | | |
| 449 | — | — | — | — | X | — | — | — | X | | | | |
| 277 | — | — | — | — | X | — | — | — | X | | 664 | 681 | ACT |
| 285 | — | — | — | — | X | — | — | X | — | OS-PGWC-8-1, | | | |
| | | | | | | | | | | OS-FLWID-3-1, | | | |
| | | | | | | | | | | OS-GPP-8-2, | | | |
| | | | | | | | | | | SMS015-9, | | | |
| | | | | | | | | | | ZM-CPC-1-3, | | | |
| | | | | | | | | | | ZM-CPC-1-5, | | | |
| | | | | | | | | | | ZM-IVDOM-1-2, | | | |
| | | | | | | | | | | ZM-IVDOM-1-3, | | | |
| | | | | | | | | | | ZM-MOIST-1-3, | | | |
| | | | | | | | | | | ZM-MOIST-1-4, | | | |
| | | | | | | | | | | ZM-MOIST-4-2, | | | |
| | | | | | | | | | | ZM-MOIST-4-3, | | | |
| | | | | | | | | | | ZM-PC-1-1, | | | |
| | | | | | | | | | | ZM-DMC-1-1, | | | |
| | | | | | | | | | | ZM-DMY-1-2, | | | |
| | | | | | | | | | | ZM-DMY-1-4, | | | |
| | | | | | | | | | | ZM-DMY-4-3, | | | |
| | | | | | | | | | | ZM-GYHA-1-1, | | | |
| | | | | | | | | | | ZM-GYLD-1-2, | | | |
| | | | | | | | | | | ZM-GYUP-1-2, | | | |
| | | | | | | | | | | M-TW-1-1 | | | |
| 325 | — | — | — | — | X | — | X | — | — | OS-PGWC-8-1, | | | |
| | | | | | | | | | | OS-FLWID-3-1, | | | |
| | | | | | | | | | | OS-GPL-8-2, | | | |
| | | | | | | | | | | OS-GPP-8-2, | | | |
| | | | | | | | | | | OS-GYLD-8-2, | | | |
| | | | | | | | | | | ZM-CPC-1-3, | | | |
| | | | | | | | | | | ZM-CPC-1-5, | | | |
| | | | | | | | | | | ZM-IVDOM-1-2 | | | |
| | | | | | | | | | | ZM-IVDOM-1-3 | | | |
| | | | | | | | | | | ZM-MOIST-1-3, | | | |
| | | | | | | | | | | ZM-MOIST-1-4, | | | |
| | | | | | | | | | | ZM-PC-1-1, | | | |
| | | | | | | | | | | ZM-DMC-1-1, | | | |
| | | | | | | | | | | ZM-DMY-1-2, | | | |
| | | | | | | | | | | ZM-DMY-1-4, | | | |
| | | | | | | | | | | ZM-GYHA-1-1, | | | |
| | | | | | | | | | | ZM-GYLD-1-2, | | | |
| | | | | | | | | | | ZM-GYUP-1-2, | | | |
| | | | | | | | | | | ZM-TW-1-1 | | | |
| 265 | — | — | — | — | X | — | — | — | X | OS-FLLEN-3-1, | 65 | 79 | CGG |
| | | | | | | | | | | OS-GPL-2-1, | | | |
| | | | | | | | | | | OS-GYLD-2-1, | | | |
| | | | | | | | | | | MAS24-21, | | | |
| | | | | | | | | | | ZM-ID-5-2, | | | |
| | | | | | | | | | | ZM-MOIST-4-3, | | | |
| | | | | | | | | | | ZM-MOIST-4-4, | | | |
| | | | | | | | | | | ZM-MOIST-5-4, | | | |
| | | | | | | | | | | ZM-PC-5-1, | | | |
| | | | | | | | | | | ZM-STC-5-1, | | | |
| | | | | | | | | | | ZM-DMC-5-1, | | | |
| | | | | | | | | | | ZM-DMY-4-2, | | | |
| | | | | | | | | | | ZM-DMY-4-3, | | | |
| | | | | | | | | | | ZM-DMY-4-4, | | | |
| | | | | | | | | | | ZM-EWT-4-2, | | | |
| | | | | | | | | | | ZM-GYLD-4-1, | | | |
| | | | | | | | | | | ZM-GYLD-5-2, | | | |
| | | | | | | | | | | ZM-HI-4-1, | | | |
| | | | | | | | | | | ZM-KNE-4-1, | | | |
| | | | | | | | | | | ZM-KW300-4-2, | | | |
| | | | | | | | | | | ZM-KWE-4-1, | | | |
| | | | | | | | | | | M-TGW-4-1 | | | |
| 327 | — | — | — | | X | — | X | | | | | | |
| 231 | — | — | X | — | X | — | — | — | — | ZM-MOIST-2-3, | | | |
| | | | | | | | | | | ZM-MOIST-4-3, | | | |
| | | | | | | | | | | ZM-STC-2-2, | | | |
| | | | | | | | | | | ZM-DMY-2-3, | | | |
| | | | | | | | | | | ZM-DMY-2-4, | | | |
| | | | | | | | | | | ZM-DMY-4-3, | | | |
| | | | | | | | | | | M-GYLD-2-3 | | | |
| 37 | — | X | — | — | X | — | — | — | — | | | | |
| 43 | — | X | — | — | X | — | — | — | — | ZM-DMY-4-1 | | | |
| 293 | — | — | — | — | X | — | — | X | — | OS-CIF-6-1, | | | |
| | | | | | | | | | | MAS13-32, | | | |
| | | | | | | | | | | ZM-CPC-1-3, | | | |
| | | | | | | | | | | ZM-CPC-1-5, | | | |
| | | | | | | | | | | ZM-IVDOM-1-2, | | | |
| | | | | | | | | | | ZM-MOIST-1-4, | | | |
| | | | | | | | | | | ZM-MOIST-2-1, | | | |
| | | | | | | | | | | ZM-MOIST-9-2, | | | |
| | | | | | | | | | | ZM-PC-1-1, | | | |
| | | | | | | | | | | ZM-DMC-1-1, | | | |
| | | | | | | | | | | ZM-DMY-1-4, | | | |
| | | | | | | | | | | ZM-DMY-2-1, | | | |
| | | | | | | | | | | ZM-GYLD-2-4, | | | |
| | | | | | | | | | | ZM-GYLD-9-1, | | | |
| | | | | | | | | | | ZM-GYUP-1-2, | | | |
| | | | | | | | | | | ZM-KW100-9-1, | | | |
| | | | | | | | | | | ZM-KW300-9-2, | | | |
| | | | | | | | | | | ZM-TW-1-1, | | | |
| | | | | | | | | | | ZM-YLD-9-1 | | | |
| 321 | X | — | — | — | X | — | X | — | — | ZM-CPC-6-2, | 536 | 550 | CTG |
| | | | | | | | | | | ZM-DMC-6-1, | | | |
| | | | | | | | | | | ZM-DMC-6-2, | | | |
| | | | | | | | | | | ZM-GYLD-6-1, | | | |
| | | | | | | | | | | ZM-GYLD-6-4, | | | |
| | | | | | | | | | | ZM-GYLD-6-4, | | | |
| | | | | | | | | | | ZM-YLD-6-1 | | | |
| 79 | X | — | — | — | X | — | — | — | — | OS-AMY-5-1 | | | |
| 211 | — | — | X | — | X | — | — | — | — | OS-APDF-9-1, | | | |
| | | | | | | | | | | OS-VGT-9-1, | | | |
| | | | | | | | | | | OS-GW-9-1 | | | |
| 177 | X | — | — | — | X | — | — | — | — | OS-CIF-6-1 | 44 | 58 | CGT |
| | | | | | | | | | | | 117 | 131 | GGA |

TABLE 3
Table 3 provides a further subset of rice genes the expression
of which is up-regulated during grain filling. Further identified
are SSR sequences in the coding region of the rice genes.

A = structural protein
B = hypothetical/unknown proteins
C = Growth/division and development
D = classification not clear
E = Cereal_Grain_Filling_QTLs (a description of the respective QTLs is provided in
Table . . . below)
F = Beginning of the SSR
G = End of the SSR
H = Nucleotide Sequence of the trinucleotide repeat unit

| SEQ ID | A | B | C | D | E | F | G | H |
|---|---|---|---|---|---|---|---|---|
| 329 | — | X | — | — | | | | |
| 331 | — | — | — | X | | | | |
| 332 | X | — | — | — | | | | |
| 333 | — | X | — | — | | | | |
| 334 | — | X | — | — | | | | |
| 335 | — | X | — | — | | | | |
| 343 | — | — | — | X | | | | |
| 23 | — | X | — | — | | | | |
| 345 | — | X | — | — | | | | |
| 351 | — | X | — | — | | | | |
| 355 | — | X | — | — | | | | |
| 357 | — | X | — | — | | | | |
| 361 | — | X | — | — | | | | |
| 363 | — | X | — | — | | | | |
| 365 | — | — | — | X | | | | |
| 369 | — | — | — | X | | | | |
| 371 | — | — | X | — | | | | |
| 373 | — | X | — | — | | | | |
| 313 | — | X | — | — | | | | |
| 375 | — | X | — | — | | | | |
| 377 | — | X | — | — | | | | |
| 379 | — | X | — | — | | | | |
| 381 | — | — | — | X | | | | |
| 383 | — | X | — | — | | | | |
| 387 | — | X | — | — | | | | |
| 389 | — | X | — | — | | | | |
| 393 | — | X | — | — | | | | |
| 395 | — | X | — | — | | | | |
| 99 | — | — | — | X | | | | |
| 397 | — | X | — | — | | | | |
| 229 | — | X | — | — | | | | |
| 403/431- | — | — | | | | 16 | 39 | CCG |
| 433 | — | X | — | — | OS-AMY-5-1, | | | |
| | | | | | MAS13-31, | | | |
| | | | | | SMS021-80, | | | |
| | | | | | ZM-CPC-5-1, | | | |
| | | | | | ZM-CPC-7-2, | | | |
| | | | | | ZM-IVDOM-5-1, | | | |
| | | | | | ZM-IVDOM-5-2, | | | |
| | | | | | ZM-MOIST-5-2, | | | |
| | | | | | ZM-MOIST-5-2, | | | |
| | | | | | ZM-MOIST-5-3, | | | |
| | | | | | ZM-MOIST-7-1, | | | |
| | | | | | ZM-BIOM-5-1, | | | |
| | | | | | ZM-BIOM-7-1, | | | |
| | | | | | ZM-DMY-5-1, | | | |
| | | | | | ZM-GWM2-7-1, | | | |
| | | | | | ZM-GYLD-5-1, | | | |
| | | | | | ZM-GYLD-5-3, | | | |
| | | | | | ZM-HI-7-1, | | | |
| | | | | | ZM-KW300-5-1, | | | |
| | | | | | ZM-TW-5-1 | | | |
| 435 | — | — | — | X | | | | |
| 437 | — | X | — | — | | | | |
| 439 | — | X | — | — | OS-YLD-3-2, | | | |
| | | | | | ZM-ID-5-1, | | | |
| | | | | | ZM-IVDOM-5-3, | | | |
| | | | | | ZM-GYLD-5-3 | | | |
| 441 | — | — | — | X | OS-REGEN-5-1, | 1912 | 1929 | CGG |
| | | | | | MAS12-18, | | | |
| | | | | | MAS24-16, | | | |
| | | | | | SMS015-16, | | | |
| | | | | | SMS021-81, | | | |
| | | | | | ZM-ID-6-1, | | | |
| | | | | | ZM-ID-6-1, | | | |
| | | | | | ZM-ID-8-1, | | | |
| | | | | | ZM-ID-8-1, | | | |
| | | | | | ZM-ID-8-1, | | | |
| | | | | | ZM-MOIST-5-1, | | | |
| | | | | | ZM-MOIST-6-2, | | | |
| | | | | | ZM-PC-8-1, | | | |
| | | | | | ZM-STC-6-1, | | | |
| | | | | | ZM-STC-8-1, | | | |
| | | | | | ZM-VT-6-1, | | | |
| | | | | | ZM-BIOM-8-1, | | | |
| | | | | | ZM-DMC-8-1, | | | |
| | | | | | ZM-DMY-8-1, | | | |
| | | | | | ZM-DMY-8-2, | | | |
| | | | | | ZM-GYHA-5-1, | | | |
| | | | | | ZM-GYHA-6-1, | | | |
| | | | | | ZM-GYLD-5-3, | | | |
| | | | | | ZM-GYLD-6-2, | | | |
| | | | | | ZM-GYLD-6-3, | | | |
| | | | | | ZM-HI-8-1, | | | |
| | | | | | ZM-KW300-6-2 | | | |
| 443 | — | — | — | X | OS-RGT-12-2, | 117 | 131 | CGG |
| | | | | | OS-GWPL-12-1 | 1962 | 1979 | CGG |
| 445 | — | X | — | — | | | | |
| 447 | — | X | — | — | OS-YLD-3-2 | | | |
| 95 | — | — | — | X | OS-CIF-8-1, | | | |
| | | | | | OS-GW-8-1, | | | |
| | | | | | MAS13-24, | | | |
| | | | | | ZM-CPC-1-3, | | | |
| | | | | | ZM-CPC-1-5, | | | |
| | | | | | ZM-IVDOM-1-2, | | | |
| | | | | | ZM-IVDOM-1-4, | | | |
| | | | | | ZM-MOIST-1-4, | | | |
| | | | | | ZM-MOIST-1-5, | | | |
| | | | | | ZM-PC-1-1, | | | |
| | | | | | ZM-DMC-1-1, | | | |
| | | | | | ZM-DMC-6-2, | | | |
| | | | | | ZM-DMY-1-4, | | | |
| | | | | | ZM-GYLD-6-4, | | | |
| | | | | | ZM-GYUP-1-2, | | | |
| | | | | | ZM-KW300-1-2, | | | |
| | | | | | ZM-TW-1-1, | | | |
| | | | | | ZM-YLD-6-1 | | | |
| 451 | — | X | — | — | OS-PGWC-12-1, | 962 | 976 | GCA |
| | | | | | OS-BDV-12-1, | | | |
| | | | | | OS-PKV-12-1 | | | |
| 453 | — | X | — | — | | 27 | 47 | CCT |
| | | | | | | 344 | 358 | GCG |
| 455 | — | X | — | — | MAS24-28, | | | |
| | | | | | ZM-ID-10-1, | | | |
| | | | | | ZM-ID-2-1, | | | |
| | | | | | ZM-MOIST-10-1, | | | |
| | | | | | ZM-MOIST-2-2, | | | |
| | | | | | ZM-MOIST-4-3, | | | |
| | | | | | ZM-MOIST-5-3, | | | |
| | | | | | ZM-STC-10-1, | | | |
| | | | | | ZM-DMC-10-1, | | | |
| | | | | | ZM-DMC-10-2, | | | |
| | | | | | ZM-DMC-2-3, | | | |
| | | | | | ZM-DMY-10-1, | | | |
| | | | | | ZM-DMY-4-3, | | | |
| | | | | | ZM-EWT-2-1, | | | |
| | | | | | ZM-GWM2-10-1, | | | |
| | | | | | ZM-GYLD-2-2, | | | |
| | | | | | ZM-GYLD-5-2, | | | |
| | | | | | ZM-HI-10-1, | | | |
| | | | | | ZM-TW-10-2, | | | |
| | | | | | ZM-TW-2-3 | | | |
| 457 | — | X | — | — | | | | |
| 459 | — | X | — | — | OS-PGWC-12-1, | 53 | 73 | CGG |
| | | | | | OS-BDV-12-1, | | | |
| | | | | | OS-PKV-12-1 | | | |
| 461 | — | X | — | — | OS-GW-11-1, | | | |
| | | | | | ZM-IVDOM-9-1, | | | |
| | | | | | ZM-IVDOM-9-2, | | | |
| | | | | | ZM-GYLD-9-2, | | | |
| | | | | | ZM-KW100-9-1, | | | |
| | | | | | ZM-TGW-9-2 | | | |

TABLE 4
Genes involved in rice grain filling, which belong to
the functional category of stress response proteins

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 1 | — | 1065 | 1182 | Similar to MPV1_HUMAN |
| | | | | P39210 HOMO SAPIENS |
| | | | | (HUMAN). MPV17 PROTEIN. |
| 3 | | | 1115 | Similar to ANRX_ANASP |
| | | | | Q44141 ANABAENA SP. |
| | | | | (STRAIN PCC 7120). |
| | | | | ANAREDOXIN. |
| 5 | 939 | 1030 | 1184 | Similar to |
| | | | | gi\|20286\|emb\|CAA46916.1\| |
| | | | | peroxidase [Oryza sativa] |
| 7 | 935 | 1037 | — | Similar to |
| | | | | gi\|1620753\|gb\|AAB17095.1\| |
| | | | | proteinase inhibitor |
| | | | | [Oryza sativa] |
| 9 | 934 | 1011 | 1110 | Similar to |
| | | | | gi\|3287683\|gb\|AAC25511.1\| |
| | | | | Similar to apoptosis |
| | | | | protein MA-3 |
| | | | | gb\|D50465 from Mus |
| | | | | musculus. [Arabidopsis |
| | | | | thaliana] |
| 11 | — | 952 | 1198 | Similar to |
| | | | | gi\|5725430\|emb\|CAB52439.1\| |
| | | | | stress responsive protein |
| | | | | homolog [Arabidopsis |
| | | | | thaliana] |
| 13 | — | 998 | 1175 | |
| 15 | — | 1015 | 1167 | |
| 17 | 899 | 1042 | 1161 | |

TABLE 5
Genes involved in rice grain filling, which belong
to the functional category of signaling molecules

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 19 | — | 1089 | — | Similar to |
| | | | | gi\|1352683\|sp\|P49599\| |
| | | | | P2C3_ARATH PROTEIN |
| | | | | PHOSPHATASE 2C PPH1 (PP2C) |
| 21 | — | 971 | — | Similar to |
| | | | | gi\|7269803\|emb\|CAB79663.1\| |
| | | | | serine/threonine-specific |
| | | | | kinase like protein |
| | | | | [Arabidopsis thaliana] |
| 23 | | | | Similar to |
| | | | | gi\|6520139\|dbj\| |
| | | | | BAA87936.1\| ZW9 |
| | | | | [Arabidopsis thaliana] |
| 25 | — | 1071 | 1120 | Similar to |
| | | | | gi\|9293975\|dbj\| |
| | | | | BAB01878.1\| receptor |
| | | | | protein kinase |
| | | | | [Arabidopsis thaliana] |
| 27 | 916 | 1049 | — | |
| 29 | — | 984 | 1186 | |

TABLE 6
Genes involved in rice grain filling, which belong to
the functional category of transmembrane proteins

| Rice | Banana | Wheat | Banana | |
|---|---|---|---|---|
| (SEQ | (SEQ | (SEQ | (SEQ | |
| ID | ID | ID | ID | |
| NO | NO) | NO) | NO) | Gene Description |
| 31 | — | 1025 | — | (nitrite transporter) |
| 33 | — | 1047 | — | (amino a selective |
| | | | | channel protein) |
| 35 | 950 | 959 | 1164 | (G6P transporter in |
| | | | | plastids) |
| 37 | | | | (PTR2 POT family) |
| 39 | 949 | 1017 | — | (Leucine rich protein) |
| 41 | 927 | 962 | 1112 | (immunoglobulin) |
| 43 | 917 | 982 | 1109 | (dehydrogenase) |
| 45 | — | 954 | 1117 | (putative transport |
| | | | | protein) |
| 47 | 921 | 1099 | 1152 | (phosphate transporter) |
| 49 | 891 | 1040 | 1128 | (monosaccarid (hexose) |
| | | | | transporter) |
| 51 | — | 994 | — | (PTR2 POT family) |
| 53 | — | 1067 | 1159 | (cation transporter |
| | | | | protein Ec) |
| 55 | — | 1047 | — | (amino a selective |
| | | | | channel protein) |
| 57 | | | | (sugar transporter) |
| 59 | — | 1077 | — | (transporter protein) |
| 61 | — | 1085 | — | Similarity[ab043024_34-1656 |
| | | | | /codon_start = 1 |
| | | | | /db_xref = “gi: 8051712” / |
| | | | | product = “sodium sulfate |
| | | | | or dicarboxylate transporter” |
| | | | | /protein_id = “baa96091.1” ] |
| | | | | Evidence[100% (1510/1510)] |
| 63 | — | 1105 | — | Similar to |
| | | | | gi\|7523692\|gb\|AAF63131.1\| |
| | | | | AC011001_1 Putative |
| | | | | chloroplast inner |
| | | | | envelope protein |
| | | | | [Arabidopsis thaliana] |
| 65 | — | 957 | 1114 | Similar to PITH_STRHA |
| | | | | P41132 STREPTOMYCES |
| | | | | HALSTEDII. PUTATIVE LOW- |
| | | | | AFFINITY INORGANIC |
| | | | | PHOSPHATE TRANSPORTER |
| | | | | (FRAGMENT) |
| 67 | 944 | 1075 | — | Similar to PTR2_YEAST |
| | | | | P32901 SACCHAROMYCES |
| | | | | CEREVISIAE (BAKER S |
| | | | | YEAST). PEPTIDE |
| | | | | TRANSPORTER PTR2 |
| | | | | (PEPTIDE PERMEASE PTR2). |

TABLE 7
Genes involved in rice grain filling, which belong to the functional category of carbohydrate metabolism
STARCH METABOLISM

| | | | | |
|---|---|---|---|---|
| Rice | Banana | Wheat | Maize | |
| (SEQ ID | (SEQ ID | (SEQ ID | (SEQ ID | |
| NO) | NO) | NO) | NO) | Gene Description |

Branching Enzyme

| | | | | |
|---|---|---|---|---|
| 69 | 888 | 1058 | — | Similar to GLGB_ORYSA Q01401 ORYZA SATIVA (RICE). |
| | | | | 1,4-ALPHA-GLUCAN BRANCHING ENZYME (EC 2.4.1.18) |
| | | | | (STARCH BRANCHINGENZYME) (Q-ENZYME). |
| 71 | — | 1026 | 1157 | Similar to gi\|4584507\|emb\|CAB40745.1\| |
| | | | | starch branching enzyme II [Solanum tuberosum] |
| 73 | — | 1018 | 1157 | gi\|3851526\|gb\|AAC72335.1\| |
| | | | | starch branching enzyme IIa [Hordeum vulgare |

Debranching Enzyme

| | | | | |
|---|---|---|---|---|
| 75 | — | 987 | — | gi\|1783306\|dbj\|BAA09167.1\| |
| | | | | starch debranching enzyme precursor [Oryza sativa] |
| 77 | — | 966 | — | Similar to gi\|3252794\|dbj\|BAA29041.1\| |
| | | | | isoamylase [Oryza sativa] |

Starch degradation Alpha - Amylases

| | | | | |
|---|---|---|---|---|
| 79 | 909 | 1083 | 1173 | Similar to AMYM_BACST P19531 BACILLUS |
| | | | | STEAROTHERMOPHILUS. MALTOGENIC |
| | | | | ALPHA-AMYLASE PRECURSOR (EC 3.2.1.133) |
| | | | | (GLUCAN 1,4-ALPHA-MALTOHYDROLASE) |
| 81 | 887 | 1035 | 1150 | Similar to gi\|426482\| Alpha-amylase |
| 83 | 887 | 1033 | 1150 | \|CAA39777.1\| Alpha- amylase |
| 85 | — | 1033 | 1150 | \|CAA39777.1\| Alpha- amylase |
| 87 | 887 | 1033 | 1151 | \|PF00128\| Alpha-amylase |
| 89; | 887 | 1032 | 1150 | gi\|426482\|aaa50161.1\| Alpha-amylase |
| 509 | | | | |
| 91 | — | 1034 | 1150 | gi\|113766\|sp\|P17654\|AMY1_ORYSA |
| | | | | ALPHA-AMYLASE PRECURSOR (1,4-ALPHA-D-GLUCAN |
| | | | | GLUCANOHYDROLASE) (ISOZYME 1B) |

alpha-Amylase Inhibitor

| | |
|---|---|
| 93 | |
| 95 | Motifs{Cereal_Tryp_Amyl_Inh Cereal trypsin/ |
| | alpha-amylase inhibitors family; |
| | Pfam6_1\|PF00234\|tryp_alpha_amyl Protease |
| | inhibitor/seed storage family} Evidence[100% (474/474)] |
| 97 | Motifs{Aldehyde_Dehydr_Cys Aldehyde |
| | dehydrogenases active sites; Cereal_Tryp_Amyl_Inh |
| | Cereal trypsin/alpha-amylase inhibitors family} |
| | Evidence[99% (486/489)] |
| 99 | Motifs{Cereal_Tryp_Amyl_Inh Cereal trypsin/ |
| | alpha-amylase inhibitors family; |
| | Pfam6_1\|PF00234\|tryp_alpha_amyl Protease |
| | inhibitor/seed storage family} Evidence[100% (501/501)] |

Beta-Amylase

| | | | | |
|---|---|---|---|---|
| 101 | — | 965 | 1107 | Similarity[y16242_1-1798 /codon_start = 2 |
| | | | | /db_xref = “gi: 4138596” /partial = true |
| | | | | /product = “beta-amylase” /protein_id = |
| | | | | “caa76131.1”] Evidence[100% (931/931)]. |
| 103 | 926 | 956 | 1156 | Similarity[z25871_48-1514 /codon_start = 1 |
| | | | | /db_xref = “swiss-prot:p55005” /ec_number = |
| | | | | “3.2.1.2” /product = “beta-amylase” |
| | | | | /protein_id = “caa81091.1”] |
| | | | | Evidence[100% (1539/1539)] |
| 105 | — | 955 | — | gi\|1703302\|sp\|P55005\|AMYB_MAIZE BETA- |
| | | | | AMYLASE (1,4-ALPHA-D-GLUCAN MALTOHYDROLASE) |
| 107 | — | 965 | 1106 | gi\|3334120\|sp\|P93594\|AMYB_WHEAT BETA- |
| | | | | AMYLASE (1,4-ALPHA-D-GLUCAN MALTOHYDROLASE) |

Pullulanase

| | | | | |
|---|---|---|---|---|
| 109 | — | 987 | — | Similarity[ab012915_2206-14924 /codon_start = 1 |
| | | | | /db_xref = “gi: 3172048” /product = “starch |
| | | | | debranching enzyme” /protein_id = “baa28632.1” |
| | | | | /note = “pullulanase”] Evidence[100% (3079/3079)] |
| | 887 | 1032 | 1150 | |

Glucosidase

| | | | | |
|---|---|---|---|---|
| 111 | — | 1005 | — | Similar to AMYG_NEUCR P14804 NEUROSPORA CRASSA. |
| | | | | GLUCOAMYLASE PRECURSOR (EC 3.2.1.3) (GLUCAN 1,4-ALPHA- |
| | | | | GLUCOSIDASE)-(1,4-ALPHA-D-GLUCAN GLUCOHYDROLASE). |
| 113 | 905 | 1021 | — | \|CAA04707.1\| Alpha-glucosidase |
| 115 | — | 1086 | 1144 | gi\|3023275\|sp\|Q43763\|AGLU_HORVU ALPHA- |
| | | | | GLUCOSIDASE PRECURSOR (MALTASE) |
| 117 | | | | gi\|544151\|sp\|Q99040\|DEXB_STRMU GLUCAN |
| | | | | 1,6-ALPHA-GLUCOSIDASE (DEXTRAN |
| | | | | GLUCOSIDASE) (EXO-1,6-ALPHA- |
| | | | | GLUCOSIDASE) (GLUCODEXTRANASE) |

Surose Synthase

| | | | | |
|---|---|---|---|---|
| 119 | 932 | 1006 | 1148 | Similar to SUS2_ARATH Q00917 ARABIDOPSIS |
| | | | | THALIANA (MOUSE-EAR CRESS). SUCROSE |
| | | | | SYNTHASE (EC 2.4.1.13) (SUCROSE-UDP |
| | | | | GLUCOSYLTRANSFERASE). |
| 121 | 930 | 1022 | 1170 | gi\|283009\|pir∥S22535 sucrose synthase |
| | | | | (EC 2.4.1.13) 1 - rice (fragment) |
| 123 | 930 | 1028 | 1170 | gi\|20366\|emb\|CAA46017.1\| sucrose synthase |
| | | | | [Oryza sativa] |
| 125 | 930 | 1054 | 1170 | gi\|267055\|sp\|Q00917\|SUS2_ARATH SUCROSE SYNTHASE |
| | | | | (SUCROSE-UDP GLUCOSYLTRANSFERASE) |
| 127 | 930 | 1054 | 1191 | gi\|66572\|pir∥YUMU sucrose synthase |
| | | | | (EC 2.4.1.13) - Arabidopsis thaliana |

Starch Synthase

| | | | | |
|---|---|---|---|---|
| 129 | — | 1066 | — | Similar to UGS3_SOLTU Q43847 SOLANUM |
| | | | | TUBEROSUM (POTATO). GLYCOGEN (STARCH) |
| | | | | SYNTHASE PRECURSOR (EC 2.4.1.11) (GBSSII) |
| | | | | (GRANULE-BOUND STARCH SYNTHASE II) (FRAGMENT) |
| 131 | 924 | 1070 | 1125 | Similar to gi\|3057122\|gb\|AAC14015.1\| starch |
| | | | | synthase DULL1 [Zea mays |
| 133 | 947 | 1055 | 1155 | Similar to gi\|5257102\|gb\|AAD41242.1\| granule |
| | | | | bound starch synthase [Oryza sativa subsp. japonica] |

ADPG pyrophosphorylase

| | | | | |
|---|---|---|---|---|
| 135 | — | 989 | 1193 | Similar to gi\|3093462\|gb\|AAC15247.1\| ADP-glucose |
| | | | | pyrophosphorylase large subunit [Oryza sativa] |
| 137 | 922 | 1098 | — | Similarity[ay028315_115-1617 /codon_start = 1 |
| | | | | /db_xref = “gi: 13508485” /product = “adp-glucose |
| | | | | pyrophosphorylase small subunit” /protein_id = |
| | | | | “aak27313.1” /note = “putative amyloplast form”] |
| | | | | Evidence[100% (1520/1520)] |
| 139 | — | 989 | 1193 | Similarity[ac007858_66917-70303 /codon_start = 1 |
| | | | | /db_xref = “gi: 5091608” /evidence = |
| | | | | “not_experimental” /gene = “10a19i.12” /protein_id = |
| | | | | “aad39597.1” /note = “identical to gb\|d50317 adp |
| | | | | glucose pyrophosphorylase large subunit from oryza sativa. |
| | | | | ests dbj\|d22125 and dbj\|d15718 come from”] |
| | | | | Evidence[100% (1615/1615)] Gene[10A19I.12 Identical to |
| | | | | gb\|D50317 ADP glucose pyrophosphorylase large subunit from |
| | | | | Oryza sativa. ESTs dbj\|D22125 and dbj\|D15718 come from] |
| 141 | 922 | 1098 | 1193 | Similar to gi\|169759\|gb\|AAA33890.1\| ADP- |
| | | | | glucose pyrophosphorylase 51 kD subunit |
| | | | | (EC 2.7.7.27) |

Triosephosphate Isomerase

| | | | | |
|---|---|---|---|---|
| 143 | 912 | 1046 | 1133 | Similarity[z32521_64-960 /codon_start = 1 |
| | | | | /db_xref = “swiss-prot: p46225” /ec_number = |
| | | | | “5.3.1.1” /product = “triosephosphate isomerase” |
| | | | | /protein_id = “caa83533.1”] Evidence[100% (822/822)] |
| 145 | 912 | 1046 | 1133 | db_xref = “swiss-prot: p46225” /ec_number = |
| | | | | “5.3.1.1” /product = “triosephosphate isomerase” |
| | | | | /protein_id = “caa83533.1”] Evidence[100% (822/822)] |
| 147 | 890 | 1003 | 1134 | Similarity[j04121_1-762 /codon_start = 1 /db_xref = |
| | | | | “gi: 556171” /product = “triosephosphate isomerase” |
| | | | | /protein_id = “aab62730.1”] Evidence[100% (683/683)] |

Other proteins involved in starch metabolism

| | | | | |
|---|---|---|---|---|
| 149 | 936 | 1043 | 1194 | Similarity[x53130_51-1127 /codon_start = 1 |
| | | | | /db_xref = “swiss-prot: p17784” /protein_id = |
| | | | | “caa37290.1” /note = “fructose-diphosphate aldolase |
| | | | | (aa 1-358)”] Evidence[100% (1078/1078)] |
| 151 | — | 963 | 1124 | \|AAA45939.1\|Alpha-1,4-glucan phosphorylase h isozyme |
| 153 | 950 | 959 | 1164 | Similarity[af020813_273-1436 /codon_start = 1 |
| | | | | /db_xref = “gi: 2997589” /function = “mediates |
| | | | | the antiport of glucose-6-phosphate against phosphate in plastids |
| | | | | of heterotrophic tissues” /gene = “gpt” /product = |
| | | | | “glucose-6-phosphate/phosphate-translocator precursor” |
| | | | | /protein_id = “aac08524.1” |
| 155; | 913 | — | 1154 | gi\|4539316\|emb\|CAB38817.1\| putative fructose- |
| 507 | | | | bisphosphate aldolase [Arabidopsis thaliana] |
| 157 | — | 1069 | — | Motifs{Pfam6_1\|PF00702\|Hydrolase haloacid |
| | | | | dehalogenase-like hydrolase} Evidence[82% (1032/1254)] |
| 159 | — | 1023 | — | Similarity[u17225_40-1743 /codon_start = 1 |
| | | | | /db_xref = “gi: 596023” /ec_number = “5.3.1.9” |
| | | | | /gene = “phil” /product = “glucose-6 phosphate |
| | | | | isomerase” /protein_id = “aaa82734.1” /note = |
| | | | | “phosphohexose isomerase”] Evidence[100% (1889/1889)] |
| | | | | Gene[phil 5.3.1.9 glucose-6 phosphate isomerase phosphohexose |
| | | | | isomerase] |
| 161 | 946 | 1103 | 1189 | Similarity[ab013353_89-1504 /codon_start = 1 |
| | | | | /db_xref = “gi: 3107931” /product = “udp-glucose |
| | | | | pyrophosphorylase” /protein_id = “baa25917.1”] |
| | | | | Evidence[100% (1582/1582)] |
| 163 | 937 | 970 | 1153 | Similarity[af372833_47-1273 /codon_start = 1 |
| | | | | /db_xref = “gi: 13991929” /product = “phos- |
| | | | | phoenolpyruvate/phosphate translocator” /protein_id = |
| | | | | “aak51561.1” /note = “ppt”] |
| | | | | Evidence[100% (1239/1239)] |
| 165 | 892 | 964 | 1179 | Similar to gi\|5231119\|gb\|AAD41079.1\|AF143202_1 |
| | | | | starch phosphorylase L [Solarium tuberosum]; |
| | | | | gi\|130172\|sp\|P27598\|PHSL_IPOBA ALPHA-1,4 |
| | | | | GLUCAN PHOSPHORYLASE, L ISOZYME, CHLOROPLAST |
| | | | | PRECURSOR (STARCH PHOSPHORYLASE L) |
| 167 | 902 | 997 | — | Motifs{Pfam6_1\|PF01591\|6PF2K 6-phosphofructo- |
| | | | | 2-kinase; Atp_Gtp_A ATP/GTP-binding site motif |
| | | | | A (P-loop)} Evidence[71% (2205/3069)] |
| 169 | 946 | 1050 | — | Similarity[ap001383_68171-73040 /codon_start = 1 |
| | | | | /db_xref = “gi: 7242911” /protein_id = |
| | | | | “baa92509.1” /note = “similar to udp-glucose |
| | | | | pyrophosphorylase. (x91347)”] Evidence[100% (1528/1528) |
| 171 | — | 1023 | — | Similarity[u17225_40-1743 /codon_start = 1 |
| | | | | /db_xref = “gi: 596023” /ec_number = “5.3.1.9” |
| | | | | /gene = “phil” /product = “glucose-6 phosphate |
| | | | | isomerase” /protein_id = “aaa82734.1” /note = |
| | | | | “phosphohexose isomerase”] Evidence[100% (1889/1889)] |
| | | | | Gene[phil 5.3.1.9 glucose-6 phosphate isomerase phosphohexose |
| | | | | isomerase] |
| 173 | — | 975 | — | Similarity[d45218_54-1760 /codon_start = 1 |
| | | | | /db_xref = “gi: 639686” /product = |
| | | | | “phosphoglucose isomerase (pgi-b)” /protein_id = |
| | | | | “baa08149.1”] Evidence[100% (1409/1409)] |
| 175 | 937 | 970 | 1153 | Similarity[af372833_47-1273 /codon_start = 1 |
| | | | | /db_xref = “gi: 13991929” /product = |
| | | | | “phosphoenolpyruvate/phosphate translocator” |
| | | | | /protein_id = “aak51561.1” /note = “ppt”] |
| | | | | Evidence[100% (1050/1050)] |
| 177 | 889 | 1081 | 1196 | Motifs{Pfam6_1\|PF00274\|glycolytic_enzy |
| | | | | Fructose-bisphosphate aldolase class-I; |
| | | | | Aldolase_Class_I Fructose-bisphosphate aldolase |
| | | | | class-I active site} Evidence[65% (1082/1650)] |
| 179 | — | 977 | 1180 | Similarity[z32850_352-4957 /codon_start = 1 |
| | | | | /db_xref = “swiss-prot: q41141” /product = |
| | | | | “pyrophosphate-dependent phosphofructokinase betasubunit” |
| | | | | /protein_id = “caa83683.1”] Evidence[100% (1698/1698)] |
| 181 | 892 | 964 | 1179 | Similarity[af095521_76-1923 /codon_start = 1 |
| | | | | /db_xref = “gi: 3790102” /ec_number = “2.7.1.90” |
| | | | | /gene = “ppi-pfka” /product = “pyrophosphate- |
| | | | | dependent phosphofructokinase alpha subunit” /protein_id = |
| | | | | “aac67587.1”] Evidence[100% (1939/1939)] |
| | | | | Gene[PPi-PFKa 2.7.1.90 pyrophosphate-dependent phosphofructo- |
| | | | | kinase alpha subunit] |
| 183 | 906 | 988 | 1113 | gi\|3122594\|sp\|Q59126\|PFP_AMYME |
| | | | | PYROPHOSPHATE--FRUCTOSE 6-PHOSPHATE |
| | | | | 1-PHOSPHOTRANSFERASE (6-PHOSPHOFRUCTOKINASE |
| | | | | (PYROPHOSPHATE)) (PYROPHOSPHATE-DEPENDENT 6- |
| | | | | PHOSPHOFRUCTOSE-1-KINASE) (PPI-PFK) |
| 185 | 896 | 1014 | 1180 | gi\|2499488\|sp\|Q41140\|PFPA_RICCO |
| | | | | PYROPHOSPHATE--FRUCTOSE 6-PHOSPHATE |
| | | | | 1-PHOSPHOTRANSFERASE ALPHA SUBUNIT |
| | | | | (PFP) (6-PHOSPHOFRUCTOKINASE (PYROPHOSPHATE)) |
| | | | | (PYROPHOSPHATE-DEPENDENT 6-PHOSPHOFRUCTOSE-1- |
| | | | | KINASE) (PPI-PFK) |
| 187; | 911 | — | 1138 | gi\|3913641\|sp\|O64422\|F16P_ORYSA |
| 511 | | | | FRUCTOSE-1,6-BISPHOSPHATASE, CHLOROPLAST PRECURSOR |
| | | | | (D-FRUCTOSE-1,6-BISPHOSPHATE 1-PHOSPHOHYDROLASE) (FBPASE) |

Non-Starch Carbohydrate Metabolism

| | | | | |
|---|---|---|---|---|
| 189 | 912 | 1046 | 1133 | Similarity[z3252l_64-960 /codon_start = 1 |
| | | | | /db_xref = “swiss-prot: p46225” /ec_number = |
| | | | | “5.3.1.1” /product = “triosephosphate isomerase” |
| | | | | /protein_id = “caa83533.1”] Evidence[100% (822/822)] |
| 191; | — | 1052 | 1121 | Similar to gi\|9294516\|dbj\|BAB02778.1\| contains |
| 503 | | | | similarity to endo-1,3-1,4-beta-D-glucanase˜gene_id: |
| | | | | MDB19.8 [Arabidopsis thaliana] |
| 193 | | | | Similar to PTSN_ECOLI P31222 ESCHERICHIA |
| | | | | COLI. NITROGEN REGULATORY IIA PROTEIN |
| | | | | (EC 2.7.1.69) (ENZYME IIA- |
| | | | | NTR)(PHOSPHOTRANSFERASE ENZYME II, A |
| | | | | COMPONENT); Motifs{Cytochrome_C Cytochrome |
| | | | | c family heme-binding site; Zinc_Finger_C2h2_1 |
| | | | | Zinc finger, C2H2 type, domain; Zinc_Finger_C2h2_1 |
| | | | | Zinc finger, C2H2 type, domain; Zinc_Finger_C2h2_1 |
| | | | | Zinc finger, C2H2 type, domain) Evidence[0% (0/2145)] |
| 195 | — | 1041 | 1137 | Similar to gi\|6714431\|gb\|AAF26119.1\|ACO12328_22 |
| | | | | putative cellulose synthase catalytic subunit |
| | | | | [Arabidopsis thaliana] |
| 197 | | | | Similar to gi\|22327\|emb\|CAA37998.1\| corn Hageman |
| | | | | factor inhibitor [Zea mays] |
| 199 | — | 1096 | — | gi\|728850\|sp\|P08640\|AMYH_YEAST |
| | | | | GLUCOAMYLASE S1/S2 PRECURSOR |
| | | | | (GLUCAN |
| 201 | | | | Elements[GC_box@16653 TATA_box@16019 |
| | | | | ATG@15968 PolyA@10370] Evidence[88% (2550/2886) |
| 203 | — | 1020 | 1140 | Similar to gi\|3850573\|gb\|AAC72113.1\| Similar to |
| | | | | gi\|1652733 glycogen operon protein GlgX from |
| | | | | Synechocystis sp. genome gb\|D90908.ESTs |
| | | | | gb\|H36690, gb\|AA712462, gb\|AA651230 and |
| | | | | gb\|N95932 come from this gene. [Arabidopsis thaliana] |
| 205 | 904 | 1095 | 1130 | Similar to gi\|5441877\|dbj\|BAA82375.1\| Similar |
| | | | | to glycogenin glucosyltransferase |
| | | | | (EC 2.4.1.186). (Z97341) [Oryza sativa] |
| 207 | 895 | 1076 | 1181 | Similar to gi\|8777412\|dbj\|BAA97002.1\|indole-3- |
| | | | | glycerol phosphate synthase [Arabidopsis thaliana] |
| 209 | — | 1101 | — | gi\|14156\|sp\|P13526\|ARLC_MAIZE |
| | | | | ANTHOCYANIN REGULATORY LC PROTEIN |

TABLE 8
Genes involved in rice grain filling, which belong
to the functional category of storage proteins

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 211 | | | | gi\|121099\|sp\| |
| | | | | P08079\|GDB0_WHEAT |
| | | | | GAMMA-GLIADIN PRECURSOR |
| 213 | — | 1044 | 1165 | Similar to GL19_ORYSA |
| | | | | P29835 ORYZA SATIVA |
| | | | | (RICE). 19 KD GLOBULIN |
| | | | | PRECURSOR (ALPHA- |
| | | | | GLOBULIN). |
| 215 | | | | Similar to |
| | | | | gi\|224389\|prf∥ |
| | | | | 1103218A glycinin A5 |
| | | | | [Glycine max] |
| 217 | | | | Similar to |
| | | | | gi\|296129\|emb\| |
| | | | | CAA46197.1\| prolamin |
| | | | | [Oryza sativa] |
| 219 | | | | Similar to |
| | | | | gi\|7209261\|emb\| |
| | | | | CAB76962.1\| alpha-gliadin |
| | | | | [Triticum aestivum] |
| | | | | Similar to |
| | | | | gi\|4126695\|dbj\| |
| | | | | BAA36699.1\| prolamin |
| | | | | [Oryza sativa] |
| 221 | | | | Similar to METC_RHILV |
| | | | | Q52811 RHIZOBIUM |
| | | | | LEGUMINOSARUM |
| | | | | (BIOVAR VICIAE). |
| | | | | PUTATIVE |
| | | | | CYSTATHIONINE |
| | | | | BETA-LYASE |
| | | | | (EC 4.4.1.8) (CBL) |
| | | | | (BETA-CYSTATHIONASE) |
| | | | | (CYSTEINE LYASE) |
| | | | | (ORF5) (FRAGMENT). |
| 223 | — | 960 | — | Similar to GU11_{—} |
| | | | | ORYSA P07728 |
| | | | | ORYZA SATIVA |
| | | | | (RICE). GLUTELIN |
| | | | | TYPE I PRECURSOR |
| | | | | (CLONE PREE 61). |
| 225 | — | 1068 | — | Similar to |
| | | | | gi\|226227\|prf∥ |
| | | | | 1502200A prolamin |
| | | | | [Avena sativa] |
| 227 | — | 1044 | 1165 | gi\|232161\|sp\| |
| | | | | P29835\|GL19_ORYSA 19 |
| | | | | KD GLOBULIN PRECURSOR |
| 229 | — | 960 | — | Similar to |
| | | | | gi\|169969\|gb\| |
| | | | | AAA33964.1\| |
| | | | | glycinin |
| 231 | 948 | 953 | 1176 | Similar to PRVA_RANCA |
| | | | | P18087 RANA CATESBEIANA |
| | | | | (BULL FROG). |
| | | | | PARVALBUMIN ALPHA |
| | | | | (PA 4.97). |
| 233 | — | 991 | — | gi\|121101\|sp\| |
| | | | | P08453\|GDB2_WHEAT |
| | | | | GAMMA-GLIADIN PRECURSOR |
| 235 | — | 960 | — | Similar to |
| | | | | gi\|20227\|emb\|CAA32566.1\| |
| | | | | preprolglutelin (AA −24 to 476) |
| | | | | [Oryza sativa] |
| 237 | — | 1073 | 1190 | Similar to PRVT_CHICK |
| | | | | P19753 GALLUS GALLUS |
| | | | | (CHICKEN). PARVALBUMIN, |
| | | | | THYMIC (AVIAN THYMIC |
| | | | | HORMONE) (ATH) (THYMUS- |
| | | | | SPECIFICANTIGEN T1). |
| 239 | | | | Similar to |
| | | | | gi\|20208\|emb\| |
| | | | | CAA38211.1\| glutelin |
| | | | | [Oryza sativa] |
| 241 | | | | Similar to |
| | | | | gi\|556407\|gb\| |
| | | | | AAA50319.1\| prolamin |
| 243 | | | | Similar to |
| | | | | gi\|166555\|gb\| |
| | | | | AAA32715.1\| avenin |
| 245 | — | 1048 | — | gi\|1170517\|sp\| |
| | | | | P45386\|IGA4_HAEIN |
| | | | | IMMUNOGLOBULIN A1 |
| | | | | PROTEASE PRECURSOR |
| 247 | | | | gi\|121090\|sp\| |
| | | | | P04721\|GDA1_WHEAT |
| | | | | ALPHA/BETA-GLIADIN A-I |
| | | | | PRECURSOR |
| 249 | | | | gi\|121101\|sp\| |
| | | | | P08453\|GDB2_WHEAT |
| | | | | GAMMA-GLIADIN |
| | | | | PRECURSOR |

TABLE 9
Genes involved in rice grain filling, which belong
to the functional category of Fatty Acid Metabolism

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 251 | 920 | 976 | 1131 | Similar to |
| | | | | PHLB_SERLI P18954 |
| | | | | SERRATIA |
| | | | | LIQUEFACIENS. PHLB |
| | | | | PROTEIN PRECURSOR. |
| 253 | — | 995 | — | Similar to LPXK_FRANO |
| | | | | Q47909 FRANCISELLA |
| | | | | NOVICIDA. PROBABLE |
| | | | | TETRAACYLDISACCHARIDE |
| | | | | 4 -KINASE (EC 2.7.1.130) |
| | | | | (LIPID A 4 -KINASE). |
| 255 | — | 972 | 1126 | Similar to |
| | | | | gi\|7339489\|emb\| |
| | | | | CAB82812.1\| phospho- |
| | | | | lipase-like protein |
| | | | | [Arabidopsis thaliana] |
| 257 | — | 1087 | 1177 | Similar to OLE2_ORYSA |
| | | | | Q40646 ORYZA SATIVA |
| | | | | (RICE). OLEOSIN 18 KD |
| | | | | (OSE721). Similar to |
| | | | | gi\|1171354\| |
| | | | | gb\|AAC02240.1\| |
| | | | | 18 kDa oleosin |
| | | | | [Oryza sativa] |
| 259 | — | 1100 | 1132 | Similar to |
| | | | | gi\|4455257\|emb\| |
| | | | | CAB36756.1\| |
| | | | | oleosin, 18.5 K |
| | | | | [Arabidopsis thaliana] |
| 261 | 910 | 1093 | 1158 | Similar to |
| | | | | KSU5_ECOLI P42216 |
| | | | | ESCHERICHIA COLI. |
| | | | | 3-DEOXY-MANNO- |
| | | | | OCTULOSONATE |
| | | | | CYTIDYLYLTRANS- |
| | | | | FERASE (EC 2.7.7.38) |
| | | | | (CMP-KDOSYNTHETASE) |
| | | | | (CMP-2-KETO-3- |
| | | | | DEOXYOCTULOSONIC |
| | | | | ACID SYNTHETASE) |
| | | | | (CKS). |
| 263 | 884 | 1038 | 1172 | Similar to |
| | | | | ACBP_GOSHI Q39779 |
| | | | | GOSSYPIUM HIRSUTUM |
| | | | | (UPLAND COTTON). |
| | | | | ACYL-COA-BINDING |
| | | | | PROTEIN (ACBP). |
| 265 | 915 | 990 | 1122 | Similar to |
| | | | | gi\|4587543\|gb\| |
| | | | | AAD25774.1\| |
| | | | | AC006577_10 Belongs |
| | | | | to the PF\|00657 |
| | | | | Lipase/Acylhydrolase |
| | | | | with GDSL-motif |
| | | | | family.EST |
| | | | | gb\|AB015099 comes |
| | | | | from this gene. |
| | | | | [Arabidopsis thaliana] |
| 267 | 897 | 1082 | 1195 | Similar to |
| | | | | GBSB_BACSU P71017 |
| | | | | BACILLUS SUBTILIS. |
| | | | | ALCOHOL DEHYDROGENASE |
| | | | | (EC 1.1.1.1). |
| 269 | — | 961 | — | Similar to |
| | | | | gi\|6714447\|gb\| |
| | | | | AAF26134.1\| |
| | | | | AC011620_10 putative |
| | | | | phospholipase D |
| | | | | [Arabidopsis thaliana] |
| 271 | — | 1100 | 1132 | Similar to |
| | | | | gi\|1171352\|gb\| |
| | | | | AAC02239.1\| 16 kDa |
| | | | | oleosin [Oryza |
| | | | | sativa] Similar to |
| | | | | gi\|944830\|emb\| |
| | | | | CAA43183.1\| soybean |
| | | | | 24 kDa oleosin isoform |
| | | | | [Glycine max] |
| 273 | 886 | 1012 | 1178 | Similar to |
| | | | | gi\|7576210\|emb\| |
| | | | | CAB87871.1\| palmitoyl- |
| | | | | protein thioesterase |
| | | | | precursor-like |
| | | | | [Arabidopsis thaliana] |
| 275 | | | | Similar to |
| | | | | 3O1D_COMTE Q06401 |
| | | | | COMAMONAS |
| | | | | TESTOSTERONI |
| | | | | (PSEUDOMONAS |
| | | | | TESTOSTERONI). |
| | | | | 3-OXOSTEROID 1- |
| | | | | DEHYDROGENASE |
| | | | | (EC 1.3.99.4). |
| 277 | — | 951 | 1160 | Similar to |
| | | | | CRTI_PHYBL P54982 |
| | | | | PHYCOMYCES |
| | | | | BLAKESLEEANUS. |
| | | | | PHYTOENE |
| | | | | DEHYDROGENASE |
| | | | | (EC 1.3.-.-) |
| | | | | (PHYTOENE DESATURASE). |
| 279 | — | 973 | — | Similar to |
| | | | | gi\|6648208\|gb\| |
| | | | | AAF21206.1\|AC013483_30 |
| | | | | putative phosphatidylinositol- |
| | | | | 4-phosphate 5-kinase |
| | | | | [Arabidopsis thaliana] |

TABLE 10
Genes involved in rice grain filling, which belong
to the functional category of amino acid metabolism

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 281 | — | 1053 | — | Similar to |
| | | | | gi\|2076884\|gb\| |
| | | | | AAB539751\| lysine- |
| | | | | ketoglutarate |
| | | | | reductase/saccharopine |
| | | | | dehydrogenase |
| | | | | [Arabidopsis thaliana] |
| 283 | — | 1036 | 1199 | Similar to |
| | | | | gi\|974605\|gb\| |
| | | | | AAA75104.1\| single- |
| | | | | stranded nucleic acid |
| | | | | binding protein |
| 285 | — | 978 | — | 68173.m01963#MAL21_29# |
| | | | | AT3g20250#RNA-binding |
| | | | | protein, putative- |
| | | | | Length = 955 |
| 287 | 918 | 1008 | 1139 | gi\|730108\|sp\| |
| | | | | Q00539\|NAM8_YEAST |
| | | | | NAM8 PROTEIN |
| 289 | 928 | 1061 | — | Similar to |
| | | | | gi\|287298\|dbj\| |
| | | | | BAA03504.1\| aspartate |
| | | | | aminotransferase |
| | | | | [Oryza sativa] |
| 291 | 923 | 980 | 1141 | Similar to MTAP_HUMAN |
| | | | | Q13126 HOMO SAPIENS |
| | | | | (HUMAN). 5-METHYLTHIO- |
| | | | | ADENOSINE PHOSPHORYLASE |
| | | | | (EC 2.4.2.28) |
| | | | | (MTAPHOSPHORYLASE) |
| | | | | (MTAPASE). |
| 293 | | | | Similar to SEPR_THESP |
| | | | | P80146 THERMUS SP. |
| | | | | (STRAIN RT41A). |
| | | | | EXTRACELLULAR SERINE |
| | | | | PROTEINASE PRECURSOR |
| | | | | (EC 3.4.21.-). |
| 295 | 903 | 1019 | — | Similar to |
| | | | | gi\|6728985\|gb\| |
| | | | | AAF26983.1\|AC018363_28 |
| | | | | putative |
| | | | | S-adenosylmethionine: |
| | | | | 2-demethylmenaquinone |
| | | | | methyltransferase |
| | | | | [A thaliana] |
| 297) | — | 1092 | — | 68173.m01963#MAL21_29# |
| | | | | AT3g20250#RNA-binding |
| | | | | protein, putative- |
| | | | | Length = 955 |
| 299 | — | 986 | 1169 | Similar to IF4H_HUMAN |
| | | | | Q15056 HOMO SAPIENS |
| | | | | (HUMAN). EUKARYOTIC |
| | | | | TRANSLATION INI- |
| | | | | TIATION FACTOR 4H |
| | | | | (EIF-4H) (KIAA0038). |

TABLE 11
Genes involved in rice grain filling, which belong
to the functional category of transcription factors

| Rice | Banana | Wheat | Maize | |
|---|---|---|---|---|
| (SEQ | (SEQ | (SEQ | (SEQ | |
| ID | ID | ID | ID | |
| NO) | NO) | NO) | NO) | Gene Description |
| 301 | | | | Similar to |
| | | | | gi\|7211973\|gb\| |
| | | | | AAF40444.1\| |
| | | | | AC004809_2 Contains |
| | | | | similarity to the |
| | | | | CREB-binding protein |
| | | | | (CBP) from Mus sp |
| | | | | gb\|S66385. |
| | | | | [Arabidopsis thaliana] |
| 303 | — | 974 | — | Similar to |
| | | | | gi\|6899934\|emb\| |
| | | | | CAB71884.1\| |
| | | | | putative zinc-finger |
| | | | | protein [A thaliana] |
| 305 | | | | gi\|2493550\|sp\| |
| | | | | Q02516\|HAP5_YEAST |
| | | | | TRANSCRIPTIONAL |
| | | | | ACTIVATOR HAP5 |
| 307 | 898 | 1091 | 1201 | Similar to |
| | | | | gi\|403418\|gb\| |
| | | | | AAA18414.1\| GBF4 |
| 309 | | | | 68170.m04237#F14G24_15# |
| | | | | At1g52880#NAM-like |
| | | | | proteinLength = 320 |
| 311 | 933 | 996 | 1129 | Myb family transcription |
| | | | | factor |
| 313 | | | | Myb family transcription |
| | | | | factor |
| 315 | 943 | 1072 | 1119 | Myb family transcription |
| | | | | factor |
| 317 | — | 1007 | — | Myb family transcription |
| | | | | factor |
| 319 | — | 1013 | 1143 | Similarity[af007269_37269- |
| | | | | 38693 /gene = “a_ig002n01.20” |
| | | | | /protein_id = “aab61027.1” |
| | | | | /note = “contains weak |
| | | | | similarity to myb-related |
| | | | | proteins” ] Evidence[100% |
| | | | | (559/559)] |
| 321 | — | 1097 | 1135 | Motifs{Myb_2 Myb |
| | | | | DNA-binding domain repeat; |
| | | | | Myb_2 Myb DNA-binding |
| | | | | domain repeat} |
| | | | | Evidence[38% (306/804)] |
| 323 | 940 | 981 | 1197 | Similar to |
| | | | | gi\|2894607\|emb\| |
| | | | | CAA17141.1\| NAM (no |
| | | | | apical meristem)-like |
| | | | | protein [Arabidopsis |
| | | | | thaliana] |
| 325 | — | — | 1171 | Similar to |
| | | | | gi\|2224929\|gb\| |
| | | | | AAC49747.1\| ethylene- |
| | | | | insensitive3-like2 |
| | | | | [Arabidopsis thaliana] |
| 327 | — | 979 | 1174 | Myb DNA-binding domain |
| | | | | repeat; Myb_2 Myb DNA- |
| | | | | binding domain repeat; |
| | | | | Myb_2 Myb DNA-binding |
| | | | | domain repeat} |
| | | | | Evidence[69% (615/879)] |

### Example 5

### Rice Orthologs of Arabidopsis Grain Filling Genes Identified by Reverse Genetics

Understanding the function of every gene is the major challenge in the age of completely sequenced eukaryotic genomes. Sequence homology can be helpful in identifying possible functions of many genes. However, reverse genetics, the process of identifying the function of a gene by obtaining and studying the phenotype of an individual containing a mutation in that gene, is another approach to identify the function of a gene.

Reverse genetics in Arabidopsis has been aided by the establishment of large publicly available collections of insertion mutants (Krysan et al., (1999) Plant Cell 11, 2283-2290; Tisser et al., (1999) Plant Cell 11, 1841-1852; Speulman et al., (1999). Plant Cell 11, 1853-1866; Parinov et al., (1999). Plant Cell 11, 2263-2270; Parinov and Sundaresan, 2000; Biotechnology 11, 157-161). Mutations in genes of interest are identified by screening the population by PCR amplification using primers derived from sequences near the insert border and the gene of interest to screen through large pools of individuals. Pools producing PCR products are confirmed by Southern hybridization and further deconvoluted into subpools until the individual is identified (Sussmnan et al., (2000) Plant Physiology 124, 1465-1467).

Recently, some groups have begun the process of sequencing insertion site flanking regions from individual plants in large insertion mutant populations, in effect prescreening a subset of lines for genomic insertion sites (Parinov et al., (1999). Plant Cell 11, 2263-2270; Tisser et al., (1999). Plant Cell 11, 1841-1852). The advantage to this approach is that the laborious and time-consuming process of PCR-based screening and deconvolution of pools is avoided.

A large database of insertion site flanking sequences from approximately 100,000 T-DNA mutagenized Arabidopsis plants of the Columbia ecotype (GARLIC lines) is prepared. T-DNA left border sequences from individual plants are amplified using a modified thermal asymmetric interlaced-polymerase chain reaction (TAIL-PCR) protocol (Liuet al., (1995). Plant J. 8, 457-463). Left border TAIL-PCR products are sequenced and assembled into a database that associates sequence tags with each of the approximately 100,000 plants in the mutant collection. Screening the collection for insertions in genes of interest involves a simple gene name or sequence BLAST query of the insertion site flanking sequence database, and search results point to individual lines. Insertions are confirmed using PCR.

Analysis of the GARLIC insert lines suggests that there are 76,856 insertions that localize to a subset of the genome representing coding regions and promoters of 22,880 genes. Of these, 49,231 insertions lie in the promoters of over 18,572 genes, and an additional 27,625 insertions are located within the coding regions of 13,612 genes. Approximately 25,000 T-DNA left border mTAIL-PCR products (25% of the total 102,765) do not have significant matches to the subset of the genome representing promoters and coding regions, and are therefore presumed to lie in noncoding and/or repetitive regions of the genome.

The Arabidopsis T-DNA GARLIC insertion collection is used to investigate the roles of certain genes in the grain filling process. Target genes are chosen using a variety of criteria, including public reports of mutant phenotypes, RNA profiling experiments, and sequence similarity to genes implicated in grain filling. Plant lines with insertions in genes of interest are then identified. Each T-DNA insertion line is represented by a seed lot collected from a plant that is hemizygous for a particular T-DNA insertion. Plants homozygous for insertions of interest are identified using a PCR assay. The seed produced by these plants is homozygous for the T-DNA insertion mutation of interest.

Homozygous mutant plants are tested for altered grain composition. The genes interrupted in these mutants contribute to the observed phenotype. The genes interrupted in these mutants interfere with the normal grain filling process.

Rice orthologs of the Arabidopsis genes affecting the grain filling process and thus grain composition are identified by similarity searching of a rice database using the Double-Affine Smith-Waterman algorithm (BLASP with e values better than ⁻¹⁰).

### Example 6

### Cloning and Sequencing of Nucleic Acid Molecules from Rice

6.1 Genomic DNA:

Plant genomic DNA samples are isolated from a collection of tissues which are listed in Table 1. Individual tissues are collected from a minimum of five plants and pooled. DNA can be isolated according to one of the three procedures, e.g., standard procedures described by Ausubel et al. (1995), a quick leaf prep described by Klimyuk et al. (1993), or using FTA paper (Life Technologies).

For the latter procedure, a piece of plant tissue such as, for example, leaf tissue is excised from the plant, placed on top of the FTA paper and covered with a small piece of parafilm that serves as a barrier material to prevent contamination of the crushing device. In order to drive the sap and cells from the plant tissue into the FTA paper matrix for effective cell lysis and nucleic acid entrapment, a crushing device is used to mash the tissue into the FTA paper. The FTA paper is air dried for an hour. For analysis of DNA, the samples can be archived on the paper until analysis. Two mm punches are removed from the specimen area on the FTA paper using a 2 mm Harris Micro Punch™ and placed into PCR tubes. Two hundred (200) microliters of FTA purification reagent is added to the tube containing the punch and vortexed at low speed for 2 seconds. The tube is then incubated at room temperature for 5 minutes. The solution is removed with a pipette so as to repeat the wash one more time. Two hundred (200) microliters of TE (10 mM Tris, 0.1 mM EDTA, pH 8.0) is added and the wash is repeated two more times. The PCR mix is added directly to the punch for subsequent PCR reactions.

6.2 Cloning of Candidate cDNA: A candidate cDNA is amplified from total RNA isolated from rice tissue after reverse transcription using primers designed against the computationally predicted cDNA. Primers designed based on the genomic sequence can be used to PCR amplify the full length cDNA (start to stop codon) from first strand cDNA prepared from rice cultivar Nipponbare tissue.

The Qiagen RNeasy kit (Qiagen, Hilden, Germany) is used for extraction of total RNA. The Superscript II kit (Onvitrogen, Carlsbad, USA) is used for the reverse transcription reaction. PCR amplification of the candidate cDNA is carried out using the reverse primer sequence located at the translation start of the candidate gene in 5′-3′ direction. This is performed with high-fidelity Taq polymerase (Invitrogen, Carlsbad, USA).

The PCR fragment is then cloned into pCR2.1-TOPO (Invitrogen) or the pGEM-T easy vector (Promega Corporation, Madison, Wis., USA) per the manufacturer's instructions, and several individual clones are subjected to sequencing analysis.

6.3 DNA sequencing: DNA preps for 2-4 independent clones are miniprepped following the manufacturer's instructions (Qiagen). DNA is subjected to sequencing analysis using the BigDye™ Terminator Kit according to manufacturer's instructions (AB). Sequencing makes use of primers designed to both strands of the predicted gene of interest. DNA sequencing is performed using standard dye-terminator sequencing procedures and automated sequencers (models 373 and 377; Applied Biosystems, Foster City, Calif.). All sequencing data are analyzed and assembled using the Phred/Phrap/Consed software package (University of Washington) to an error ratio equal to or less than 10⁻⁴ at the consensus sequence level.

The consensus sequence from the sequencing analysis is then to be validated as being intact and the correct gene in several ways. The coding region is checked for being full length (predicted start and stop codons present) and uninterrupted (no internal stop codons). Alignment with the gene prediction and BLAST analysis is used to ascertain that this is in fact the right gene.

The clones are sequenced to verify their correct amplification.

### Example 7

### Functional Analysis in Plants

A plant complementation assay can be used for the functional characterization of the grain filing genes according to the invention.

Rice and Arabidopsis putative orthologue pairs are identified using BLAST comparisons, TFASTXY comparisons, and Double-Affine Smith-Waterman similarity searches. Constructs containing a rice cDNA or genomic clone inserted between the promoter and terminator of the Arabidopsis orthologue are generated using overlap PCR (Gene 77, 61-68 (1989)) and GATEWAY cloning (Life Technologies Invitrogen). For ease of cloning, rice cDNA clones are preferred to rice genomic clones. A three stage PCR strategy is used to make these constructs.

(1) In the first stage, primers are used to PCR amplify: (i) 2 Kb upstream of the translation start site of the Arabidopsis orthologue, (ii) the coding region or cDNA of the rice orthologue, and (iii) the 500 bp immediately downstream of the Arabidopsis orthogue's translation stop site. Primers are designed to incorporate onto their 5′ ends at least 16 bases of the 3′ end of the adjacent fragment, except in the case of the most distal primers which flank the gene construct (the forward primer of the promoter and the reverse primer of the terminator). The forward primer of the promoters contains on their 5′ ends partial AttB1 sites, and the reverse primer of the terminators contains on their 5′ ends partial AttB2 sites, for Gateway cloning.

(2) In the second stage, overlap PCR is used to join either the promoter and the coding region, or the coding region and the terminator.

(3) In the third stage either the promoter-coding region product can be joined to the terminator or the coding regionterminator product can be joined to the promoter, using overlap PCR and amplification with full Att site-containing primers, to link all three fragments, and put full Att sites at the construct termini.

The fused three-fragment piece flanked by Gateway cloning sites are introduced into the LTI donor vector pDONR201 (Invitrogen) using the BP clonase reaction, for confirmation by sequencing. Confirmed sequenced constructs are introduced into a binary vector containing Gateway cloning sites, using the LR clonase reaction such as, for example, pAS200.

The pAS200 vector was created by inserting the Gateway cloning cassette RfA into the Acc651 site of pNOV3510.

pNOV3510 was created by ligation of inverted pNOV2114 VSI binary into pNOV3507, a vector containing a PTX5′ Arab Protox promoter driving the PPO gene with the Nos terminator.

pNOV2114 was created by insertion of virN54D (Pazour et al. 1992, J. Bacteriol. 174:4169-4174) from pAD1289 (Hansen et al. 1994, PNAS 91:7603-7607) into pHiNK085.

pHiNK085 was created by deleting the 35S:PMI cassette and M13 ori in pVictor HiNK.

pPVictor HiNK was created by modifying the T-DNA of pVictor (described in WO 97/04112) to delete M13 derived sequences and to improve its cloning versatility by introducing the BIGLINK polylinker.

The sequence of the pVictor HiNK vector is disclosed in SEQ ID NO: 5 in WO 00/6837, which is incorporated herein by reference. The pVictor HiNK vector contains the following constituents that are of functional importance:
The origin of replication (OR1) functional in Agrobacterium is derived from the Pseudomonas aeruginosa plasmid pVS1 (Itoh et al. 1984. Plasmid 11: 206-220; Itoh and Haas, 1985. Gene 36: 27-36). The pVS1 OR1 is only functional in Agrobacterium and can be mobilised by the helper plasmid pRK2013 from E. coli into A. tumefaciens by means of a triparental mating procedure (Ditta et al., 1980. Proc. Natl. Acad. Sci USA 77: 7347-7351).
The ColE1 origin of replication functional in E. coli is derived from pUC19 (Yannisch Perron et al., 1985. Gene 33: 103-119).
The bacterial resistance to spectinomycin and streptomycin encoded by a 0.93 kb fragment from transposon Tn7 (Fling et al., 1985. Nucl. Acids Res. 13: 7095) functions as selectable marker for maintenance of the vector in E. coli and Agrobacterium. The gene is fused to the tac promoter for efficient bacterial expression (Amman et al., 1983. Gene 25: 167-178).
The right and left T-DNA border fragments of 1.9 kb and 0.9 kb that comprise the 24 bp border repeats, have been derived from the Ti-plasmid of the nopaline type Agrobacterium tumefaciens strains pTiT37 (Yadav et al., 1982. Proc. Natl. Acad. Sci. USA. 79: 6322-6326).

The plasmid is introduced into Agrobacterium tumefaciens GV3101 pMP90 by electroporation. The positive bacterial transformants are selected on LB medium containing 50 μg/μl kanamycin and 25 μg/μd gentamycin. Plants are transformed by standard methodology (e.g., by dipping flowers into a solution containing the Agrobacterium) except that 0.02% Silwet—77 (Lehle Seeds, Round Rock, Tex.) is added to the bacterial suspension and the vacuum step omitted. Five hundred (500) mg of seeds are planted per 2 ft² flat of soil and, and progeny seeds are selected for transformants using PPO selection.

Primary transformants are analyzed for complementation. Primary transformants are genotyped for the Arabidopsis mutation and presence of the transgene. When possible, >50 mutants harboring the transgene should be phenotyped to observe variation due to transgene copy number and expression

### Example 8

### Vector Construction for Overexpression and Gene “Knockout” Experiments

8.1 Overexpression

Vectors used for expression of full-length “grain filling candidate genes” of interest in plants (overexpression) are designed to overexpress the protein of interest and are of two general types, biolistic and binary, depending on the plant transformation method to be used.

For biolistic transformation (biolistic vectors), the requirements are as follows:
1. a backbone with a bacterial selectable marker (typically, an antibiotic resistance gene) and origin of replication functional in Escherichia coli (E. coli; eg. ColE1), and
2. a plant-specific portion consisting of
   a. a gene expression cassette consisting of a promoter (eg. ZmUBlint MOD), the gene of interest (typically, a full-length cDNA) and a transcriptional terminator (eg. Agrobacterium tumefaciens nos terminator);
   b. a plant selectable marker cassette, consisting of a promoter (eg. rice Act1D-BV MOD), selectable marker gene (eg. phosphomannose isomerase, PMI) and transcriptional terminator (eg. CaMV terminator).
      Vectors designed for transformation by Agrobacterium tumefaciens (A. tumefaciens; binary vectors) consist of:
1. a backbone with a bacterial selectable marker functional in both E. coli and A. tumefaciens (eg. spectinomycin resistance mediated by the aadA gene) and two origins of replication, functional in each of aforementioned bacterial hosts, plus the A. tumefaciens virG gene;
2. a plant-specific portion as described for biolistic vectors above, except in this instance this portion is flanked by A. tumefaciens right and left border sequences which mediate transfer of the DNA flanked by these two sequences to the plant.

8.2 Knockout Vectors

Vectors designed for reducing or abolishing expression of a single gene or of a family or related genes (knockout vectors) are also of two general types corresponding to the methodology used to downregulate gene expression: antisense or double-stranded RNA interference (dsRNAi).

(a) Anti-Sense

For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the promoter, thus, mRNA will be made from the non-coding (antisense) strand in planta.

(b) dsRNAi

For dsRNAi vectors, a partial gene fragment (typically, 300 to 500 basepairs long) is used in the gene expression cassette, and is expressed in both the sense and antisense orientations, separated by a spacer region (typically, a plant intron, eg. the OsSH1 intron 1, or a selectable marker, eg. conferring kanamycin resistance). Vectors of this type are designed to form a double-stranded mRNA stem, resulting from the basepairing of the two complementary gene fragments in planta.

Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning). An important variant is the nature of the gene expression cassette promoter driving expression of the gene or gene fragment of interest in most tissues of the plants (constitutive, eg. ZmUBlint MOD), in specific plant tissues (eg. maize ADP-gpp for endosperm-specific expression), or in an inducible fashion (eg. GAL4bsBzl for estradiol-inducible expression in lines constitutively expressing the cognate transcriptional activator for this promoter).

### Example 9

### Insertion of a “Grain Filling Candidate Gene” 1 into Expression Vector

A validated rice cDNA clone in pCR2.1-TOPO or the pGEM-T easy vector is subcloned using conventional restriction enzyme-based cloning into a vector, downstream of the maize ubiquitin promoter and intron, and upstream of the Agrobacterium tumefaciens nos 3′ end transcriptional terminator. The resultant gene expression cassette (promoter, “grain filling candidate gene” and terminator) is further subcloned, using conventional restriction enzyme-based cloning, into the pNOV2117 binary vector (Negrotto et al (2000) Plant Cell Reports 19, 798-803; plasmid pNOV117 discosed in this article corresponds to pNOV2117 described herein; the nucleotide sequence of pNOV2117 is provided in SEQ ID NO: 44 of WO 0173087), generating pNOVCAND.

The pNOVCAND binary vector is designed for transformation and over-expression of the “grain filling candidate gene” in monocots. It consists of a binary backbone containing the sequences necessary for selection and growth in Escherichia coli DH-5α (Invitrogen) and Agrobacterium tumefaciens LBA4404 (pAL4404; pSB1), including the bacterial spectinomycin antibiotic resistance aadA gene from E. coli transposon Tn7, origins of replication for E. coli (ColE1) and A. tumefaciens (VS1), and the A. tumefaciens virG gene. In addition to the binary backbone, which is identical to that of pNOV2114 described herein previously (see Example 7 above), pNOV2117 contains the T-DNA portion flanked by the right and left border sequences, and including the Positech™ (Syngenta) plant selectable marker (WO 94/20627) and the “grain filling candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Plasmid pNOV2117 is introduced into Agrobacterium tumefaciens LBA4404 (pAL4404; pSB1) by electroporation. Plasmid pAL4404 is a disarmed helper plasmid (Ooms et al (1982) Plasmid 7, 15-29). Plasmid pSB1 is a plasmid with a wide host range that contains a region of homology to pNOV2117 and a 15.2 kb KpnI fragment from the virulence region of pTiBo542 (Ishida et al (1996) Nat Biotechnol 14, 745-750). Introduction of plasmid pNOV2117 into Agrobacterium strain LBA4404 results in a co-integration of pNOV2117 and pSB1.

Alternatively, plasmid pCIB7613, which contains the hygromycin phosphotransferase (hpt) gene (Gritz and Davies, Gene 25, 179-188, 1983) as a selectable marker, may be employed for transformation.

Plasmid pCIB7613 (see WO 98/06860, incorporated herein by reference in its entirety) is selected for rice transformation. In pCIB7613, the transcription of the nucleic acid sequence coding hygromycin-phosphotransferase (HYG genc) is driven by the corn ubiquitin promoter (ZmUbi) and enhanced by corn ubiquitin intron 1. The 3′polyadenylation signal is provided by NOS 3′ nontranslated region.

Other useful plasmids include pNADII002 (GALA-ER-VP16) which contains the yeast GAL4 DNA Binding domain (Keegan et al., Science, 231:699 (1986)), the mammalian estrogen receptor ligand binding domain (Greene et al., Science, 231 :1150 (1986)) and the transcriptional activation domain of the HSV VP16 protein (Triezenberg et al., 1988). Both hpt and GAIA-ER-VP16 are constitutively expressed using the maize Ubiquitin promoter, and pSGCDL1 (GAL4BS Bzl Luciferase), which carries the firefly luciferase reporter gene under control of a minimal maize Bronzel (Bzl) promoter with 10 upstream synthetic GAL4 binding sites. All constructs use termination signals from the nopaline synthase gene.

### Example 10

### Plant Transformation

10.1 Rice Transformation

pNOVCAND is transformed into a rice cultivar (Kaybonnet) using Agrobacterium-mediated transformation, and mannose-resistant calli are selected and regenerated.

Agrobacterium is grown on YPC solid plates for 2-3 days prior to experiment initiation. Agrobacterial colonies are suspended in liquid MS media to an OD of 0.2 at λ600 nm. Acetosyringone is added to the agrobacterial suspension to a concentration of 200 μM and agro is induced for 30 min.

Three-week-old calli which are induced from the scutellum of mature seeds in the N6 medium (Chu, C. C. et al., Sci, Sin., 18, 659-668(1975)) are incubated in the agrobacterium solution in a 100×25 petri plate for 30 minutes with occasional shaking. The solution is then removed with a pipet and the callus transfered to a MSAs medium which is overlayed with sterile filter paper.

Co-Cultivation is continued for 2 days in the dark at 22° C.

Calli are then placed on MS-Timetin plates for 1 week. After that they are tranferred to PAA+ mannose selection media for 3 weeks.

Growing calli (putative events) are picked and transfered to PAA+ mannose media and cultivated for 2 weeks in light.

Colonies are tranferred to MS20SorbKinTim regeneration media in plates for 2 weeks in light. Small plantlets are transferred to MS20SorbKinTim regeneration media in GA7 containers. When they reach the lid, they are transfered to soil in the greenhouse.

Expression of the “grain filling candidate gene” in transgenic To plants is analyzed. Additional rice cultivars, such as but not limited to, Nipponbare, Taipei 309 and Fuzisaka 2 are also transformed and assayed for expression of the “grain filling candidate gene” product and enhanced protein expression.

10.2 Maize Transformation

Transformation of immature maize embryos is performed essentially as described in Negrotto et al., (2000) Plant Cell Reports 19: 798-803. For this example, all media constituents are as described in Negrotto et al., supra. However, various media constituents described in the literature may be substituted.

1. Transformation Plasmids and Selectable Marker

The genes used for transformation are cloned into a vector suitable for maize transformation as described in Example 17. Vectors used contain the phosphomannose isomerase (PMI) gene (Negrotto et al. (2000) Plant Cell Reports 19: 798-803).

2. Preparation of Agrobacterium tumefaciens

Agrobacterium strain LBA4404 (pSB1) containing the plant transformation plasmid is grown on YEP (yeast extract (5 g/L), peptone (10 g/L), NaCl (5 g/L), 15 g/l agar, pH 6.8) solid medium for 2 to 4 days at 28° C. Approximately 0.8×10⁹ Agrobacteria are suspended in LS-inf media supplemented with 100 μM acetosyringone (As) (Negrotto et al.,(2000) Plant Cell Rep 19: 798-803). Bacteria are pre-induced in this medium for 30-60 minutes.

3. Inoculation

Immature embryos from A188 or other suitable maize genotypes are excised from 8-12 day old ears into liquid LS-inf+100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) and cultured in the dark for 28° C. for 10 days.

4. Selection of Transformed Cells and Regeneration of Transformed Plants

Immature embryos producing embryogenic callus are transferred to LSDIM0.5S medium. The cultures are selected on this medium for 6 weeks with a subculture step at 3 weeks. Surviving calli are transferred either to LSDIM0.5S medium to be bulked-up or to Reg1 medium. Following culturing in the light (16 hour light/8 hour dark regiment), green tissues are then transferred to Reg2 medium without growth regulators and incubated for 1-2 weeks. Plantlets are transferred to Magenta GA-7 boxes (Magenta Corp, Chicago Ill.) containing Reg3 medium and grown in the light. Plants that are PCR positive for the promoter-reporter cassette are transferred to soil and grown in the greenhouse.

### Example 11

### Promoter Analysis

The gene chip experiment described above in Examples 3 and 4 are designed to uncover genes that are expressed in seed tissue during grain filling. Candidate promoters are identified based upon the expression profiles of the associated transcripts representatives of which are provided in SEQ ID NOs: 643-883.

Candidate promoters are obtained by PCR and fused to a GUS reporter gene containing an intron. Both histocherical and fluometric GUS assays are carried out on stably transformed rice and maize plants and GUS activity is detected in the transformants.

Further, transient assays with the promoter:GUS constructs are carried out in rice embryogenic callus and GUS activity is detected by histochemical staining according the protocol described below (see Example 12).

Construction of Binary Promoter::Reporter Plasmids

To construct a binary promoter: reporter plasmid for rice transformation a vector containing a promoter of interest (i.e., the DNA sequence 5′ of the initiation codon for the gene of interest) is used, which results from recombination in a BP reaction between a PCR product using the promoter of interest as a template and pDONR201™, producing an entry vector. The regulatory/promoter sequence is fused to the GUS reporter gene (Jefferson et al, 1987) by recombination using GATEWAY™ Technology according to manufacturers protocol as described in the Instruction Manual (GATEWAY™ Cloning Technology, GIBCO BRL, Rockville, Md. http://www.lifetech.com/).

Briefly, the Gateway Gus-intronGus (GIG)/NOS expression cassette is ligated into pNOV2117 binary vector in 5′ to 3′ orientation. The 4.1 kB expression cassette is ligated into the Kpn-1 site of pNOV2117, then clones are screened for orientation to obtain pNOV2346, a GATEWAY™ adapted binary destination vector.

The promoter fragment in the entry vector is recombined via the LR reaction with the binary destination vector containing the GUS coding region with an intron that has an attR site 5′ to the GUS reporter, producing a binary vector with a promoter fused to the GUS reporter (pNOVCANDProm). The orientation of the inserted fragment is maintained by the alt sequences and the final construct is verified by sequencing. The construct is then transformed into Agrobacterium tumefaciens strains by electroporation as described herein previously (see Example 9).

### Example 12

### Transient Expression Analysis of Candidate Promoters in Rice Embryogenic Callus

Materials:

Embryopenic rice callus (Kaybonett cultivar)
LBA 4404 Agrobacterium strains
KCMS liquid media for re-suspending bacterial pellet
200 mM stock (40 mg/ml) Acetosyringone
Sterile filter paper discs (8.5 mm in diameter)
LB spec liquid culture
MS-CIM media plates
MS-AS plates (co-cultivation plates)
MS-Tim plates (recovery plates)
Gus staining solution
   Methods:
   Induction of Embryogenic Callus:
1. Sterilize mature Kayboneu rice seeds in 40% ultra Clorox, 1 drop Tween 20, for 40 min.
2. Rinse with sterile water and plate on MS-CIM media (12 seeds/plate)
3. Grow in dark for four weeks.
4. Isolate embryogenic calli from scutellum to MS-CIM
5. Let grow in dark 8 days before use for transformation
   Agrobacterium Preparation and Induction:
1. Start 6 mL shaking cultures of LBA4404 Agrobacterium strains harboring rice promoter binary plasmids.
2. Grow the cultures at room temperature for 48 hrs in the rotary shaker.
3. Spin down the cultures at 8,000 rpm at 4° C. and re-suspend bacterial pellets in 10 ml of KCMS media supplemented with 100?M Acetosyringone.
4. Place in the shaker at room temp for 1 hr for induction of Agrobacterium virulence ones.
5. In a sterile hood dilute Agrobacterium cultures 1:3 in KSMS media and transfer diluted cultures into deep petri dishes.
   Inoculation of Plant Material and Staining:
6. In a sterile hood transfer embryogenic callus into diluted Agrobacerium solution and incubate for 30 minutes.
7. In a sterile hood blot callus tissue on sterile filter paper and transfer on MS-AS plates.
8. Co-culture plates in 22° C. growth chamber in the dark for two days.
9. In a sterile hood transfer callus tissue to MS-Tim plates for the tissue recovery (the presence of Timentin will prevent Agrobacterium growth).
10. Incubate tissue on MS-Tim media for two days at 22° C. in the dark.
11. Remove callus tissue from the plates and stain for 48 hrs. in GUS staining solution.
12. De-stain tissue in 70% EtOH for 24 hours.
   Recipes:

KCMS media (liquid), pH to 5.5
100 ml/l MS Major Salts, 10 ml/l MS Minor Salts, 5 ml/l MS iron stock, 0.5M K₂HPO₄, 0.1 mg/ml Myo-Inositol,
1.3 μg/ml Thiamine, 0.2 g/ml 2,4-D (1 mg/ml), 0.1 g/ml Kinetin, 3% Sucrose, 100?M Acetosyringo

MS-CIM media. pH 5.8
MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2? g/ml 2,4-D, Phytagel (3 g/L)

MS-As Medium, pH 5.8
MS Basal salt (4.3 g/L), B5 Vitamins (200×) (5 m/L), 2% Sucrose (20 g/L), Proline (500 mg/l), Glutamine (500 mg/L), Casein Hydrolysate (300 mg/L), 2? g/ml 2,4-D, Phytagel (3 g/L), 200 ? M Acetosyringone

MS-Tim media, pH 5.8
MS Basal salt (4.3 g/L), B5 Vitamins (200×) (5 m/L), 2% Sucrose (20 g/L), Proline (500 mg/L), Glutamine (500 mg/L), Casein Hydrolysate (300 mg(L), 2? g/ml 2,4-D, Phytagel (3 g/L), 400 mg/l Timentin

Gus staining solution, pH 7
0.3 M Mannitol; 0.02 M EDTA, pH=7.0; 0.04 NaH₂PO₄; 1 mM x-gluc
   The binary Promoter::Reporter Plasmids described in Example 9 above can also be used for stable transformation of rice and maize plants according to the protocols provided in Examples 10.1 and 10.2, respectively.

### Example 13

### Analysis of Mutant and transgenic Plant Material

Two tiers of assays are can be used for analysis of the mutant and transgenic plant material.
Near InfraRed (NIR) spectrophometric analysis of seeds.
NIR enables evaluation of changes in starch, oil, protein and fiber content at very high throughput (I sample/sec).
DIA or MRJ Imaging

DIA or MRI imaging allows observation of gross morphology and surface area of major seed tissues and compartments (embryo, aleurone, endosperm, seed coat). Transgenic lines can also be physically sectioned and directly observed for changes in seed compartment morphology.

Lines showing alterations in grain composition will be advanced to a second tier of assays dependent upon the nature of the change detected:
1) Protein track: 1-D and 2-D protein gels Protein profiles
HPLC Amino acid profiles
DNTB or papain staining Protein redox status
GC N/C/S ratios
2) Starch track: Iodine staining Content, branching
Glucose-6-P analysis Phosphorylation level
3) Oils track: GC Oil, fatty acid profile

### Example 14

### Chromosomal Markers to Identify the Location of a Nucleic Acid Sequence

The sequences of the present invention can also be used for SSR mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within a sequence are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs can be used to map a molecular marker closely linked to functional gene, as described by Akagi et al. (Genome 39:205 (1996)).

The sequences of the present invention can be used to identify and develop a variety of microsatellite markers, including the SSRs described above, as genetic markers for comparative analysis and mapping of genomes.

Many of the polynucleotides listed in Tables 2 to 11 contain at least 3 consecutive di-, tri- or tetranucleotide repeat units in their coding region that can potentially be developed into SSR markers. Trinucleotide motifs that can be commonly found in the coding regions of said polynucleotides and easily identified by screening the polynucleotides sequences for said motifs are, for example: CGG; GCC, CGC, GGC, etc. Once such a repeat unit has been found, primers can be designed which are complementary to the region flanking the repeat unit and used in any of the methods described below.

Sequences of the present invention can also be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers as disclosed herein, or substantially similar sequences or allelic variants thereof, may be used to detect the appearance or disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers are useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

### Example 15

### Quantitative Trait Linked Breeding

Various types of maps can be used with the sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in a trait, enables marker-assisted breeding to enhance desirable traits or suppress undesirable traits. The sequences disclosed herein can be used as markers for QTLs to assist marker-assisted breeding. The sequences of the invention can be used to identify QTLs and isolate alleles as described by Li et al. in a study of QTLs involved in resistance to a pathogen of rice. (Li et al., Mol Gen Genet 261:58 (1990)). In addition to isolating QTL alleles in rice, other cereals, and other monocot and dicot crop species, the sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

### Example 16

### Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

A multiplex assay is designed providing SSRs specific to each of the markers of interest. The SSRs are linked to different classes of beads. All of the relevant markers may be expressed genes, so RNA or cDNA techniques are appropriate. RNA is extracted from root tissue of 1000 different individual plants and hybridized in parallel reactions with the different classes of beads. Each class of beads is analyzed for each sample using a microfluidics analyzer. For the classes of beads corresponding to qualitative traits, qualitative measures of presence or absence of the target gene are recorded. For the classes of beads corresponding to quantitative traits, quantitative measures of gene activity are recorded. Individuals showing activity of all of the qualitative genes and highest expression levels of the quantitative traits are selected for further breeding steps. In procedures wherein no individuals have desirable results for all the measured genes, individuals having the most desirable, and fewest undesirable, results are selected for further breeding steps. In either case, progeny are screened to further select for homozygotes with high quantitative levels of expression of the quantitative traits.

### Example 17

### Method of Modifying the Gene Frequency

The invention further provides a method of modifying the frequency of a gene in a plant population, including the steps of: identifying an SSR within a coding region of a gene; screening a plurality of plants using the SSR as a marker to determine the presence or absence of the gene in an individual plant; selecting at least one individual plant for breeding based on the presence or absence of the gene; and breeding at least one plant thus selected to produce a population of plants having a modified frequency of the gene. The identification of the SSR within the coding region of a gene can be accomplished based on sequence similarity between the nucleic acid molecules of the invention and the region within the gene of interest flanking the SSR.

Supporting TABLES

TABLE 12
This table illustrates the correlation between
rice sequences in sub-groups I and III that show
homologies between 80% and 99.9% to each other

| Sub-Group II | Sub-Group I |
|---|---|
| Sequences | Sequences |
| SEQ ID NO | SEQ ID NO |
| 513 | 121, 123 |
| 515 | 333 |
| 517 | 441; 443 |
| 519 | 151 |
| 521 | 9 |
| 523 | 73 |
| 525 | 203 |
| 527 | 215 |
| 529 | 209 |
| 531 | 103 |
| 533 | 407 |
| 535 | 115 |
| 537 | 165 |
| 539 | 1 |
| 541 | 325 |
| 543 | 397 |
| 545 | 61 |
| 547 | 455 |
| 549 | 255 |
| 551 | 351 |
| 553 | 225 |
| 555 | 139 |
| 557 | 25 |
| 559 | 3 |
| 561 | 17 |
| 563 | 279 |
| 565 | 191 |
| 567 | 451 |
| 569 | 417 |
| 571 | 99; 95; 435 |
| 573 | 91; 81 |
| 575 | 95; 99 |
| 577 | 85 |
| 579 | 229; 223 |
| 581 | 83 |
| 583 | 401; 235 |
| 585 | 283 |
| 587 | 179 |
| 589 | 135 |
| 591 | 141 |
| 595 | 5 |
| 597 | 311 |
| 599 | 379 |
| 601 | 123; 121 |
| 603 | 335 |
| 605 | 287 |
| 607 | 161 |
| 609 | 69 |
| 611 | 177 |
| 615 | 413 |
| 617 | 143 |
| 619 | 251 |
| 621 | 331 |
| 623 | 375 |
| 625 | 67 |
| 627 | 387 |
| 629 | 81; 91 |
| 631 | 89 |
| 633 | 181 |
| 635 | 297 |
| 637 | 309 |
| 639 | 329 |
| 641 | 229 |
| 593, 613 | 221 |

TABLE 13
This table illustrates the correlation between
rice sequences in subgroups I and II

| | |
|---|---|
| 155 | 507 |
| 191 | 503 |
| 89 | 509 |
| 187 | 505 |
| 299 | 501 |
| 447 | 511 |

TABLE 14
Description of “Grain Filling”
QTLs identified in Tables 2 and 3

QTL: OS-AE-1-1
Species: Oryza sativa
General Trait: DEVELOPMENT
Specific Trait: Allelopathic effect
Citation: BREEDING SCIENCE (2001) 51: 47-51
Chromosome: 1
Flanking Markers(s):
QTL: OS-AE-11-1
Species: Oryza sativa
General Trait: DEVELOPMENT
Specific Trait: Allelopathic effect
Citation: BREEDING SCIENCE (2001) 51: 47-51
Chromosome: 11
Flanking Markers(s):
QTL: OS-AE-12-1
Species: Oryza sativa
General Trait: DEVELOPMENT
Specific Trait: Allelopathic effect
Citation: BREEDING SCIENCE (2001) 51: 47-51
Chromosome: 12
Flanking Markers(s):
QTL: OS-AE-5-1
Species: Oryza sativa
General Trait: DEVELOPMENT
Specific Trait: Allelopathic effect
Citation: BREEDING SCIENCE (2001) 51: 47-51
Chromosome: 5
Flanking Markers(s):
QTL: OS-AMY-5-1
Species: Oryza sativa
General Trait: QUALITY
Specific Trait: Amylose content
Citation: THEOR APPL GENET (1999) 98: 502-508
Chromosome: 5
Flanking Markers(s):
QTL: OS-AMY-6-1
Species: Oryza sativa
General Trait: QUALITY
Specific Trait: Amylose content
Citation: THEOR APPL GENET (1999) 99: 642-648
Chromosome: 6
Flanking Markers(s):
QTL: OS-AMY-6-2
Species: Oryza sativa
General Trait: QUALITY
Specific Trait: Amylose content
Citation: THEOR APPL GENET (1999) 98: 502-508
Chromosome: 6
Flanking Markers(s):
QTL: OS-APDF-9-1
Species: Oryza sativa
General Trait: DEVELOPMENT
Specific Trait: Albino plantlet differentiation frequency
Citation: MOLECULAR BREEDING (1998) 4: 165-172
Chromosome: 9
Flanking Markers(s):
QTL: OS-ASS-6-1
Species: Oryza sativa
General Trait: QUALITY
Specific Trait: Alkali spreading score
Citation: THEOR APPL GENET (1999) 98: 502-508
Chromosome: 6
Flanking Markers(s):
QTL: OS-ASS-6-2
Species: Oryza sativa
General Trait: QUALITY
Specific Trait: Alkali spreading score
Citation: THEOR APPL GENET (1999) 98: 502-508
Chromosome: 6
Flanking Markers(s):
QTL: OS-BDV-1-1
Species: Oryza sativa
General Trait: QUALITY
Specific Trait: Breakdown viscosity
Citation: THEOR APPL GENET (2000) 100: 280-284
Chromosome: 1
Flanking Markers(s):
QTL: OS-BDV-6-1
Species: Oryza sativa
General Trait: QUALITY
Specific Trait: Breakdown viscosity
Citation: THEOR APPL GENET (2000) 100: 280-284
Chromosome: 6
Flanking Markers(s):
QTL: OS-CHALK-1-1
Species: Oryza sativa
General Trait: QUALITY
Specific Trait: Grain chalkiness
Citation: THEOR APPL GENET (2000) 101: 823-829
Chromosome: 1
Flanking Markers(s): 0
QTL: OS-CHALK-10-1
Species: Oryza sativa
General Trait: QUALITY
Specific Trait: Grain chalkiness
Citation: THEOR APPL GENET (2000) 101: 823-829
Chromosome: 10
Flanking Markers(s): 83.5
QTL: OS-CHALK-6-1
Species: Oryza sativa
General Trait: QUALITY
Specific Trait: Grain chalkiness
Citation: THEOR APPL GENET (2000) 101: 823-829
Chromosome: 6
Flanking Markers(s): 12.5
QTL: OS-CIF-6-1
Species: Oryza sativa
General Trait: DEVELOPMENT
Specific Trait: Callus induction frequency
Citation: MOLECULAR BREEDING (1998) 4: 165-172
Chromosome: 6
Flanking Markers(s):
QTL: OS-CPV-1-1
Species: Oryza sativa
General Trait: QUALITY
Specific Trait: Cool paste viscosity
Citation: THEOR APPL GENET (2000) 100: 280-284
Chromosome: 1
Flanking Markers(s):
QTL: OS-CPV-6-1
Species: Oryza sativa
General Trait: QUALITY
Specific Trait: Cool paste viscosity
Citation: THEOR APPL GENET (2000) 100: 280-284
Chromosome: 6
Flanking Markers(s):
QTL: OS-CPV-6-2
Species: Oryza sativa
General Trait: QUALITY
Specific Trait: Cool paste viscosity
Citation: THEOR APPL GENET (2000) 100: 280-284
Chromosome: 6
Flanking Markers(s):
QTL: OS-CSV-1-1
Species: Oryza sativa
General Trait: QUALITY
Specific Trait: Consistency viscosity
Citation: THEOR APPL GENET (2000) 100: 280-284
Chromosome: 1
Flanking Markers(s):
QTL: OS-CSV-6-1
Species: Oryza sativa
General Trait: QUALITY
Specific Trait: Consistency viscosity
Citation: THEOR APPL GENET (2000) 100: 280-284
Chromosome: 6
Flanking Markers(s):
QTL: OS-CSV-6-2
Species: Oryza sativa
General Trait: QUALITY
Specific Trait: Consistency viscosity
Citation: THEOR APPL GENET (2000) 100: 280-284
Chromosome: 6
Flanking Markers(s):
QTL: OS-DM-6-1
Species: Oryza sativa
General Trait: YIELD
Specific Trait: Dry Mass
Citation: PLANT PHYSIOLOGY (2001) 125: 406-422
Chromosome: 6
Flanking Markers(s): 16.7
QTL: OS-FLLEN-3-1
Species: Oryza sativa
General Trait: YIELD
Specific Trait: Source-sink capacity
Citation: MOLECULAR BREEDING (1998) 4: 419-426
Chromosome: 2
Flanking Markers(s): 160
QTL: OS-FLLEN-9-1
Species: Oryza sativa
General Trait: YIELD
Specific Trait: Source-sink capacity
Citation: MOLECULAR BREEDING (1998) 4: 419-426
Chromosome: 4
Flanking Markers(s):
QTL: OS-FLWID-3-1
Species: Oryza sativa
General Trait: YIELD
Specific Trait: Source-sink capacity
Citation: MOLECULAR BREEDING (1998) 4: 419-426
Chromosome: 8
Flanking Markers(s):
QTL: OS-GC-2-1
Species: Oryza sativa
General Trait: QUALITY
Specific Trait: Gel consistency
Citation: THEOR APPL GENET (1999) 98: 502-508
Chromosome: 2
Flanking Markers(s):
QTL: OS-GC-6-1
Species: Oryza sativa
General Trait: QUALITY
Specific Trait: Gel consistency
Citation: THEOR APPL GENET (1999) 99: 642-648
Chromosome: 6
Flanking Markers(s):
QTL: OS-GP-1-1
Species: Oryza sativa
General Trait: YIELD
Specific Trait: Grains per panicle
Citation: THEOR APPL GENET (2000) 101: 248-254
Chromosome: 1
Flanking Markers(s):
QTL: OS-GP-6-1
Species: Oryza sativa
General Trait: YIELD
Specific Trait: Grains per panicle
Citation: THEOR APPL GENET (2000) 101: 248-254
Chromosome: 6
Flanking Markers(s):
QTL: OS-GPDF-1-1
Species: Oryza sativa
General Trait: DEVELOPMENT
Specific Trait: Green plantlet differentiation frequency
Citation: MOLECULAR BREEDING (1998) 4: 165-172
Chromosome: 1
Flanking Markers(s):
QTL: OS-GPL-1-1
Species: Oryza sativa
General Trait: YIELD
Specific Trait: Grains per plant
Citation: GENETICS (1998) 150: 899-909
Chromosome: 1
Flanking Markers(s):
QTL: OS-GPL-2-1
Species: Oryza sativa
General Trait: YIELD
Specific Trait: Grains per plant
Citation: GENETICS (1998) 150: 899-909
Chromosome: 2
Flanking Markers(s):
QTL: OS-GPL-4-1
Species: Oryza sativa
General Trait: YIELD
Specific Trait: Grains per plant
Citation: GENETICS (1998) 150: 899-909
Chromosome: 4
Flanking Markers(s):
QTL: OS-GPL-8-2
Species: Oryza sativa
General Trait: YIELD
Specific Trait: Grains per plant
Citation: GENETICS (1998) 150: 899-909
Chromosome: 8
Flanking Markers(s):
QTL: OS-GPP-4-1
Species: Oryza sativa
General Trait: YIELD
Specific Trait: Grains per panicle
Citation: GENETICS (1998) 150: 899-909
Chromosome: 4
Flanking Markers(s):
QTL: OS-GPP-8-2
Species: Oryza sativa
General Trait: YIELD
Specific Trait: Grains per panicle
Citation: GENETICS (1998) 150: 899-909
Chromosome: 8
Flanking Markers(s):
QTL: OS-GPYF-1-1
Species: Oryza sativa
General Trait: DEVELOPMENT
Specific Trait: Green plantlet yield frequency
Citation: MOLECULAR BREEDING (1998) 4: 165-172
Chromosome: 1
Flanking Markers(s):
QTL: OS-GW-1-2
Species: Oryza sativa
General Trait: YIELD
Specific Trait: 1000 grain weight
Citation: THEOR APPL GENET (2001) 102: 41-52
Chromosome: 1
Flanking Markers(s):
QTL: OS-GW-3-1
Species: Oryza sativa
General Trait: YIELD
Specific Trait: Grain weight - 1000 grains
Citation: GENETICS (1998) 150: 899-909
Chromosome: 3
Flanking Markers(s):
QTL: OS-GW-3-1
Species: Oryza sativa
General Trait: YIELD
Specific Trait: Grain weight
Citation: THEOR APPL GENET (2000) 101: 248-254
Chromosome: 3
Flanking Markers(s):
QTL: OS-GW-3-1
Species: Oryza sativa
General Trait: YIELD
Specific Trait: 1000 grain weight
Citation: THEOR APPL GENET (2001) 102: 41-52
Chromosome: 3
Flanking Markers(s):
QTL: OS-GW-5-1
Species: Oryza sativa
General Trait: YIELD
Specific Trait: Grain weight - 1000 grains
Citation: GENETICS (1998) 150: 899-909
Chromosome: 5
Flanking Markers(s):
QTL: OS-GW-5-1
Species: Oryza sativa
General Trait: YIELD
Specific Trait: Grain weight
Citation: THEOR APPL GENET (2000) 101: 248-254
Chromosome: 5
Flanking Markers(s):
QTL: OS-GW-9-1
Species: Oryza sativa
General Trait: YIELD
Specific Trait: Grain weight - 1000 grains
Citation: GENETICS (1998) 150: 899-909
Chromosome: 9
Flanking Markers(s):
QTL: OS-GW100-4-1
Species: Oryza sativa
General Trait: YIELD
Specific Trait: Grain weight - 100 grains
Citation: THEOR APPL GENET (1998) 96: 957-963
Chromosome: 4
Flanking Markers(s): 100
QTL: OS-GYLD-1-1
Species: Oryza sativa
General Trait: YIELD
Specific Trait: Grain yield - tons/ha
Citation: GENETICS (1998) 150: 899-909
Chromosome: 1
Flanking Markers(s):
QTL: OS-GYLD-2-1
Species: Oryza sativa
General Trait: YIELD
Specific Trait: Grain yield - tons/ha
Citation: GENETICS (1998) 150: 899-909
Chromosome: 2
Flanking Markers(s):
QTL: OS-GYLD-4-1
Species: Oryza sativa
General Trait: YIELD
Specific Trait: Grain yield - tons/ha
Citation: GENETICS (1998) 150: 899-909
Chromosome: 4
Flanking Markers(s):
QTL: OS-GYLD-8-2
Species: Oryza sativa
General Trait: YIELD
Specific Trait: Grain yield - tons/ha
Citation: GENETICS (1998) 150: 899-909
Chromosome: 8
Flanking Markers(s):
QTL: OS-HPV-6-1
Species: Oryza sativa
General Trait: QUALITY
Specific Trait: Hot paste viscosity
Citation: THEOR APPL GENET (2000) 100: 280-284
Chromosome: 6
Flanking Markers(s):
QTL: OS-HPV-6-2
Species: Oryza sativa
General Trait: QUALITY
Specific Trait: Hot paste viscosity
Citation: THEOR APPL GENET (2000) 100: 280-284
Chromosome: 6
Flanking Markers(s):
QTL: OS-PGWC-8-1
Species: Oryza sativa
General Trait: QUALITY
Specific Trait: Percentage of grain with white core
Citation: THEOR APPL GENET (1999) 98: 502-508
Chromosome: 8
Flanking Markers(s):
QTL: OS-REGEN-3-1
Species: Oryza sativa
General Trait: DEVELOPMENT
Specific Trait: Regeneration ability
Citation: THEOR APPL GENET (1999) 98: 243-251
Chromosome: 3
Flanking Markers(s): 9
QTL: OS-RGT-2-1
Species: Oryza sativa
General Trait: DEVELOPMENT
Specific Trait: Reproductive growth time
Citation: THEOR APPL GENET (2001) 102: 1236-1242
Chromosome: 2
Flanking Markers(s):
QTL: OS-RGT-5-1
Species: Oryza sativa
General Trait: DEVELOPMENT
Specific Trait: Reproductive growth time
Citation: THEOR APPL GENET (2001) 102: 1236-1242
Chromosome: 5
Flanking Markers(s):
QTL: OS-SBV-1-1
Species: Oryza sativa
General Trait: QUALITY
Specific Trait: Setback viscosity
Citation: THEOR APPL GENET (2000) 100: 280-284
Chromosome: 1
Flanking Markers(s):
QTL: OS-SBV-6-1
Species: Oryza sativa
General Trait: QUALITY
Specific Trait: Setback viscosity
Citation: THEOR APPL GENET (2000) 100: 280-284
Chromosome: 6
Flanking Markers(s):
QTL: OS-VGT-2-1
Species: Oryza sativa
General Trait: DEVELOPMENT
Specific Trait: Vegetative growth time
Citation: THEOR APPL GENET (2001) 102: 1236-1242
Chromosome: 2
Flanking Markers(s):
QTL: OS-VGT-2-2
Species: Oryza sativa
General Trait: DEVELOPMENT
Specific Trait: Vegetative growth time
Citation: THEOR APPL GENET (2001) 102: 1236-1242
Chromosome: 2
Flanking Markers(s):
QTL: OS-VGT-5-1
Species: Oryza sativa
General Trait: DEVELOPMENT
Specific Trait: Vegetative growth time
Citation: THEOR APPL GENET (2001) 102: 1236-1242
Chromosome: 5
Flanking Markers(s):
QTL: OS-VGT-9-1
Species: Oryza sativa
General Trait: DEVELOPMENT
Specific Trait: Vegetative growth time
Citation: THEOR APPL GENET (2001) 102: 1236-1242
Chromosome: 9
Flanking Markers(s):
QTL: OS-WC-6-1
Species: Oryza sativa
General Trait: QUALITY
Specific Trait: Grain white core
Citation: THEOR APPL GENET (2000) 101: 823-829
Chromosome: 6
Flanking Markers(s): 13.5
QTL: OS-Y-6-1
Species: Oryza sativa
General Trait: YIELD
Specific Trait: Yield
Citation: THEOR APPL GENET (2000) 101: 248-254
Chromosome: 6
Flanking Markers(s):
QTL: OS-YLD-1-1
Species: Oryza sativa
General Trait: YIELD
Specific Trait: Yield
Citation: THEOR APPL GENET (2001) 102: 41-52
Chromosome: 1
Flanking Markers(s):
QTL: OS-YLD-5-1
Species: Oryza sativa
General Trait: YIELD
Specific Trait: Yield
Citation: THEOR APPL GENET (2001) 102: 793-800
Chromosome: 5
Flanking Markers(s):
QTL: ZM-BIOM-3-1
Species: Zea mays
General Trait: YIELD
Specific Trait: “Biomass, above ground”
Citation: THEOR APPL GENET (1999) 99: 1106-1119
Chromosome: 3
Flanking Markers(s): “UMC3, UMC96”
QTL: ZM-BIOM-5-1
Species: Zea mays
General Trait: YIELD
Specific Trait: “Biomass, above ground”
Citation: THEOR APPL GENET (1999) 99: 1106-1119
Chromosome: 5
Flanking Markers(s): UMC166
QTL: ZM-BIOM-7-1
Species: Zea mays
General Trait: YIELD
Specific Trait: “Biomass, above ground”
Citation: THEOR APPL GENET (1999) 99: 1106-1119
Chromosome: 7
Flanking Markers(s): “UMC116, BNL14.07”
QTL: ZM-BIOM-8-1
Species: Zea mays
General Trait: YIELD
Specific Trait: “Biomass, above ground”
Citation: THEOR APPL GENET (1999) 99: 1106-1119
Chromosome: 8
Flanking Markers(s): “UMC138L, UMC12”
QTL: ZM-CL-9-1
Species: Zea mays
General Trait: QUALITY
Specific Trait: Cellulose content
Citation: THEOR APPL GENET (2001) 102: 591-599
Chromosome: 9
Flanking Markers(s):
QTL: ZM-CPC-1-2
Species: Zea mays
General Trait: QUALITY
Specific Trait: Crude protein concentration
Citation: CROP SCI (1998) 38: 1278-1289
Chromosome: 1
Flanking Markers(s): UMC76
QTL: ZM-CPC-1-2
Species: Zea mays
General Trait: QUALITY
Specific Trait: Crude protein content
Citation: CROP SCI (2001) 41: 690-697
Chromosome: 1
Flanking Markers(s): 224
QTL: ZM-CPC-1-3
Species: Zea mays
General Trait: QUALITY
Specific Trait: Crude protein concentration
Citation: CROP SCI (1998) 38: 1278-1289
Chromosome: 1
Flanking Markers(s): UMC58
QTL: ZM-CPC-1-4
Species: Zea mays
General Trait: QUALITY
Specific Trait: Crude protein concentration
Citation: CROP SCI (1998) 38: 1278-1289
Chromosome: 1
Flanking Markers(s): UMC128
QTL: ZM-CPC-1-5
Species: Zea mays
General Trait: QUALITY
Specific Trait: Crude protein concentration
Citation: CROP SCI (1998) 38: 1278-1289
Chromosome: 1
Flanking Markers(s): UMC67
QTL: ZM-CPC-1-6
Species: Zea mays
General Trait: QUALITY
Specific Trait: Crude protein concentration
Citation: CROP SCI (1998) 38: 1278-1289
Chromosome: 1
Flanking Markers(s): UMC83
QTL: ZM-CPC-10-1
Species: Zea mays
General Trait: QUALITY
Specific Trait: Crude protein concentration
Citation: CROP SCI (1998) 38: 1278-1289
Chromosome: 10
Flanking Markers(s): UMC130
QTL: ZM-CPC-3-1
Species: Zea mays
General Trait: QUALITY
Specific Trait: Crude protein concentration
Citation: CROP SCI (1998) 38: 1278-1289
Chromosome: 3
Flanking Markers(s): UMC154
QTL: ZM-CPC-3-2
Species: Zea mays
General Trait: QUALITY
Specific Trait: Crude protein concentration
Citation: CROP SCI (1998) 38: 1278-1289
Chromosome: 3
Flanking Markers(s): BNL1.297
QTL: ZM-CPC-3-3
Species: Zea mays
General Trait: QUALITY
Specific Trait: Crude protein concentration
Citation: CROP SCI (1998) 38: 1278-1289
Chromosome: 3
Flanking Markers(s): UMC10
QTL: ZM-CPC-5-1
Species: Zea mays
General Trait: QUALITY
Specific Trait: Crude protein concentration
Citation: CROP SCI (1998) 38: 1278-1289
Chromosome: 5
Flanking Markers(s): BNL6.22
QTL: ZM-CPC-6-2
Species: Zea mays
General Trait: QUALITY
Specific Trait: Crude protein concentration
Citation: CROP SCI (1998) 38: 1278-1289
Chromosome: 6
Flanking Markers(s): UMC85
QTL: ZM-CPC-7-2
Species: Zea mays
General Trait: QUALITY
Specific Trait: Crude protein concentration
Citation: CROP SCI (1998) 38: 1278-1289
Chromosome: 7
Flanking Markers(s): UMC98B
QTL: ZM-CPC-7-3
Species: Zea mays
General Trait: QUALITY
Specific Trait: Crude protein concentration
Citation: CROP SCI (1998) 38: 1278-1289
Chromosome: 7
Flanking Markers(s): UMC56
QTL: ZM-CPC-8-1
Species: Zea mays
General Trait: QUALITY
Specific Trait: Crude protein concentration
Citation: CROP SCI (1998) 38: 1278-1289
Chromosome: 8
Flanking Markers(s): UMC71
QTL: ZM-DMC-1-1
Species: Zea mays
General Trait: YIELD
Specific Trait: Dry matter concentration
Citation: CROP SCI (1998) 38: 1278-1289
Chromosome: 1
Flanking Markers(s): UMC33
QTL: ZM-DMC-1-1
Species: Zea mays
General Trait: YIELD
Specific Trait: Dry matter concentration
Citation: THEOR APPL GENET (2001) 102: 230-243
Chromosome: 1
Flanking Markers(s):
QTL: ZM-DMC-1-2
Species: Zea mays
General Trait: YIELD
Specific Trait: Dry matter concentration
Citation: CROP SCI (1998) 38: 1278-1289
Chromosome: 1
Flanking Markers(s): UMC128
QTL: ZM-DMC-10-1
Species: Zea mays
General Trait: YIELD
Specific Trait: Dry matter concentration
Citation: CROP SCI (1998) 38: 1278-1289
Chromosome: 10
Flanking Marker(s): UMC146
QTL: ZM-DMC-10-1
Species: Zea mays
General Trait: YIELD
Specific Trait: Dry matter concentration
Citation: THEOR APPL GENET (2001) 102: 230-243
Chromosome: 10
Flanking Markers(s):
QTL: ZM-DMC-10-2
Species: Zea mays
General Trait: YIELD
Specific Trait: Dry matter concentration
Citation: CROP SCI (1998) 38: 1278-1289
Chromosome: 10
Flanking Markers(s): UMC146
QTL: ZM-DMC-2-3
Species: Zea mays
General Trait: YIELD
Specific Trait: Dry matter concentration
Citation: THEOR APPL GENET (2001) 102: 230-243
Chromosome: 2
Flanking Markers(s):
QTL: ZM-DMC-5-1
Species: Zea mays
General Trait: YIELD
Specific Trait: Dry matter concentration
Citation: CROP SCI (1998) 38: 1278-1289
Chromosome: 5
Flanking Markers(s): UMC68
QTL: ZM-DMC-5-1
Species: Zea mays
General Trait: YIELD
Specific Trait: Dry matter content
Citation: CROP SCI (2001) 41: 690-697
Chromosome: 5
Flanking Markers(s): 116
QTL: ZM-DMC-5-1
Species: Zea mays
General Trait: YIELD
Specific Trait: Dry matter concentration
Citation: THEOR APPL GENET (2001) 102: 230-243
Chromosome: 5
Flanking Markers(s):
QTL: ZM-DMC-6-1
Species: Zea mays
General Trait: YIELD
Specific Trait: Dry matter concentration
Citation: CROP SCI (1998) 38: 1278-1289
Chromosome: 6
Flanking Markers(s): UMC85
QTL: ZM-DMC-6-1
Species: Zea mays
General Trait: YIELD
Specific Trait: Dry matter concentration
Citation: THEOR APPL GENET (2001) 102: 230-243
Chromosome: 6
Flanking Markers(s):
QTL: ZM-DMC-6-2
Species: Zea mays
General Trait: YIELD
Specific Trait: Dry matter concentration
Citation: CROP SCI (1998) 38: 1278-1289
Chromosome: 6
Flanking Marker(s): UMC59
QTL: ZM-DMC-8-1
Species: Zea mays
General Trait: YIELD
Specific Trait: Dry matter concentration
Citation: CROP SCI (1998) 38: 1278-1289
Chromosome: 8
Flanking Markers(s): UMC117
QTL: ZM-DMC-8-1
Species: Zea mays
General Trait: YIELD
Specific Trait: Dry matter content
Citation: CROP SCI (2001) 41: 690-697
Chromosome: 8
Flanking Markers(s): 132
QTL: ZM-DMC-8-1
Species: Zea mays
General Trait: YIELD
Specific Trait: Dry matter concentration
Citation: THEOR APPL GENET (2001) 102: 230-243
Chromosome: 8
Flanking Markers(s):
QTL: ZM-DMC-8-2
Species: Zea mays
General Trait: YIELD
Specific Trait: Dry matter concentration
Citation: CROP SCI (1998) 38: 1278-1289
Chromosome: 8
Flanking Markers(s): UMC71
QTL: ZM-DMC-8-2
Species: Zea mays
General Trait: YIELD
Specific Trait: Dry matter content
Citation: CROP SCI (2001) 41: 690-697
Chromosome: 8
Flanking Markers(s): 176
QTL: ZM-DMY-1-2
Species: Zea mays
General Trait: YIELD
Specific Trait: Dry matter yield
Citation: CROP SCI (1998) 38: 1278-1289
Chromosome: 1
Flanking Markers(s): UMC167
QTL: ZM-DMY-1-3
Species: Zea mays
General Trait: YIELD
Specific Trait: Dry matter yield
Citation: CROP SCI (1998) 38: 1278-1289
Chromosome: 1
Flanking Markers(s): UMC83A
QTL: ZM-DMY-1-4
Species: Zea mays
General Trait: YIELD
Specific Trait: Dry matter yield
Citation: CROP SCI (1998) 38: 1278-1289
Chromosome: 1
Flanking Markers(s): BNL5.59
QTL: ZM-DMY-1-5
Species: Zea mays
General Trait: YIELD
Specific Trait: Dry matter yield
Citation: CROP SCI (1998) 38: 1278-1289
Chromosome: 1
Flanking Markers(s): UMC83
QTL: ZM-DMY-10-1
Species: Zea mays
General Trait: YIELD
Specific Trait: Dry matter yield
Citation: CROP SCI (1998) 38: 1278-1289
Chromosome: 10
Flanking Markers(s): UMC64
QTL: ZM-DMY-10-1
Species: Zea mays
General Trait: YIELD
Specific Trait: Dry matter yield
Citation: CROP SCI (2001) 41: 690-697
Chromosome: 10
Flanking Markers(s): 56
QTL: ZM-DMY-2-1
Species: Zea mays
General Trait: YIELD
Specific Trait: Dry matter yield
Citation: CROP SCI (1998) 38: 1278-1289
Chromosome: 2
Flanking Markers(s): UMC53
QTL: ZM-DMY-2-3
Species: Zea mays
General Trait: YIELD
Specific Trait: Dry matter yield
Citation: CROP SCI (1998) 38: 1278-1289
Chromosome: 2
Flanking Markers(s): UMC4
QTL: ZM-DMY-2-4
Species: Zea mays
General Trait: YIELD
Specific Trait: Dry matter yield
Citation: CROP SCI (1998) 38: 1278-1289
Chromosome: 2
Flanking Markers(s): UMC36
QTL: ZM-DMY-3-1
Species: Zea mays
General Trait: YIELD
Specific Trait Dry matter yield
Citation: CROP SCI (1998) 38: 1278-1289
Chromosome: 3
Flanking Markers(s): BNL6.16
QTL: ZM-DMY-3-2
Species: Zea mays
General Trait: YIELD
Specific Trait: Dry matter yield
Citation: CROP SCI (1998) 38: 1278-1289
Chromosome: 3
Flanking Markers(s): UMC154
QTL: ZM-DMY-3-3
Species: Zea mays
General Trait: YIELD
Specific Trait: Dry matter yield
Citation: CROP SCI (1998) 38: 1278-1289
Chromosome: 3
Flanking Markers(s): UMC10
QTL: ZM-DMY-4-1
Species: Zea mays
General Trait: YIELD
Specific Trait: Dry matter yield
Citation: CROP SCI (1998) 38: 1278-1289
Chromosome: 4
Flanking Markers(s): UMC31
QTL: ZM-DMY-4-2
Species: Zea mays
General Trait: YIELD
Specific Trait: Dry matter yield
Citation: CROP SCI (1998) 38: 1278-1289
Chromosome: 4
Flanking Markers(s): BNL7.65
QTL: ZM-DMY-4-3
Species: Zea mays
General Trait: YIELD
Specific Trait: Dry matter yield
Citation: CROP SCI (1998) 38: 1278-1289
Chromosome: 4
Flanking Markers(s): UMC42
QTL: ZM-DMY-4-4
Species: Zea mays
General Trait: YIELD
Specific Trait: Dry matter yield
Citation: CROP SCI (1998) 38: 1278-1289
Chromosome: 4
Flanking Markers(s): UMC127B
QTL: ZM-DMY-5-1
Species: Zea mays
General Trait: YIELD
Specific Trait: Dry matter yield
Citation: CROP SCI (1998) 38: 1278-1289
Chromosome: 5
Flanking Markers(s): BNL7.71
QTL: ZM-DMY-8-1
Species: Zea mays
General Trait: YIELD
Specific Trait: Dry matter yield
Citation: CROP SCI (1998) 38: 1278-1289
Chromosome: 8
Flanking Markers(s): UMC120
QTL: ZM-DMY-8-1
Species: Zea mays
General Trait: YIELD
Specific Trait: Dry matter yield
Citation: CROP SCI (2001) 41: 690-697
Chromosome: 8
Flanking Markers(s): 172
QTL: ZM-DMY-8-2
Species: Zea mays
General Trait: YIELD
Specific Trait: Dry matter yield
Citation: CROP SCI (1998) 38: 1278-1289
Chromosome: 8
Flanking Markers(s): UMC12A
QTL: ZM-DMY-9-1
Species: Zea mays
General Trait: YIELD
Specific Trait: Dry matter yield
Citation: CROP SCI (1998) 38: 1278-1289
Chromosome: 9
Flanking Markers(s): UMC95
QTL: ZM-EWT-2-1
Species: Zea mays
General Trait: YIELD
Specific Trait: Ear weight
Citation: THEOR APPL GENET (1999) 99: 280-288
Chromosome: 2
Flanking Markers(s): PHI083
QTL: ZM-EWT-4-2
Species: Zea mays
General Trait: YIELD
Specific Trait: Ear weight
Citation: THEOR APPL GENET (1999) 99: 280-288
Chromosome: 4
Flanking Markers(s): PHI093
QTL: ZM-GWE-9-1
Species: Zea mays
General Trait: YIELD
Specific Trait: Grain weight per ear
Citation: THEOR APPL GENET (2001) 102: 591-599
Chromosome: 9
Flanking Markers(s):
QTL: ZM-GWM2-1-1
Species: Zea mays
General Trait: YIELD
Specific Trait: “Yield, grain weight per square meter”
Citation: THEOR APPL GENET (1999) 99: 1106-1119
Chromosome: 1
Flanking Markers(s): “UMC163, UMC161”
QTL: ZM-GWM2-10-1
Species: Zea mays
General Trait: YIELD
Specific Trait: “Yield, grain weight per square meter”
Citation: THEOR APPL GENET (1999) 99: 1106-1119
Chromosome: 10
Flanking Markers(s): “UMC146, UMC44”
QTL: ZM-GWM2-3-1
Species: Zea mays
General Trait: YIELD
Specific Trait: “Yield, grain weight per square meter”
Citation: THEOR APPL GENET (1999) 99: 1106-1119
Chromosome: 3
Flanking Markers(s): “UMC92, UMC10”
QTL: ZM-GWM2-3-2
Species: Zea mays
General Trait: YIELD
Specific Trait: “Yield, grain weight per square meter”
Citation: THEOR APPL GENET (1999) 99: 1106-1119
Chromosome: 3
Flanking Markers(s): “UMC3, UMC96”
QTL: ZM-GWM2-7-1
Species: Zea mays
General Trait: YIELD
Specific Trait: “Yield, grain weight per square meter”
Citation: THEOR APPL GENET (1999) 99: 1106-1119
Chromosome: 7
Flanking Markers(s): “BNL15.40, UMC116”
QTL: ZM-GYHA-1-1
Species: Zea mays
General Trait: YIELD
Specific Trait: Grain yield per hectare
Citation: CROP SCI (1998) 38: 1296-1308
Chromosome: 1
Flanking Markers(s):
QTL: ZM-GYHA-1-2
Species: Zea mays
General Trait: YIELD
Specific Trait: Grain yield per hectare
Citation: CROP SCI (1998) 38: 1296-1308
Chromosome: 1
Flanking Markers(s):
QTL: ZM-GYHA-1-3
Species: Zea mays
General Trait: YIELD
Specific Trait: Grain yield per hectare
Citation: CROP SCI (1998) 38: 1296-1308
Chromosome: 1
Flanking Markers(s):
QTL: ZM-GYHA-1-4
Species: Zea mays
General Trait: YIELD
Specific Trait: Grain yield per hectare
Citation: CROP SCI (1998) 38: 1296-1308
Chromosome: 1
Flanking Markers(s):
QTL: ZM-GYHA-3-1
Species: Zea mays
General Trait: YIELD
Specific Trait: Grain yield per hectare
Citation: CROP SCI (1998) 38: 1296-1308
Chromosome: 3
Flanking Markers(s):
QTL: ZM-GYHA-5-1
Species: Zea mays
General Trait: YIELD
Specific Trait: Grain yield per hectare
Citation: CROP SCI (1998) 38: 1296-1308
Chromosome: 5
Flanking Markers(s):
QTL: ZM-GYHA-6-1
Species: Zea mays
General Trait: YIELD
Specific Trait: Grain yield per hectare
Citation: CROP SCI (1998) 38: 1296-1308
Chromosome: 6
Flanking Markers(s):
QTL: ZM-GYHA-8-1
Species: Zea mays
General Trait: YIELD
Specific Trait: Grain yield per hectare
Citation: CROP SCI (1998) 38: 1296-1308
Chromosome: 8
Flanking Markers(s):
QTL: ZM-GYLD-1-1
Species: Zea mays
General Trait: YIELD
Specific Trait: Grain yield
Citation: CROP SCI (2000) 40: 30-39
Chromosome: 1
Flanking Markers(s):
QTL: ZM-GYLD-1-2
Species: Zea mays
General Trait: YIELD
Specific Trait: Grain yield
Citation: CROP SCI (2000) 40: 30-39
Chromosome: 1
Flanking Markers(s):
QTL: ZM-GYLD-2-1
Species: Zea mays
General Trait: YIELD
Specific Trait: Grain yield
Citation: PLANT BREEDING (1998) 117: 193-202
Chromosome: 2
Flanking Markers(s): “CDOCMT202, CSU75C”
QTL: ZM-GYLD-2-2
Species: Zea mays
General Trait: YIELD
Specific Trait: Grain yield
Citation: CROP SCI (2000) 40: 30-39
Chromosome: 2
Flanking Markers(s):
QTL: ZM-GYLD-2-3
Species: Zea mays
General Trait: YIELD
Specific Trait: Grain yield
Citation: CROP SCI (2000) 40: 30-39
Chromosome: 2
Flanking Markers(s):
QTL: ZM-GYLD-2-4
Species: Zea mays
General Trait: YIELD
Specific Trait: Grain yield
Citation: CROP SCI (2000) 40: 30-39
Chromosome: 2
Flanking Markers(s):
QTL: ZM-GYLD-3-3
Species: Zea mays
General Trait: YIELD
Specific Trait: Grain yield
Citation: CROP SCI (2000) 40: 30-39
Chromosome: 3
Flanking Markers(s):
QTL: ZM-GYLD-4-1
Species: Zea mays
General Trait: YIELD
Specific Trait: Grain yield
Citation: CROP SCI (2000) 40: 30-39
Chromosome: 4
Flanking Markers(s):
QTL: ZM-GYLD-5-1
Species: Zea mays
General Trait: YIELD
Specific Trait: Grain yield
Citation: CROP SCI (2000) 40: 30-39
Chromosome: 5
Flanking Markers(s):
QTL: ZM-GYLD-5-2
Species: Zea mays
General Trait: YIELD
Specific Trait: Grain yield
Citation: CROP SCI (2000) 40: 30-39
Chromosome: 5
Flanking Markers(s):
QTL: ZM-GYLD-5-3
Species: Zea mays
General Trait: YIELD
Specific Trait: Grain yield
Citation: CROP SCI (2000) 40: 30-39
Chromosome: 5
Flanking Markers(s):
QTL: ZM-GYLD-6-1
Species: Zea mays
General Trait: YIELD
Specific Trait: Grain yield
Citation: PLANT BREEDING (1998) 117: 193-202
Chromosome: 6
Flanking Markers(s): “CSU70, CDO580B”
QTL: ZM-GYLD-6-2
Species: Zea mays
General Trait: YIELD
Specific Trait: Grain yield
Citation: CROP SCI (2000) 40: 30-39
Chromosome: 6
Flanking Markers(s):
QTL: ZM-GYLD-6-3
Species: Zea mays
General Trait: YIELD
Specific Trait: Grain yield
Citation: CROP SCI (2000) 40: 30-39
Chromosome: 6
Flanking Markers(s):
QTL: ZM-GYLD-6-4
Species: Zea mays
General Trait: YIELD
Specific Trait: Grain yield
Citation: CROP SCI (2000) 40: 30-39
Chromosome: 6
Flanking Markers(s):
QTL: ZM-GYLD-7-3
Species: Zea mays
General Trait: YIELD
Specific Trait: Grain yield
Citation: CROP SCI (2000) 40: 30-39
Chromosome: 7
Flanking Markers(s):
QTL: ZM-GYLD-8-2
Species: Zea mays
General Trait: YIELD
Specific Trait: Grain yield
Citation: CROP SCI (2000) 40: 30-39
Chromosome: 8
Flanking Markers(s):
QTL: ZM-GYLD-9-1
Species: Zea mays
General Trait: YIELD
Specific Trait: Grain yield
Citation: CROP SCI (2000) 40: 30-39
Chromosome: 9
Flanking Markers(s):
QTL: ZM-GYLD-9-2
Species: Zea mays
General Trait: YIELD
Specific Trait: Grain yield
Citation: CROP SCI (2000) 40: 30-39
Chromosome: 9
Flanking Markers(s):
QTL: ZM-GYUI-9-1
Species: Zea mays
General Trait: YIELD
Specific Trait: Yield under corn borer infestation
Citation: THEOR APPL GENET (2000) 101: 907-917
Chromosome: 9
Flanking Markers(s):
QTL: ZM-GYUI-9-2
Species: Zea mays
General Trait: YIELD
Specific Trait: Yield under corn borer infestation
Citation: THEOR APPL GENET (2000) 101: 907-917
Chromosome: 9
Flanking Markers(s):
QTL: ZM-GYUP-1-1
Species: Zea mays
General Trait: YIELD
Specific Trait: Yield under corn borer protection
Citation: THEOR APPL GENET (2000) 101: 907-917
Chromosome: 1
Flanking Markers(s):
QTL: ZM-GYUP-1-2
Species: Zea mays
General Trait: YIELD
Specific Trait: Yield under corn borer protection
Citation: THEOR APPL GENET (2000) 101: 907-917
Chromosome: 1
Flanking Markers(s):
QTL: ZM-GYUP-9-1
Species: Zea mays
General Trait: YIELD
Specific Trait: Yield under corn borer protection
Citation: THEOR APPL GENET (2000) 101: 907-917
Chromosome: 9
Flanking Markers(s):
QTL: ZM-GYUP-9-2
Species: Zea mays
General Trait: YIELD
Specific Trait: Yield under corn borer protection
Citation: THEOR APPL GENET (2000) 101: 907-917
Chromosome: 9
Flanking Markers(s):
QTL: ZM-HI-1-1
Species: Zea mays
General Trait: YIELD
Specific Trait: Harvest index
Citation: THEOR APPL GENET (1999) 99: 1106-1119
Chromosome: 1
Flanking Markers(s): “UMC94, UMC76”
QTL: ZM-HI-1-2
Species: Zea mays
General Trait: YIELD
Specific Trait: Harvest index
Citation: THEOR APPL GENET (1999) 99: 1106-1119
Chromosome: 1
Flanking Markers(s): “UMC163, UMC161”
QTL: ZM-HI-10-1
Species: Zea mays
General Trait: YIELD
Specific Trait: Harvest index
Citation: THEOR APPL GENET (1999) 99: 1106-1119
Chromosome: 10
Flanking Markers(s): “UMC146, UMC44”
QTL: ZM-HI-3-1
Species: Zea mays
General Trait: YIELD
Specific Trait: Harvest index
Citation: THEOR APPL GENET (1999) 99: 1106-1119
Chromosome: 3
Flanking Markers(s): “UMC92, UMC10”
QTL: ZM-HI-4-1
Species: Zea mays
General Trait: YIELD
Specific Trait: Harvest index
Citation: THEOR APPL GENET (1999) 99: 1106-1119
Chromosome: 4
Flanking Markers(s): “UMC28.1, UMC19”
QTL: ZM-HI-7-1
Species: Zea mays
General Trait: YIELD
Specific Trait: Harvest index
Citation: THEOR APPL GENET (1999) 99: 1106-1119
Chromosome: 7
Flanking Markers(s): “BNL15.40, UMC116”
QTL: ZM-HI-8-1
Species: Zea mays
General Trait: YIELD
Specific Trait: Harvest index
Citation: THEOR APPL GENET (1999) 99: 1106-1119
Chromosome: 8
Flanking Markers(s): “UMC138L, UMC12”
QTL: ZM-ID-10-1
Species: Zea mays
General Trait: QUALITY
Specific Trait: In vitro digestibility of organic stover
Citation: THEOR APPL GENET (2000) 101: 907-917
Chromosome: 10
Flanking Markers(s):
QTL: ZM-ID-2-1
Species: Zea mays
General Trait: QUALITY
Specific Trait: In vitro digestibility of organic stover
Citation: THEOR APPL GENET (2000) 101: 907-917
Chromosome: 2
Flanking Markers(s):
QTL: ZM-ID-5-1
Species: Zea mays
General Trait: QUALITY
Specific Trait: In vitro digestibility of organic stover
Citation: THEOR APPL GENET (2000) 101: 907-917
Chromosome: 5
Flanking Markers(s):
QTL: ZM-ID-5-2
Species: Zea mays
General Trait: QUALITY
Specific Trait: In vitro digestibility of organic stover
Citation: THEOR APPL GENET (2000) 101: 907-917
Chromosome: 5
Flanking Markers(s):
QTL: ZM-ID-8-1
Species: Zea mays
General Trait: QUALITY
Specific Trait: In vitro digestibility of organic stover
Citation: THEOR APPL GENET (2000) 101: 907-917
Chromosome: 8
Flanking Markers(s):
QTL: ZM-IVDOM-1-1
Species: Zea mays
General Trait: QUALITY
Specific Trait: In vitro digestible organic matter
Citation: CROP SCI (1998) 38: 1278-1289
Chromosome: 1
Flanking Markers(s): UMC76
QTL: ZM-IVDOM-1-2
Species: Zea mays
General Trait: QUALITY
Specific Trait: In vitro digestible organic matter
Citation: CROP SCI (1998) 38: 1278-1289
Chromosome: 1
Flanking Markers(s): UMC58
QTL: ZM-IVDOM-1-3
Species: Zea mays
General Trait: QUALITY
Specific Trait: In vitro digestible organic matter
Citation: CROP SCI (1998) 38: 1278-1289
Chromosome: 1
Flanking Markers(s): UMC167
QTL: ZM-IVDOM-1-4
Species: Zea mays
General Trait: QUALITY
Specific Trait: In vitro digestible organic matter
Citation: CROP SCI (1998) 38: 1278-1289
Chromosome: 1
Flanking Markers(s): UMC37
QTL: ZM-IVDOM-10-1
Species: Zea mays
General Trait: QUALITY
Specific Trait: In vitro digestible organic matter
Citation: CROP SCI (1998) 38: 1278-1289
Chromosome: 10
Flanking Markers(s): UMC130
QTL: ZM-IVDOM-10-2
Species: Zea mays
General Trait: QUALITY
Specific Trait: In vitro digestible organic matter
Citation: CROP SCI (1998) 38: 1278-1289
Chromosome: 10
Flanking Markers(s): UMC18
QTL: ZM-IVDOM-3-1
Species: Zea mays
General Trait: QUALITY
Specific Trait: In vitro digestible organic matter
Citation: CROP SCI (1998) 38: 1278-1289
Chromosome: 3
Flanking Markers(s): UMC97
QTL: ZM-IVDOM-3-3
Species: Zea mays
General Trait: QUALITY
Specific Trait: In vitro digestible organic matter
Citation: CROP SCI (1998) 38: 1278-1289
Chromosome: 3
Flanking Markers(s): UMC97
QTL: ZM-IVDOM-5-1
Species: Zea mays
General Trait: QUALITY
Specific Trait: In vitro digestible organic matter
Citation: CROP SCI (1998) 38: 1278-1289
Chromosome: 5
Flanking Markers(s): UMC43
QTL: ZM-IVDOM-5-2
Species: Zea mays
General Trait: QUALITY
Specific Trait: In vitro digestible organic matter
Citation: CROP SCI (1998) 38: 1278-1289
Chromosome: 5
Flanking Markers(s): BNL7.71
QTL: ZM-IVDOM-5-3
Species: Zea mays
General Trait: QUALITY
Specific Trait In vitro digestible organic matter
Citation: CROP SCI (1998) 38: 1278-1289
Chromosome: 5
Flanking Markers(s): UMC90
QTL: ZM-IVDOM-9-1
Species: Zea mays
General Trait: QUALITY
Specific Trait: In vitro digestible organic matter
Citation: CROP SCI (1998) 38: 1278-1289
Chromosome: 9
Flanking Markers(s): BNL5.09
QTL: ZM-IVDOM-9-2
Species: Zea mays
General Trait: QUALITY
Specific Trait: In vitro digestible organic matter
Citation: CROP SCI (1998) 38: 1278-1289
Chromosome: 9
Flanking Markers(s): BNL14.28
QTL: ZM-KNE-4-1
Species: Zea mays
General Trait: YIELD
Specific Trait: Kernel number per ear
Citation: THEOR APPL GENET (1999) 99: 280-288
Chromosome: 4
Flanking Markers(s): PHI093
QTL: ZM-KW100-1-2
Species: Zea mays
General Trait: YIELD
Specific Trait: Kernel weight per 100 kernels
Citation: THEOR APPL GENET (1999) 99: 1106-1119
Chromosome: 1
Flanking Markers(s): “UMC157, BNL8.29”
QTL: ZM-KW100-3-1
Species: Zea mays
General Trait: YIELD
Specific Trait: Kernel weight per 100 kernels
Citation: THEOR APPL GENET (1999) 99: 1106-1119
Chromosome: 3
Flanking Markers(s): UMC60
QTL: ZM-KW100-9-1
Species: Zea mays
General Trait: YIELD
Specific Trait: Kernel weight per 100 kernels
Citation: THEOR APPL GENET (1999) 99: 1106-1119
Chromosome: 9
Flanking Markers(s): “UMC153, BNL5.09”
QTL: ZM-KW300-1-2
Species: Zea mays
General Trait: YIELD
Specific Trait: Kernel weight per 300 kernels
Citation: CROP SCI (1998) 38: 1296-1308
Chromosome: 1
Flanking Markers(s):
QTL: ZM-KW300-3-2
Species: Zea mays
General Trait: YIELD
Specific Trait: Kernel weight per 300 kernels
Citation: CROP SCI (1998) 38: 1296-1308
Chromosome: 3
Flanking Markers(s):
QTL: ZM-KW300-3-3
Species: Zea mays
General Trait: YIELD
Specific Trait: Kernel weight per 300 kernels
Citation: CROP SCI (1998) 38: 1296-1308
Chromosome: 3
Flanking Markers(s):
QTL: ZM-KW300-4-2
Species: Zea mays
General Trait: YIELD
Specific Trait: Kernel weight per 300 kernels
Citation: CROP SCI (1998) 38: 1296-1308
Chromosome: 4
Flanking Markers(s):
QTL: ZM-KW300-5-1
Species: Zea mays
General Trait: YIELD
Specific Trait: Kernel weight per 300 kernels
Citation: CROP SCI (1998) 38: 1296-1308
Chromosome: 5
Flanking Markers(s):
QTL: ZM-KW300-6-2
Species: Zea mays
General Trait: YIELD
Specific Trait: Kernel weight per 300 kernels
Citation: CROP SCI (1998) 38: 1296-1308
Chromosome: 6
Flanking Markers(s):
QTL: ZM-KW300-8-2
Species: Zea mays
General Trait: YIELD
Specific Trait: Kernel weight per 300 kernels
Citation: CROP SCI (1998) 38: 1296-1308
Chromosome: 8
Flanking Markers(s):
QTL: ZM-KW300-9-1
Species: Zea mays
General Trait: YIELD
Specific Trait: Kernel weight per 300 kernels
Citation: CROP SCI (1998) 38: 1296-1308
Chromosome: 9
Flanking Markers(s):
QTL: ZM-KW300-9-2
Species: Zea mays
General Trait: YIELD
Specific Trait: Kernel weight per 300 kernels
Citation: CROP SCI (1998) 38: 1296-1308
Chromosome: 9
Flanking Markers(s):
QTL: ZM-KWE-4-1
Species: Zea mays
General Trait: YIELD
Specific Trait: Kernel weight per ear
Citation: THEOR APPL GENET (1999) 99: 280-288
Chromosome: 4
Flanking Markers(s): PHI093
QTL: ZM-MOIST-1-1
Species: Zea mays
General Trait: QUALITY
Specific Trait: Grain moisture
Citation: CROP SCI (2000) 40: 30-39
Chromosome: 1
Flanking Markers(s):
QTL: ZM-MOIST-1-2
Species: Zea mays
General Trait: QUALITY
Specific Trait: Grain moisture
Citation: CROP SCI (2000) 40: 30-39
Chromosome: 1
Flanking Markers(s):
QTL: ZM-MOIST-1-3
Species: Zea mays
General Trait: QUALITY
Specific Trait: Grain moisture
Citation: CROP SCI (2000) 40: 30-39
Chromosome: 1
Flanking Markers(s):
QTL: ZM-MOIST-1-4
Species: Zea mays
General Trait: QUALITY
Specific Trait: Grain moisture
Citation: CROP SCI (2000) 40: 30-39
Chromosome: 1
Flanking Markers(s):
QTL: ZM-MOIST-1-5
Species: Zea mays
General Trait: QUALITY
Specific Trait: Grain moisture
Citation: CROP SCI (2000) 40: 30-39
Chromosome: 1
Flanking Markers(s):
QTL: ZM-MOIST-1-6
Species: Zea mays
Genera) Trait: QUALITY
Specific Trait: Grain moisture
Citation: CROP SCI (2000) 40: 30-39
Chromosome: 1
Flanking Markers(s):
QTL: ZM-MOIST-10-1
Species: Zea mays
General Trait: QUALITY
Specific Trait: Grain moisture
Citation: CROP SCI (2000) 40: 30-39
Chromosome: 10
Flanking Markers(s):
QTL: ZM-MOIST-2-1
Species: Zea mays
General Trait: QUALITY
Specific Trait: Grain moisture
Citation: CROP SCI (2000) 40: 30-39
Chromosome: 2
Flanking Markers(s):
QTL: ZM-MOIST-2-2
Species: Zea mays
General Trait: QUALITY
Specific Trait: Grain moisture
Citation: CROP SCI (2000) 40: 30-39
Chromosome: 2
Flanking Markers(s):
QTL: ZM-MOIST-2-3
Species: Zea mays
General Trait: QUALITY
Specific Trait: Grain moisture
Citation: CROP SCI (2000) 40: 30-39
Chromosome: 2
Flanking Markers(s):
QTL: ZM-MOIST-3-2
Species: Zea mays
General Trait: QUALITY
Specific Trait: Grain moisture
Citation: CROP SCI (2000) 40: 30-39
Chromosome: 3
Flanking Markers(s):
QTL: ZM-MOIST-3-3
Species: Zea mays
General Trait: QUALITY
Specific Trait: Grain moisture
Citation: CROP SCI (2000) 40: 30-39
Chromosome: 3
Flanking Markers(s):
QTL: ZM-MOIST-4-2
Species: Zea mays
General Trait: QUALITY
Specific Trait: Grain moisture
Citation: CROP SCI (2000) 40: 30-39
Chromosome: 4
Flanking Markers(s):
QTL: ZM-MOIST-4-3
Species: Zea mays
General Trait: QUALITY
Specific Trait: Grain moisture
Citation: CROP SCI (2000) 40: 30-39
Chromosome: 4
Flanking Markers(s):
QTL: ZM-MOIST-4-4
Species: Zea mays
General Trait: QUALITY
Specific Trait: Grain moisture
Citation: CROP SCI (2000) 40: 30-39
Chromosome: 4
Flanking Markers(s):
QTL: ZM-MOIST-5-1
Species: Zea mays
General Trait: QUALITY
Specific Trait: Grain moisture
Citation: CROP SCI (2000) 40: 30-39
Chromosome: 5
Flanking Markers(s):
QTL: ZM-MOIST-5-2
Species: Zea mays
General Trait: QUALITY
Specific Trait: Grain moisture
Citation: CROP SCI (2000) 40: 30-39
Chromosome: 5
Flanking Markers(s):
QTL: ZM-MOIST-5-3
Species: Zea mays
General Trait: QUALITY
Specific Trait: Grain moisture
Citation: CROP SCI (2000) 40: 30-39
Chromosome: 5
Flanking Markers(s):
QTL: ZM-MOIST-5-4
Species: Zea mays
General Trait: QUALITY
Specific Trait: Grain moisture
Citation: CROP SCI (2000) 40: 30-39
Chromosome: 5
Flanking Markers(s):
QTL: ZM-MOIST-6-2
Species: Zea mays
General Trait: QUALITY
Specific Trait: Grain moisture
Citation: CROP SCI (2000) 40: 30-39
Chromosome: 6
Flanking Markers(s):
QTL: ZM-MOIST-7-1
Species: Zea mays
General Trait: QUALITY
Specific Trait: Grain moisture
Citation: CROP SCI (2000) 40: 30-39
Chromosome: 7
Flanking Markers(s):
QTL: ZM-MOIST-7-2
Species: Zea mays
General Trait: QUALITY
Specific Trait: Grain moisture
Citation: CROP SCI (2000) 40: 30-39
Chromosome: 7
Flanking Markers(s):
QTL: ZM-MOIST-7-3
Species: Zea mays
General Trait: QUALITY
Specific Trait: Grain moisture
Citation: CROP SCI (2000) 40: 30-39
Chromosome: 7
Flanking Markers(s):
QTL: ZM-MOIST-7-4
Species: Zea mays
General Trait: QUALITY
Specific Trait: Grain moisture
Citation: CROP SCI (2000) 40: 30-39
Chromosome: 7
Flanking Markers(s):
QTL: ZM-MOIST-8-1
Species: Zea mays
General Trait: QUALITY
Specific Trait: Grain moisture
Citation: CROP SCI (2000) 40: 30-39
Chromosome: 8
Flanking Markers(s):
QTL: ZM-MOIST-8-2
Species: Zea mays
General Trait: QUALITY
Specific Trait: Grain moisture
Citation: CROP SCI (2000) 40: 30-39
Chromosome: 8
Flanking Markers(s):
QTL: ZM-MOIST-9-2
Species: Zea mays
General Trait: QUALITY
Specific Trait: Grain moisture
Citation: CROP SCI (2000) 40: 30-39
Chromosome: 9
Flanking Markers(s):
QTL: ZM-MOIST-9-3
Species: Zea mays
General Trait: QUALITY
Specific Trait: Grain moisture
Citation: CROP SCI (2000) 40: 30-39
Chromosome: 9
Flanking Markers(s):
QTL: ZM-PC-1-1
Species: Zea mays
General Trait: QUALITY
Specific Trait: Protein concentration
Citation: CROP SCI (1998) 38: 1062-1072
Chromosome: 1
Flanking Markers(s): “CSU92, CSUCMT11B”
QTL: ZM-PC-1-2
Species: Zea mays
General Trait: QUALITY
Specific Trait: Protein concentration
Citation: CROP SCI (1998) 38: 1062-1072
Chromosome: 1
Flanking Markers(s): “BNL8.29A, BNL6.32”
QTL: ZM-PC-5-1
Species: Zea mays
General Trait: QUALITY
Specific Trait: Protein concentration
Citation: CROP SCI (1998) 38: 1062-1072
Chromosome: 5
Flanking Markers(s): “UMC51A, UMC127B”
QTL: ZM-PC-8-1
Species: Zea mays
General Trait: QUALITY
Specific Trait: Protein concentration
Citation: CROP SCI (1998) 38: 1062-1072
Chromosome: 8
Flanking Markers(s): “CSU75D, CDO580A”
QTL: ZM-PC-9-1
Species: Zea mays
General Trait: QUALITY
Specific Trait: Protein concentration
Citation: CROP SCI (1998) 38: 1062-1072
Chromosome: 9
Flanking Markers(s): “CSU158, CSU147”
QTL: ZM-PR-9-1
Species: Zea mays
General Trait: QUALITY
Specific Trait: Protein content
Citation: THEOR APPL GENET (2001) 102: 591-599
Chromosome: 9
Flanking Markers(s):
QTL: ZM-STC-10-1
Species: Zea mays
General Trait: QUALITY
Specific Trait: Starch concentration
Citation: CROP SCI (1998) 38: 1278-1289
Chromosome: 10
Flanking Markers(s): UMC146
QTL: ZM-STC-10-2
Species: Zea mays
General Trait: QUALITY
Specific Trait: Starch concentration
Citation: CROP SCI (1998) 38: 1278-1289
Chromosome: 10
Flanking Markers(s): UMC18
QTL: ZM-STC-2-2
Species: Zea mays
General Trait: QUALITY
Specific Trait: Starch concentration
Citation: CROP SCI (1998) 38: 1278-1289
Chromosome: 2
Flanking Markers(s): UMC36
QTL: ZM-STC-5-1
Species: Zea mays
General Trait: QUALITY
Specific Trait: Starch concentration
Citation: CROP SCI (1998) 38: 1278-1289
Chromosome: 5
Flanking Markers(s): BNL5.40
QTL: ZM-STC-5-1
Species: Zea mays
General Trait: QUALITY
Specific Trait: Starch content
Citation: CROP SCI (2001) 41: 690-697
Chromosome: 5
Flanking Markers(s): 60
QTL: ZM-STC-6-1
Species: Zea mays
General Trait: QUALITY
Specific Trait: Starch concentration
Citation: CROP SCI (1998) 38: 1278-1289
Chromosome: 6
Flanking Markers(s): UMC46
QTL: ZM-STC-7-2
Species: Zea mays
General Trait: QUALITY
Specific Trait: Starch concentration
Citation: CROP SCI (1998) 38: 1278-1289
Chromosome: 7
Flanking Markers(s): UMC110
QTL: ZM-STC-8-1
Species: Zea mays
General Trait: QUALITY
Specific Trait: Starch concentration
Citation: CROP SCI (1998) 38: 1278-1289
Chromosome: 8
Flanking Markers(s): UMC124
QTL: ZM-STC-8-1
Species: Zea mays
General Trait: QUALITY
Specific Trait: Starch content
Citation: CROP SCI (2001) 41: 690-697
Chromosome: 8
Flanking Markers(s): 54
QTL: ZM-TGW-4-1
Species: Zea mays
General Trait: YIELD
Specific Trait: Thousand grain weight
Citation: THEOR APPL GENET (2001) 102: 591-599
Chromosome: 4
Flanking Markers(s):
QTL: ZM-TGW-9-1
Species: Zea mays
General Trait: YIELD
Specific Trait: Thousand grain weight
Citation: THEOR APPL GENET (2001) 102: 591-599
Chromosome: 9
Flanking Markers(s):
QTL: ZM-TGW-9-2
Species: Zea mays
General Trait: YIELD
Specific Trait: Thousand grain weight
Citation: THEOR APPL GENET (2001) 102: 591-599
Chromosome: 9
Flanking Markers(s):
QTL: ZM-TW-1-1
Species: Zea mays
General Trait: YIELD
Specific Trait: Test weight
Citation: THEOR APPL GENET (2001) 102: 230-243
Chromosome: 1
Flanking Markers(s):
QTL: ZM-TW-10-2
Species: Zea mays
General Trait: YIELD
Specific Trait: Test weight
Citation: THEOR APPL GENET (2001) 102: 230-243
Chromosome: 10
Flanking Markers(s):
QTL: ZM-TW-2-3
Species: Zea mays
General Trait: YIELD
Specific Trait: Test weight
Citation: THEOR APPL GENET (2001) 102: 230-243
Chromosome: 2
Flanking Markers(s):
QTL: ZM-TW-5-1
Species: Zea mays
General Trait: YIELD
Specific Trait Test weight
Citation: THEOR APPL GENET (2001) 102: 230-243
Chromosome: 5
Flanking Markers(s):
QTL: ZM-TW-8-1
Species: Zea mays
General Trait: YIELD
Specific Trait: Test weight
Citation: THEOR APPL GENET (2001) 102: 230-243
Chromosome: 8
Flanking Markers(s):
QTL: ZM-TW-9-1
Species: Zea mays
General Trait: YIELD
Specific Trait: Test weight
Citation: THEOR APPL GENET (2001) 102: 230-243
Chromosome: 9
Flanking Markers(s):
QTL: ZM-VT-6-1
Species: Zea mays
General Trait: QUALITY
Specific Trait: Vitreousness
Citation: THEOR APPL GENET (2001) 102: 591-599
Chromosome: 6
Flanking Markers(s):
QTL: ZM-YLD-1-1
Species: Zea mays
General Trait: YIELD
Specific Trait: Grain yield
Citation: THEOR APPL GENET (2001) 102: 230-243
Chromosome: 1
Flanking Markers(s):
QTL: ZM-YLD-2-1
Species: Zea mays
General Trait: YIELD
Specific Trait: Grain yield
Citation: THEOR APPL GENET (2001) 102: 230-243
Chromosome: 2
Flanking Markers(s):
QTL: ZM-YLD-2-2
Species: Zea mays
General Trait: YIELD
Specific Trait: Grain yield
Citation: THEOR APPL GENET (2001) 102: 230-243
Chromosome: 2
Flanking Markers(s):
QTL: ZM-YLD-4-1
Species: Zea mays
General Trait: YIELD
Specific Trait: Grain yield
Citation: THEOR APPL GENET (2001) 102: 230-243
Chromosome: 4
Flanking Markers(s):
QTL: ZM-YLD-6-1
Species: Zea mays
General Trait: YIELD
Specific Trait: Grain yield
Citation: THEOR APPL GENET (2001) 102: 230-243
Chromosome: 6
Flanking Markers(s):
QTL: ZM-YLD-9-1
Species: Zea mays
General Trait: YIELD
Specific Trait: Grain yield
Citation: THEOR APPL GENET (2001) 102: 230-243
Chromosome: 9
Flanking Markers(s):

TABLE 15
Swiss-Prot Data

| | | | | |
|---|---|---|---|---|
| 101 | Accession: | Swissprot_id: | Gi_number: | Description: BETA-AMYLASE |
| | P10538 | AMYB_SOYBN | 231541 | (1,4-ALPHA-D-GLUCAN MALTOHYDROLASE) |
| 113 | Accession: | Swissprot_id: | Gi_number: | Description: Alpha-glucosidase II |
| | Q9F234 | AGL2_BACTQ | 14423647 | |
| 1 | Accession: | Swissprot_id: | Gi_number: | Description: MPV17 protein |
| | P39210 | MPV1_HUMAN | 730059 | |
| 317 | Accession: | Swissprot_id: | Gi_number: | Description: Myb protein |
| | Q08759 | MYB_XENLA | 730090 | |
| 329 | Accession: | Swissprot_id: | Gi_number: | Description: MATERNAL PUMILIO |
| | P25822 | PUM_DROME | 131605 | PROTEIN |
| 173 | Accession: | Swissprot_id: | Gi_number: | Description: Glucose-6-phosphate |
| | P42862 | G6PA_ORYSA | 1169797 | isomerase, cytosolic A (GPI-A) |
| | | | | (Phosphoglucose isomerase A) (PGI-A) |
| | | | | (Phosphohexose isomerase A) (PHI-A) |
| 333 | Accession: | Swissprot_id: | Gi_number: | Description: ACTIN 1 |
| | P02582 | ACT1_MAIZE | 113220 | |
| 233 | Accession: | Swissprot_id: | Gi_number: | Description: GLYCOPROTEIN X |
| | P28968 | VGLX_HSVEB | 138350 | PRECURSOR |
| 335 | Accession: | Swissprot_id: | Gi_number: | Description: DEVELOPMENTAL PROTEIN |
| | Q05201 | EYA_DROME | 544271 | EYES ABSENT (PROTEIN CLIFT) |
| 119 | Accession: | Swissprot_id: | Gi_number: | Description: Sucrose synthase 2 |
| | O24301 | SUS2_PEA | 3915037 | (Sucrose-UDP glucosyltransferase 2) |
| 311 | Accession: | Swissprot_id: | Gi_number: | Description: Anthocyanin regulatory |
| | P10290 | MYBC_MAIZE | 127585 | C1 protein |
| 149 | Accession: | Swissprot_id: | Gi_number: | Description: FRUCTOSE-BISPHOSPHATE |
| | P17784 | ALF_ORYSA | 113622 | ALDOLASE, CYTOPLASMIC ISOZYME |
| 155 | Accession: | Swissprot_id: | Gi_number: | Description: FRUCTOSE-BISPHOSPHATE |
| | Q40677 | ALFC_ORYSA | 3913018 | ALDOLASE, CHLOROPLAST PRECURSOR (ALDP) |
| 143 | Accession: | Swissprot_id: | Gi_number: | Description: Triosephosphate isomerase, |
| | P46225 | TPIC_SECCE | 1174745 | chloroplast precursor (TIM) |
| 307 | Accession: | Swissprot_id: | Gi_number: | Description: G-box binding factor 4 |
| | P42777 | GBF4_ARATH | 1169863 | |
| 341 | Accession: | Swissprot_id: | Gi_number: | Description: DNA-DIRECTED RNA |
| | P16356 | RPB1_CAEEL | 133322 | POLYMERASE II LARGEST SUBUNIT |
| 193 | Accession: | Swissprot_id: | Gi_number: | Description: MYRISTOYLATED ALANINE-RICH |
| | P12624 | MACS_BOVIN | 585447 | C-KINASE SUBSTRATE (MARCKS) (ACAMP-81) |
| 131 | Accession: | Swissprot_id: | Gi_number: | Description: Soluble glycogen |
| | Q43846 | UGS4_SOLTU | 2833389 | [starch] synthase, chloroplast |
| | | | | precursor (SS III) |
| 199 | Accession: | Swissprot_id: | Gi_number: | Description: GLUCOAMYLASE |
| | P08640 | AMYH_YEAST | 728850 | S1/S2 PRECURSOR (GLUCAN 1,4-ALPHA- |
| | | | | GLUCOSIDASE) (1,4-ALPHA-D-GLUCAN |
| | | | | GLUCOHYDROLASE) |
| 343 | Accession: | Swissprot_id: | Gi_number: | Description: Transacting transcriptional |
| | P28284 | ICP0_HSV2H | 124135 | protein ICP0 (VMW118 protein) |
| 287 | Accession: | Swissprot_id: | Gi_number: | Description: Cell cycle control |
| | O59800 | CWF5_SCHPO | 18202094 | protein cwf5 |
| 191 | Accession: | Swissprot_id: | Gi_number: | Description: Endo-1,3; 1,4-beta-D- |
| | Q9ZT66 | E134_MAIZE | 8928122 | glucanase precursor |
| 215 | Accession: | Swissprot_id: | Gi_number: | Description: GLUTELIN |
| | P07730 | GLU2_ORYSA | 121475 | TYPE II PRECURSOR |
| 23 | Accession: | Swissprot_id: | Gi_number: | Description: Speckle-type |
| | O43791 | SPOP_HUMAN | 8134708 | POZ protein |
| 147 | Accession: | Swissprot_id: | Gi_number: | Description: Triosephosphate |
| | P48494 | TPIS_ORYSA | 1351270 | isomerase, cytosolic (TIM) |
| 347 | Accession: | Swissprot_id: | Gi_number: | Description: FRUCTOKINASE |
| | P37829 | SCRK_SOLTU | 585973 | |
| 157 | Accession: | Swissprot_id: | Gi_number: | Description: Phosphoglycolate |
| | P32662 | GPH_ECOLI | 418445 | phosphatase (PGP) |
| 349 | Accession: | Swissprot_id: | Gi_number: | Description: CALPHOTIN |
| | Q02910 | CPN_DROME | 416833 | |
| 139 | Accession: | Swissprot_id: | Gi_number: | Description: Glucose-1-phosphate |
| | P12299 | GLG2_WHEAT | 1707930 | adenylyltransferase large subunit, |
| | | | | chloroplast precursor (ADP-glucose |
| | | | | synthase) (ADP-glucose |
| | | | | pyrophosphorylase) (AGPASE S) |
| | | | | (Alpha-D-glucose-1-phosphate adenyl |
| | | | | transferase) |
| 175 | Accession: | Swissprot_id: | Gi_number: | Description: Triose phosphate/phosphate |
| | P52178 | CPT2_BRAOL | 1706110 | translocator, non-green plastid, |
| | | | | chloroplast precursor (CTPT) |
| 5 | Accession: | Swissprot_id: | Gi_number: | Description: Peroxidase P7 |
| | P00434 | PERX_BRARA | 464365 | |
| 351 | Accession: | Swissprot_id: | Gi_number: | Description: ZINC FINGER PROTEIN |
| | P38682 | GLO3_YEAST | 729595 | GLO3 |
| 353 | Accession: | Swissprot_id: | Gi_number: | Description: FRUCTOKINASE |
| | P37829 | SCRK_SOLTU | 585973 | |
| 255 | Accession: | Swissprot_id: | Gi_number: | Description: MUCIN 2 PRECURSOR |
| | Q02817 | MUC2_HUMAN | 2506877 | |
| | | | | (INTESTINAL MUCIN 2) |
| 75 | Accession: | Swissprot_id: | Gi_number: | Description: Pullulanase precursor |
| | P07206 | PULA_KLEPN | 131589 | (Alpha-dextrin endo-1,6-alpha- |
| | | | | glucosidase) (Pullulan 6- |
| | | | | glucanohydrolase) |
| 357 | Accession: | Swissprot_id: | Gi_number: | Description: IMMEDIATE-EARLY |
| | P33479 | IE18_PRVKA | 462387 | PROTEIN IE 180 |
| 359 | Accession: | Swissprot_id: | Gi_number: | Description: LINE-1 |
| | P08547 | LIN1_HUMAN | 126295 | REVERSE TRANSCRIPTASE HOMOLOG |
| 361 | Accession: | Swissprot_id: | Gi_number: | Description: EBNA-1 NUCLEAR |
| | P03211 | EBN1_EBV | 119110 | PROTEIN |
| 363 | Accession: | Swissprot_id: | Gi_number: | Description: MUCIN 2 PRECURSOR |
| | Q02817 | MUC2_HUMAN | 2506877 | (INTESTINAL MUCIN 2) |
| 365 | Accession: | Swissprot_id: | Gi_number: | Description: LINE-1 REVERSE |
| | P08548 | LIN1_NYCCO | 126296 | TRANSCRIPTASE HOMOLOG |
| 181 | Accession: | Swissprot_id: | Gi_number: | Description: PYROPHOSPHATE--FRUCTOSE |
| | Q41140 | PFPA_RICCO | 2499488 | 6-PHOSPHATE 1-PHOSPHOTRANSFERASE |
| | | | | ALPHA SUBUNIT (PFP) (6-PHOS- |
| | | | | PHOFRUCTOKINASE (PYROPHOSPHATE)) |
| | | | | (PYROPHOSPHATE-DEPENDENT 6- |
| | | | | PHOSPHOFRUCTOSE-1-KINASE) (PPI-PFK) |
| 367 | Accession: | Swissprot_id: | Gi_number: | Description: RETINAL DEGENERATION B |
| | P43125 | RDGB_DROME | 1172875 | PROTEIN (PROBABLE CALCIUM |
| | | | | TRANSPORTER RDGB) |
| 261 | Accession: | Swissprot_id: | Gi_number: | Description: 3-DEOXY-MANNO- |
| | Q59320 | KDSB_CHLTR | 7387818 | OCTULOSONATE CYTIDYLYLTRANSFERASE |
| | | | | (CMP-KDO SYNTHETASE) (CMP-2-KETO-3- |
| | | | | DEOXYOCTULOSONIC ACID SYNTHETASE) (CKS) |
| 221 | Accession: | Swissprot_id: | Gi_number: | Description: CYSTATHIONINE GAMMA- |
| | P55217 | METB_ARATH | 2507422 | SYNTHASE, CHLOROPLAST PRECURSOR |
| | | | | (CGS) (O-SUCCINYLHOMOSERINE (THIOL)- |
| | | | | LYASE) |
| 57 | Accession: | Swissprot_id: | Gi_number: | Description: ARABINOSE-PROTON |
| | P09830 | ARAE_ECOLI | 114102 | SYMPORTER (ARABINOSE TRANSPORTER) |
| 25 | Accession: | Swissprot_id: | Gi_number: | Description: RECEPTOR PROTEIN |
| | Q9SYQ8 | CLV1_ARATH | 12643323 | KINASE CLAVATA1 PRECURSOR |
| 369 | Accession: | Swissprot_id: | Gi_number: | Description: REGULATORY PROTEIN E2 |
| | P06921 | VE2_HPV05 | 1352839 | |
| 39 | Accession: | Swissprot_id: | Gi_number: | Description: LEUCINE-RICH REPEAT |
| | Q9UQ13 | SHO2_HUMAN | 14423936 | PROTEIN SHOC-2 (RAS-BINDING PROTEIN |
| | | | | SUR-8) |
| 87 | Accession: | Swissprot_id: | Gi_number: | Description: Alpha-amylase isozyme |
| | P27935 | AM2A_ORYSA | 113678 | 2A precursor (1,4-alpha-D-glucan |
| | | | | glucanohydrolase) |
| 371 | Accession: | Swissprot_id: | Gi_number: | Description: EARLY NODULIN 93 |
| | Q02921 | NO93_SOYBN | 730165 | (N-93) |
| 163 | Accession: | Swissprot_id: | Gi_number: | Description: Triose phosphate/phosphate |
| | P52178 | CPT2_BRAOL | 1706110 | translocator, non-green plastid, |
| | | | | chloroplast precursor (CTPT) |
| 375 | Accession: | Swissprot_id: | Gi_number: | Description: BEM46 PROTEIN |
| | P54069 | BE46_SCHPO | 12644312 | |
| 315 | Accession: | Swissprot_id: | Gi_number: | Description: Myb-related protein |
| | P20025 | MYB3_MAIZE | 127582 | Zm38 |
| 89 | Accession: | Swissprot_id: | Gi_number: | Description: ALPHA-AMYLASE ISOZYME |
| | P27934 | AM3E_ORYSA | 113683 | 3E PRECURSOR (1,4-ALPHA-D-GLUCAN |
| | | | | GLUCANOHYDROLASE) |
| 289 | Accession: | Swissprot_id: | Gi_number: | Description: ASPARTATE AMINOTRANSFERASE, |
| | P37833 | AATC_ORYSA | 584706 | CYTOPLASMIC (TRANSAMINASE A) |
| 49 | Accession: | Swissprot_id: | Gi_number: | Description: SUGAR CARRIER |
| | Q41144 | STC_RICCO | 3915039 | PROTEIN C |
| 153 | Accession: | Swissprot_id: | Gi_number: | Description: TRIOSE PHOSPHATE/ |
| | P21727 | CPTR_PEA | 117290 | PHOSPHATE TRANSLOCATOR, CHLOROPLAST |
| | | | | PRECURSOR (CTPT) (P36) (E30) |
| 81 | Accession: | Swissprot_id: | Gi_number: | Description: ALPHA-AMYLASE |
| | P17654 | AMY1_ORYSA | 113766 | PRECURSOR (1,4-ALPHA-D-GLUCAN |
| | | | | GLUCANOHYDROLASE) (ISOZYME 1B) |
| 379 | Accession: | Swissprot_id: | Gi_number: | Description: WISKOTT-ALDRICH |
| | O43516 | WAIP_HUMAN | 13124642 | SYNDROME PROTEIN INTERACTING |
| | | | | PROTEIN (WASP INTERACTING PROTEIN) |
| | | | | (PRPL-2 PROTEIN) |
| 305 | Accession: | Swissprot_id: | Gi_number: | Description: TRANSCRIPTIONAL |
| | Q02516 | HAP5_YEAST | 2493550 | ACTIVATOR HAP5 |
| 381 | Accession: | Swissprot_id: | Gi_number: | Description: Retrovirus-related |
| | P10978 | POLX_TOBAC | 130582 | Pol polyprotein from transposon |
| | | | | TNT 1-94 [Contains: Protease; |
| | | | | Reverse transcriptase; Endonuclease] |
| 197 | Accession: | Swissprot_id: | Gi_number: | Description: Alpha-amylase/trypsin |
| | P01087 | IAAT_ELECO | 2851515 | inhibitor (RBI) (RATI) |
| 45 | Accession: | Swissprot_id: | Gi_number: | Description: Organic cation/ |
| | O76082 | OCN2_HUMAN | 8928257 | carnitine transporter 2 (Solute |
| | | | | carrier family 22, member 5) |
| | | | | (High-affinity sodium-dependent |
| | | | | carnitine cotransporter) |
| 97 | Accession: | Swissprot_id: | Gi_number: | Description: SEED ALLERGENIC |
| | Q01885 | RAG2_ORYSA | 548671 | PROTEIN RAG2 PRECURSOR |
| 383 | Accession: | Swissprot_id: | Gi_number: | Description: RING FINGER PROTEIN |
| | Q9WTV7 | RNFB_MOUSE | 13124535 | 12 (LIM DOMAIN INTERACTING RING |
| | | | | FINGER PROTEIN) (RING FINGER LIM |
| | | | | DOMAIN-BINDING PROTEIN) (R-LIM) |
| 135 | Accession: | Swissprot_id: | Gi_number: | Description: Glucose-1-phosphate |
| | P55241 | GLG1_MAIZE | 1707924 | adenylyltransferase large subunit 1, |
| | | | | chloroplast precursor (ADP-glucose |
| | | | | synthase) (ADP-glucose |
| | | | | pyrophosphorylase) (AGPASE S) |
| | | | | (Alpha-D-glucose-1-phosphate adenyl |
| | | | | transferase) (Shrunken-2) |
| 267 | Accession: | Swissprot_id: | Gi_number: | Description: PROLINE-RICH |
| | P05143 | PRP3_MOUSE | 131002 | PROTEIN MP-3 |
| 385 | Accession: | Swissprot_id: | Gi_number: | Description: CYSTEINE PROTEINASE |
| | Q10993 | CYTB_HELAN | 1706277 | INHIBITOR B (CYSTATIN B) (SCB) |
| 283 | Accession: | Swissprot_id: | Gi_number: | Description: Glycine-rich RNA- |
| | P49311 | GRP2_SINAL | 1346181 | binding protein GRP2A |
| 53 | Accession: | Swissprot_id: | Gi_number: | Description: CATION TRANSPORT |
| | P39163 | CHAC_ECOLI | 12644253 | PROTEIN CHAC |
| 253 | Accession: | Swissprot_id: | Gi_number: | Description: Tetraacyldisaccharide |
| | Q9KQX0 | LPXK_VIBCH | 14423750 | 4′-kinase (Lipid A 4′-kinase) |
| 295- | Accession: | Swissprot_id: | Gi_number: | Description: S-adenosylmethionine: |
| | Q9I2W7 | MENG_PSEAE | 17369015 | 2-demethylmenaquinone methyl- |
| | | | | transferase |
| 389 | Accession: | Swissprot_id: | Gi_number: | Description: Extensin precursor |
| | P13983 | EXTN_TOBAC | 119714 | (Cell wall hydroxyproline-rich |
| | | | | glycoprotein) |
| 225 | Accession: | Swissprot_id: | Gi_number: | Description: GLUTELIN PRECURSOR |
| | P14323 | GLU4_ORYSA | 121476 | |
| 391 | Accession: | Swissprot_id: | Gi_number: | Description: GAMMA-GLIADIN |
| | P08453 | GDB2_WHEAT | 121101 | PRECURSOR |
| 167 | Accession: | Swissprot_id: | Gi_number: | Description: Fructose-2,6- |
| | P32604 | F26_YEAST | 1169587 | bisphosphatase |
| 137 | Accession: | Swissprot_id: | Gi_number: | Description: Glucose-1-phosphate |
| | P55238 | GLGS_HORVU | 1707940 | adenylyltransferase small subunit, |
| | | | | chloroplast precursor (ADP-glucose |
| | | | | synthase) (ADP-glucose |
| | | | | pyrophosphorylase) (AGPASE B) |
| | | | | (Alpha-D-glucose-1-phosphate adenyl |
| | | | | transferase) |
| 195 | Accession: | Swissprot_id: | Gi_number: | Description: MUCIN 2 PRECURSOR |
| | Q02817 | MUC2_HUMAN | 2506877 | (INTESTINAL MUCIN 2) |
| 263 | Accession: | Swissprot_id: | Gi_number: | Description: ACYL-COA-BINDING |
| | O22643 | ACBP_FRIAG | 5902717 | PROTEIN (ACBP) |
| 223 | Accession: | Swissprot_id: | Gi_number: | Description: GLUTELIN TYPE I |
| | P07728 | GU11_ORYSA | 121469 | PRECURSOR (CLONE PREE 61) |
| 85 | Accession: | Swissprot_id: | Gi_number: | Description: ALPHA-AMYLASE ISOZYME |
| | P27937 | AM3B_ORYSA | 113680 | 3B PRECURSOR (1,4-ALPHA-D-GLUCAN |
| | | | | GLUCANOHYDROLASE) |
| 129 | Accession: | Swissprot_id: | Gi_number: | Description: Glycogen [starch] |
| | Q43093 | UGS3_PEA | 2833384 | synthase, chloroplast precursor |
| | | | | (GBSSII) (Granule-bound starch |
| | | | | synthase II) |
| 103 | Accession: | Swissprot_id: | Gi_number: | Description: BETA-AMYLASE (1,4- |
| | P93594 | AMYB_WHEAT | 3334120 | ALPHA-D-GLUCAN MALTOHYDROLASE) |
| 51 | Accession: | Swissprot_id: | Gi_number: | Description: Peptide transporter |
| | P46032 | PT2B_ARATH | 1172704 | PTR2-B (Histidine transporting |
| | | | | protein) |
| 99 | Accession: | Swissprot_id: | Gi_number: | Description: SEED ALLERGENIC |
| | Q01885 | RAG2_ORYSA | 548671 | PROTEIN RAG2 PRECURSOR |
| 69 | Accession: | Swissprot_id: | Gi_number: | Description: 1,4-ALPHA-GLUCAN |
| | Q01401 | GLGB_ORYSA | 399544 | BRANCHING ENZYME (STARCH BRANCHING |
| | | | | ENZYME) (Q-ENZYME) |
| 229 | Accession: | Swissprot_id: | Gi_number: | Description: GLUTELIN TYPE II |
| | P07730 | GLU2_ORYSA | 121475 | PRECURSOR |
| 241 | Accession: | Swissprot_id: | Gi_number: | Description: 10 KD PROLAMIN |
| | P15839 | PRO1_ORYSA | 130946 | PRECURSOR |
| 91 | Accession: | Swissprot_id: | Gi_number: | Description: ALPHA-AMYLASE PRECURSOR |
| | P17654 | AMY1_ORYSA | 113766 | (1,4-ALPHA-D-GLUCAN GLUCANOHYDROLASE) |
| | | | | (ISOZYME 1B) |
| 401 | Accession: | Swissprot_id: | Gi_number: | Description: GLUTELIN PRECURSOR |
| | P14323 | GLU4_ORYSA | 121476 | |
| 121 | Accession: | Swissprot_id: | Gi_number: | Description: Sucrose synthase 2 |
| | P31924 | SUS2_ORYSA | 401140 | (Sucrose-UDP glucosyltransferase 2) |
| 403 | Accession: | Swissprot_id: | Gi_number: | Description: Mago |
| | O65806 | MGN_EUPLA | 6016561 | nashi protein homolog |
| 187 | Accession: | Swissprot_id: | Gi_number: | Description: FRUCTOSE-1,6- |
| | O64422 | F16P_ORYSA | 3913641 | BISPHOSPHATASE, CHLOROPLAST |
| | | | | PRECURSOR (D-FRUCTOSE-1,6- |
| | | | | BISPHOSPHATE 1-PHOSPHOHYDROLASE) |
| | | | | (FBPASE) |
| 13 | Accession: | Swissprot_id: | Gi_number: | Description: Blue copper protein |
| | Q41001 | BCP_PEA | 2493318 | precursor |
| 243 | Accession: | Swissprot_id: | Gi_number: | Description: PROLAMIN PPROL 17 |
| | P20698 | PRO7_ORYSA | 130959 | PRECURSOR |
| 203 | Accession: | Swissprot_id: | Gi_number: | Description: Glycogen operon |
| | Q10767 | GLGX_MYCTU | 1707945 | protein glgX homolog |
| 407 | Accession: | Swissprot_id: | Gi_number: | Description: Vegetatible |
| | Q00808 | HET1_PODAN | 3023956 | incompatibility protein HET-E-1 |
| 409 | Accession: | Swissprot_id: | Gi_number: | Description: O-METHYLTRANSFERASE |
| | P47917 | ZRP4_MAIZE | 1353193 | ZRP4 (OMT) |
| 411 | Accession: | Swissprot_id: | Gi_number: | Description: GLUCOAMYLASE |
| | P08640 | AMYH_YEAST | 728850 | S1/S2 PRECURSOR (GLUCAN 1,4-ALPHA- |
| | | | | GLUCOSIDASE) (1,4-ALPHA-D-GLUCAN |
| | | | | GLUCOHYDROLASE) |
| 105 | Accession: | Swissprot_id: | Gi_number: | Description: BETA-AMYLASE (1,4- |
| | P55005 | AMYB_MAIZE | 1703302 | ALPHA-D-GLUCAN MALTOHYDROLASE) |
| 107 | Accession: | Swissprot_id: | Gi_number: | Description: BETA-AMYLASE (1,4- |
| | P10538 | AMYB_SOYBN | 231541 | ALPHA-D-GLUCAN MALTOHYDROLASE) |
| 115 | Accession: | Swissprot_id: | Gi_number: | Description: ALPHA-GLUCOSIDASE |
| | Q43763 | AGLU_HORVU | 3023275 | PRECURSOR (MALTASE) |
| 15 | Accession: | Swissprot_id: | Gi_number: | Description: DnaJ homolog subfamily |
| | P25685 | DJB1_HUMAN | 1706473 | B member 1 (Heat shock 40 kDa |
| | | | | protein 1) (Heat shock protein 40) |
| | | | | (HSP40) (DnaJ protein homolog 1) |
| | | | | (HDJ-1) |
| 165 | Accession: | Swissprot_id: | Gi_number: | Description: Alpha-1,4 glucan |
| | P27598 | PHSL_IPOBA | 130172 | phosphorylase, L isozyme, |
| | | | | chloroplast precursor (Starch |
| | | | | phosphorylase L) |
| 123 | Accession: | Swissprot_id: | Gi_number: | Description: Sucrose synthase 3 |
| | Q43009 | SUS3_ORYSA | 3915054 | (Sucrose-UDP glucosyltransferase 3) |
| 205 | Accession: | Swissprot_id: | Gi_number: | Description: MUCIN 2 PRECURSOR |
| | Q02817 | MUC2_HUMAN | 2506877 | (INTESTINAL MUCIN 2) |
| 413 | Accession: | Swissprot_id: | Gi_number: | Description: ANTER-SPECIFIC PROLINE- |
| | P40603 | APG_BRANA | 728868 | RICH PROTEIN APG (PROTEIN CEX) |
| 209 | Accession: | Swissprot_id: | Gi_number: | Description: ANTHOCYANIN REGULATORY |
| | P13526 | ARLC_MAIZE | 114156 | LC PROTEIN |
| 323 | Accession: | Swissprot_id: | Gi_number: | Description: Wiskott-Aldrich syndrome |
| | P70315 | WASP_MOUSE | 2499130 | protein homolog (WASP) |
| 415 | Accession: | Swissprot_id: | Gi_number: | Description: SPIDROIN 1 |
| | P19837 | SPD1_NEPCL | 1174414 | (DRAGLINE SILK FIBROIN 1) |
| 141 | Accession: | Swissprot_id: | Gi_number: | Description: Glucose-1-phosphate |
| | P55238 | GLGS_HORVU | 1707940 | adenylyltransferase small subunit, |
| | | | | chloroplast precursor (ADP-glucose |
| | | | | synthase) (ADP-glucose |
| | | | | pyrophosphorylase)(AGPASE B) |
| | | | | (Alpha-D-glucose-1-phosphate adenyl |
| | | | | transferase) |
| 27 | Accession: | Swissprot_id: | Gi_number: | Description: Carbon catabolite |
| | Q02723 | RKI1_SECCE | 400982 | derepressing protein kinase |
| 65 | Accession: | Swissprot_id: | Gi_number: | Description: PHOSPHATE-REPRESSIBLE |
| | P15710 | PHO4_NEUCR | 130117 | PHOSPHATE PERMEASE |
| 185 | Accession: | Swissprot_id: | Gi_number: | Description: PYROPHOSPHATE-- |
| | Q41140 | PFPA_RICCO | 2499488 | FRUCTOSE 6-PHOSPHATE 1- |
| | | | | PHOSPHOTRANSFERASE ALPHA SUBUNIT |
| | | | | (PFP) (6-PHOSPHOFRUCTOKINASE |
| | | | | (PYROPHOSPHATE)) (PYROPHOSPHATE- |
| | | | | DEPENDENT 6-PHOSPHOFRUCTOSE-1- |
| | | | | KINASE) (PPI-PFK) |
| 299 | Accession: | Swissprot_id: | Gi_number: | Description: Heterogeneous nuclear |
| | P09651 | ROA1_HUMAN | 133254 | ribonucleoprotein A1 (Helix- |
| | | | | destabilizing protein) (Single- |
| | | | | strand binding protein) (hnRNP |
| | | | | core protein A1) |
| 67 | Accession: | Swissprot_id: | Gi_number: | Description: Peptide transporter |
| | P46032 | PT2B_ARATH | 1172704 | PTR2-B (Histidine transporting |
| | | | | protein) |
| 17 | Accession: | Swissprot_id: | Gi_number: | Description: Stromal 70 kDa heat |
| | Q02028 | HS7S_PEA | 399942 | shock-related protein, chloroplast |
| | | | | precursor |
| 279 | Accession: | Swissprot_id: | Gi_number: | Description: Probable |
| | P38994 | MSS4_YEAST | 1709144 | phosphatidylinositol-4-phosphate 5- |
| | | | | kinase MSS4 (1-phosphatidylinositol- |
| | | | | 4-phosphate kinase) (PIP5K) |
| | | | | (PtdIns(4)P-5-kinase) |
| | | | | (Diphosphoinositide kinase) |
| 71 | Accession: | Swissprot_id: | Gi_number: | Description: 1,4-alpha-glucan |
| | Q08047 | GLGB_MAIZE | 1169911 | branching enzyme IIB, chloroplast |
| | | | | precursor (Starch branching enzyme |
| | | | | IIB) (Q-enzyme) |
| 207 | Accession: | Swissprot_id: | Gi_number: | Description: Indole-3-glycerol |
| | P49572 | TRPC_ARATH | 1351303 | phosphate synthase, chloroplast |
| | | | | precursor (IGPS) |
| 417 | Accession: | Swissprot_id: | Gi_number: | Description: Transacting tran- |
| | P28284 | ICP0_HSV2H | 124135 | scriptional protein ICP0 (VMW118 |
| | | | | protein) |
| 127 | Accession: | Swissprot_id: | Gi_number: | Description: Sucrose synthase 2 |
| | O24301 | SUS2_PEA | 3915037 | (Sucrose-UDP glucosyltransferase 2) |
| 125 | Accession: | Swissprot_id: | Gi_number: | Description: Sucrose synthase 2 |
| | O24301 | SUS2_PEA | 3915037 | (Sucrose-UDP glucosyltransferase 2) |
| 183 | Accession: | Swissprot_id: | Gi_number: | Description: Pyrophosphate-- |
| | Q59126 | PFP_AMYME | 3122594 | fructose 6-phosphate 1-phospho- |
| | | | | transferase (6-phosphofructokinase |
| | | | | (Pyrophosphate)) (Pyrophosphate- |
| | | | | dependent 6-phosphofructose-1- |
| | | | | kinase) (PPI-PFK) |
| 419 | Accession: | Swissprot_id: | Gi_number: | Description: GLUTELIN TYPE-B |
| | Q02897 | GLUC_ORYSA | 544400 | 2 PRECURSOR |
| 421 | Accession: | Swissprot_id: | Gi_number: | Description: Goliath protein |
| | Q06003 | GOLI_DROME | 462193 | (G1 protein) |
| 29 | Accession: | Swissprot_id: | Gi_number: | Description: Calcium-dependent |
| | P53682 | CDP1_ORYSA | 1705733 | protein kinase, isoform 1 (CDPK 1) |
| 297 | Accession: | Swissprot_id: | Gi_number: | Description: MATERNAL PUMILIO |
| | P25822 | PUM_DROME | 131605 | PROTEIN |
| 245 | Accession: | Swissprot_id: | Gi_number: | Description: IMMUNOGLOBULIN A1 |
| | P45386 | IGA4_HAEIN | 1170517 | PROTEASE PRECURSOR (IGA1 PROTEASE) |
| 427 | Accession: | Swissprot_id: | Gi_number: | Description: RETROTRANSPOSABLE |
| | Q05654 | RDPO_SCHPO | 1710054 | ELEMENT TF2 155 KDA PROTEIN |
| 159/171 | Accession: | Swissprot_id: | Gi_number: | Description: Glucose-6-phosphate |
| X | P42862 | G6PA_ORYSA | 1169797 | isomerase, cytosolic A (GPI-A) |
| | | | | (Phosphoglucose isomerase A) (PGI-A) |
| | | | | (Phosphohexose isomerase A) (PHI-A) |
| 31 | Accession: | Swissprot_id: | Gi_number: | Description: Peptide transporter |
| | P46032 | PT2B_ARATH | 1172704 | PTR2-B (Histidine transporting |
| | | | | protein) |
| 403/431- | Accession: | Swissprot_id: | Gi_number: | Description: VITELLOGENIN II |
| | P02845 | VIT2_CHICK | 138595 | PRECURSOR (MAJOR VITELLOGENIN) |
| | | | | [CONTAINS: LIPOVITELLIN I |
| | | | | (LVI); PHOSVITIN (PV); LIPOVITELLIN |
| | | | | II (LVII); YGP40] |
| 275 | Accession: | Swissprot_id: | Gi_number: | Description: TRANSCRIPTIONAL |
| | P15276 | ALGP_PSEAE | 13959675 | REGULATORY PROTEIN ALGP (ALGINATE |
| | | | | REGULATORY PROTEIN ALGR3) |
| 19 | Accession: | Swissprot_id: | Gi_number: | Description: Protein phosphatase 2C |
| | O62830 | P2CB_BOVIN | 10720178 | beta isoform (PP2C-beta) |
| 151 | Accession: | Swissprot_id: | Gi_number: | Description: Alpha-glucan phos- |
| | Q9LKJ3 | PHSH_WHEAT | 14916632 | phorylase, H isozyme (Starch |
| | | | | phosphorylase H) |
| 213/227- | Accession: | Swissprot_id: | Gi_number: | Description: 19 KD GLOBULIN |
| | P29835 | GL19_ORYSA | 232161 | PRECURSOR (ALPHA-GLOBULIN) |
| 237 | Accession: | Swissprot_id: | Gi_number: | Description: CALMODULIN |
| | P02595 | CALM_PATSP | 115518 | |
| 133 | Accession: | Swissprot_id: | Gi_number: | Description: Granule-bound glycogen |
| | Q42968 | UGST_ORYGL | 2833382 | [starch] synthase, chloroplast |
| | | | | precursor |
| 239 | Accession: | Swissprot_id: | Gi_number: | Description: GLUTELIN TYPE-A |
| | Q09151 | GLU3_ORYSA | 1707986 | III PRECURSOR |
| 161 | Accession: | Swissprot_id: | Gi_number: | Description: UTP--GLUCOSE-1- |
| | Q43772 | UDPG_HORVU | 6136111 | PHOSPHATE URIDYLYLTRANSFERASE |
| | | | | (UDP-GLUCOSE PYROPHOSPHORYLASE) |
| | | | | (UDPGP) (UGPASE) |
| 61 | Accession: | Swissprot_id: | Gi_number: | Description: Intestinal |
| | P70545 | NDC2_RAT | 2499525 | sodium/dicarboxylate cotransporter |
| | | | | Na(+)/dicarboxylate cotransporter) |
| 47 | Accession: | Swissprot_id: | Gi_number: | Description: INORGANIC PHOSPHATE |
| | P25297 | PH84_YEAST | 1346710 | TRANSPORTER PHO84 |
| 219 | Accession: | Swissprot_id: | Gi_number: | Description: PROLAMIN PPROL 17 |
| | P20698 | PRO7_ORYSA | 130959 | PRECURSOR |
| 435 | Accession: | Swissprot_id: | Gi_number: | Description: SEED ALLERGENIC |
| | Q01881 | RA05_ORYSA | 548657 | PROTEIN RA5 PRECURSOR |
| 259/271- | Accession: | Swissprot_id: | Gi_number: | Description: OLEOSIN 16 KD |
| | Q42980 | OLE1_ORYSA | 3334280 | (OSE701) |
| 93 | Accession: | Swissprot_id: | Gi_number: | Description: PROTEIN KINASE APK1B |
| | P46573 | APKB_ARATH | 12644274 | |
| 441 | Accession: | Swissprot_id: | Gi_number: | Description: Luminal binding protein |
| | Q03685 | BIP5_TOBAC | 729623 | 5 precursor (BiP 5) (78 kDa glucose- |
| | | | | regulated protein homolog 5) |
| | | | | (GRP 78-5) |
| 111 | Accession: | Swissprot_id: | Gi_number: | Description: ATP-binding cassette, |
| | Q99758 | ABC3_HUMAN | 7387524 | subfamily A, member 3 (ATP-binding |
| | | | | cassette transporter 3) (ATP-binding |
| | | | | cassette 3) (ABC-C transporter) |
| 73 | Accession: | Swissprot_id: | Gi_number: | Description: 1,4-alpha-glucan |
| | Q08047 | GLGB_MAIZE | 1169911 | branching enzyme IIB, chloroplast |
| | | | | precursor (Starch branching enzyme |
| | | | | IIB) (Q-enzyme) |
| 443 | Accession: | Swissprot_id: | Gi_number: | Description: Luminal binding protein |
| | Q03685 | BIP5_TOBAC | 729623 | 5 precursor (BiP 5) (78 kDa glucose- |
| | | | | regulated protein homolog 5) |
| | | | | (GRP 78-5) |
| 235 | Accession: | Swissprot_id: | Gi_number: | Description: |
| | P14614 | GLU5_ORYSA | 121477 | GLUTELIN PRECURSOR |
| 217 | Accession: | Swissprot_id: | Gi_number: | Description: 13 KD PROLAMIN PRECURSOR |
| | P17048 | PRO2_ORYSA | 6174927 | |
| 257 | Accession: | Swissprot_id: | Gi_number: | Description: OLEOSIN 18 KD (OSE721) |
| | Q40646 | OLE2_ORYSA | 3334279 | |
| 201 | Accession: | Swissprot_id: | Gi_number: | Description: Receptor-like protein |
| | P47735 | RLK5_ARATH | 1350783 | kinase 5 precursor |
| 445 | Accession: | Swissprot_id: | Gi_number: | Description: SULFATED SURFACE |
| | P21997 | SSGP_VOLCA | 134920 | GLYCOPROTEIN 185 (SSG 185) |
| 281 | Accession: | Swissprot_id: | Gi_number: | Description: SACCHAROPINE |
| | P38999 | LYS9_YEAST | 729968 | DEHYDROGENASE [NADP+, L-GLUTAMATE |
| | | | | FORMING] |
| 251 | Accession: | Swissprot_id: | Gi_number: | Description: Cyclic-nucleotide-gated |
| | Q00195 | CNG2_RAT | 116574 | olfactory channel (Cyclic-nucleotide- |
| | | | | gated cation channel 2) (CNG channel |
| | | | | 2) (CNG2) (CNG-2) (OCNC1) |
| 3 | Accession: | Swissprot_id: | Gi_number: | Description: Receptor-like protein |
| | P47735 | RLK5_ARATH | 1350783 | kinase 5 precursor |
| 447 | Accession: | Swissprot_id: | Gi_number: | Description: Peroxisome assembly |
| | O60683 | PEXA_HUMAN | 3914299 | protein 10 (Peroxin-10) |
| 21 | Accession: | Swissprot_id: | Gi_number: | Description: PROTEIN KINASE APK1B |
| | P46573 | APKB_ARATH | 12644274 | |
| 179 | Accession: | Swissprot_id: | Gi_number: | Description: Prophosphate-- |
| | P21343 | PFPB_SOLTU | 2507174 | fructose 6-phosphate |
| | | | | 1-phosphotransferase beta subunit |
| | | | | (PFP) (6-phosphofructokinase |
| | | | | (Pyrophosphate)) (Pyrophosphate- |
| | | | | dependent 6-phosphofructose-1- |
| | | | | kinase) (PPI-PFK) |
| 319 | Accession: | Swissprot_id: | Gi_number: | Description: GLYCERALDEHYDE |
| | Q64467 | G3PT_MOUSE | 2494630 | 3-PHOSPHATE DEHYDROGENASE, |
| | | | | ESTIS-SPECIFIC (GAPDH) |
| 7 | Accession: | Swissprot_id: | Gi_number: | Description: Probable protease |
| | P20346 | P322_SOLTU | 129350 | inhibitor P322 precursor |
| 291 | Accession: | Swissprot_id: | Gi_number: | Description: Neural Wiskott-Aldrich |
| | O08816 | WASL_RAT | 13431956 | syndrome protein (N-WASP) |
| 169 | Accession: | Swissprot_id: | Gi_number: | Description: UTP--glucose-1-phosphate |
| | O64459 | UDPG_PYRPY | 6136112 | uridylyltransferase (UDP-glucose |
| | | | | pyrophosphorylase) (UDPGP) (UGPase) |
| 83 | Accession: | Swissprot_id: | Gi_number: | Description: ALPHA-AMYLASE ISOZYME |
| | P27933 | AM3D_ORYSA | 113682 | 3D PRECURSOR (1,4-ALPHA-D-GLUCAN |
| | | | | GLUCANOHYDROLASE) |
| 269 | Accession: | Swissprot_id: | Gi_number: | Description: PHOSPHOLIPASE D2 |
| | O14939 | PLD2_HUMAN | 13124441 | (PLD 2) CHOLINE PHOSPHATASE 2) |
| | | | | PHOSPHATIDYLCHOLINE-HYDROLYZING |
| | | | | PHOSPHOLIPASE D2) (PLD1C) |
| 95 | Accession: | Swissprot_id: | Gi_number: | Description: SEED ALLERGEN1C |
| | Q01885 | RAG2_ORYSA | 548671 | PROTEIN RAG2 PRECURSOR |
| 9 | Accession: | Swissprot_id: | Gi_number: | Description: Eukaryotic initiation |
| | Q03387 | IF41_WHEAT | 1170504 | factor (iso)4F subunit P82-34 |
| | | | | (eIF-(iso)4F P82-34) |
| 449 | Accession: | Swissprot_id: | Gi_number: | Description: Palmitoyl-protein |
| | P50897 | PPT_HUMAN | 1709747 | thioesterase precursor (Palmitoyl- |
| | | | | protein hydrolase) |
| 451 | Accession: | Swissprot_id: | Gi_number: | Description: Cell wall protein DAN4 |
| | P47179 | DAN4_YEAST | 1352944 | precursor |
| 277 | Accession: | Swissprot_id: | Gi_number: | Description: MUCIN 2 PRECURSOR |
| | Q02817 | MUC2_HUMAN | 2506877 | (INTESTINAL MUCIN 2) |
| 285 | Accession: | Swissprot_id: | Gi_number: | Description: MATERNAL PUMILIO PROTEIN |
| | P25822 | PUM_DROME | 131605 | |
| 453 | Accession: | Swissprot_id: | Gi_number: | Description: REGULATORY PROTEIN E2 |
| | P06921 | VE2_HPV05 | 1352839 | |
| 265 | Accession: | Swissprot_id: | Gi_number: | Description: ANTER-SPECIFIC PROLINE- |
| | P40602 | APG_ARATH | 728867 | RICH PROTEIN APG PRECURSOR |
| 327 | Accession: | Swissprot_id: | Gi_number: | Description: Myb protein |
| | Q08759 | MYB_XENLA | 730090 | |
| 231 | Accession: | Swissprot_id: | Gi_number: | Description: CALMODULIN-RELATED |
| | P27164 | CAL3_PETHY | 115492 | PROTEIN |
| 37 | Accession: | Swissprot_id: | Gi_number: | Description: Peptide transporter |
| | P46032 | PT2B_ARATH | 1172704 | PTR2-B (Histidine transporting |
| | | | | protein) |
| 455 | Accession: | Swissprot_id: | Gi_number: | Description: VITELLOGENIN II |
| | P02845 | VIT2_CHICK | 138595 | PRECURSOR (MAJOR VITELLOGENIN) |
| | | | | [CONTAINS: LIPOVITELLIN I |
| | | | | (LVI); PHOSVITIN (PV); |
| | | | | LIPOVITELLIN II (LVII); YGP40] |
| 43 | Accession: | Swissprot_id: | Gi_number: | Description: MLO PROTEIN |
| | P93766 | MLO_HORVU | 6016588 | |
| 457 | Accession: | Swissprot_id: | Gi_number: | Description: VACUOLAR PROTEIN |
| | Q07878 | VP13_YEAST | 2499125 | SORTING-ASSOCIATED PROTEIN VPS 13 |
| 459 | Accession: | Swissprot_id: | Gi_number: | Description: Protein-export |
| | Q50634 | SECD_MYCTU | 2498898 | membrane protein secD |
| 293 | Accession: | Swissprot_id: | Gi_number: | Description: Minor extracellular |
| | P29141 | SUBV_BACSU | 135023 | protease VPR precursor |
| 321 | Accession: | Swissprot_id: | Gi_number: | Description: Myb proto-oncogene |
| | P01103 | MYB_CHICK | 127591 | protein (C-myb) |
| 79 | Accession: | Swissprot_id: | Gi_number: | Description: GLUCOAMYLASE |
| | P08640 | AMYH_YEAST | 728850 | S1/S2 PRECURSOR (GLUCAN 1,4-ALPHA- |
| | | | | GLUCOSIDASE) (1,4-ALPHA-D-GLUCAN |
| | | | | GLUCOHYDROLASE) |
| 211 | Accession: | Swissprot_id: | Gi_number: | Description: GAMMA-GLIADIN |
| | P08079 | GDB0_WHEAT | 121099 | PRECURSOR |
| 177 | Accession: | Swissprot_id: | Gi_number: | Description: FRUCTOSE-BISPHOSPHATE |
| | P46256 | ALF1_PEA | 1168408 | ALDOLASE, CYTOPLASMIC ISOZYME 1 |
| 461 | Accession: | Swissprot_id: | Gi_number: | Description: MUCIN 2 PRECURSOR |
| | Q02817 | MUC2_HUMAN | 2506877 | (INTESTINAL MUCIN 2) |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A polynucleotide comprising a nucleotide sequence encoding a polypeptide the activity of which is involved in or associated with the synthesis, metabolism or degradation of carbohydrates in the plant grain and the expression of which is up-regulated during grain filling, which nucleotide sequence is substantially similar to a sequence encoding a polypeptide as given in SEQ ID NOS: 70-210 or a partial-length polypeptide having substantially the same activity as the full-length polypeptide, e.g., at least 50%, more preferably at least 80%, even more preferably at least 90% to 95% the activity of the full-length polypeptide.

2. The polynucleotide of claim 1 comprising a nucleotide sequence
a) as given in any one of SEQ ID NOs: 69-209 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NO: 69-209, or the complement thereof;
e) complementary to (a), (b) or (c); and
f) which is the reverse complement of (a), (b) or (c).

3. A polynucleotide according to claim 1 comprising a nucleotide sequence encoding a polypeptide which is involved in associated with starch biosynthsis and up-regulated during grain filling, which nucleic acid molecule is substantially similar to a nucleic acid encoding a polypeptide as given in SEQ ID NOs: 70-188 or a partial-length polypeptide having substantially the same activity as the full-length polypeptide, e.g., at least 50%, more preferably at least 80%, even more preferably at least 90% to 95% the activity of the full-length polypeptide.

4. The polynucleotide of claim 3 comprising a nucleotide sequence a) as given in any one of the SEQ ID NOs of table 7 such as SEQ ID NOs: 69-187 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NOs: 69-187, or the complement thereof;
e) complementary to (a), (b) or (c); and
f) which is the reverse complement of (a), (b) or (c).

5. The polynucleotide of claim 3 comprising a nucleotide sequence encoding a polypeptide with an activity of a small and large subunit ADPG pyrophosphorylase, respectively, which nucleotide sequence is substantially similar to a nucleic acid sequence encoding a polypeptide as given in SEQ ID NOs: 136-142 or a partial-length polypeptide having substantially the same activity as the full-length polypeptide, e.g., at least 50%, more preferably at least 80%, even more preferably at least 90% to 95% the activity of the full-length polypeptide.

6. The polynucleotide of claim 5 comprising a nucleotide sequence
a) as given in any one of SEQ ID NOs: 135-141 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of nucleotides given in SEQ ID NO: 135-141, or the complement thereof;
e) complementary to (a), (b) or (c); and
f) which is the reverse complement of (a), (b) or (c).

7. A polynucleotide according to claim 3 comprising a nucleotide sequence encoding a polypeptide involved in starch structure rearrangement, which nucleic acid molecule is substantially similar to a nucleic acid encoding a polypeptide as given in SEQ ID NOs: 76-78 exhibiting isoamylase debranching enzyme activity; 70-74 exhibiting a branching enzyme activity, 80-92 exhibiting an α-amylase activity; 94-100 exhibiting an α-amylase inhibitor activity; 110 exhibiting a pullulanase activity;
102-108 exhibiting a O-amylase activity; 112-118 exhibiting a a-glucosidase activity, or a partial-length polypeptide having substantially the same activity as the full-length polypeptide, e.g., at least 50%, more preferably at least 80%, even more preferably at least 90% to 95% the activity of the full-length polypeptide.

8. The polynucleotide of claim 7, comprising a nucleotide sequence
a) as given in any one of SEQ ID NOs: 75-77 exhibiting isoamylase debranching enzyme activity; 69-73 exhibiting a branching enzyme activity, 79-91 exhibiting an α-amylase activity; 93-99 exhibiting an α-amylase inhibitor activity; 109 exhibiting a pullulanase activity; 101-107, exhibiting a β-amylase activity; 111-117 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NOs: 75-77 exhibiting isoamylase debranching enzyme activity; 69-73 exhibiting a branching enzyme activity, 79-91 exhibiting an α-amylase activity; 93-99 exhibiting an α-amylase inhibitor activity; 109 exhibiting a pullulanase activity; 101-107, exhibiting a β-amylase activity; 111-117;
e) complementary to (a), (b) or (c); and
f) which is the reverse complement of (a), (b) or (c).

9. A polynucleotide according to claim 3 comprising a nucleotide sequence encoding a polypeptide exhibiting an amylase or an amylase inhibitor activity, which nucleic acid molecule is substantially similar to a nucleic acid encoding a polypeptide as given in SEQ ID NOs: 80-92 exhibiting an α-amylase activity; and 94-100 exhibiting an α-amylase inhibitor activity, or a partial-length polypeptide having substantially the same activity as the full-length polypeptide, e.g., at least 50%, more preferably at least 80%, even more preferably at least 90% to 95% the activity of the full-length polypeptide.

10. The polynucleotide of claim 9 comprising a nucleotide sequence
a) as given in any one of SEQ ID NOs: 79-91 exhibiting an α-amylase activity; and 93-99 exhibiting an α-amylase inhibitor activity or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ED NOs: 79-91 exhibiting an α-amylase activity; and 93-99 exhibiting an α-amylase inhibitor activity, or the complement thereof;
e) complementary to (a), (b) or (c); and
f) which is the reverse complement of (a), (b) or (c).

11. A polynucleotide according to claim 3 comprising a nucleotide sequence encoding a polypeptide exhibiting a sucrose synthase activity, which nucleic acid molecule is substantially similar to a nucleic acid encoding a polypeptide as given in SEQ ID NOs: 120-128 or a partial-length polypeptide having substantially the same activity as the full-length polypeptide, e.g., at least 50%, more preferably at least 80%, even more preferably at least 90% to 95% the activity of the full-length polypeptide.

12. The polynucleotide of claim 11 comprising a nucleotide sequence
a) as given in any one of SEQ ID NOs: 119-127 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NOs: 119-127 or the complement thereof;
e) complementary to (a), (b) or (c); and
f) which is the reverse complement of (a), (b) or (c).

13. A polynucleotide according to claim 3 comprising a nucleotide sequence encoding a polypeptide exhibiting a glucanase activity, which nucleic acid molecule is substantially similar to a nucleic acid encoding a polypeptide as given in SEQ ID NOs: 192 or a partial-length polypeptide having substantially the same activity as the full-length polypeptide, e.g., at least 50%, more preferably at least 80%, even more preferably at least 90% to 95% the activity of the full-length polypeptide.

14. The polynucleotide of claim 13 comprising a nucleotide sequence
a) as given in SEQ ID NO: 191 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of nucleotides given in SEQ ID NO: 191 or the complement thereof;
e) complementary to (a), (b) or (c); and
f) which is the reverse complement of (a), (b) or (c).

15. A polynucleotide comprising a nucleotide sequence encoding a seed storage protein, which nucleic acid molecule is substantially similar to a nucleic acid encoding a polypeptide as given in SEQ ID NOs: 212-250 or a partial-length polypeptide having substantially the same activity as the full-length polypeptide, e.g., at least 50%, more preferably at least 80%, even more preferably at least 90% to 95% the activity of the full-length polypeptide.

16. The polynucleotide of claim 15 comprising a nucleotide sequence
a) as given in any one of SEQ ID NOs: 211-249 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 211-249 or the complement thereof;
e) complementary to (a), (b) or (c); and
f) which is the reverse complement of (a), (b) or (c).

17. The polynucleotide of claim 15 comprising a nucleotide sequence encoding a glutelin protein the expression of which is up-regulated during grain filling, which nucleic acid molecule is substantially similar to a nucleic acid encoding a polypeptide as given in SEQ ID NOs: 224, 236, and 240 or a partial-length polypeptide having substantially the same activity as the full-length polypeptide, e.g., at least 50%, more preferably at least 80%, even more preferably at least 90% to 95% the activity of the full-length polypeptide.

18. The polynucleotide of claim 17 comprising a nucleotide sequence
a) as given in any one of SEQ ID NOs: 223, 235, and 239 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 223, 235, and 239, or the complement thereof;
e) complementary to (a), (b) or (c); and
f) which is the reverse complement of (a), (b) or (c).

19. A polynucleotide according to claim 15 comprising a nucleotide sequence encoding a prolamin protein the expression of which is up-regulated during grain filling, which nucleotide sequence is substantially similar to a nucleic acid sequence encoding a polypeptide as given in SEQ HD NOs: 218, 220, 226 and 242 or a partial-length polypeptide having substantially the same activity as the full-length polypeptide, e.g., at least 50%, more preferably at least 80%, even more preferably at least 90% to 95% the activity of the full-length polypeptide.

20. The polynucleotide of claim 19 comprising a nucleotide sequence
a) as given in any one of SEQ ID NOs: 217, 219, 225 and 241 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 217, 219, 225 and 241, or the complement thereof;
e) complementary to (a), (b) or (c); and
f) which is the reverse complement of (a), (b) or (c).

21. A polynucleotide according to claim 15 comprising a nucleotide sequence encoding a gliadin protein, the expression of which is up-regulated during grain filling, which nucleotide sequence is substantially similar to a nucleic acid sequence encoding a polypeptide as given in SEQ ID NOs: 212, 219; 234, 248; and 250 or a partial-length polypeptide having substantially the same activity as the full-length polypeptide, e.g., at least 50%, more preferably at least 80%, even more preferably at least 90% to 95% the activity of the full-length polypeptide.

22. The polynucleotide of claim 21 comprising a nucleotide sequence
a) as given in any one of SEQ ID NOs: 211,220; 233, 247; and 249 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 135325; 135133; 10825,135101; and 135103, or the complement thereof;
e) complementary to (a), (b) or (c); and
f) which is the reverse complement of (a), (b) or (c).

23. A polynucleotide the expression of which is up-regulated during grain filling comprising a nucleotide sequence encoding a polypeptide that is involved in or associated with fatty acid synthesis or lipid metabolism, which nucleotide sequence is substantially similar to a nucleic acid sequence encoding a polypeptide as given in SEQ ID NOs: 252-280 or a partial-length polypeptide having substantially the same activity as the full-length polypeptide, e.g., at least 50%, more preferably at least 80%, even more preferably at least 90% to 95% the activity of the full-length polypeptide.

24. The polynucleotide of claim 23 comprising a nucleotide sequence
a) as given in any one of SEQ ID NOs: 251-279 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of nucleotides given in any one of SEQ ID NOs: 251-279 or the complement thereof;
e) complementary to (a), (b) or (c); and
f) which is the reverse complement of (a), (b) or (c).

25. A polynucleotide according to claim 23 comprising a nucleotide sequence encoding an oleosin protein, which nucleotide sequence is substantially similar to a nucleic acid sequence encoding a polypeptide as given in SEQ ID NOs: 258 and 260 or a partial-length polypeptide having substantially the same activity as the full-length polypeptide, e.g., at least 50%, more preferably at least 80%, even more preferably at least 90% to 95% the activity of the full-length polypeptide.

26. The polynucleotide of claim 25 comprising a nucleotide sequence
a) as given in any one of SEQ ID NOs: 257 and 259 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 257 and 259, or the complement thereof;
e) complementary to (a), (b) or (c); and
f) which is the reverse complement of (a), (b) or (c).

27. A polynucleotide according to claim 23 comprising a nucleotide sequence encoding a polypeptide the activity of which is involved in or associated with the dehydrogenation of phytoene and the expression of which is up-regulated during grain filling, which nucleotide sequence is substantially similar to a nucleic acid sequence encoding a polypeptide as given in SEQ ID NO: 278 or a partial-length polypeptide having substantially the same activity as the full-length polypeptide, e.g., at least 50%, more preferably at least 80%, even more preferably at least 90% to 95% the activity of the full-length polypeptide.

28. The polynucleotide of claim 27 comprising a nucleotide sequence
a) as given in any one of SEQ ID NOs: 277 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide-sequence given in any one of SEQ ID NOs: 277, or the complement thereof;
e) complementary to (a), (b) or (c); and
f) which is the reverse complement of (a), (b) or (c).

29. A polynucleotide comprising a nucleotide sequence that encodes a polypeptide that acts as a transcription factor and the expression of which is up-regulates during grain filling, which nucleotide sequence is substantially similar to a nucleic acid sequence encoding a polypeptide as given in SEQ ID NOs: 302-328 or a partial-length polypeptide having substantially the same activity as the full-length polypeptide, e.g., at least 50%, more preferably at least 80%, even more preferably at least 90% to 95% the activity of the full-length polypeptide.

30. The polynucleotide of claim 29 comprising a nucleotide sequence
a) as given in any one of SEQ ID NOs: 301-327 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: 301-327, or the complement thereof;
e) complementary to (a), (b) or (c); and
f) which is the reverse complement of (a), (b) or (c).

31. A polynucleotide comprising a nucleotide sequence encoding a polypeptide the activity of which is involved or associated with the metabolism of amino acids and the expression of which is up-regulated during grain filling, which nucleotide sequence is substantially similar to a nucleic acid sequence encoding a polypeptide as given in SEQ ID NOs: 282-300 or a partial-length polypeptide having substantially the same activity as the full-length polypeptide, e.g., at least 50%, more preferably at least 80%, even more preferably at least 90% to 95% the activity of the full-length polypeptide.

32. The polynucleotide of claim 31 comprising a nucleotide sequence
a) as given in any one of SEQ ID NOs: 281-299 or a part thereof which still encodes a partial-length polypeptide having substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in any one of SEQ ID NOs: δ 281-299, or the complement thereof;
e) complementary to (a), (b) or (c); and
f) which is the reverse complement of (a), (b) or (c).

33. A polypeptide which has an amino acid sequence encoded by any one of the polynucleotides according to claim 1.

34. A polypeptide according to claim 33, which has an amino acid sequence encoded by a polynucleotide selected from the group consisting of SEQ ID NOs: 1 to 461, 501-511, and 513-641.

35. A polypeptide according to claim 33 wherein said polypeptide has at least 90% amino acid sequence identity to a polynucleotide selected from the group consisting of SEQ ID NOs: 2-462, 502-512, and 514-642.

36. An isolated nucleic acid molecule comprising a nucleotid sequence, which nucleotide sequence is obtained or obtainable from plant genomic DNA comprising a gene having an open reading frame (ORF) encoding a polypeptide which has at least between 70%, and 99% amino acid sequence identity to a polypeptide encoded by an Oryza, e.g., Oryza saliva, gene comprising a nucleotide sequence as given in SEQ ID NOs: 1 to 461, 501-511, and 513-641.

37. A recombinant vector comprising a polynucleotide of claim 1.

38. An expression cassette comprising as operably linked components, a promoter, a polynucleotide of claim 1 and a termination sequence.

39. A host cell comprising the expression cassette of claim 38.

40. The host cell of claim 39 wherein said host cell is a bacterial cell, a yeast cell, an animal cell or a plant cell.

41. The host cell of claim 40, wherein said plant cell is from a cereal plant.

42. A plant comprising a host cell of claim 39.

43. A plant according to claim 42, wherein said plant is selected from the group consisting of maize, soybean, barley, alfalfa, sunflower, tomato, banana, canola, cotton, peanut, sorghum, tobacco, sugarbeet, wheat, and rice.

44. A method of modulating carbohydrate composition of the plant grain, comprising functionally integrating an isolated nucleic acid molecule according to claim 1 comprising a nucleic acid sequence encoding a polypeptide, which is involved in or associated with the synthesis, metabolism or degradation of carbohydrates in the plant grain and the expression of which is up-regulated during grain filling, into a cell, group of cells, tissue or organ of a plant.

45. A method of modulating the protein content and composition of the plant grain, comprising functionally integrating an isolated nucleic acid molecule according to claim 15 comprising a nucleic acid sequence encoding a polypeptide, which is involved in or associated with the synthesis, metabolism or degradation of seed storage proteins in the plant grain and the expression of which is up-regulated during grain filling, into a cell, group of cells, tissue or organ of a plant.

46. A method of modulating the fatty acid and/or lipid content and composition of the plant grain, comprising functionally integrating an isolated nucleic acid molecule according to claim 23 comprising a nucleic acid sequence encoding a polypeptide, which is involved in or associated with fatty acid synthesis or lipid metabolism in the plant grain and the expression of which is up-regulated during grain filling, into a cell, group of cells, tissue or organ of a plant.

47. A method of modulating the grain filling process of the plant grain, comprising functionally integrating an isolated nucleic acid molecule according to claim 28 comprising a nucleic acid sequence encoding a transcription factor polypeptide, which is involved in or associated with the regulation and coordination of grain filling in plants and the expression of which is up-regulated during grain filling, into a cell, group of cells, tissue or organ of a plant.

48. A method of modulating the amino acid content and composition of the plant grain, comprising functionally integrating an isolated nucleic acid molecule according to claim 31 comprising a nucleic acid sequence encoding a polypeptide the activity of which is involved or associated with the metabolism of amino acids and the expression of which is up-regulated during grain filling, into a cell, group of cells, tissue or organ of a plant.

49. A method of modulating nutrient content and composition of the plant grain, comprising:
a) functionally integrating
i. an isolated nucleic acid molecule according to claim 1, or a portion thereof in an anti-sense orientation; or
ii. an dsRNAi construct comprising an isolated nucleic acid molecule according to claim 1, or a portion thereof in both a sense and an anti-sense orientation, which, optionally, may be separated by a spacer region;
under the transcriptional control of regulatory sequences required for expression in plants, into a cell, group of cells, tissue or organ of a plant; and
b) expressing the constructs as provided in a) above in a cell, group of cells, a tissue or organ of a plant to produce a RNA transcript.

50. A method of identifying or isolating polynucleotide sequences that are orthologous to a nucleic acid molecule according to claim 1 comprising a nucleic acid fragment encoding a polypeptide that is up-regulated during grain filling, from the genome of another plant, wherein all or a portion of a particular nucleic acid sequence according to claim 1 is used as a probe that selectively hybridizes to gene sequences present in a population of cloned genomic DNA fragments or cDNA fragments from a chosen source organism.

51. A method to identify a nucleic acid molecule encoding a polypeptide the expression of which is up-regulated during grain filling
a) contacting a plurality of isolated nucleic acid samples comprising all or a portion of a particular nucleic acid sequence according to claim 1 on a solid substrate with a probe comprising plant nucleic acid corresponding to RNA isolated from a specific plant tissue during grain filling so as to form a complex, wherein each sample comprises a plurality of oligonucleotides corresponding to at least a portion of one plant gene; and
b) contacting a second plurality of isolated nucleic acid samples comprising all or a portion of a particular nucleic acid sequence according to claim 1 to on a solid substrate with a second probe comprising plant nucleic acid corresponding to RNA that is taken at a different development stage of the plant;
c) comparing complex formation in a) with complex formation in b) so as to identify which samples correspond to genes that are expressed during grain filling.

52. A method for detecting the presence of a polynucleotide according to claim 1, or a fragment or a variant thereof, or a complementary sequence thereto in a sample, the method including the following steps of:
a) bringing into contact a nucleotide probe or a plurality of nucleotide probes which can hybridize with a polynucleotide according to claim 1, or a fragment or a variant thereof, or a complementary sequence thereto and the sample to be assayed.
b) detecting the hybrid complex formed between the probe and a nucleotide in the sample.

53. A kit for detecting the presence of a polynucleotide according to claim 1, or a fragment or a variant thereof, or a complementary sequence thereto in a sample, the kit including a nucleotide probe or a plurality of nucleotide probes which can hybridize with a nucleotide sequence comprised within a polynucleotide according to claim 1, or a fragment or a variant thereof, or a complementary sequence thereto and, optionally, the reagents necessary for performing the hybridization reaction.

54. A method of modifying the frequency of a grain filling gene in a plant population, comprising the steps of:
a) screening a plurality of plants using an oligonucleotide as a marker to determine the presence or absence of a grain filling gene in an individual plant, the oligonucleotide consisting of not more than 300 bases of a nucleotide sequence selected from the group consisting of SEQ ID NOs 1 to SEQ ID NO: 461,
b) selecting at least one individual plant for breeding based on the presence or absence of the grain filling gene; and
c) breeding at least one plant thus selected to produce a population of plants having a modified frequency of the grain filling gene.

55. A method according to claim 54, wherein the oligonucleotide comprises a simple sequence repeat (SSR) sequence comprising at least two consecutive repeat units of an SSR, the start and end points of which are provided in Tables 2 and 3, and a flanking sequence of at least about 14 nucleic acids immediately adjacent to said at least two consecutive repeat units.

56. A method of plant breeding to select for or against a trait of interest which is associated with grain filling in plants, comprising the steps of:
a. identifying the trait of interest; identifying at least one oligonucleotide that can be used as a marker for the trait, the oligonucleotide consisting of not more than 300 bases of a nucleotide sequence selected from the group consisting of SEQ ID NOs: 1 to SEQ ID NO: 461,
b. screening at least one plant for the presence of the at least one oligonucleotide;
c. selecting at least one plant based on presence or absence of the at least one oligonucleotide;
d. breeding at least one plant thus selected to produce a population of plants having a modified frequency of the at least one oligonucleotide; and
e. screening at least one plant of the population for the presence or absence of the grain filling trait.

57. A method according to claim 56, wherein the oligonucleotide comprises a simple sequence repeat (SSR) sequence comprising at least two consecutive repeat units of an SSR, the start and end points of which are provided in Tables 2 and 3, and a flanking sequence of at least about 14 nucleic acids immediately adjacent to said at least two consecutive repeat units.

58. A method of determining a varietal identity of a plant, comprising:
a) obtaining a nucleic acid sample from a plant;
b) identifying at least one oligonucleotide to obtain an oligonucleotide profile for the plant, wherein the oligonucleotide consists of not more than 300 bases of a nucleotide sequence selected from the group consisting of SEQ ID NOs: 1 to SEQ ID NO: 461, the oligonucleotide comprising a simple sequence repeat (SSR) sequence comprising at least two consecutive repeat units of an SSR, the start and end points of which are provided in Tables 2 and 3, and a flanking sequence of at least about 14 nucleic acids immediately adjacent to said at least two consecutive repeat units in the sample; and
c) comparing the SSR profile to at least one known SSR profile corresponding to at least one known variety to determine the varietal identity of the plant.

59. An oligonucleotide primer consisting of between 8 and 150 bases which comprises at least 14 bases selected from the group of flanking sequences obtainable from a nucleotide sequence provided in SEQ ID NOs: 3435 to SEQ ID NO: 150133, which at least 14 bases are immediately adjacent to at least two consecutive repeat units of an SSR, the start and end points of which are provided in Tables 2 and 3.

60. A computer-readable medium having stored thereon a data structure comprising:
a) Sequence information of a polynucleotide according to claim 1; 15-22; 23-28; 28-30 and 31 to 32 and/or; and a polynucleotide according to any one of claims . . . to . . . .
b) a module receiving the nucleic acid molecule which compares the nucleic acid sequence of the molecule to at least one other nucleic acid sequence.
